(19) United States
(12) Patent Application Publication
(10) Pub. No.: US 2024/0276872 A1
(43) Pub. Date: Aug. 15, 2024

(54) PLURALITY OF HOST MATERIALS AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: So-Young JUNG, Gyeonggi-do (KR); Tae-Jun HAN, Gyeonggi-do (KR); YeJin JEON, Gyeonggi-do (KR); Seung-Hyun YOON, Gyeonggi-do (KR); Young-Jae KIM, Gyeonggi-do (KR); Hyun-Ju KANG, Gyeonggi-do (KR); Bitnari KIM, Gyeonggi-do (KR); Sang-Hee CHO, Gyeonggi-do (KR)

(21) Appl. No.: 18/404,236

(22) Filed: Jan. 4, 2024

(30) Foreign Application Priority Data
Jan. 16, 2023 (KR) 10-2023-0006086
Dec. 13, 2023 (KR) 10-2023-0180260

(51) Int. Cl. H10K 85/60; C07D 209/88; C07D 405/04; C07D 405/12; C09K 11/06

(52) U.S. Cl. CPC H10K 85/622; C07D 209/88; C07D 405/04; C07D 405/12; C09K 11/06; H10K 85/626; H10K 85/633; H10K 85/636; H10K 85/654; H10K 85/6574; H10K 2101/90

(57) ABSTRACT

The present disclosure relates to a plurality of host materials comprising at least one first host compound represented by Formula 1 and at least one second host compound represented by Formula 3 different from the first host compound, and an organic electroluminescent device comprising the same. By comprising a specific combination of compounds according to the present disclosure as host materials, an organic electroluminescent device having low driving voltage, high luminous efficiency, and/or significantly improved lifespan characteristic, can be provided.

### TECHNICAL FIELD

The present disclosure relates to a plurality of host materials and an organic electroluminescent device comprising the same.

### BACKGROUND ART

The TPD/Alq₃ bilayer small molecule organic electroluminescent device (OLED) with green-emission, which is constituted with a light-emitting layer and a charge transport layer, was first developed by Tang, et al., of Eastman Kodak in 1987. Thereafter, the studies on an OLED have been rapidly affected and OLEDs have been commercialized. At present, an organic electroluminescent device mainly includes phosphorescent materials having excellent luminous efficiency in panel realization. In many applications such as TVs and lightings, OLED lifetime is insufficient, and a high efficiency of the OLEDs is still required. Typically, the higher the luminance of an OLED corresponds to a shorter lifetime of the OLED. Therefore, an OLED having high luminous efficiency and/or long lifespan characteristics is required for long time use and high resolution of a display.

Korean Patent Application Laid-open No. 2022-0118960 discloses an organic electroluminescent device comprising a chrysene derivative as one of the light-emitting layer materials. However, said reference does not specifically disclose the specific combination of host materials as described in the present disclosure. In addition, there is a need for the development of a light-emitting material with improved performance, such as high luminous efficiency and/or improved lifespan characteristics, by combining the compound(s) disclosed in said reference with a specific compound(s).

### DISCLOSURE OF THE INVENTION

### Problems to be Solved

The object of the present disclosure is firstly, to provide a plurality of host materials which is able to produce an organic electroluminescent device with low driving voltage, high luminous efficiency, and/or long lifespan characteristics; secondly, to provide an organic electroluminescent device with low driving voltage, high luminous efficiency, and/or long lifespan characteristics by comprising a specific combination of compounds according to the present disclosure as a host material.

### Solution to Problems

As a result of intensive studies to solve the technical problem above, the present inventors found that the aforementioned objective can be achieved by a plurality of host materials comprising at least one first host compound represented by the following Formula 1 and at least one second host compound represented by the following Formula 3 which is different from the first host compound so that the present invention was completed.

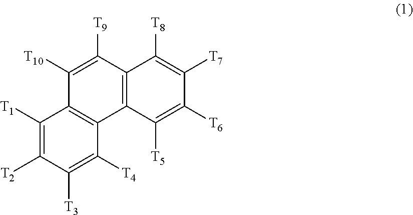

in Formula 1,
T₅ and T₆ are connected to each other to form a ring of the following Formula 2; or T₇ and T₈ are connected to each other to form a ring of the following Formula 2; or T₅ and T₆ are connected to each other to form a ring of the following Formula 2, and T₇ and T₈ are connected to each other to form a ring of the following Formula 2;

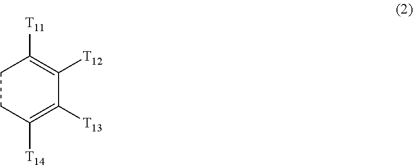

in formulas 1 and 2,
T₁ to T₄, T₉ to T₁₄, and T₅ to T₈, which do not form a ring, each independently represent, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of (C3-C30) aliphatic ring and (C6-C30) aromatic ring, a substituted or unsubstituted (C6-C30)aryl, a substituted or unsubstituted (3- to 30-membered)heteroaryl, —SiR₁₁R₁₂R₁₃, or -L₁-NAr₁Ar₂; provided that at least one of T₁ to T₁₄ is -L₁-NAr₁Ar₂;
L₁ represents a single bond, a substituted or unsubstituted (C6-C30)arylene, or a substituted or unsubstituted (3- to 30-membered)heteroarylene;
R₁₁ to R₁₃, Ar₁, and Ar₂ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and
[symbol] represents a site fused with Formula 1;

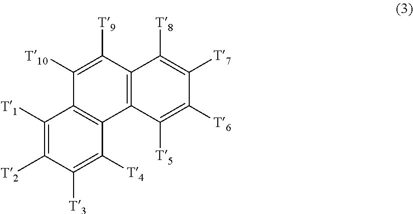

in Formula 3,
T′₅ and T′₆ are connected to each other to form a ring of the following Formula 4; or T′₇ and T′₈ are connected to each other to form a ring of the following Formula 4; or T′₅ and T′₆ are connected to each other to form a ring of the following Formula 4, and T′₇ and T′₈ are connected to each other to form a ring of the following Formula 4;

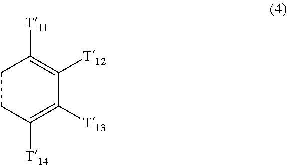

in formulas 3 and 4,
T′₁ to T′₄, T′₉ to T′₁₄, and T′₅ to T's, which do not form a ring, each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy a substituted or unsubstituted fused ring of (C3-C30) aliphatic ring and (C6-C30) aromatic ring, —SiR₁₁R₁₂R₁₃, —NR₁₄R₁₅, or -L₂-HAr₁; provided that at least one of T₁ to T′₁₄ is -L₂-HAr₁;
R₁₁ to R₁₅ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
L₂ represents single bond, a substituted or unsubstituted (C6-C30)arylene, or a substituted or unsubstituted (3- to 30-membered)heteroarylene;
HAr₁ represents a substituted or unsubstituted (3- to 30-membered)heteroaryl comprising at least one nitrogen (N) atom; and
[symbol] represents a site fused with the Formula 3.

### Advantageous Effects of Invention

By comprising a specific combination of compounds according to the present disclosure as host materials, an organic electroluminescent device having low driving voltage, high luminous efficiency and/or long lifespan characteristics can be provided.

### EMBODIMENTS OF THE INVENTION

Hereinafter, the present disclosure will be described in detail. However, the following description is intended to explain the invention, and is not meant in any way to restrict the scope of the invention.

The present disclosure relates to a plurality of host materials comprising at least one first host compound represented by Formula 1 and at least one a second host compound represented by Formula 3, which is different from the first host compound, and an organic electroluminescent device comprising the same.

The term “organic electroluminescent compound” in the present disclosure means a compound that may be used in an organic electroluminescent device, and may be comprised in any material layer constituting an organic electroluminescent device, as necessary.

Herein, the term “organic electroluminescent material” means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, or an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. As such at least two compounds may be comprised in the same layer or in different layers, and may be mixture-evaporated or co-evaporated, or may be individually evaporated.

Herein, the term “a plurality of host materials” means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The at least two compounds comprised in a plurality of host materials may be comprised together in one light-emitting layer, or may each be comprised in separate light-emitting layers. When at least two compounds are comprised in one light-emitting layer, the at least two compounds may be mixture-evaporated to form a layer or may be individually and simultaneously co-evaporated to form a layer.

Herein, “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, see-butyl, etc. Herein, the term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. Herein, “(3- to 7-membered)heterocycloalkyl” is meant to be a cycloalkyl having 3 to 7 ring backbone atoms and including at least one heteroatoms selected from the group consisting of B, N, O, S, Si, and P, preferably the group consisting of O, S, and N, in which the number of the ring backbone carbon atoms is preferably 5 to 7, for example, tetrahydrofuran, pyrrolidine, thiolane, tetrahydropyran, etc. Herein, “(C6-C30)aryl(ene)” is a monocyclic or fused ring radical derived from an aromatic hydrocarbon having 6 to 30 ring backbone carbon atoms, in which the number of the ring backbone carbon atoms is preferably 6 to 20, more preferably 6 to 15, may be partially saturated, and may include a spiro structure. Examples of the aryl specifically may be phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluoren-fluoren]yl, spiro[fluoren-benzofluoren]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. Herein, “(3- to 30-membered)heteroaryl(ene)” is an aryl having 3 to 30 ring backbone atoms and including at least one, preferably 1 to 4 heteroatoms selected from the group consisting of B, N, O, S, Si, P, Se, and Ge, in which the number of the ring backbone carbon atoms is preferably 3 to 30, and more preferably 5 to 20. The above heteroaryl(ene) may be a monocyclic ring, or a fused ring condensed with at least one benzene ring; and may be partially saturated. Also, the above heteroaryl or heteroarylene herein may be one formed by linking at least one heteroaryl or aryl group to a heteroaryl group via a single bond(s), and may comprise a spiro structure. Examples of the heteroaryl specifically may be a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Additionally, “heteroaryl(ene)” can be classified into a heteroaryl(ene) with electronic properties and a heteroaryl(ene) with hole properties. A heteroaryl(ene) with electronic properties is a substituent with relatively abundant electrons in the parent nucleus, and for example, it may be a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. A heteroaryl(ene), which has hole properties, is a substituent with a relative lack of electrons in the parent nucleus, and for example, it may be a substituted or unsubstituted carbazolyl, a substituted or unsubstituted dibenzofuranyl, or a substituted or unsubstituted dibenzothiophenyl. Herein, the term “a fused ring of (C3-C30) aliphatic ring and (C6-C30) aromatic ring” means a ring formed by fusing at least one aliphatic ring having 3 to 30 ring backbone carbon atoms in which the carbon atoms number is preferably 3 to 25, more preferably 3 to 18, and at least one aromatic ring having 6 to 30 ring backbone carbon atoms in which the carbon atoms number is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of (C3-C30) aliphatic ring and (C6-C30) aromatic ring may be replaced with at least one heteroatom selected from B, N, O, S, Si, and P, preferably at least one heteroatom selected from N, O, and S. The term “Halogen” in the present disclosure includes F, Cl, Br, and I.

In addition, “ortho (o),” “meta (m),” and “para (p)” are meant to signify the substitution position of all substituents. Ortho position is a compound with substituents, which are adjacent to each other, e.g., at the 1 and 2 positions on benzene. Meta position is the next substitution position of the immediately adjacent substitution position, e.g., a compound with substituents at the 1 and 3 positions on benzene. Para position is the next substitution position of the meta position, e.g., a compound with substituents at the 1 and 4 positions on benzene.

Herein, the term “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, preferably may be a substituted or unsubstituted (5- to 25-membered) mono- or polycyclic, alicyclic, aromatic ring, or a combination thereof. Further, the formed ring may include at least one heteroatom selected from the group consisting of B, N, O, S, Si, and P, preferably, N, O, and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzofluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, “substituted” in the expression “substituted or unsubstituted” means that a hydrogen atom in a certain functional group is replaced with another atom or functional group, i.e., a substituent, and substituted with a group to which two or more substituents are connected among the substituents. For example, “a substituent to which two or more substituents are connected” may be pyridine-triazine. That is, pyridine-triazine may be a heteroaryl or may be interpreted as one substituent in which two heteroaryls are connected. Unless otherwise specified, the substituent may replace hydrogen at a position where the substituent can be substituted without limitation, and when two or more hydrogen atoms in a functional group are each replaced with a substituent, each substituent may be the same or different. The maximum number of substituents that can be substituted for a certain functional group may be the total number of valences that can be substituted for each atom forming the functional group. The substituted alkyl, the substituted alkenyl, the substituted cycloalkyl, the substituted alkoxy, the substituted aryl(ene), the substituted heteroaryl(ene) and the substituted fused ring of aliphatic ring and aromatic ring in the formulas of the present disclosure, each independently may be substituted with at least one selected from the group consisting of deuterium; halogen; cyano; carboxyl; nitro; hydroxyl; phosphine oxide; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (3- to 30-membered)heteroaryl unsubstituted or substituted with at least one of (C1-C30)alkyl and (C6-C30)aryl; (C6-C30)aryl unsubstituted or substituted with at least one of deuterium, cyano, (C1-C30)alkyl, (C3-C30)cycloalkyl, tri(C1-C30)alkylsilyl and (3- to 30-membered)heteroaryl; tri(C1-C30)alkylsilyl; tri(C6-C30)arylsilyl; di(C1-C30)alkyl(C6-C30)arylsilyl; (C1-C30)alkyldi(C6-C30)arylsilyl; fused ring of (C3-C30) aliphatic ring and (C6-C30) aromatic ring; amino; mono- or di-(C1-C30)alkylamino; mono- or di-(C2-C30)alkenylamino; (C1-C30)alkyl(C2-C30)alkenylamino; mono- or di-(C6-C30)arylamino; (C1-C30)alkyl(C6-C30)arylamino; mono- or di-(3- to 30-membered)heteroarylamino; (C1-C30)alkyl(3- to 30-membered)heteroarylamino; (C2-C30)alkenyl(C6-C30)arylamino; (C2-C30)alkenyl(3- to 30-membered)heteroarylamino; (C6-C30)aryl(3- to 30-membered)heteroarylamino; (C1-C30)alkylcarbonyl; (C1-C30)alkoxycarbonyl; (C6-C30)arylcarbonyl; (C6-C30)arylphosphine; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl. For example, the substituents may be substituted with phenyl unsubstituted or substituted with at least one of cyano; fluorine (F); tert-butyl; cyclohexyl; naphthyl; trimethylsilyl; and triphenylsilyl, naphthyl, p-biphenyl, m-biphenyl, o-biphenyl, p-terphenyl, m-terphenyl, o-terphenyl, phenanthrenyl, chrysenyl, triphenylenyl, anthracenyl, dimethylfluorenyl, diphenylfluorenyl, spirobifluorenyl, pyridyl unsubstituted or substituted with phenyl, dibenzofuranyl unsubstituted or substituted with phenyl or biphenyl, a substituted or unsubstituted dibenzothiophenyl, carbazolyl, phenoxazinyl, benzothiophenyl, or naphthooxazolyl unsubstituted or substituted with phenyl, etc.

In formulas of the present disclosure, when a plurality of substituents represented by the same symbol are present, each of these substituents, represented by the same symbol, may be the same or different.

Hereinafter, the plurality of host materials according to one embodiment will be described.

The plurality of host materials according to one embodiment comprise at least one first host compound and at least one second host compound, which is different from the first host compound, wherein the first host compound is represented by Formula 1 and the second host compound is represented by Formula 3, and the plurality of host materials may be included in the light-emitting layer of an organic electroluminescent device according to one embodiment.

The first host compound as the host material according to one embodiment is represented by the following Formula 1.

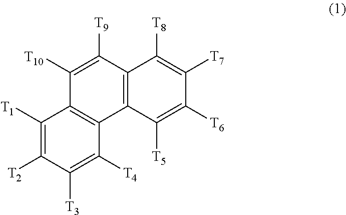

in Formula 1,
T₅ and T₆ are connected to each other to form a ring of the following Formula 2; or T₇ and T₈ are connected to each other to form a ring of the following Formula 2; and T₅ and T₆ are connected to each other to form a ring of the following Formula 2, and T₇ and T₈ are connected to each other to form a ring of the following Formula 2;

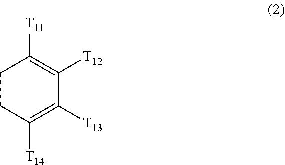

in formulas 1 and 2,
T₁ to T₄, T₉ to T₁₄, and T₅ to T₈, which do not form a ring, each independently represent, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of (C3-C30) aliphatic ring and (C6-C30) aromatic ring, a substituted or unsubstituted (C6-C30)aryl, a substituted or unsubstituted (3- to 30-membered)heteroaryl, —SiR₁₁R₁₂R₁₃, or -L₁-NAr₁Ar₂; provided that at least one of T₁ to T₁₄ is -L₁-NAr₁Ar₂;
L₁ represents a single bond, a substituted or unsubstituted (C6-C30)arylene, or a substituted or unsubstituted (3- to 30-membered)heteroarylene;
R₁₁ to R₁₃, Ar₁, and Ar₂ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
[symbol] represents a site fused with the Formula 1.

In one embodiment, T₅ and T₆, in the Formula 1, may be connected to each other to form a ring of the Formula 2.

In one embodiment, T₇ and T₈, in the Formula 1, may be connected to each other to form a ring of the Formula 2.

In one embodiment, T₅ and T₆, and T₇ and T₈, in the Formula 1, may be connected to each other to form a ring of the Formula 2.

According to one embodiment, the first host compound represented by Formula 1 may be represented by any one of the following formulas 1-1 to 1-3.

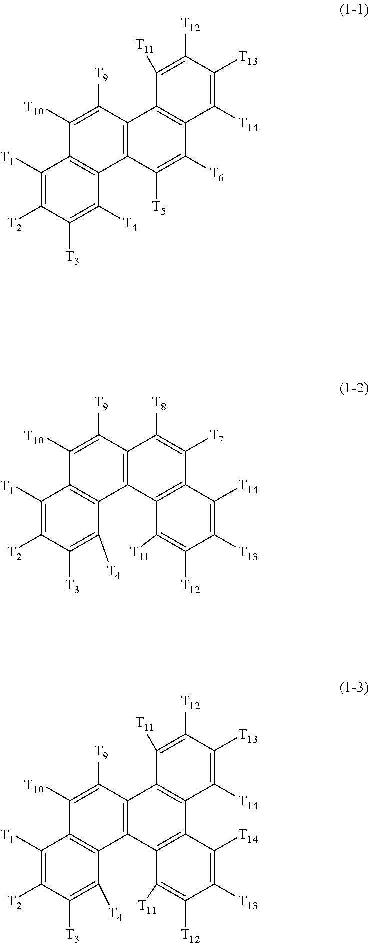

In formulas 1-1 to 1-3,
T₁ to T₁₄ are as defined as T₁ to T₄, T₉ to T₁₄, and T₅ to T₈, which do not form a ring in formula 1.

In one embodiment, T₁ to T₄, T₉ to T₁₄, and T₅ to T₈, which do not form a ring, each independently may be, hydrogen, deuterium, a substituted or unsubstituted (C6-C30)aryl, a substituted or unsubstituted (5- to 30-membered)heteroaryl, or -L₁-NAr₁Ar₂, preferably hydrogen, deuterium, a substituted or unsubstituted (C6-C25)aryl, a substituted or unsubstituted (5- to 25-membered)heteroaryl, or -L₁-NAr₁Ar₂, more preferably hydrogen, deuterium, a substituted or unsubstituted (C6-C18)aryl, a substituted or unsubstituted (5- to 18-membered)heteroaryl, or -L₁-NAr₁Ar₂. For example, T₁ to T₄, T₉ to T₁₄, and T₅ to T₈, which do not form a ring, each independently may be, hydrogen, deuterium, a substituted or unsubstituted phenyl, a substituted or unsubstituted dibenzofuranyl, or -L₁-NAr₁Ar₂.

In one embodiment, at least one of T₁ to T₁₄ may be -L₁-NAr₁Ar₂, for example, at least two of T₁ to T₁₄ may be -L₁-NAr₁Ar₂, for example, at least one of T₁ to T₁₄ may be -L₁-NAr₁Ar₂.

In one embodiment, L₁ may be a single bond, a substituted or unsubstituted (C6-C30)arylene, or a substituted or unsubstituted (5- to 30-membered)heteroarylene, preferably a single bond, a substituted or unsubstituted (C6-C25)arylene, or a substituted or unsubstituted (5- to 25-membered)heteroarylene, more preferably a single bond, a substituted or unsubstituted (C6-C18)arylene, or a substituted or unsubstituted (5- to 18-membered)heteroarylene. For example, L₁ may be phenylene unsubstituted or substituted with phenyl, a substituted or unsubstituted naphthylene, or a substituted or unsubstituted pyridylene.

In one embodiment, Ar₁ and Ar₂ each independently may be, a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C25)aryl or a substituted or unsubstituted (5- to 18-membered)heteroaryl. For example, Ar₁ and Ar₂ each independently may be phenyl unsubstituted or substituted with at least one selected from deuterium; cyano; tert-butyl; naphthyl; dibenzofuranyl; diphenylamino; triphenylsilyl; pyridyl substituted with phenyl; carbazolyl substituted with phenyl; benzofuranyl substituted with phenyl; and dibenzofuranyl, naphthyl unsubstituted or substituted with at least one selected from phenyl; and dibenzofuranyl, a substituted or unsubstituted p-biphenyl, a substituted or unsubstituted m-biphenyl, a substituted or unsubstituted o-biphenyl, a substituted or unsubstituted o-terphenyl, a substituted or unsubstituted m-terphenyl, phenanthrenyl unsubstituted or substituted with phenyl, a substituted or unsubstituted dimethylfluorenyl, a substituted or unsubstituted diphenylfluorenyl, a substituted or unsubstituted spirobifluorenyl, pyridyl unsubstituted or substituted with phenyl, carbazolyl unsubstituted or substituted with at least one selected from phenyl; naphthyl; biphenyl; dibenzofuranyl; and dibenzothiophenyl, dibenzofuranyl unsubstituted or substituted with phenyl, dibenzothiophenyl unsubstituted or substituted with phenyl, or a substituted or unsubstituted benzonaphthofuranyl. For example, the substitutent in the substituted groups may be further substituted with at least one deuterium.

In one embodiment, at least one of Ar₁ and Ar₂ may be a substituted or unsubstituted 1-carbazolyl, a substituted or unsubstituted 2-carbazolyl, a substituted or unsubstituted 3-carbazolyl, a substituted or unsubstituted 4-carbazolyl, or a substituted or unsubstituted 9-carbazolyl.

According to one embodiment, the first host compound represented by Formula 1 may be more specifically illustrated by the following compounds, but is not limited thereto.

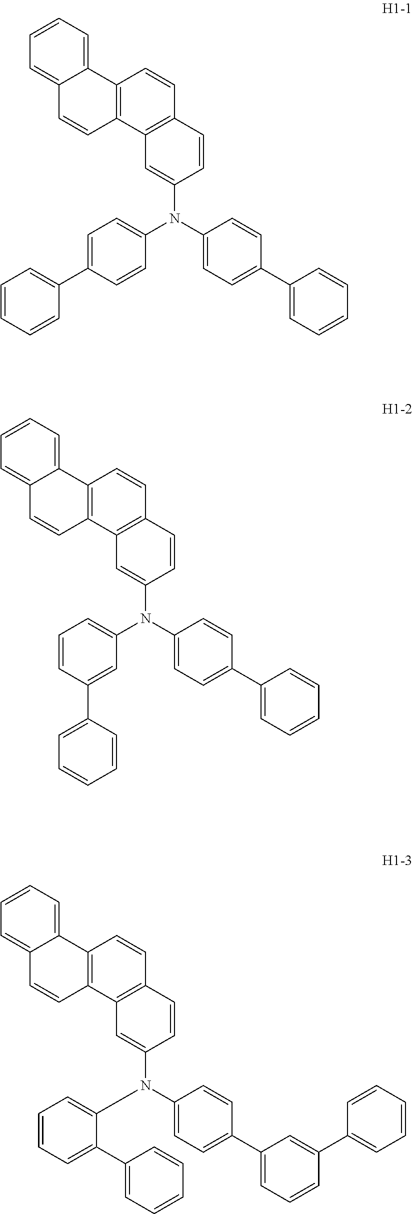 [SMILES: c1ccc(-c2ccc(N(c3ccc(-c4ccccc4)cc3)c3ccc4ccc5c6ccccc6ccc5c4c3)cc2)cc1 ; c1ccc(-c2ccc(N(c3cccc(-c4ccccc4)c3)c3ccc4ccc5c6ccccc6ccc5c4c3)cc2)cc1 ; c1ccc(-c2cccc(-c3ccc(N(c4ccc5ccc6c7ccccc7ccc6c5c4)c4ccccc4-c4ccccc4)cc3)c2)cc1]

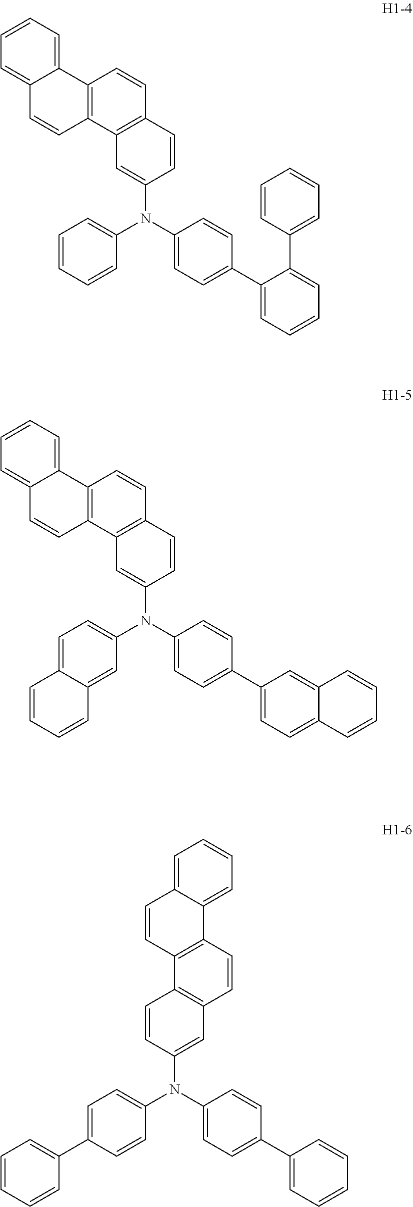 [SMILES: c1ccc(-c2ccccc2-c2ccc(N(c3ccccc3)c3ccc4ccc5c6ccccc6ccc5c4c3)cc2)cc1 ; c1ccc2cc(-c3ccc(N(c4ccc5ccccc5c4)c4ccc5ccc6c7ccccc7ccc6c5c4)cc3)ccc2c1 ; c1ccc(-c2ccc(N(c3ccc(-c4ccccc4)cc3)c3ccc4c(ccc5c6ccccc6ccc45)c3)cc2)cc1]

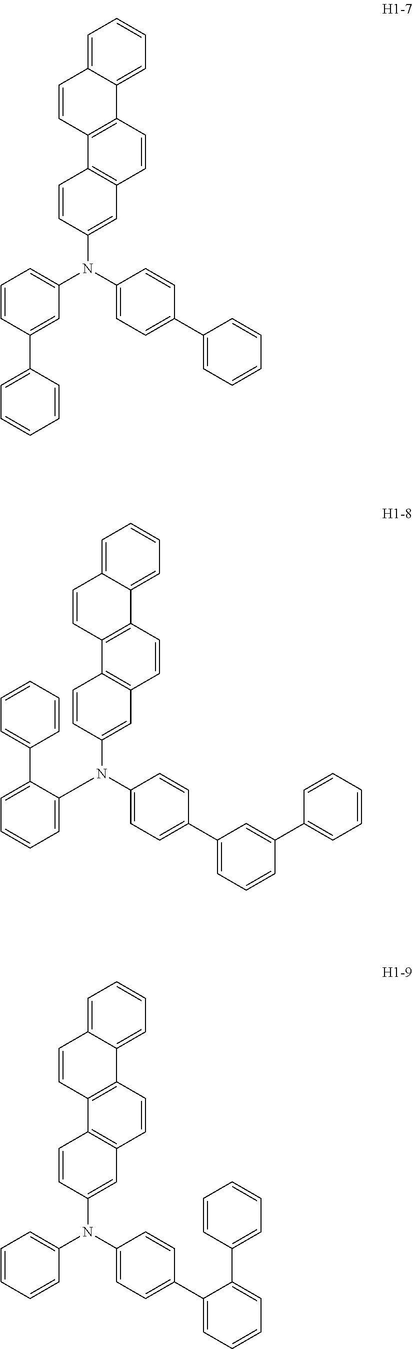 [SMILES: c1ccc(-c2ccc(N(c3cccc(-c4ccccc4)c3)c3ccc4c(ccc5c6ccccc6ccc45)c3)cc2)cc1 ; c1ccc(-c2cccc(-c3ccc(N(c4ccc5c(ccc6c7ccccc7ccc56)c4)c4ccccc4-c4ccccc4)cc3)c2)cc1 ; c1ccc(-c2ccccc2-c2ccc(N(c3ccccc3)c3ccc4c(ccc5c6ccccc6ccc45)c3)cc2)cc1]

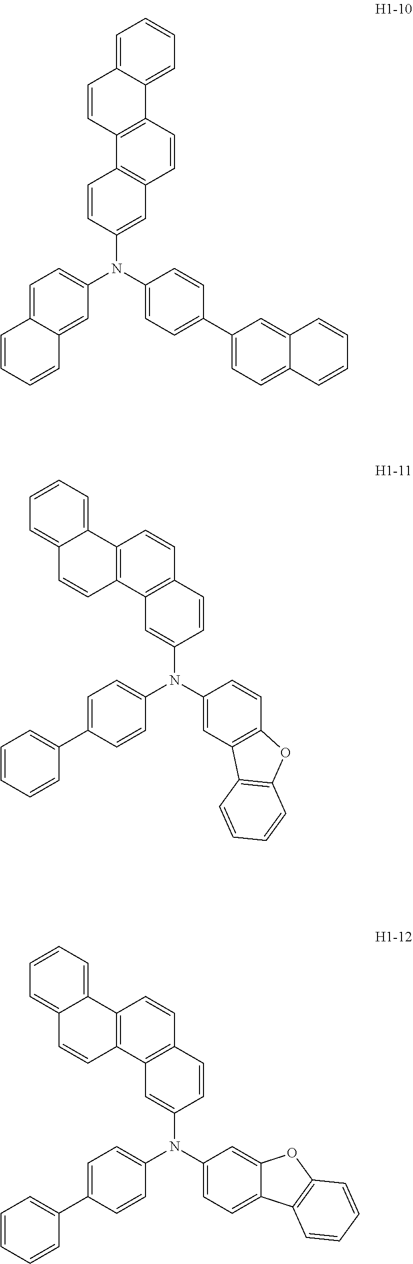 [SMILES: c1ccc2cc(-c3ccc(N(c4ccc5ccccc5c4)c4ccc5c(ccc6c7ccccc7ccc56)c4)cc3)ccc2c1 ; c1ccc(-c2ccc(N(c3ccc4ccc5c6ccccc6ccc5c4c3)c3ccc4oc5ccccc5c4c3)cc2)cc1 ; c1ccc(-c2ccc(N(c3ccc4c(c3)oc3ccccc34)c3ccc4ccc5c6ccccc6ccc5c4c3)cc2)cc1]

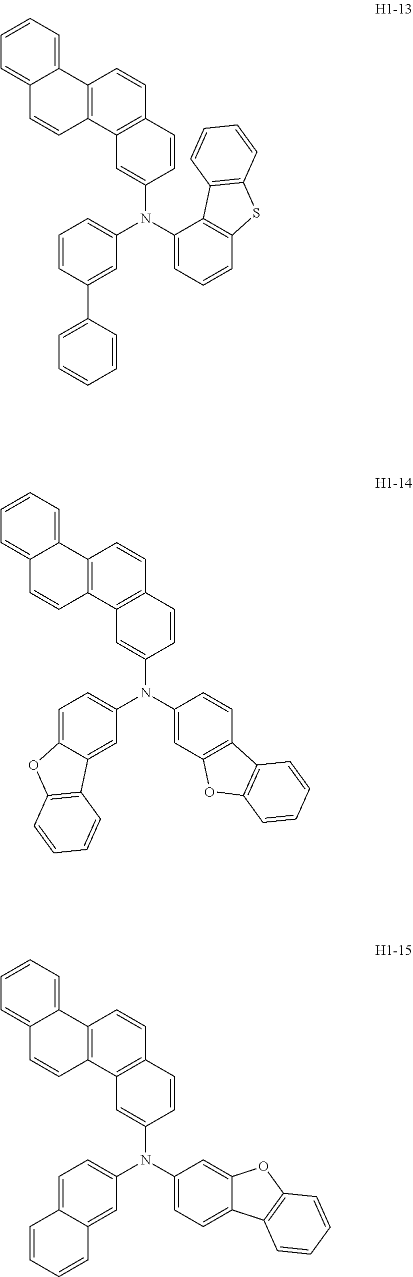 [SMILES: c1ccc(-c2cccc(N(c3ccc4ccc5c6ccccc6ccc5c4c3)c3cccc4sc5ccccc5c34)c2)cc1 ; c1ccc2c(c1)ccc1c3cc(N(c4ccc5c(c4)oc4ccccc45)c4ccc5oc6ccccc6c5c4)ccc3ccc21 ; c1ccc2cc(N(c3ccc4c(c3)oc3ccccc34)c3ccc4ccc5c6ccccc6ccc5c4c3)ccc2c1]

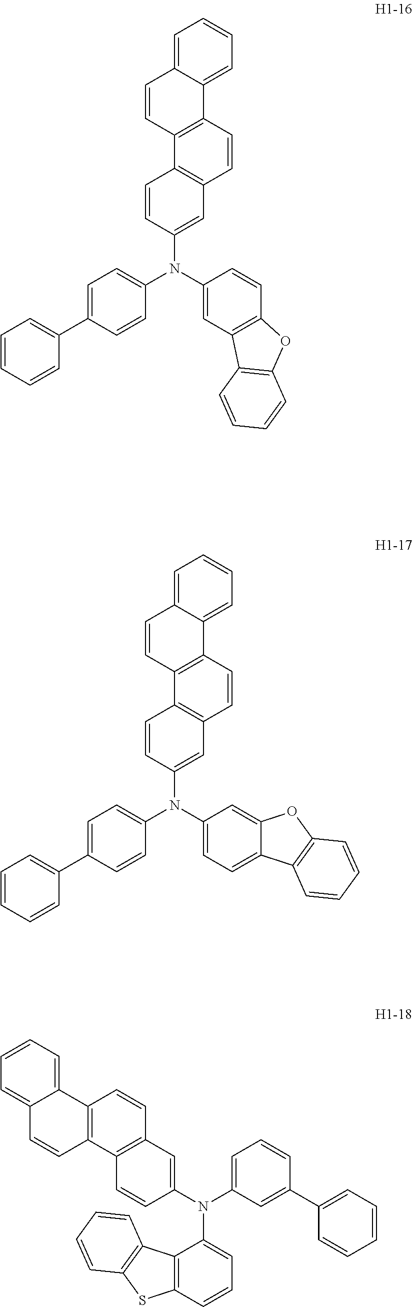 [SMILES: c1ccc(-c2ccc(N(c3ccc4c(ccc5c6ccccc6ccc45)c3)c3ccc4oc5ccccc5c4c3)cc2)cc1 ; c1ccc(-c2ccc(N(c3ccc4c(ccc5c6ccccc6ccc45)c3)c3ccc4c(c3)oc3ccccc34)cc2)cc1 ; c1ccc(-c2cccc(N(c3ccc4c(ccc5c6ccccc6ccc45)c3)c3cccc4sc5ccccc5c34)c2)cc1]

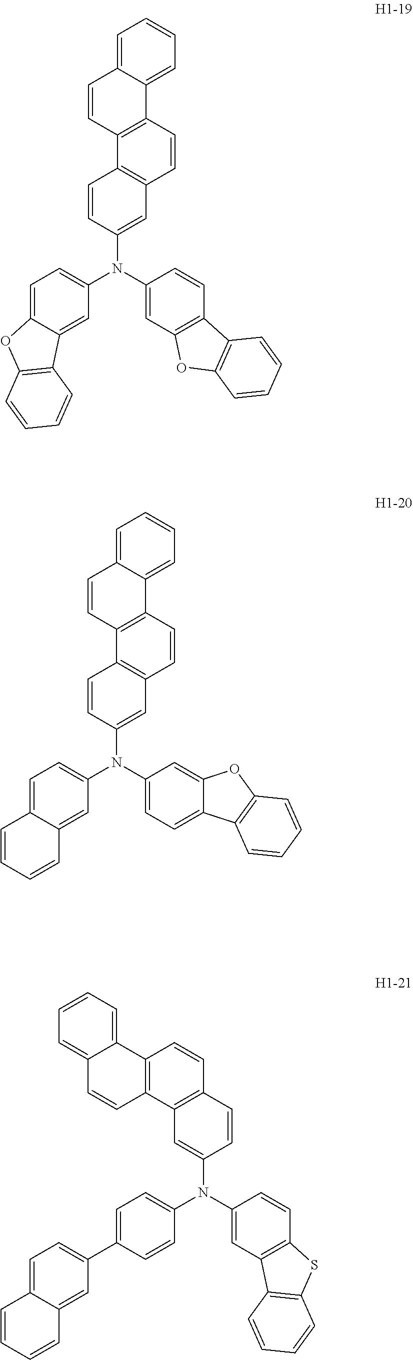 [SMILES: c1ccc2c(c1)ccc1c3ccc(N(c4ccc5c(c4)oc4ccccc45)c4ccc5oc6ccccc6c5c4)cc3ccc21 ; c1ccc2cc(N(c3ccc4c(ccc5c6ccccc6ccc45)c3)c3ccc4c(c3)oc3ccccc34)ccc2c1 ; c1ccc2cc(-c3ccc(N(c4ccc5ccc6c7ccccc7ccc6c5c4)c4ccc5sc6ccccc6c5c4)cc3)ccc2c1]

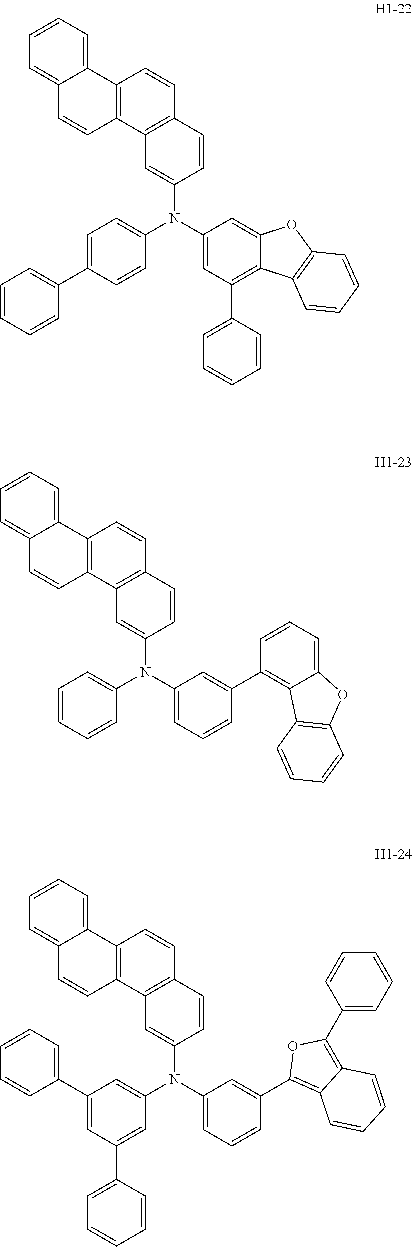 [SMILES: c1ccc(-c2ccc(N(c3cc(-c4ccccc4)c4c(c3)oc3ccccc34)c3ccc4ccc5c6ccccc6ccc5c4c3)cc2)cc1 ; c1ccc(N(c2cccc(-c3cccc4oc5ccccc5c34)c2)c2ccc3ccc4c5ccccc5ccc4c3c2)cc1 ; c1ccc(-c2cc(-c3ccccc3)cc(N(c3cccc(-c4oc(-c5ccccc5)c5ccccc45)c3)c3ccc4ccc5c6ccccc6ccc5c4c3)c2)cc1]

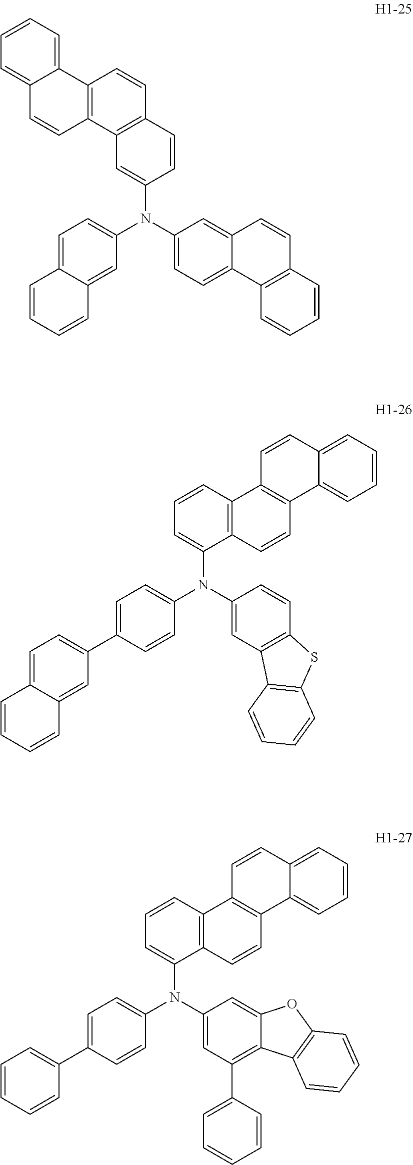 [SMILES: c1ccc2cc(N(c3ccc4c(ccc5ccccc54)c3)c3ccc4ccc5c6ccccc6ccc5c4c3)ccc2c1 ; c1ccc2cc(-c3ccc(N(c4ccc5sc6ccccc6c5c4)c4cccc5c4ccc4c6ccccc6ccc54)cc3)ccc2c1 ; c1ccc(-c2ccc(N(c3cc(-c4ccccc4)c4c(c3)oc3ccccc34)c3cccc4c3ccc3c5ccccc5ccc43)cc2)cc1]

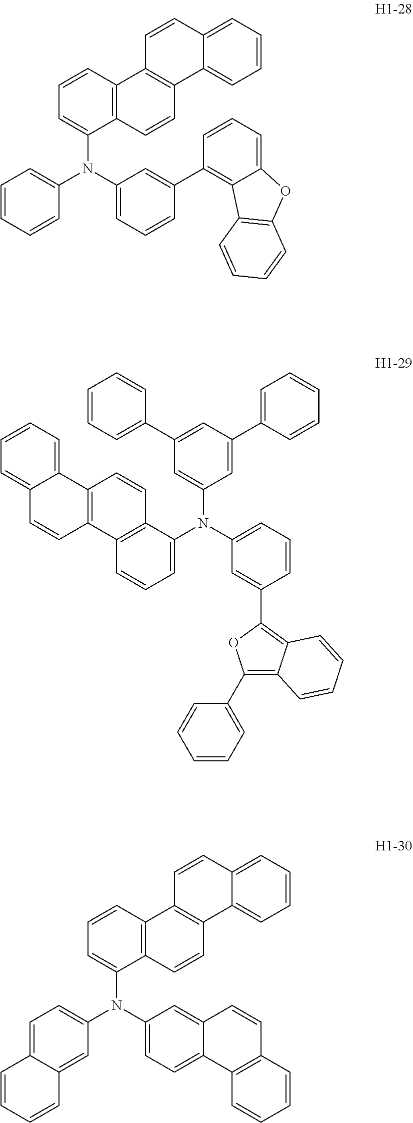 [SMILES: c1ccc(N(c2cccc(-c3cccc4oc5ccccc5c34)c2)c2cccc3c2ccc2c4ccccc4ccc32)cc1 ; c1ccc(-c2cc(-c3ccccc3)cc(N(c3cccc(-c4oc(-c5ccccc5)c5ccccc45)c3)c3cccc4c3ccc3c5ccccc5ccc43)c2)cc1 ; c1ccc2cc(N(c3ccc4c(ccc5ccccc54)c3)c3cccc4c3ccc3c5ccccc5ccc43)ccc2c1]

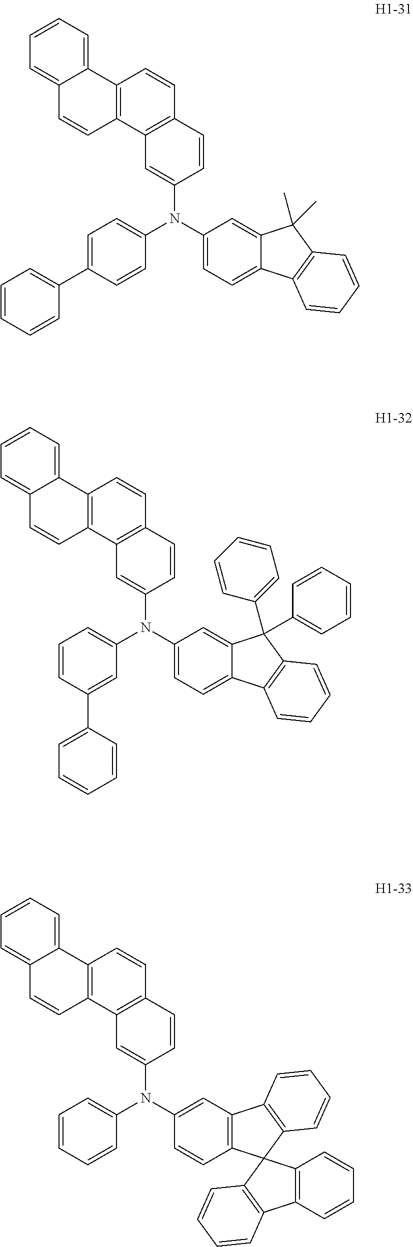 [SMILES: CC1(C)c2ccccc2-c2ccc(N(c3ccc(-c4ccccc4)cc3)c3ccc4ccc5c6ccccc6ccc5c4c3)cc21 ; c1ccc(-c2cccc(N(c3ccc4c(c3)C(c3ccccc3)(c3ccccc3)c3ccccc3-4)c3ccc4ccc5c6ccccc6ccc5c4c3)c2)cc1 ; c1ccc(N(c2ccc3c(c2)-c2ccccc2C32c3ccccc3-c3ccccc32)c2ccc3ccc4c5ccccc5ccc4c3c2)cc1]

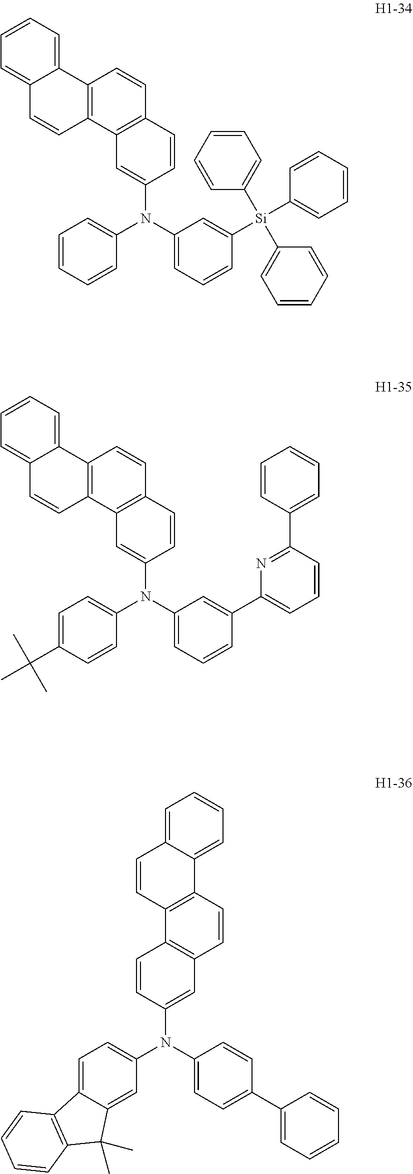 [SMILES: c1ccc(N(c2cccc([Si](c3ccccc3)(c3ccccc3)c3ccccc3)c2)c2ccc3ccc4c5ccccc5ccc4c3c2)cc1 ; CC(C)(C)c1ccc(N(c2cccc(-c3cccc(-c4ccccc4)n3)c2)c2ccc3ccc4c5ccccc5ccc4c3c2)cc1 ; CC1(C)c2ccccc2-c2ccc(N(c3ccc(-c4ccccc4)cc3)c3ccc4c(ccc5c6ccccc6ccc45)c3)cc21]

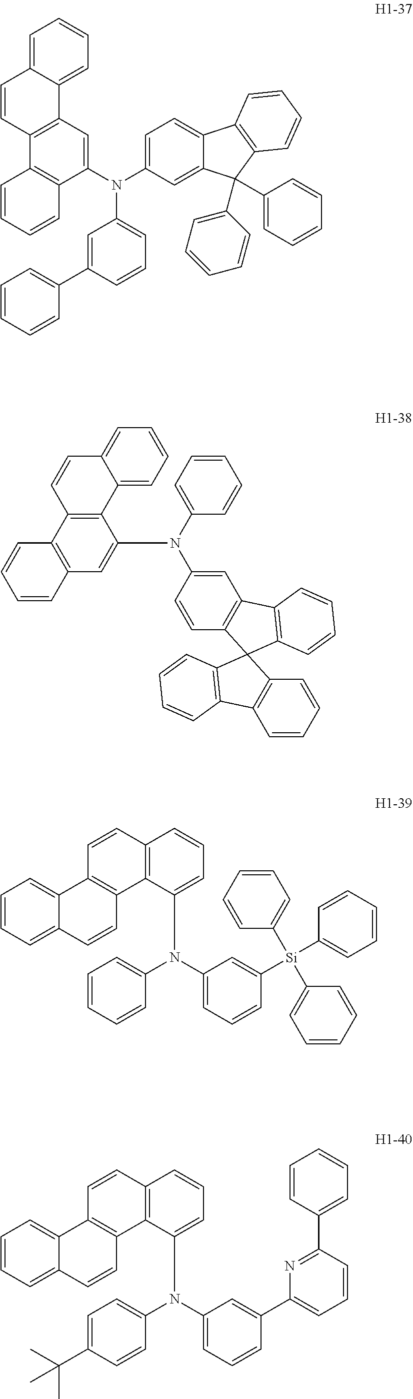 [SMILES: c1ccc(-c2cccc(N(c3ccc4c(c3)C(c3ccccc3)(c3ccccc3)c3ccccc3-4)c3cc4c5ccccc5ccc4c4ccccc34)c2)cc1 ; c1ccc(N(c2ccc3c(c2)-c2ccccc2C32c3ccccc3-c3ccccc32)c2cc3ccccc3c3ccc4ccccc4c23)cc1 ; c1ccc(N(c2cccc([Si](c3ccccc3)(c3ccccc3)c3ccccc3)c2)c2cccc3ccc4c5ccccc5ccc4c23)cc1 ; CC(C)(C)c1ccc(N(c2cccc(-c3cccc(-c4ccccc4)n3)c2)c2cccc3ccc4c5ccccc5ccc4c23)cc1]

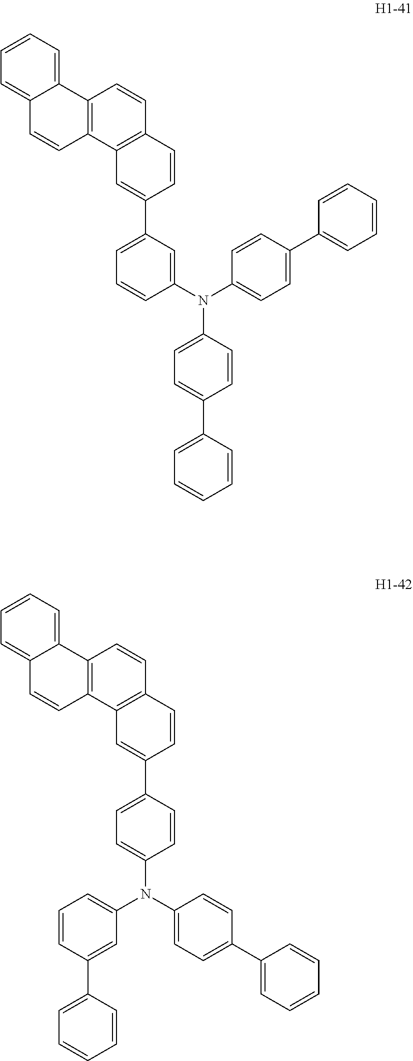 [SMILES: c1ccc(-c2ccc(N(c3ccc(-c4ccccc4)cc3)c3cccc(-c4ccc5ccc6c7ccccc7ccc6c5c4)c3)cc2)cc1 ; c1ccc(-c2ccc(N(c3ccc(-c4ccc5ccc6c7ccccc7ccc6c5c4)cc3)c3cccc(-c4ccccc4)c3)cc2)cc1]

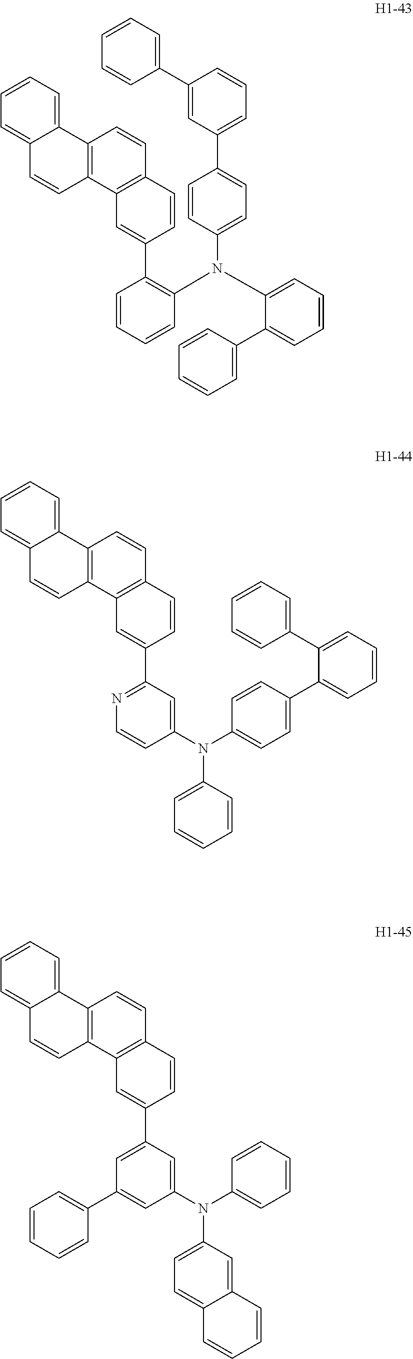 [SMILES: c1ccc(-c2cccc(-c3ccc(N(c4ccccc4-c4ccccc4)c4ccccc4-c4ccc5ccc6c7ccccc7ccc6c5c4)cc3)c2)cc1 ; c1ccc(-c2ccccc2-c2ccc(N(c3ccccc3)c3ccnc(-c4ccc5ccc6c7ccccc7ccc6c5c4)c3)cc2)cc1 ; c1ccc(-c2cc(-c3ccc4ccc5c6ccccc6ccc5c4c3)cc(N(c3ccccc3)c3ccc4ccccc4c3)c2)cc1]

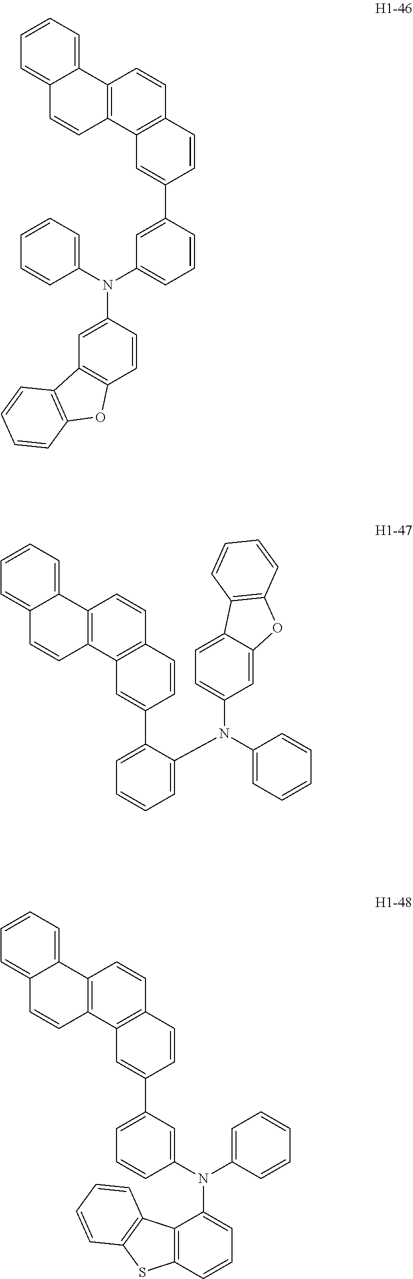 [SMILES: c1ccc(N(c2cccc(-c3ccc4ccc5c6ccccc6ccc5c4c3)c2)c2ccc3oc4ccccc4c3c2)cc1 ; c1ccc(N(c2ccc3c(c2)oc2ccccc23)c2ccccc2-c2ccc3ccc4c5ccccc5ccc4c3c2)cc1 ; c1ccc(N(c2cccc(-c3ccc4ccc5c6ccccc6ccc5c4c3)c2)c2cccc3sc4ccccc4c23)cc1]

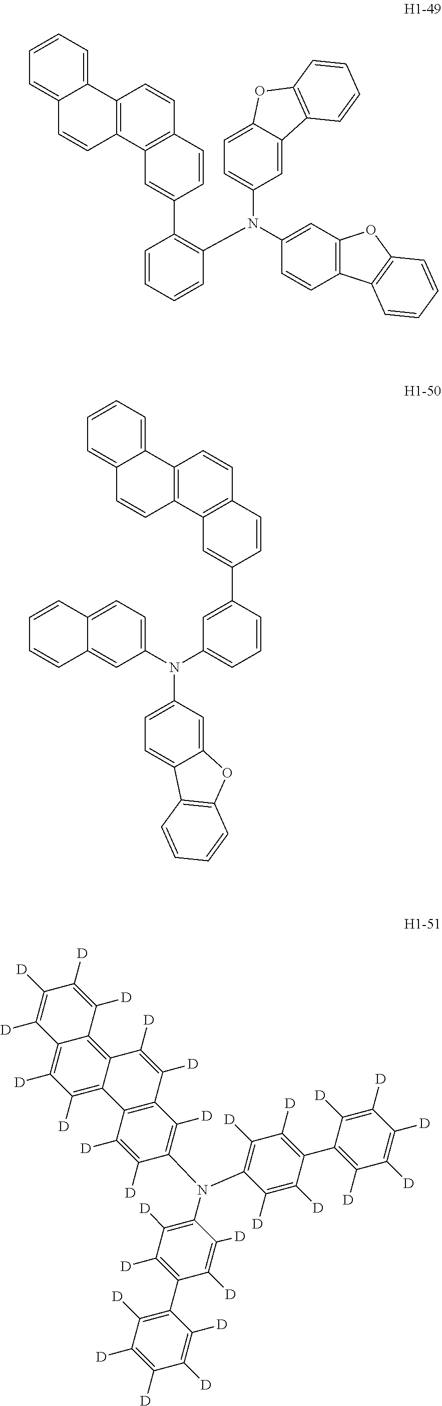 [SMILES: c1ccc(N(c2ccc3c(c2)oc2ccccc23)c2ccc3oc4ccccc4c3c2)c(-c2ccc3ccc4c5ccccc5ccc4c3c2)c1 ; c1cc(-c2ccc3ccc4c5ccccc5ccc4c3c2)cc(N(c2ccc3ccccc3c2)c2ccc3c(c2)oc2ccccc23)c1 ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c5c([2H])c([2H])c([2H])c([2H])c5c([2H])c([2H])c43)c([2H])c2[2H])c([2H])c1[2H]]

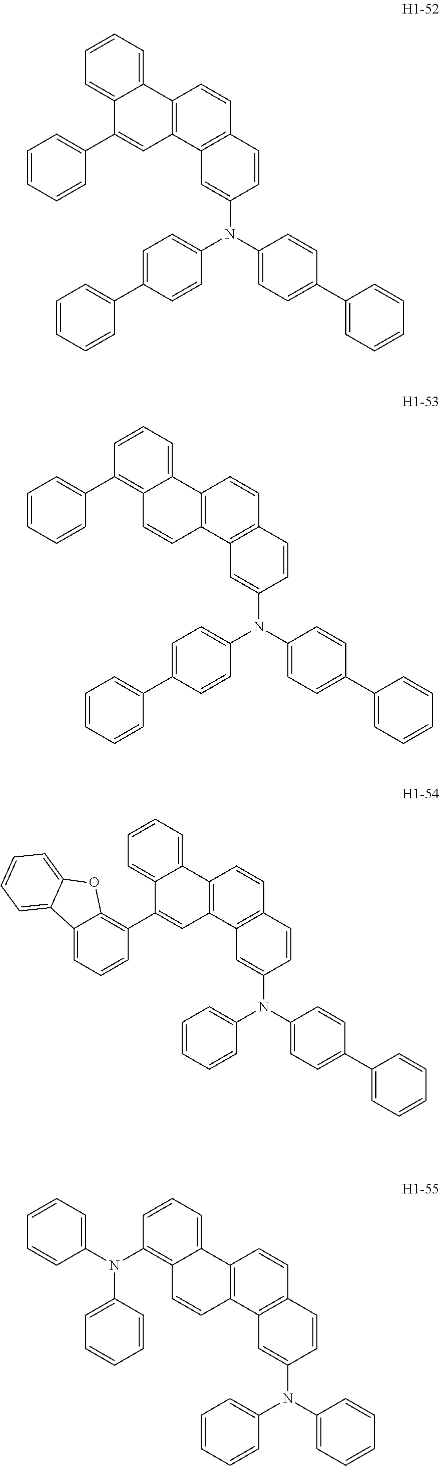 [SMILES: c1ccc(-c2ccc(N(c3ccc(-c4ccccc4)cc3)c3ccc4ccc5c6ccccc6c(-c6ccccc6)cc5c4c3)cc2)cc1 ; c1ccc(-c2ccc(N(c3ccc(-c4ccccc4)cc3)c3ccc4ccc5c6cccc(-c7ccccc7)c6ccc5c4c3)cc2)cc1 ; c1ccc(-c2ccc(N(c3ccccc3)c3ccc4ccc5c6ccccc6c(-c6cccc7c6oc6ccccc67)cc5c4c3)cc2)cc1 ; c1ccc(N(c2ccccc2)c2ccc3ccc4c5cccc(N(c6ccccc6)c6ccccc6)c5ccc4c3c2)cc1]

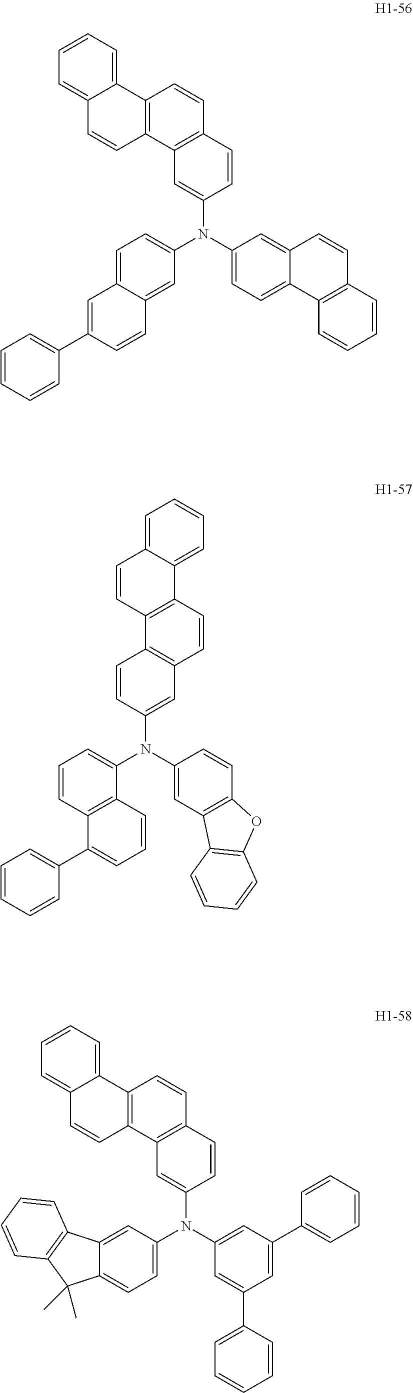 [SMILES: c1ccc(-c2ccc3cc(N(c4ccc5c(ccc6ccccc65)c4)c4ccc5ccc6c7ccccc7ccc6c5c4)ccc3c2)cc1 ; c1ccc(-c2cccc3c(N(c4ccc5c(ccc6c7ccccc7ccc56)c4)c4ccc5oc6ccccc6c5c4)cccc23)cc1 ; CC1(C)c2ccccc2-c2cc(N(c3cc(-c4ccccc4)cc(-c4ccccc4)c3)c3ccc4ccc5c6ccccc6ccc5c4c3)ccc21]

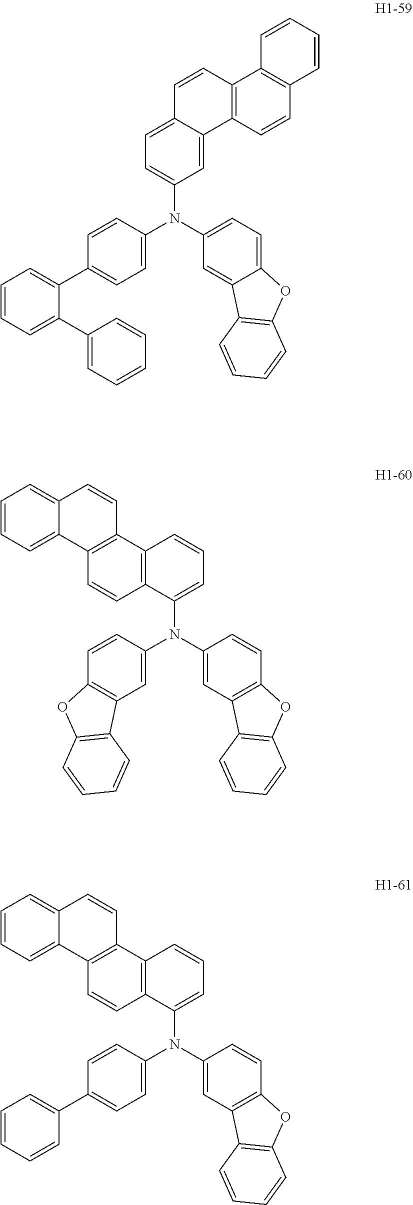 [SMILES: c1ccc(-c2ccccc2-c2ccc(N(c3ccc4ccc5c6ccccc6ccc5c4c3)c3ccc4oc5ccccc5c4c3)cc2)cc1 ; c1ccc2c(c1)ccc1c3cccc(N(c4ccc5oc6ccccc6c5c4)c4ccc5oc6ccccc6c5c4)c3ccc21 ; c1ccc(-c2ccc(N(c3ccc4oc5ccccc5c4c3)c3cccc4c3ccc3c5ccccc5ccc43)cc2)cc1]

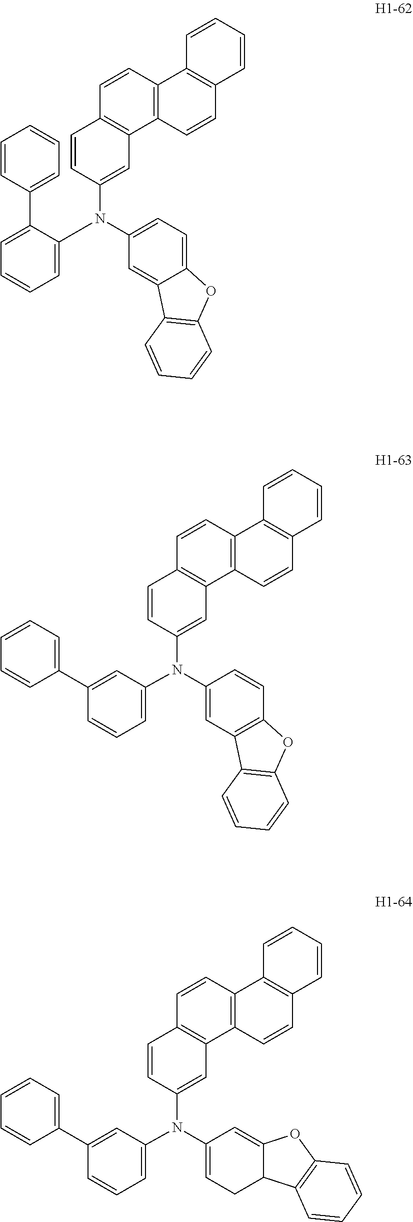 [SMILES: c1ccc(-c2ccccc2N(c2ccc3ccc4c5ccccc5ccc4c3c2)c2ccc3oc4ccccc4c3c2)cc1 ; c1ccc(-c2cccc(N(c3ccc4ccc5c6ccccc6ccc5c4c3)c3ccc4oc5ccccc5c4c3)c2)cc1 ; C1=C2Oc3ccccc3C2CC=C1N(c1cccc(-c2ccccc2)c1)c1ccc2ccc3c4ccccc4ccc3c2c1]

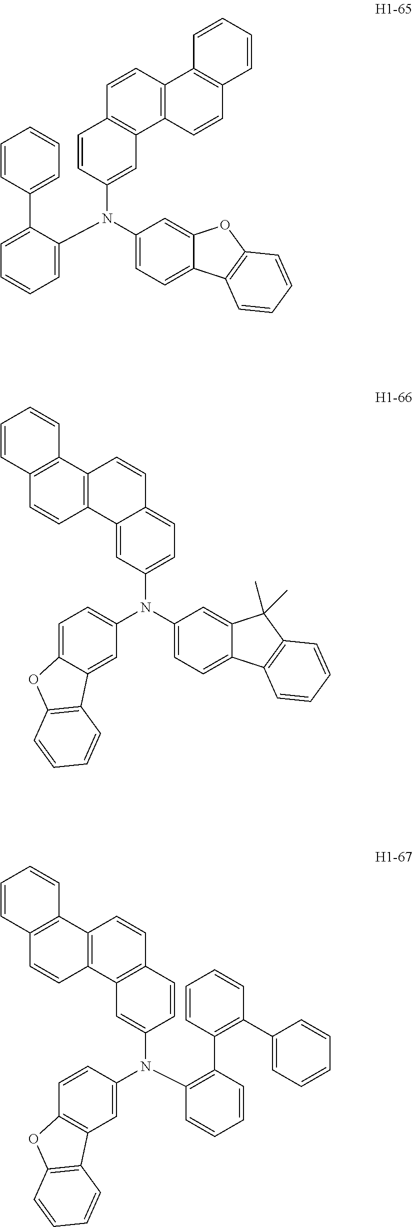 [SMILES: c1ccc(-c2ccccc2N(c2ccc3c(c2)oc2ccccc23)c2ccc3ccc4c5ccccc5ccc4c3c2)cc1 ; CC1(C)c2ccccc2-c2ccc(N(c3ccc4ccc5c6ccccc6ccc5c4c3)c3ccc4oc5ccccc5c4c3)cc21 ; c1ccc(-c2ccccc2-c2ccccc2N(c2ccc3ccc4c5ccccc5ccc4c3c2)c2ccc3oc4ccccc4c3c2)cc1]

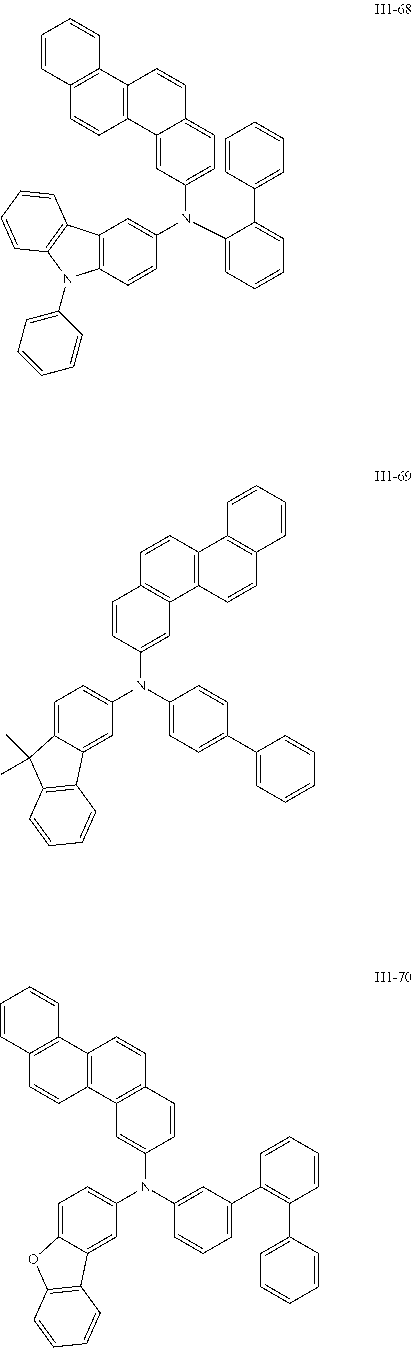 [SMILES: c1ccc(-c2ccccc2N(c2ccc3ccc4c5ccccc5ccc4c3c2)c2ccc3c(c2)c2ccccc2n3-c2ccccc2)cc1 ; CC1(C)c2ccccc2-c2cc(N(c3ccc(-c4ccccc4)cc3)c3ccc4ccc5c6ccccc6ccc5c4c3)ccc21 ; c1ccc(-c2ccccc2-c2cccc(N(c3ccc4ccc5c6ccccc6ccc5c4c3)c3ccc4oc5ccccc5c4c3)c2)cc1]

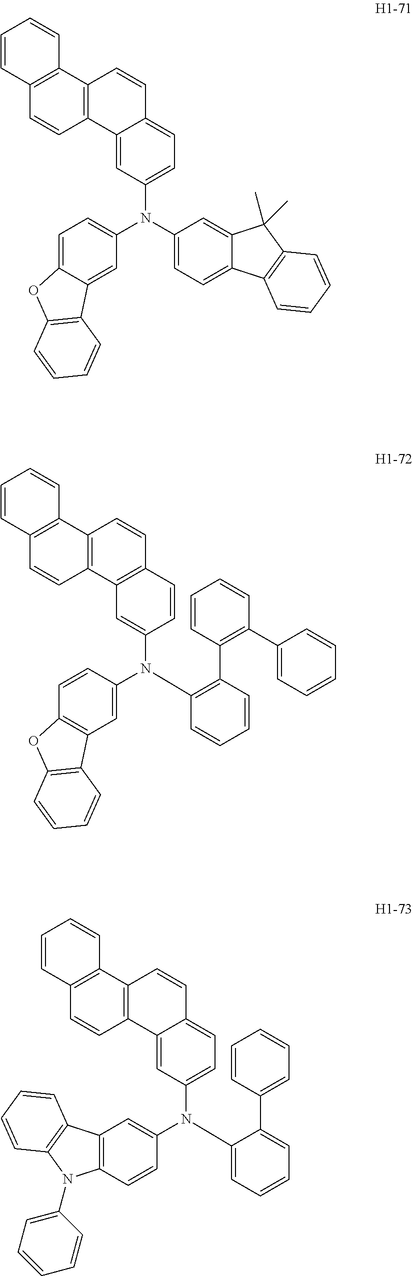 [SMILES: CC1(C)c2ccccc2-c2ccc(N(c3ccc4ccc5c6ccccc6ccc5c4c3)c3ccc4oc5ccccc5c4c3)cc21 ; c1ccc(-c2ccccc2-c2ccccc2N(c2ccc3ccc4c5ccccc5ccc4c3c2)c2ccc3oc4ccccc4c3c2)cc1 ; c1ccc(-c2ccccc2N(c2ccc3ccc4c5ccccc5ccc4c3c2)c2ccc3c(c2)c2ccccc2n3-c2ccccc2)cc1]

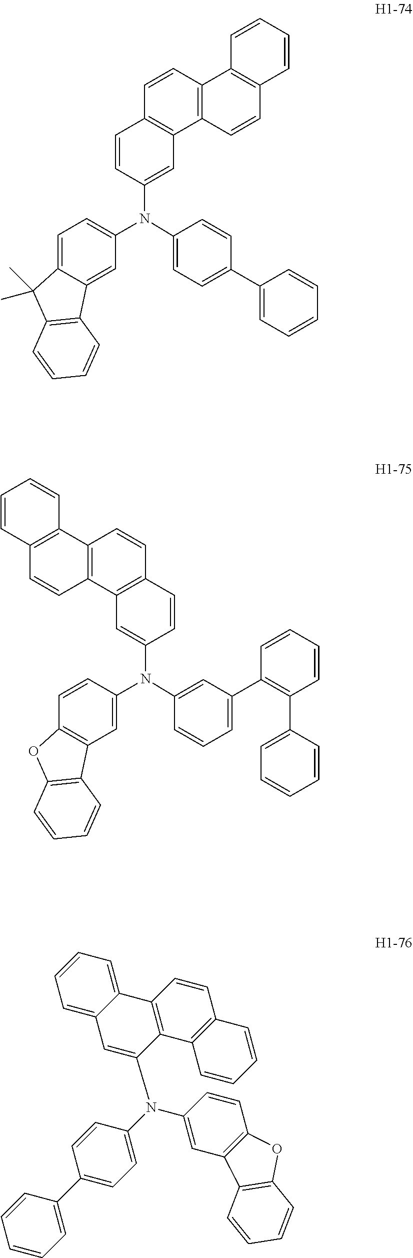 [SMILES: CC1(C)c2ccccc2-c2cc(N(c3ccc(-c4ccccc4)cc3)c3ccc4ccc5c6ccccc6ccc5c4c3)ccc21 ; c1ccc(-c2ccccc2-c2cccc(N(c3ccc4ccc5c6ccccc6ccc5c4c3)c3ccc4oc5ccccc5c4c3)c2)cc1 ; c1ccc(-c2ccc(N(c3ccc4oc5ccccc5c4c3)c3cc4ccccc4c4ccc5ccccc5c34)cc2)cc1]

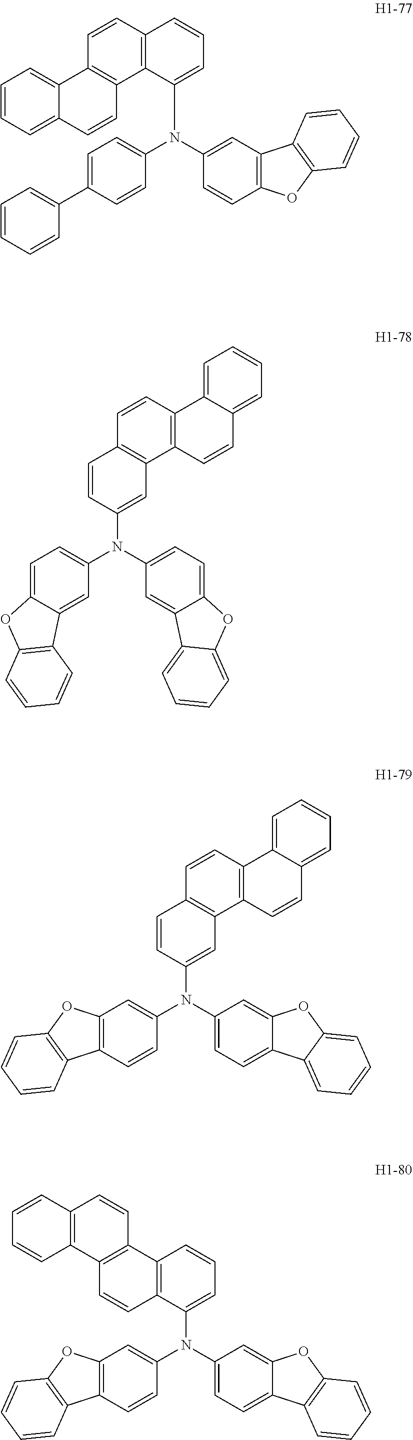 [SMILES: c1ccc(-c2ccc(N(c3ccc4oc5ccccc5c4c3)c3cccc4ccc5c6ccccc6ccc5c34)cc2)cc1 ; c1ccc2c(c1)ccc1c3cc(N(c4ccc5oc6ccccc6c5c4)c4ccc5oc6ccccc6c5c4)ccc3ccc21 ; c1ccc2c(c1)ccc1c3cc(N(c4ccc5c(c4)oc4ccccc45)c4ccc5c(c4)oc4ccccc45)ccc3ccc21 ; c1ccc2c(c1)ccc1c3cccc(N(c4ccc5c(c4)oc4ccccc45)c4ccc5c(c4)oc4ccccc45)c3ccc21]

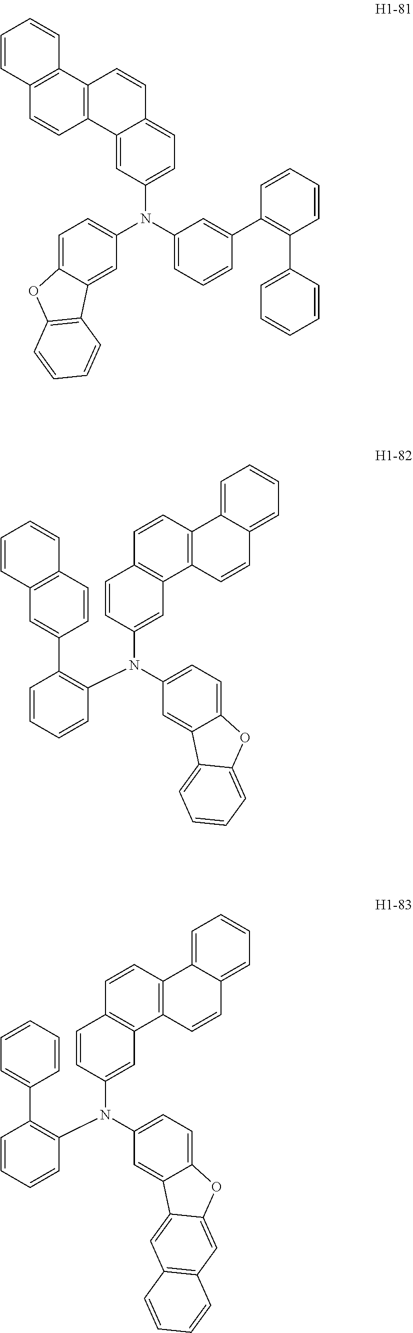 [SMILES: c1ccc(-c2ccccc2-c2cccc(N(c3ccc4ccc5c6ccccc6ccc5c4c3)c3ccc4oc5ccccc5c4c3)c2)cc1 ; c1ccc(N(c2ccc3ccc4c5ccccc5ccc4c3c2)c2ccc3oc4ccccc4c3c2)c(-c2ccc3ccccc3c2)c1 ; c1ccc(-c2ccccc2N(c2ccc3ccc4c5ccccc5ccc4c3c2)c2ccc3oc4cc5ccccc5cc4c3c2)cc1]

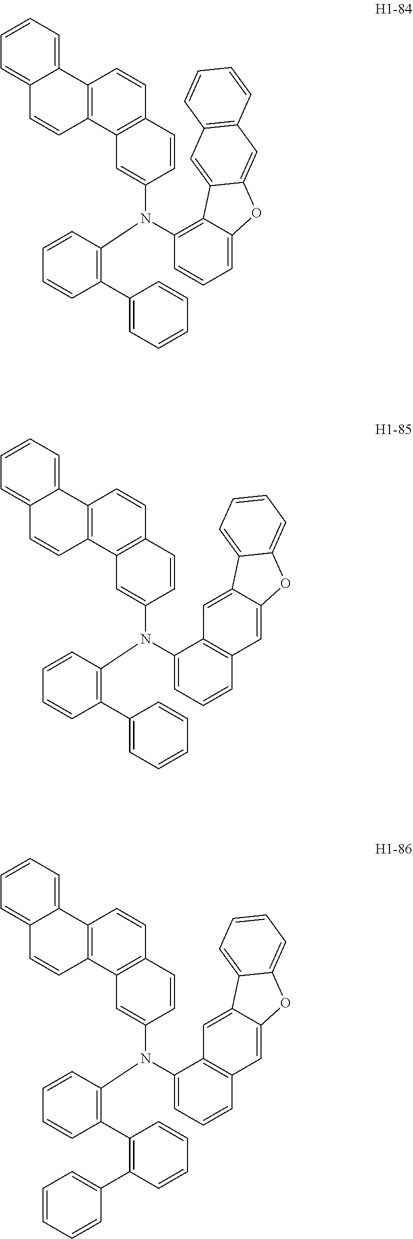 [SMILES: c1ccc(-c2ccccc2N(c2ccc3ccc4c5ccccc5ccc4c3c2)c2cccc3oc4cc5ccccc5cc4c23)cc1 ; c1ccc(-c2ccccc2N(c2ccc3ccc4c5ccccc5ccc4c3c2)c2cccc3cc4oc5ccccc5c4cc23)cc1 ; c1ccc(-c2ccccc2-c2ccccc2N(c2ccc3ccc4c5ccccc5ccc4c3c2)c2cccc3cc4oc5ccccc5c4cc23)cc1]

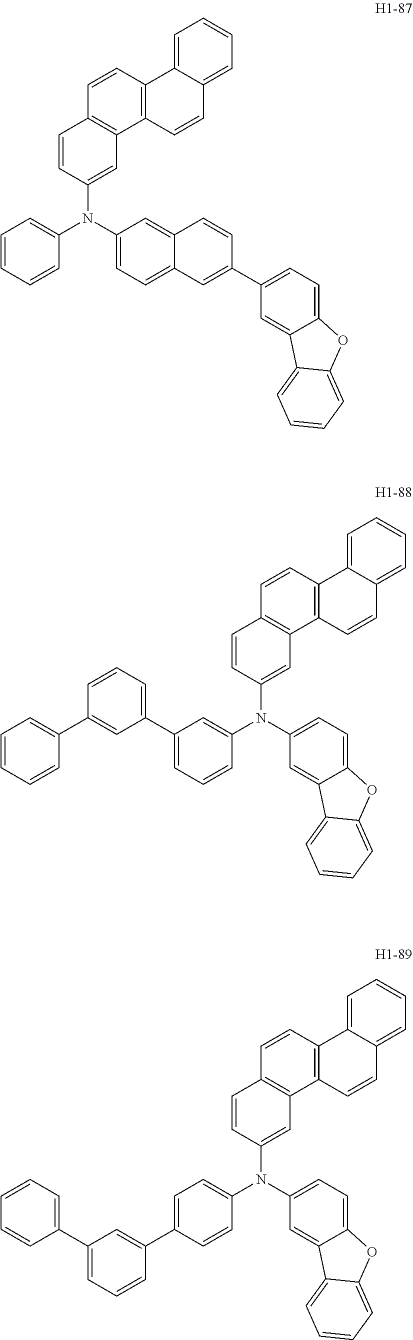 [SMILES: c1ccc(N(c2ccc3cc(-c4ccc5oc6ccccc6c5c4)ccc3c2)c2ccc3ccc4c5ccccc5ccc4c3c2)cc1 ; c1ccc(-c2cccc(-c3cccc(N(c4ccc5ccc6c7ccccc7ccc6c5c4)c4ccc5oc6ccccc6c5c4)c3)c2)cc1 ; c1ccc(-c2cccc(-c3ccc(N(c4ccc5ccc6c7ccccc7ccc6c5c4)c4ccc5oc6ccccc6c5c4)cc3)c2)cc1]

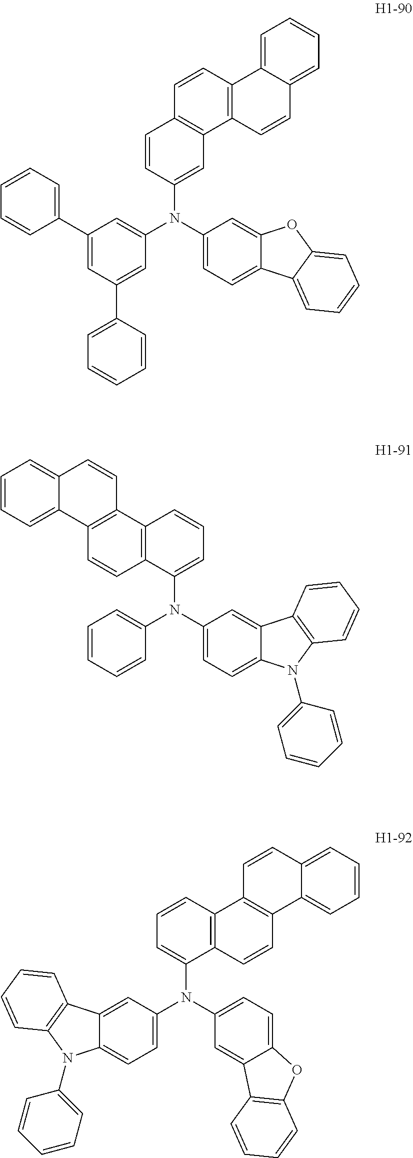 [SMILES: c1ccc(-c2cc(-c3ccccc3)cc(N(c3ccc4c(c3)oc3ccccc34)c3ccc4ccc5c6ccccc6ccc5c4c3)c2)cc1 ; c1ccc(N(c2ccc3c(c2)c2ccccc2n3-c2ccccc2)c2cccc3c2ccc2c4ccccc4ccc32)cc1 ; c1ccc(-n2c3ccccc3c3cc(N(c4ccc5oc6ccccc6c5c4)c4cccc5c4ccc4c6ccccc6ccc54)ccc32)cc1]

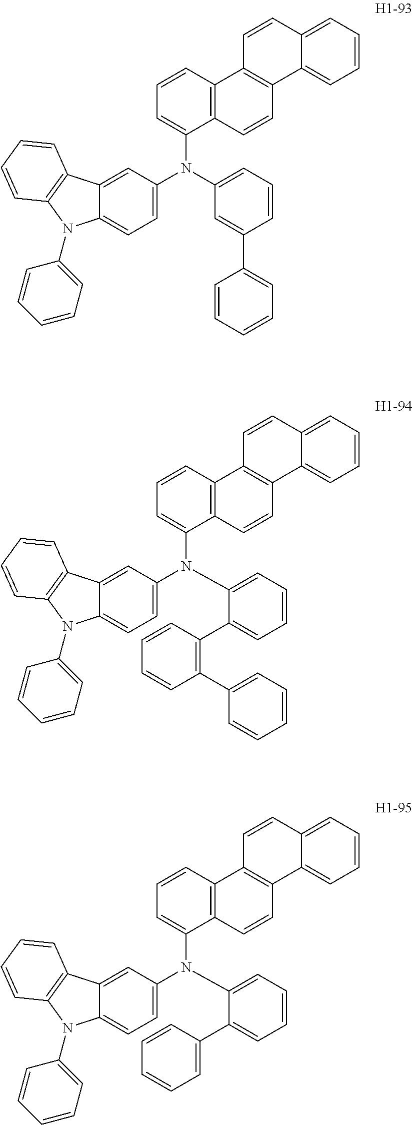 [SMILES: c1ccc(-c2cccc(N(c3ccc4c(c3)c3ccccc3n4-c3ccccc3)c3cccc4c3ccc3c5ccccc5ccc43)c2)cc1 ; c1ccc(-c2ccccc2-c2ccccc2N(c2ccc3c(c2)c2ccccc2n3-c2ccccc2)c2cccc3c2ccc2c4ccccc4ccc32)cc1 ; c1ccc(-c2ccccc2N(c2ccc3c(c2)c2ccccc2n3-c2ccccc2)c2cccc3c2ccc2c4ccccc4ccc32)cc1]

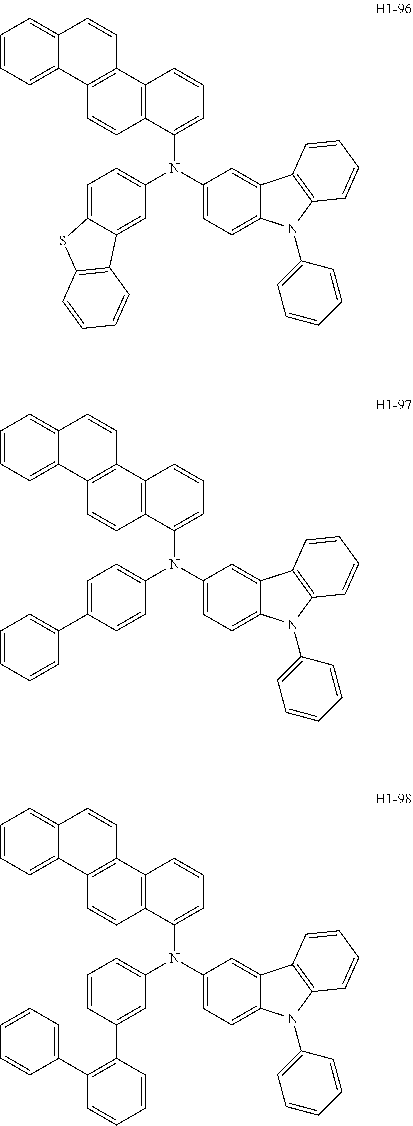 [SMILES: c1ccc(-n2c3ccccc3c3cc(N(c4ccc5sc6ccccc6c5c4)c4cccc5c4ccc4c6ccccc6ccc54)ccc32)cc1 ; c1ccc(-c2ccc(N(c3ccc4c(c3)c3ccccc3n4-c3ccccc3)c3cccc4c3ccc3c5ccccc5ccc43)cc2)cc1 ; c1ccc(-c2ccccc2-c2cccc(N(c3ccc4c(c3)c3ccccc3n4-c3ccccc3)c3cccc4c3ccc3c5ccccc5ccc43)c2)cc1]

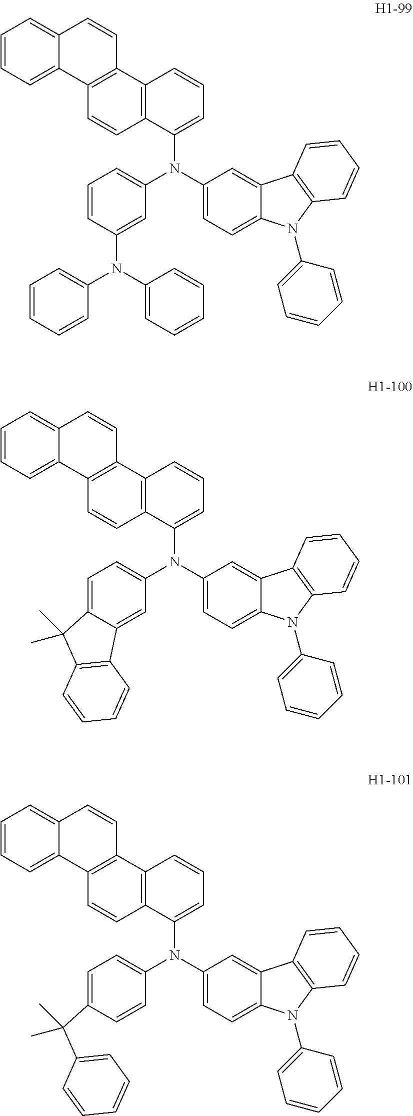 [SMILES: c1ccc(N(c2ccccc2)c2cccc(N(c3ccc4c(c3)c3ccccc3n4-c3ccccc3)c3cccc4c3ccc3c5ccccc5ccc43)c2)cc1 ; CC1(C)c2ccccc2-c2cc(N(c3ccc4c(c3)c3ccccc3n4-c3ccccc3)c3cccc4c3ccc3c5ccccc5ccc43)ccc21 ; CC(C)(c1ccccc1)c1ccc(N(c2ccc3c(c2)c2ccccc2n3-c2ccccc2)c2cccc3c2ccc2c4ccccc4ccc32)cc1]

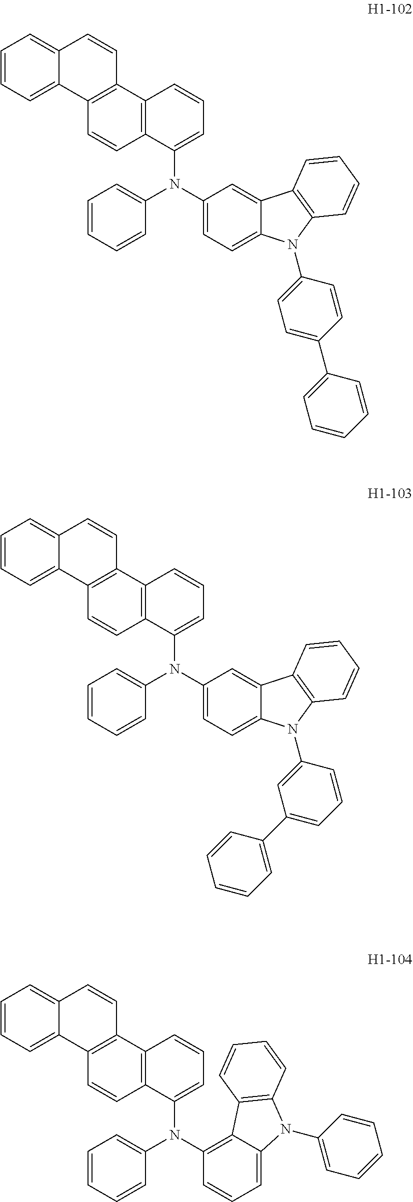 [SMILES: c1ccc(-c2ccc(-n3c4ccccc4c4cc(N(c5ccccc5)c5cccc6c5ccc5c7ccccc7ccc65)ccc43)cc2)cc1 ; c1ccc(-c2cccc(-n3c4ccccc4c4cc(N(c5ccccc5)c5cccc6c5ccc5c7ccccc7ccc65)ccc43)c2)cc1 ; c1ccc(N(c2cccc3c2ccc2c4ccccc4ccc32)c2cccc3c2c2ccccc2n3-c2ccccc2)cc1]

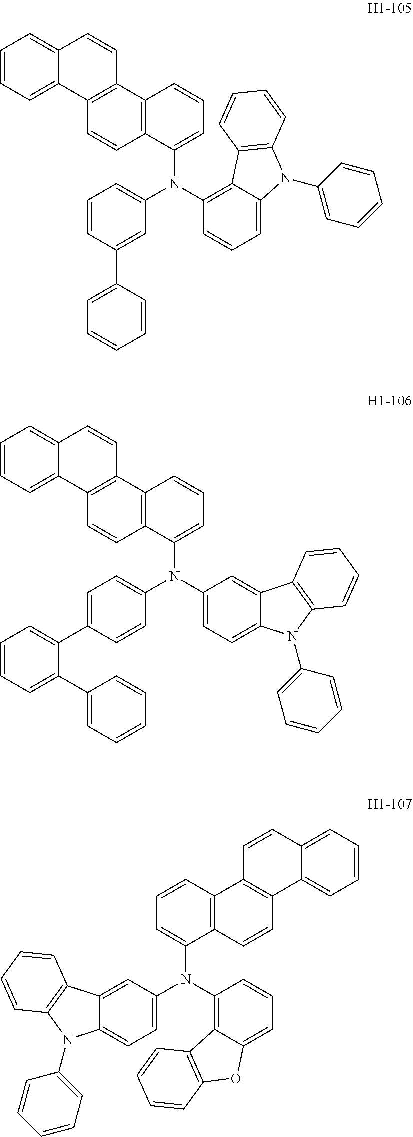 [SMILES: c1ccc(-c2cccc(N(c3cccc4c3ccc3c5ccccc5ccc43)c3cccc4c3c3ccccc3n4-c3ccccc3)c2)cc1 ; c1ccc(-c2ccccc2-c2ccc(N(c3ccc4c(c3)c3ccccc3n4-c3ccccc3)c3cccc4c3ccc3c5ccccc5ccc43)cc2)cc1 ; c1ccc(-n2c3ccccc3c3cc(N(c4cccc5c4ccc4c6ccccc6ccc54)c4cccc5oc6ccccc6c45)ccc32)cc1]

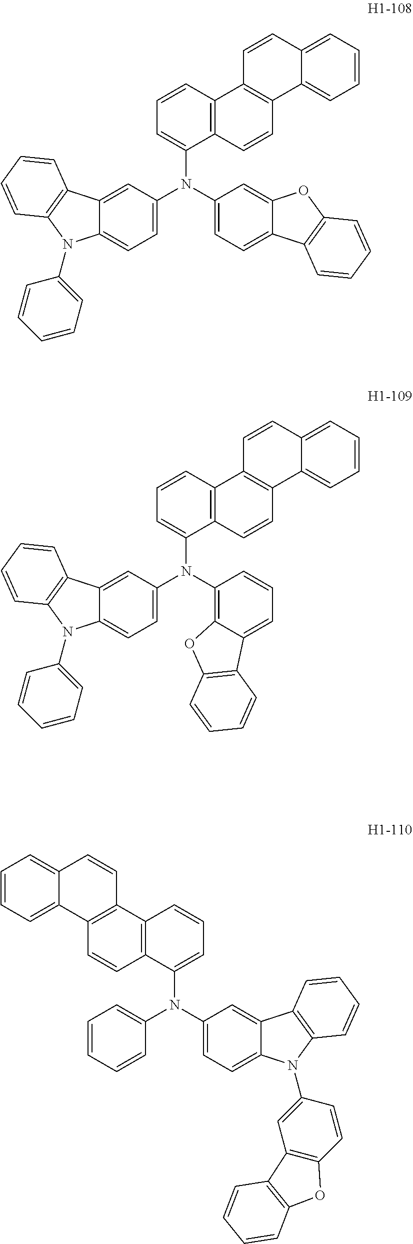 [SMILES: c1ccc(-n2c3ccccc3c3cc(N(c4ccc5c(c4)oc4ccccc45)c4cccc5c4ccc4c6ccccc6ccc54)ccc32)cc1 ; c1ccc(-n2c3ccccc3c3cc(N(c4cccc5c4ccc4c6ccccc6ccc54)c4cccc5c4oc4ccccc45)ccc32)cc1 ; c1ccc(N(c2ccc3c(c2)c2ccccc2n3-c2ccc3oc4ccccc4c3c2)c2cccc3c2ccc2c4ccccc4ccc32)cc1]

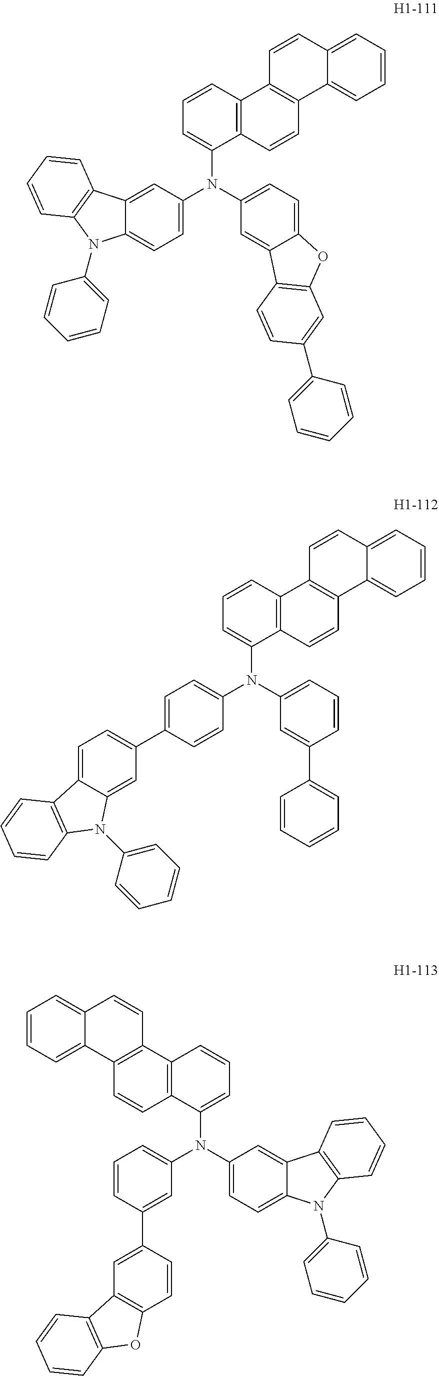 [SMILES: c1ccc(-c2ccc3c(c2)oc2ccc(N(c4ccc5c(c4)c4ccccc4n5-c4ccccc4)c4cccc5c4ccc4c6ccccc6ccc54)cc23)cc1 ; c1ccc(-c2cccc(N(c3ccc(-c4ccc5c6ccccc6n(-c6ccccc6)c5c4)cc3)c3cccc4c3ccc3c5ccccc5ccc43)c2)cc1 ; c1ccc(-n2c3ccccc3c3cc(N(c4cccc(-c5ccc6oc7ccccc7c6c5)c4)c4cccc5c4ccc4c6ccccc6ccc54)ccc32)cc1]

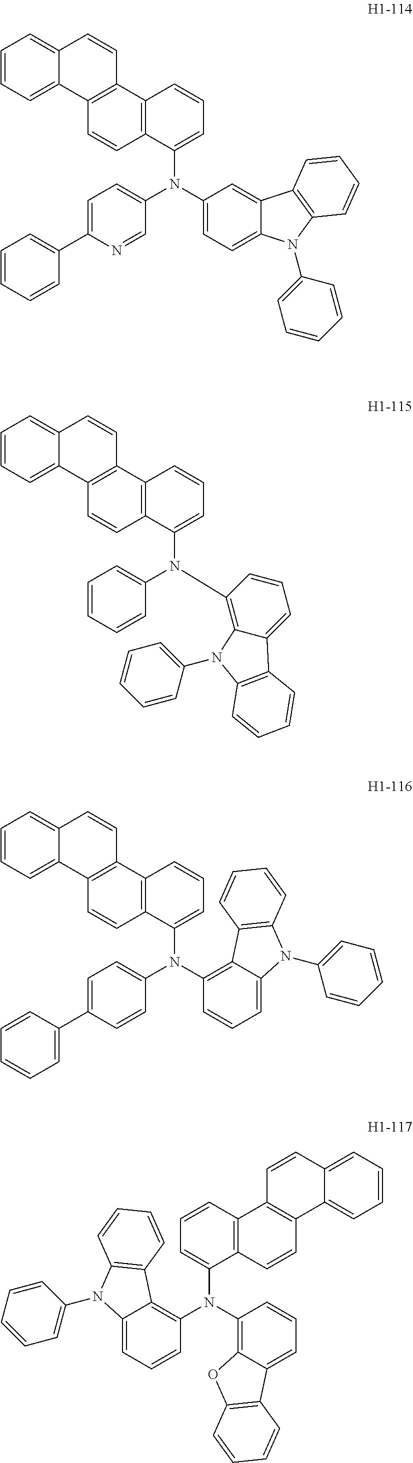 [SMILES: c1ccc(-c2ccc(N(c3ccc4c(c3)c3ccccc3n4-c3ccccc3)c3cccc4c3ccc3c5ccccc5ccc43)cn2)cc1 ; c1ccc(N(c2cccc3c2ccc2c4ccccc4ccc32)c2cccc3c4ccccc4n(-c4ccccc4)c23)cc1 ; c1ccc(-c2ccc(N(c3cccc4c3ccc3c5ccccc5ccc43)c3cccc4c3c3ccccc3n4-c3ccccc3)cc2)cc1 ; c1ccc(-n2c3ccccc3c3c(N(c4cccc5c4ccc4c6ccccc6ccc54)c4cccc5c4oc4ccccc45)cccc32)cc1]

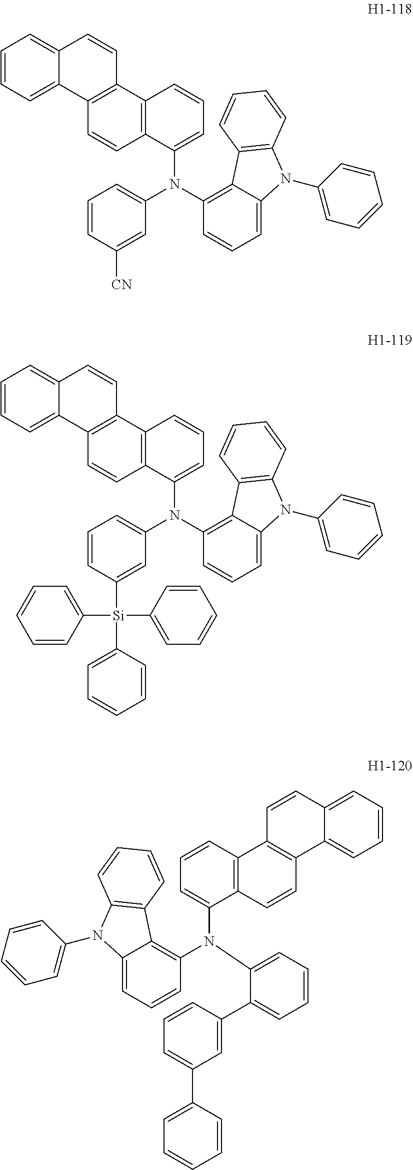 [SMILES: N#Cc1cccc(N(c2cccc3c2ccc2c4ccccc4ccc32)c2cccc3c2c2ccccc2n3-c2ccccc2)c1 ; c1ccc(-n2c3ccccc3c3c(N(c4cccc([Si](c5ccccc5)(c5ccccc5)c5ccccc5)c4)c4cccc5c4ccc4c6ccccc6ccc54)cccc32)cc1 ; c1ccc(-c2cccc(-c3ccccc3N(c3cccc4c3ccc3c5ccccc5ccc43)c3cccc4c3c3ccccc3n4-c3ccccc3)c2)cc1]

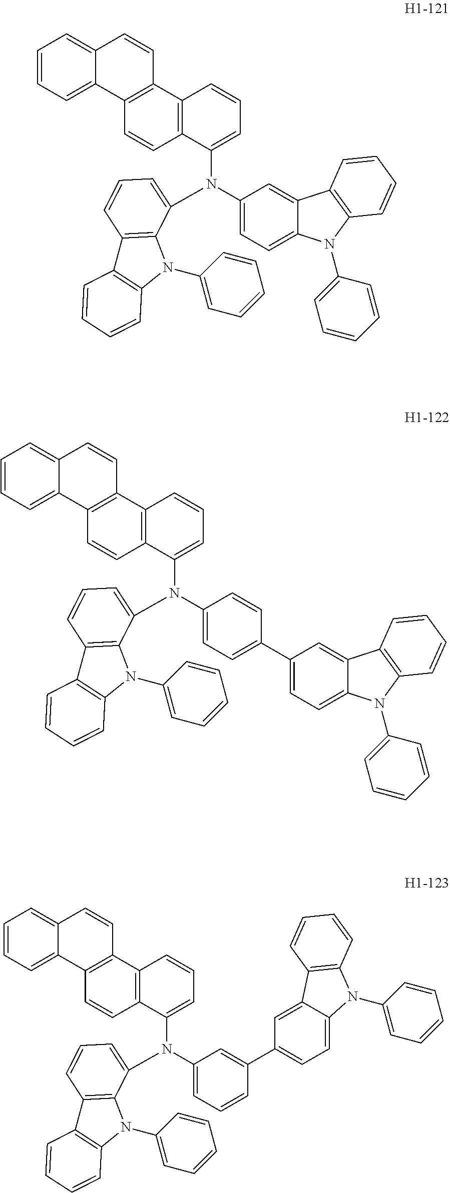 [SMILES: c1ccc(-n2c3ccccc3c3cc(N(c4cccc5c4ccc4c6ccccc6ccc54)c4cccc5c6ccccc6n(-c6ccccc6)c45)ccc32)cc1 ; c1ccc(-n2c3ccccc3c3cc(-c4ccc(N(c5cccc6c5ccc5c7ccccc7ccc65)c5cccc6c7ccccc7n(-c7ccccc7)c56)cc4)ccc32)cc1 ; c1ccc(-n2c3ccccc3c3cc(-c4cccc(N(c5cccc6c5ccc5c7ccccc7ccc65)c5cccc6c7ccccc7n(-c7ccccc7)c56)c4)ccc32)cc1]

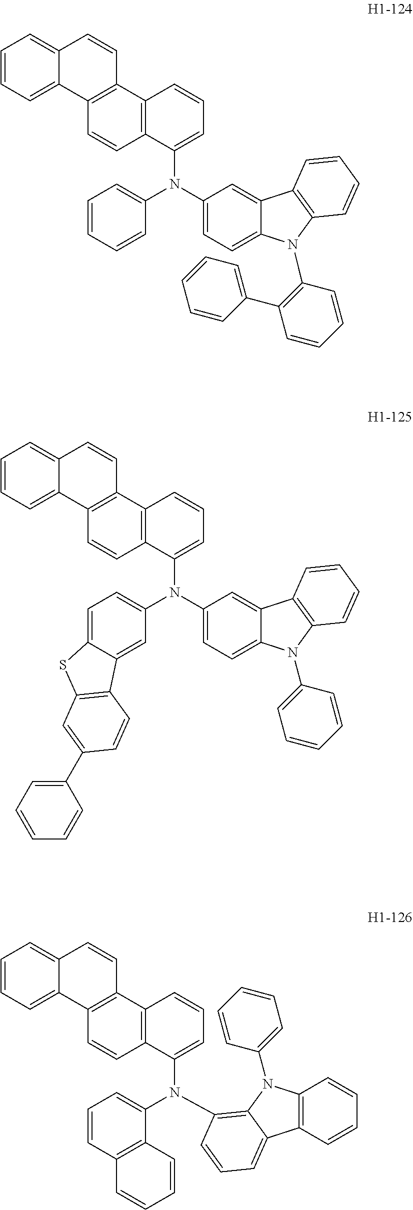 [SMILES: c1ccc(-c2ccccc2-n2c3ccccc3c3cc(N(c4ccccc4)c4cccc5c4ccc4c6ccccc6ccc54)ccc32)cc1 ; c1ccc(-c2ccc3c(c2)sc2ccc(N(c4ccc5c(c4)c4ccccc4n5-c4ccccc4)c4cccc5c4ccc4c6ccccc6ccc54)cc23)cc1 ; c1ccc(-n2c3ccccc3c3cccc(N(c4cccc5ccccc45)c4cccc5c4ccc4c6ccccc6ccc54)c32)cc1]

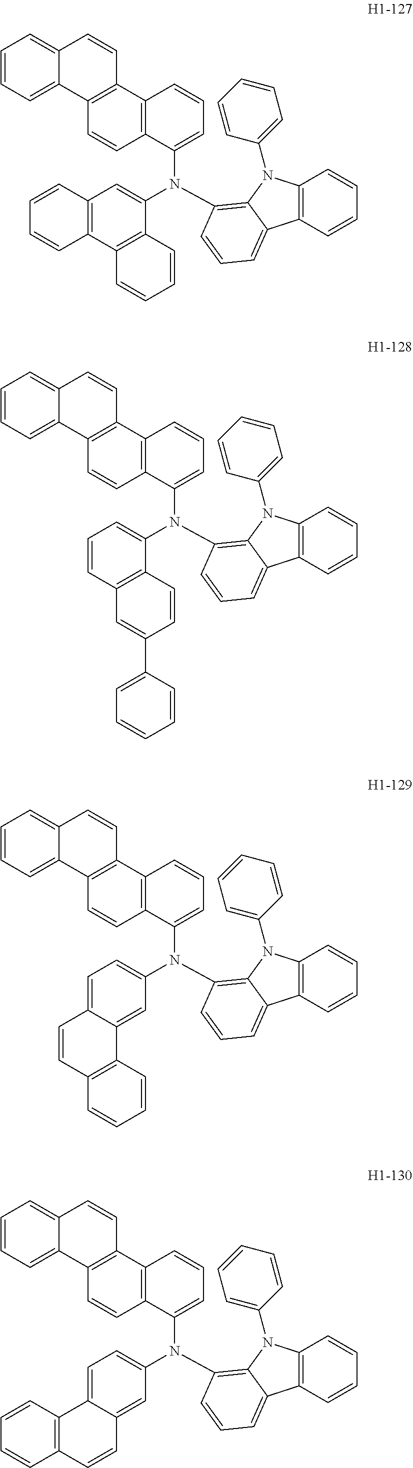 [SMILES: c1ccc(-n2c3ccccc3c3cccc(N(c4cc5ccccc5c5ccccc45)c4cccc5c4ccc4c6ccccc6ccc54)c32)cc1 ; c1ccc(-c2ccc3c(N(c4cccc5c4ccc4c6ccccc6ccc54)c4cccc5c6ccccc6n(-c6ccccc6)c45)cccc3c2)cc1 ; c1ccc(-n2c3ccccc3c3cccc(N(c4ccc5ccc6ccccc6c5c4)c4cccc5c4ccc4c6ccccc6ccc54)c32)cc1 ; c1ccc(-n2c3ccccc3c3cccc(N(c4ccc5c(ccc6ccccc65)c4)c4cccc5c4ccc4c6ccccc6ccc54)c32)cc1]

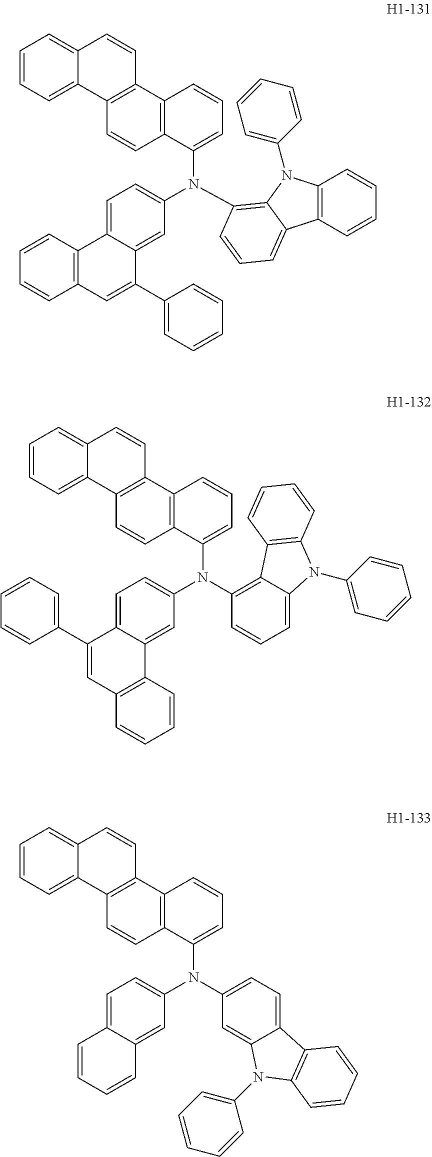 [SMILES: c1ccc(-c2cc3ccccc3c3ccc(N(c4cccc5c4ccc4c6ccccc6ccc54)c4cccc5c6ccccc6n(-c6ccccc6)c45)cc23)cc1 ; c1ccc(-c2cc3ccccc3c3cc(N(c4cccc5c4ccc4c6ccccc6ccc54)c4cccc5c4c4ccccc4n5-c4ccccc4)ccc23)cc1 ; c1ccc(-n2c3ccccc3c3ccc(N(c4ccc5ccccc5c4)c4cccc5c4ccc4c6ccccc6ccc54)cc32)cc1]

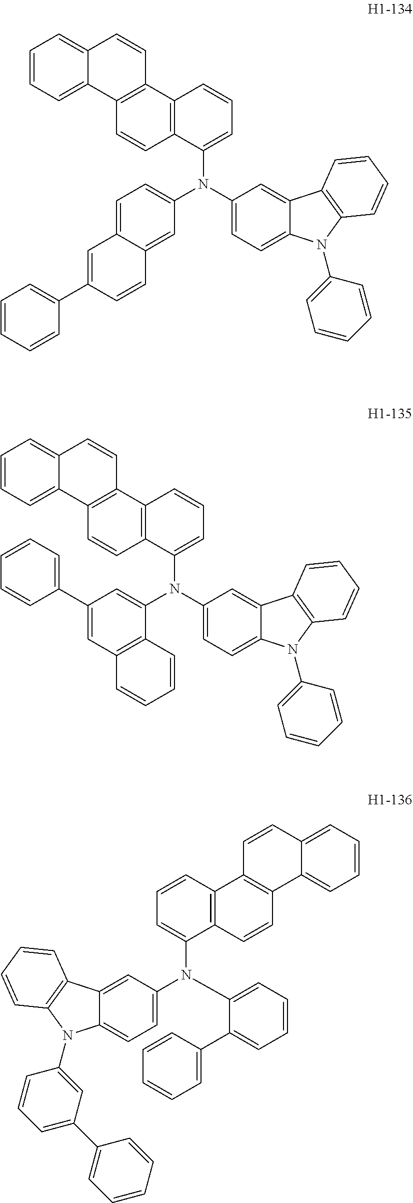 [SMILES: c1ccc(-c2ccc3cc(N(c4ccc5c(c4)c4ccccc4n5-c4ccccc4)c4cccc5c4ccc4c6ccccc6ccc54)ccc3c2)cc1 ; c1ccc(-c2cc(N(c3ccc4c(c3)c3ccccc3n4-c3ccccc3)c3cccc4c3ccc3c5ccccc5ccc43)c3ccccc3c2)cc1 ; c1ccc(-c2cccc(-n3c4ccccc4c4cc(N(c5ccccc5-c5ccccc5)c5cccc6c5ccc5c7ccccc7ccc65)ccc43)c2)cc1]

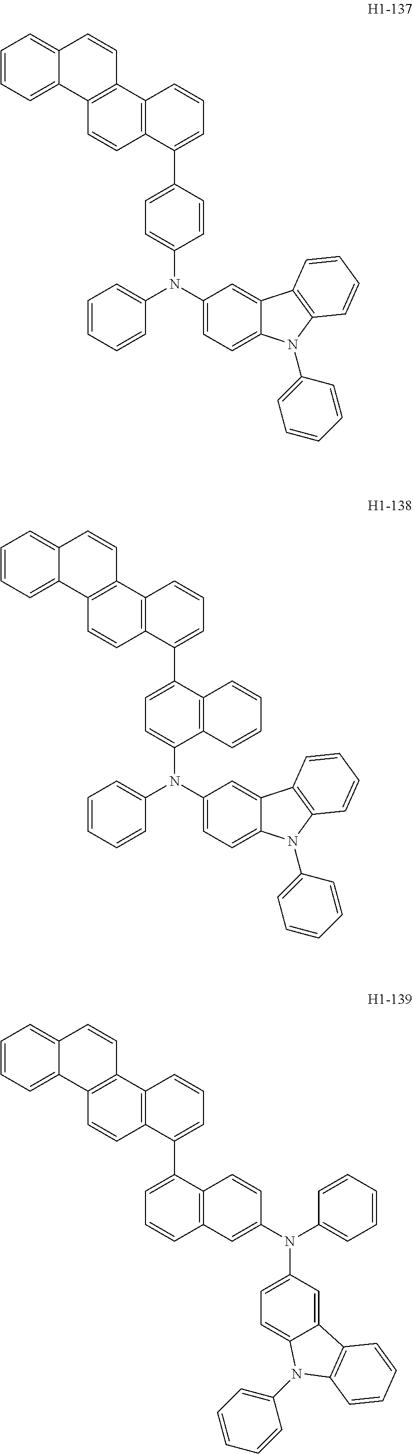 [SMILES: c1ccc(N(c2ccc(-c3cccc4c3ccc3c5ccccc5ccc43)cc2)c2ccc3c(c2)c2ccccc2n3-c2ccccc2)cc1 ; c1ccc(N(c2ccc3c(c2)c2ccccc2n3-c2ccccc2)c2ccc(-c3cccc4c3ccc3c5ccccc5ccc43)c3ccccc23)cc1 ; c1ccc(N(c2ccc3c(-c4cccc5c4ccc4c6ccccc6ccc54)cccc3c2)c2ccc3c(c2)c2ccccc2n3-c2ccccc2)cc1]

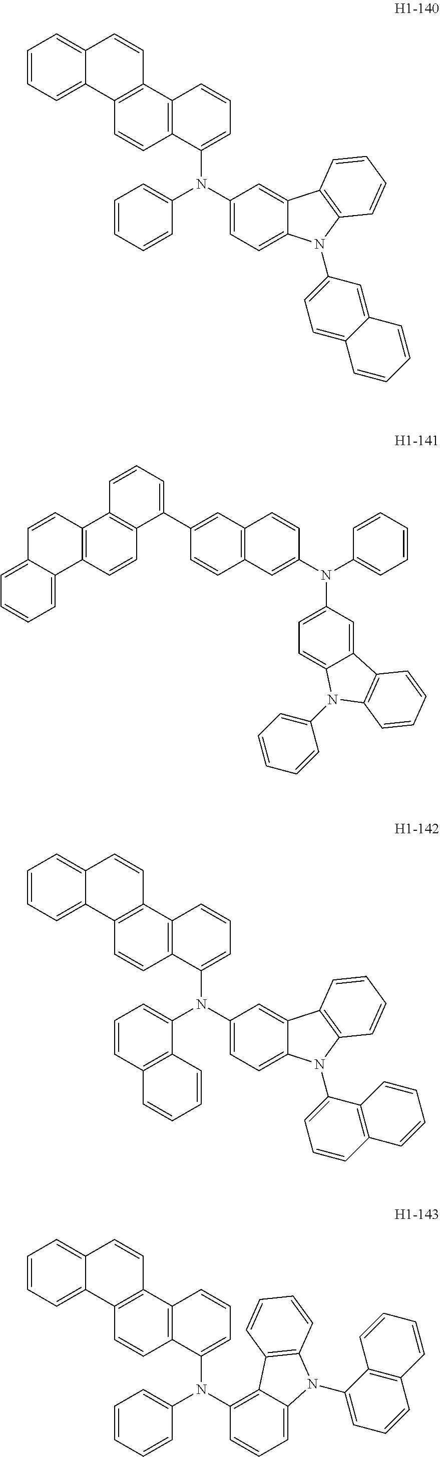 [SMILES: c1ccc(N(c2ccc3c(c2)c2ccccc2n3-c2ccc3ccccc3c2)c2cccc3c2ccc2c4ccccc4ccc32)cc1 ; c1ccc(N(c2ccc3cc(-c4cccc5c4ccc4c6ccccc6ccc54)ccc3c2)c2ccc3c(c2)c2ccccc2n3-c2ccccc2)cc1 ; c1ccc2c(N(c3ccc4c(c3)c3ccccc3n4-c3cccc4ccccc34)c3cccc4c3ccc3c5ccccc5ccc43)cccc2c1 ; c1ccc(N(c2cccc3c2ccc2c4ccccc4ccc32)c2cccc3c2c2ccccc2n3-c2cccc3ccccc23)cc1]

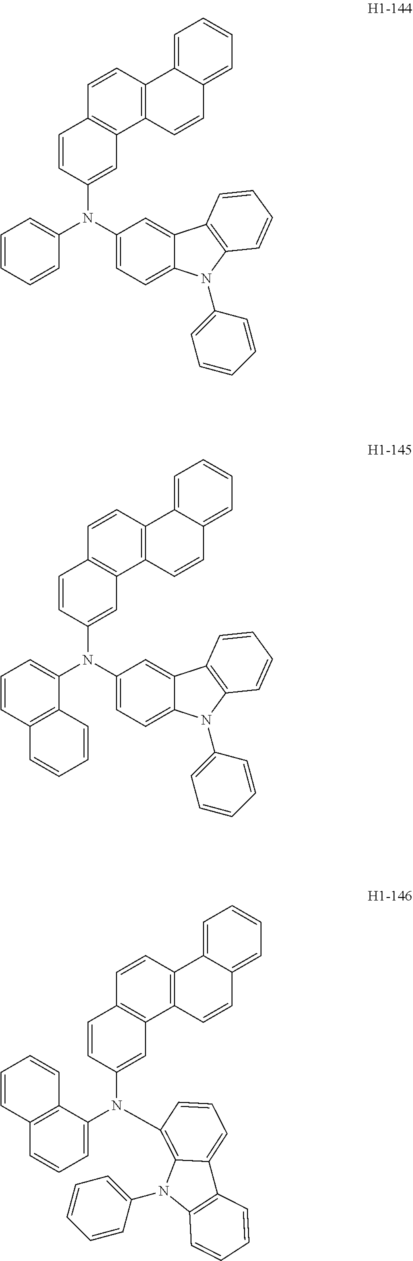 [SMILES: c1ccc(N(c2ccc3ccc4c5ccccc5ccc4c3c2)c2ccc3c(c2)c2ccccc2n3-c2ccccc2)cc1 ; c1ccc(-n2c3ccccc3c3cc(N(c4ccc5ccc6c7ccccc7ccc6c5c4)c4cccc5ccccc45)ccc32)cc1 ; c1ccc(-n2c3ccccc3c3cccc(N(c4ccc5ccc6c7ccccc7ccc6c5c4)c4cccc5ccccc45)c32)cc1]

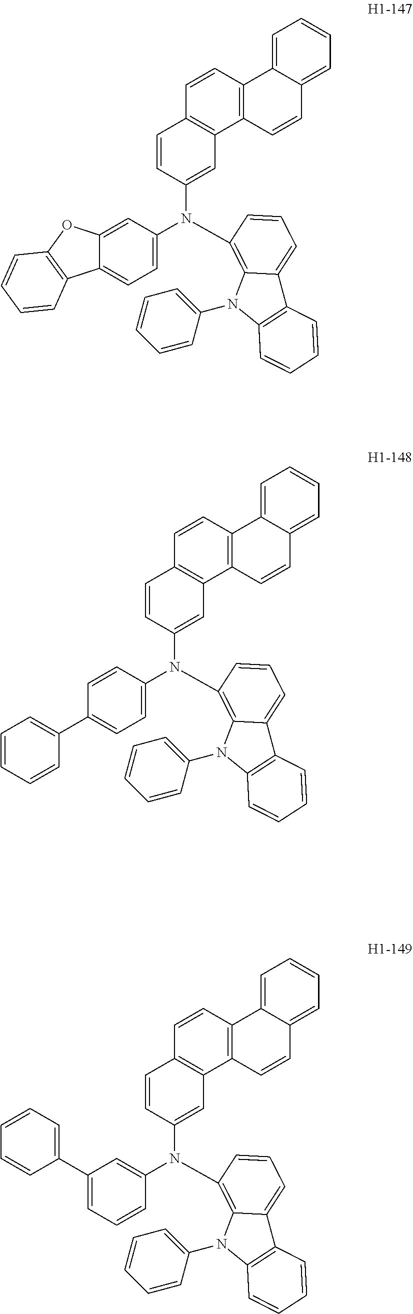 [SMILES: c1ccc(-n2c3ccccc3c3cccc(N(c4ccc5c(c4)oc4ccccc45)c4ccc5ccc6c7ccccc7ccc6c5c4)c32)cc1 ; c1ccc(-c2ccc(N(c3ccc4ccc5c6ccccc6ccc5c4c3)c3cccc4c5ccccc5n(-c5ccccc5)c34)cc2)cc1 ; c1ccc(-c2cccc(N(c3ccc4ccc5c6ccccc6ccc5c4c3)c3cccc4c5ccccc5n(-c5ccccc5)c34)c2)cc1]

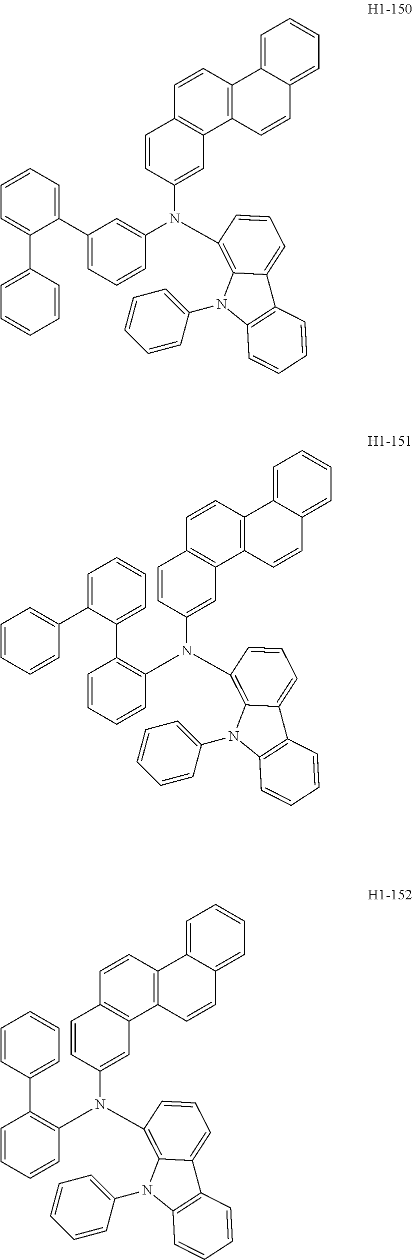 [SMILES: c1ccc(-c2ccccc2-c2cccc(N(c3ccc4ccc5c6ccccc6ccc5c4c3)c3cccc4c5ccccc5n(-c5ccccc5)c34)c2)cc1 ; c1ccc(-c2ccccc2-c2ccccc2N(c2ccc3ccc4c5ccccc5ccc4c3c2)c2cccc3c4ccccc4n(-c4ccccc4)c23)cc1 ; c1ccc(-c2ccccc2N(c2ccc3ccc4c5ccccc5ccc4c3c2)c2cccc3c4ccccc4n(-c4ccccc4)c23)cc1]

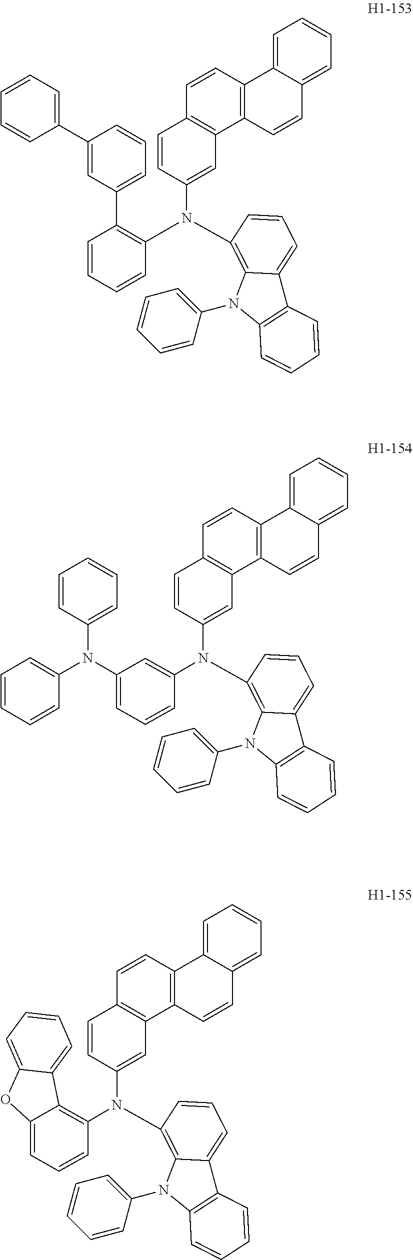 [SMILES: c1ccc(-c2cccc(-c3ccccc3N(c3ccc4ccc5c6ccccc6ccc5c4c3)c3cccc4c5ccccc5n(-c5ccccc5)c34)c2)cc1 ; c1ccc(N(c2ccccc2)c2cccc(N(c3ccc4ccc5c6ccccc6ccc5c4c3)c3cccc4c5ccccc5n(-c5ccccc5)c34)c2)cc1 ; c1ccc(-n2c3ccccc3c3cccc(N(c4ccc5ccc6c7ccccc7ccc6c5c4)c4cccc5oc6ccccc6c45)c32)cc1]

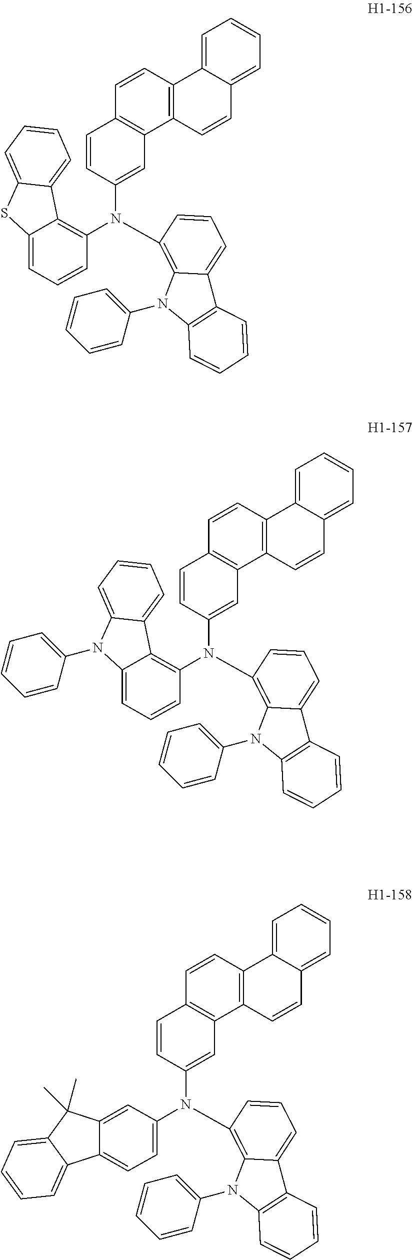 [SMILES: c1ccc(-n2c3ccccc3c3cccc(N(c4ccc5ccc6c7ccccc7ccc6c5c4)c4cccc5sc6ccccc6c45)c32)cc1 ; c1ccc(-n2c3ccccc3c3c(N(c4ccc5ccc6c7ccccc7ccc6c5c4)c4cccc5c6ccccc6n(-c6ccccc6)c45)cccc32)cc1 ; CC1(C)c2ccccc2-c2ccc(N(c3ccc4ccc5c6ccccc6ccc5c4c3)c3cccc4c5ccccc5n(-c5ccccc5)c34)cc21]

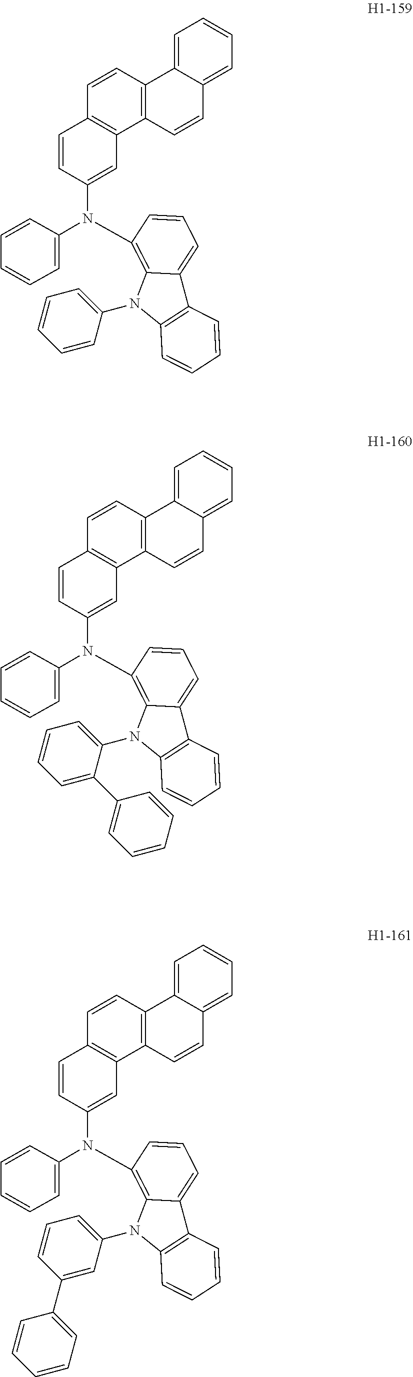 [SMILES: c1ccc(N(c2ccc3ccc4c5ccccc5ccc4c3c2)c2cccc3c4ccccc4n(-c4ccccc4)c23)cc1 ; c1ccc(-c2ccccc2-n2c3ccccc3c3cccc(N(c4ccccc4)c4ccc5ccc6c7ccccc7ccc6c5c4)c32)cc1 ; c1ccc(-c2cccc(-n3c4ccccc4c4cccc(N(c5ccccc5)c5ccc6ccc7c8ccccc8ccc7c6c5)c43)c2)cc1]

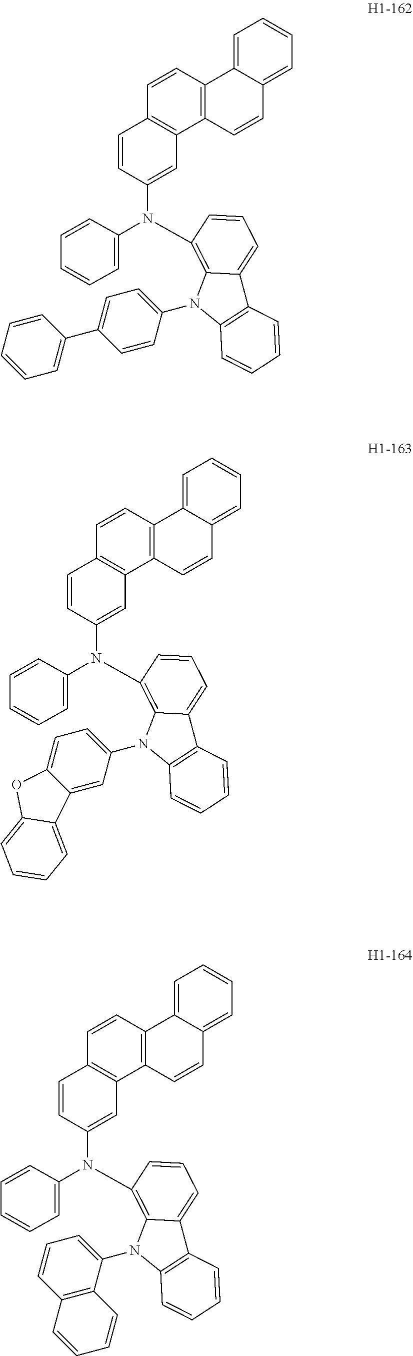 [SMILES: c1ccc(-c2ccc(-n3c4ccccc4c4cccc(N(c5ccccc5)c5ccc6ccc7c8ccccc8ccc7c6c5)c43)cc2)cc1 ; c1ccc(N(c2ccc3ccc4c5ccccc5ccc4c3c2)c2cccc3c4ccccc4n(-c4ccc5oc6ccccc6c5c4)c23)cc1 ; c1ccc(N(c2ccc3ccc4c5ccccc5ccc4c3c2)c2cccc3c4ccccc4n(-c4cccc5ccccc45)c23)cc1]

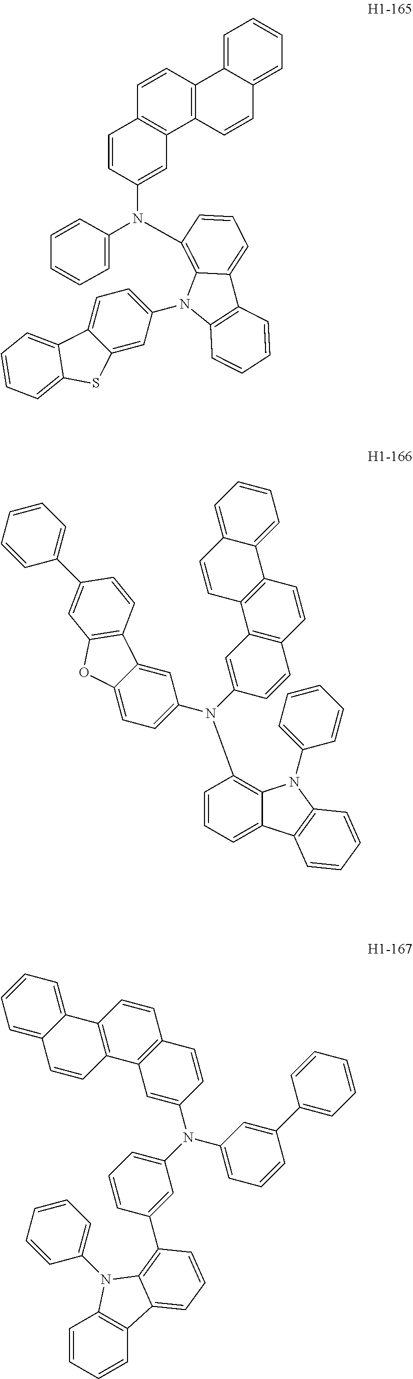 [SMILES: c1ccc(N(c2ccc3ccc4c5ccccc5ccc4c3c2)c2cccc3c4ccccc4n(-c4ccc5c(c4)sc4ccccc45)c23)cc1 ; c1ccc(-c2ccc3c(c2)oc2ccc(N(c4ccc5ccc6c7ccccc7ccc6c5c4)c4cccc5c6ccccc6n(-c6ccccc6)c45)cc23)cc1 ; c1ccc(-c2cccc(N(c3cccc(-c4cccc5c6ccccc6n(-c6ccccc6)c45)c3)c3ccc4ccc5c6ccccc6ccc5c4c3)c2)cc1]

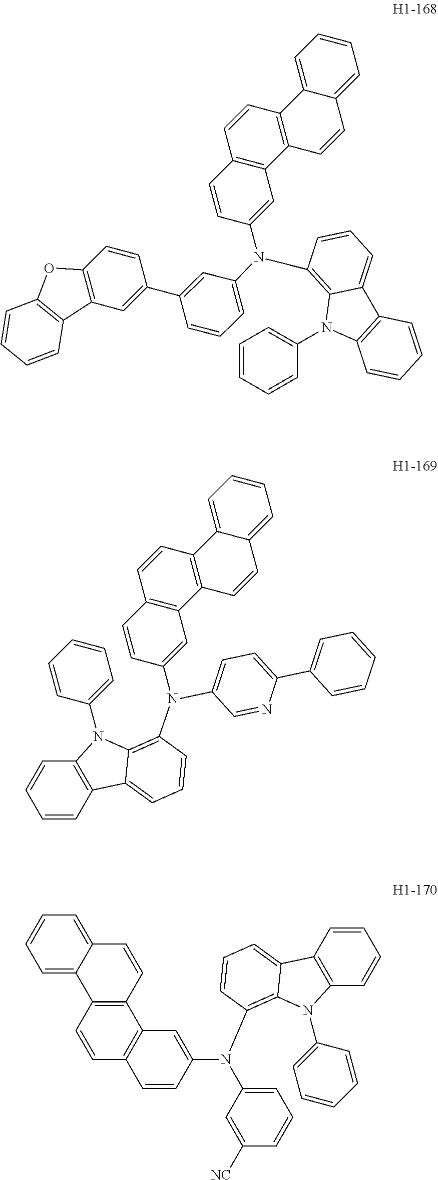 [SMILES: c1ccc(-n2c3ccccc3c3cccc(N(c4cccc(-c5ccc6oc7ccccc7c6c5)c4)c4ccc5ccc6c7ccccc7ccc6c5c4)c32)cc1 ; c1ccc(-c2ccc(N(c3ccc4ccc5c6ccccc6ccc5c4c3)c3cccc4c5ccccc5n(-c5ccccc5)c34)cn2)cc1 ; N#Cc1cccc(N(c2ccc3ccc4c5ccccc5ccc4c3c2)c2cccc3c4ccccc4n(-c4ccccc4)c23)c1]

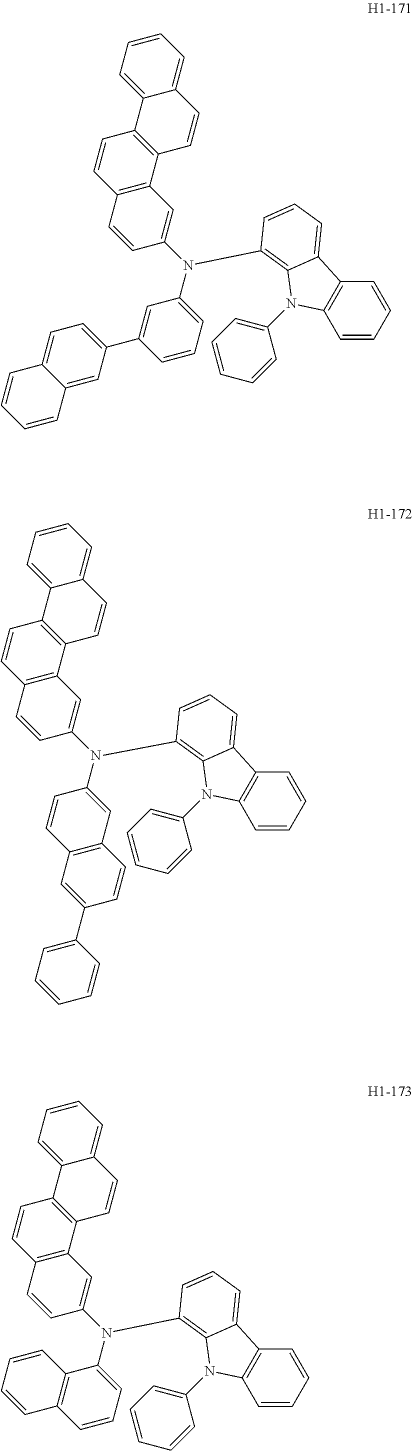 [SMILES: c1ccc(-n2c3ccccc3c3cccc(N(c4cccc(-c5ccc6ccccc6c5)c4)c4ccc5ccc6c7ccccc7ccc6c5c4)c32)cc1 ; c1ccc(-c2ccc3cc(N(c4ccc5ccc6c7ccccc7ccc6c5c4)c4cccc5c6ccccc6n(-c6ccccc6)c45)ccc3c2)cc1 ; c1ccc(-n2c3ccccc3c3cccc(N(c4ccc5ccc6c7ccccc7ccc6c5c4)c4cccc5ccccc45)c32)cc1]

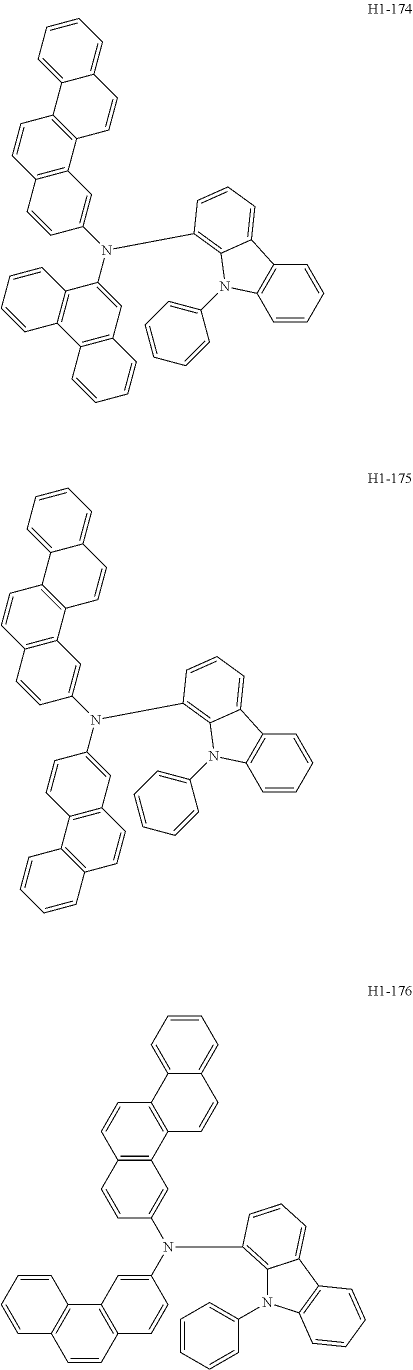 [SMILES: c1ccc(-n2c3ccccc3c3cccc(N(c4ccc5ccc6c7ccccc7ccc6c5c4)c4cc5ccccc5c5ccccc45)c32)cc1 ; c1ccc(-n2c3ccccc3c3cccc(N(c4ccc5c(ccc6ccccc65)c4)c4ccc5ccc6c7ccccc7ccc6c5c4)c32)cc1 ; c1ccc(-n2c3ccccc3c3cccc(N(c4ccc5ccc6ccccc6c5c4)c4ccc5ccc6c7ccccc7ccc6c5c4)c32)cc1]

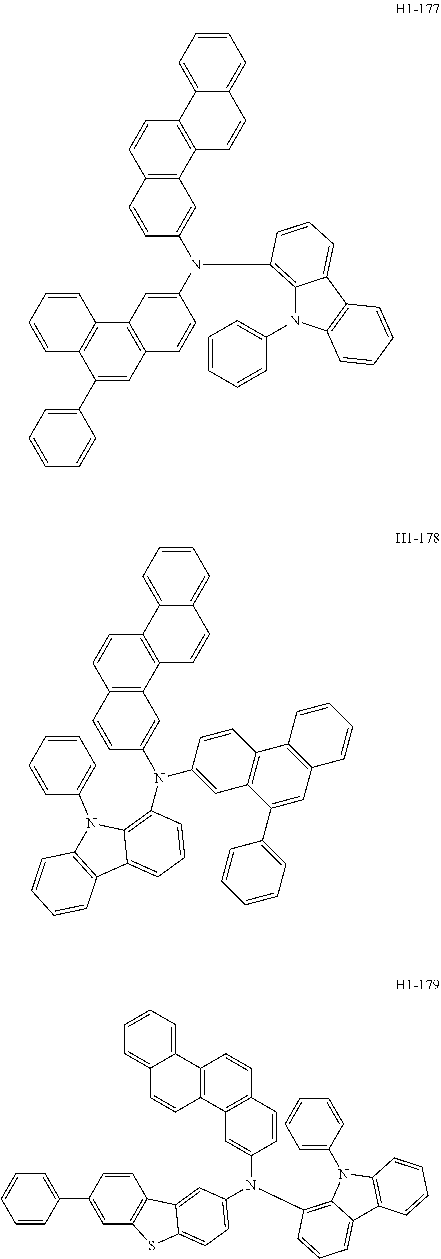 [SMILES: c1ccc(-c2cc3ccc(N(c4ccc5ccc6c7ccccc7ccc6c5c4)c4cccc5c6ccccc6n(-c6ccccc6)c45)cc3c3ccccc23)cc1 ; c1ccc(-c2cc3ccccc3c3ccc(N(c4ccc5ccc6c7ccccc7ccc6c5c4)c4cccc5c6ccccc6n(-c6ccccc6)c45)cc23)cc1 ; c1ccc(-c2ccc3c(c2)sc2ccc(N(c4ccc5ccc6c7ccccc7ccc6c5c4)c4cccc5c6ccccc6n(-c6ccccc6)c45)cc23)cc1]

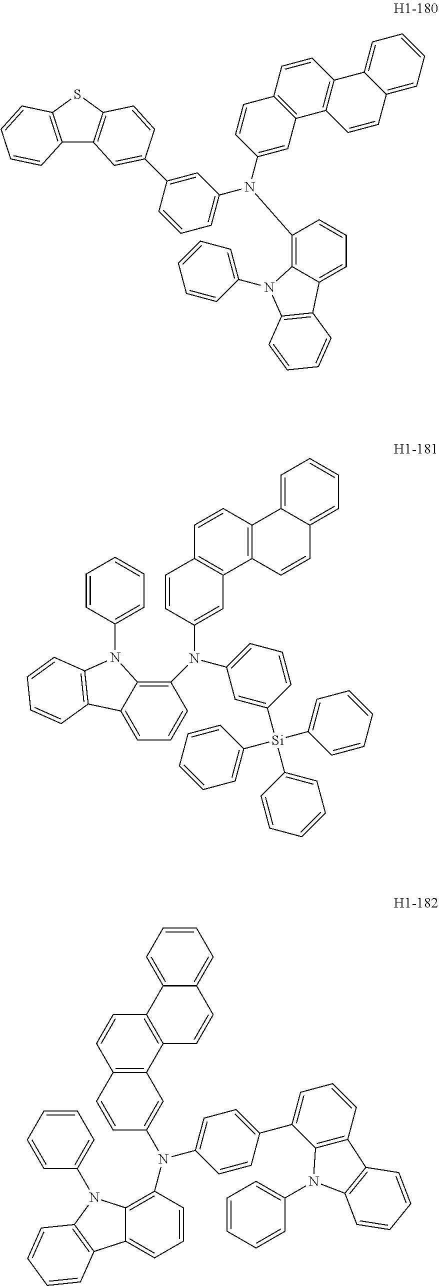 [SMILES: c1ccc(-n2c3ccccc3c3cccc(N(c4cccc(-c5ccc6sc7ccccc7c6c5)c4)c4ccc5ccc6c7ccccc7ccc6c5c4)c32)cc1 ; c1ccc(-n2c3ccccc3c3cccc(N(c4cccc([Si](c5ccccc5)(c5ccccc5)c5ccccc5)c4)c4ccc5ccc6c7ccccc7ccc6c5c4)c32)cc1 ; c1ccc(-n2c3ccccc3c3cccc(-c4ccc(N(c5ccc6ccc7c8ccccc8ccc7c6c5)c5cccc6c7ccccc7n(-c7ccccc7)c56)cc4)c32)cc1]

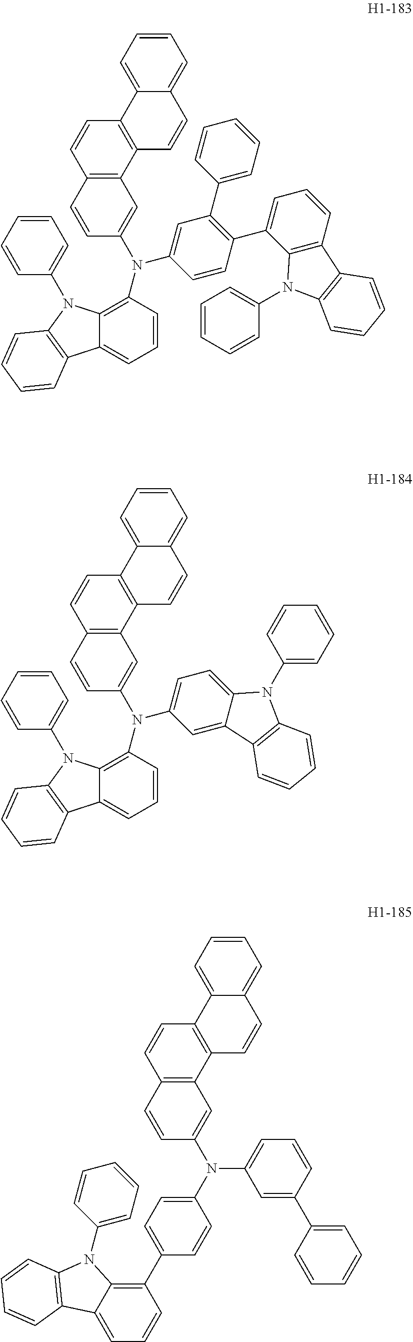 [SMILES: c1ccc(-c2cc(N(c3ccc4ccc5c6ccccc6ccc5c4c3)c3cccc4c5ccccc5n(-c5ccccc5)c34)ccc2-c2cccc3c4ccccc4n(-c4ccccc4)c23)cc1 ; c1ccc(-n2c3ccccc3c3cc(N(c4ccc5ccc6c7ccccc7ccc6c5c4)c4cccc5c6ccccc6n(-c6ccccc6)c45)ccc32)cc1 ; c1ccc(-c2cccc(N(c3ccc(-c4cccc5c6ccccc6n(-c6ccccc6)c45)cc3)c3ccc4ccc5c6ccccc6ccc5c4c3)c2)cc1]

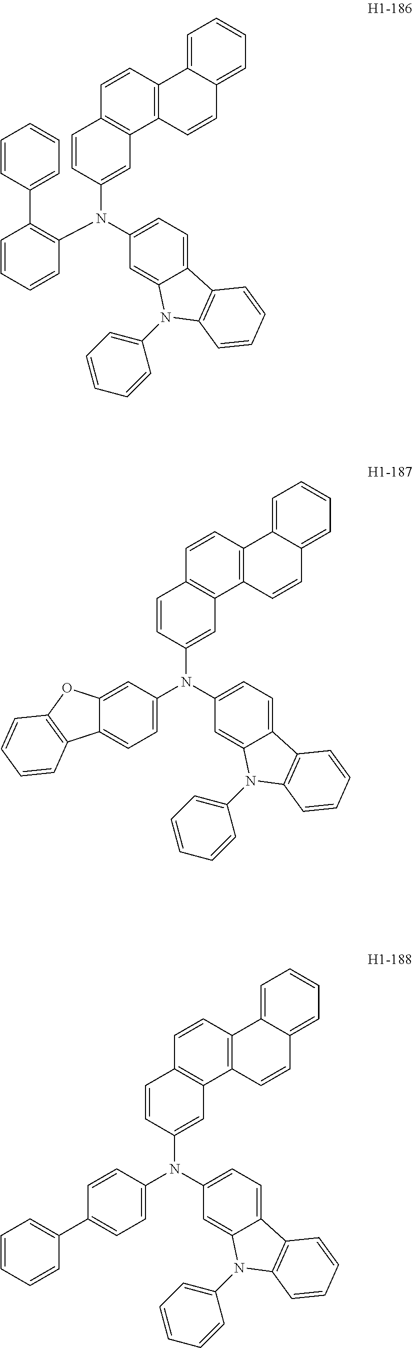 [SMILES: c1ccc(-c2ccccc2N(c2ccc3ccc4c5ccccc5ccc4c3c2)c2ccc3c4ccccc4n(-c4ccccc4)c3c2)cc1 ; c1ccc(-n2c3ccccc3c3ccc(N(c4ccc5c(c4)oc4ccccc45)c4ccc5ccc6c7ccccc7ccc6c5c4)cc32)cc1 ; c1ccc(-c2ccc(N(c3ccc4ccc5c6ccccc6ccc5c4c3)c3ccc4c5ccccc5n(-c5ccccc5)c4c3)cc2)cc1]

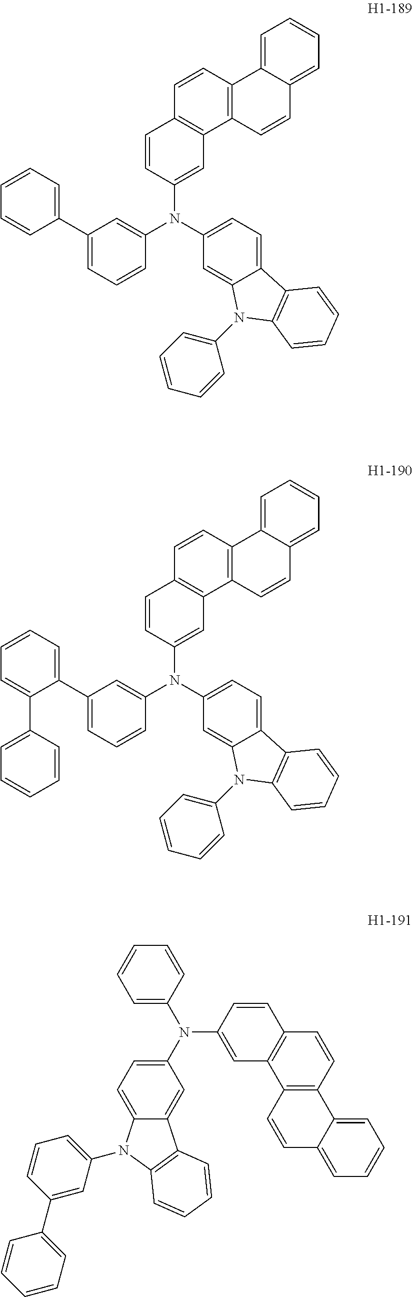 [SMILES: c1ccc(-c2cccc(N(c3ccc4ccc5c6ccccc6ccc5c4c3)c3ccc4c5ccccc5n(-c5ccccc5)c4c3)c2)cc1 ; c1ccc(-c2ccccc2-c2cccc(N(c3ccc4ccc5c6ccccc6ccc5c4c3)c3ccc4c5ccccc5n(-c5ccccc5)c4c3)c2)cc1 ; c1ccc(-c2cccc(-n3c4ccccc4c4cc(N(c5ccccc5)c5ccc6ccc7c8ccccc8ccc7c6c5)ccc43)c2)cc1]

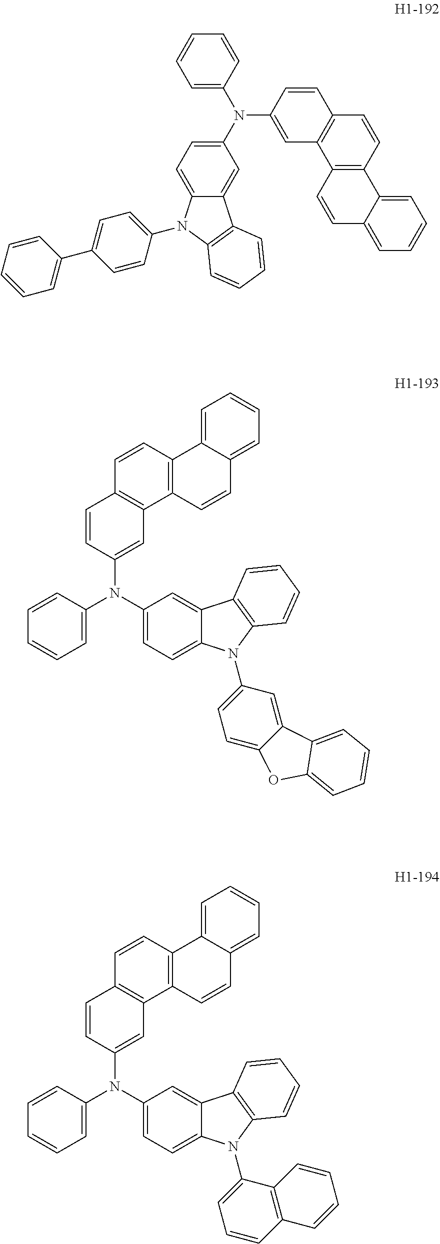 [SMILES: c1ccc(-c2ccc(-n3c4ccccc4c4cc(N(c5ccccc5)c5ccc6ccc7c8ccccc8ccc7c6c5)ccc43)cc2)cc1 ; c1ccc(N(c2ccc3ccc4c5ccccc5ccc4c3c2)c2ccc3c(c2)c2ccccc2n3-c2ccc3oc4ccccc4c3c2)cc1 ; c1ccc(N(c2ccc3ccc4c5ccccc5ccc4c3c2)c2ccc3c(c2)c2ccccc2n3-c2cccc3ccccc23)cc1]

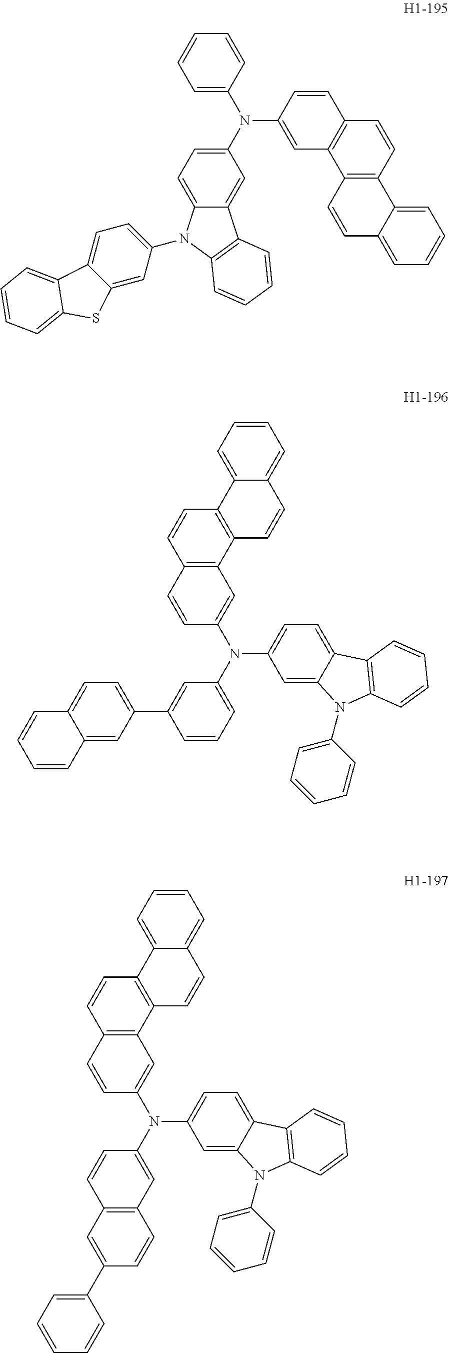 [SMILES: c1ccc(N(c2ccc3ccc4c5ccccc5ccc4c3c2)c2ccc3c(c2)c2ccccc2n3-c2ccc3c(c2)sc2ccccc23)cc1 ; c1ccc(-n2c3ccccc3c3ccc(N(c4cccc(-c5ccc6ccccc6c5)c4)c4ccc5ccc6c7ccccc7ccc6c5c4)cc32)cc1 ; c1ccc(-c2ccc3cc(N(c4ccc5ccc6c7ccccc7ccc6c5c4)c4ccc5c6ccccc6n(-c6ccccc6)c5c4)ccc3c2)cc1]

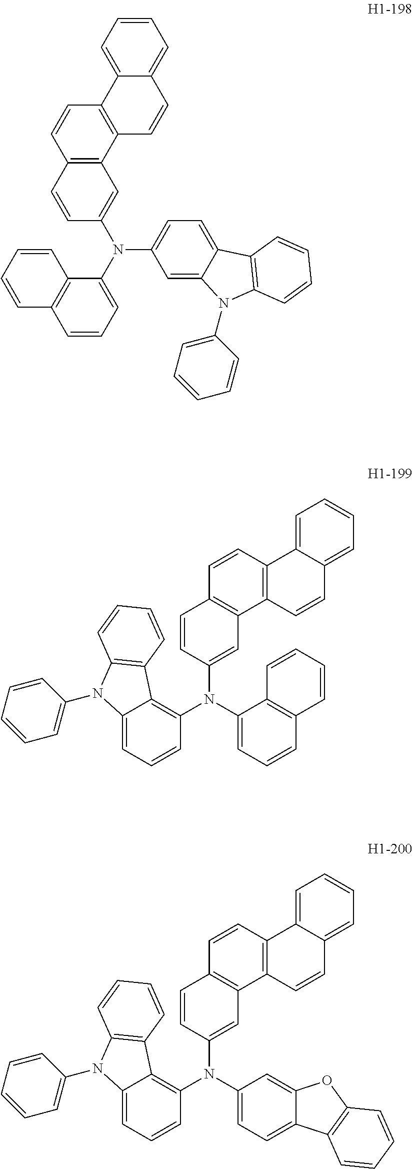 [SMILES: c1ccc(-n2c3ccccc3c3ccc(N(c4ccc5ccc6c7ccccc7ccc6c5c4)c4cccc5ccccc45)cc32)cc1 ; c1ccc(-n2c3ccccc3c3c(N(c4ccc5ccc6c7ccccc7ccc6c5c4)c4cccc5ccccc45)cccc32)cc1 ; c1ccc(-n2c3ccccc3c3c(N(c4ccc5c(c4)oc4ccccc45)c4ccc5ccc6c7ccccc7ccc6c5c4)cccc32)cc1]

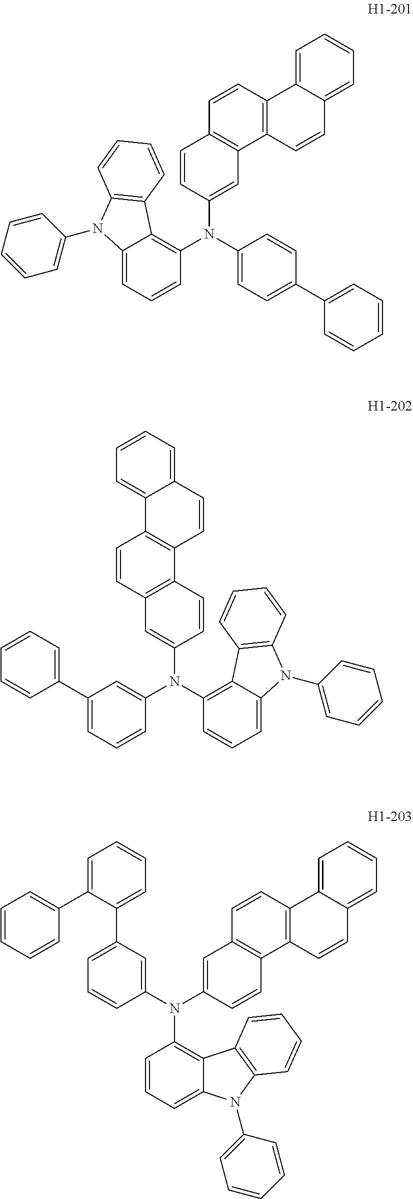 [SMILES: c1ccc(-c2ccc(N(c3ccc4ccc5c6ccccc6ccc5c4c3)c3cccc4c3c3ccccc3n4-c3ccccc3)cc2)cc1 ; c1ccc(-c2cccc(N(c3ccc4c(ccc5c6ccccc6ccc45)c3)c3cccc4c3c3ccccc3n4-c3ccccc3)c2)cc1 ; c1ccc(-c2ccccc2-c2cccc(N(c3ccc4c(ccc5c6ccccc6ccc45)c3)c3cccc4c3c3ccccc3n4-c3ccccc3)c2)cc1]

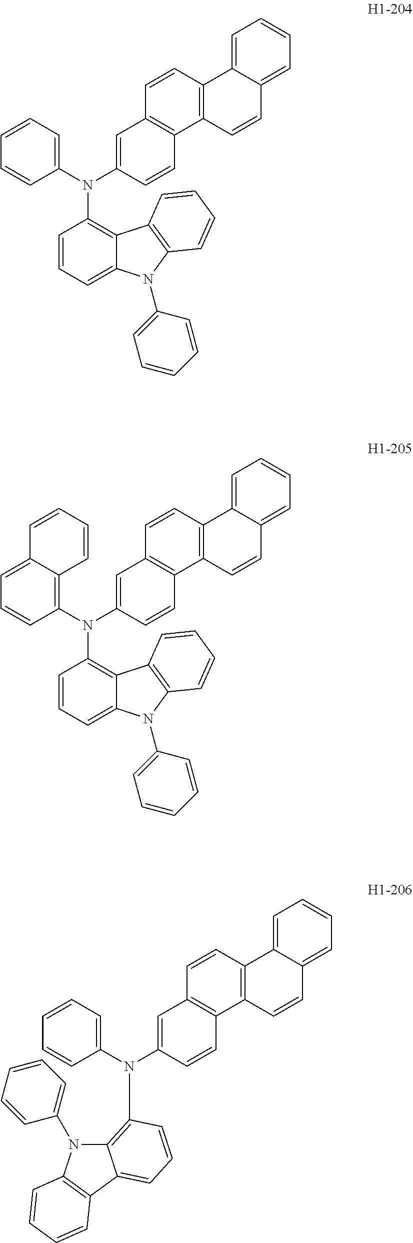 [SMILES: c1ccc(N(c2ccc3c(ccc4c5ccccc5ccc34)c2)c2cccc3c2c2ccccc2n3-c2ccccc2)cc1 ; c1ccc(-n2c3ccccc3c3c(N(c4ccc5c(ccc6c7ccccc7ccc56)c4)c4cccc5ccccc45)cccc32)cc1 ; c1ccc(N(c2ccc3c(ccc4c5ccccc5ccc34)c2)c2cccc3c4ccccc4n(-c4ccccc4)c23)cc1]

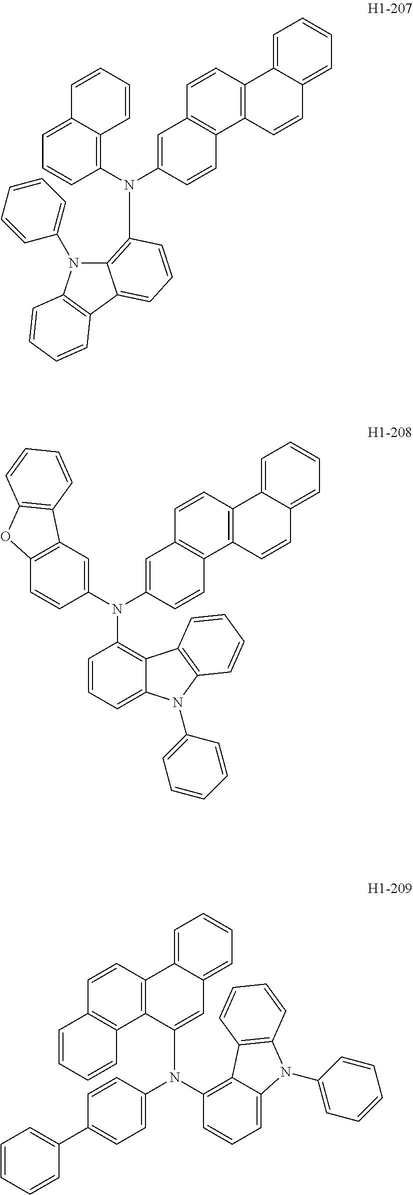 [SMILES: c1ccc(-n2c3ccccc3c3cccc(N(c4ccc5c(ccc6c7ccccc7ccc56)c4)c4cccc5ccccc45)c32)cc1 ; c1ccc(-n2c3ccccc3c3c(N(c4ccc5c(ccc6c7ccccc7ccc56)c4)c4ccc5oc6ccccc6c5c4)cccc32)cc1 ; c1ccc(-c2ccc(N(c3cc4ccccc4c4ccc5ccccc5c34)c3cccc4c3c3ccccc3n4-c3ccccc3)cc2)cc1]

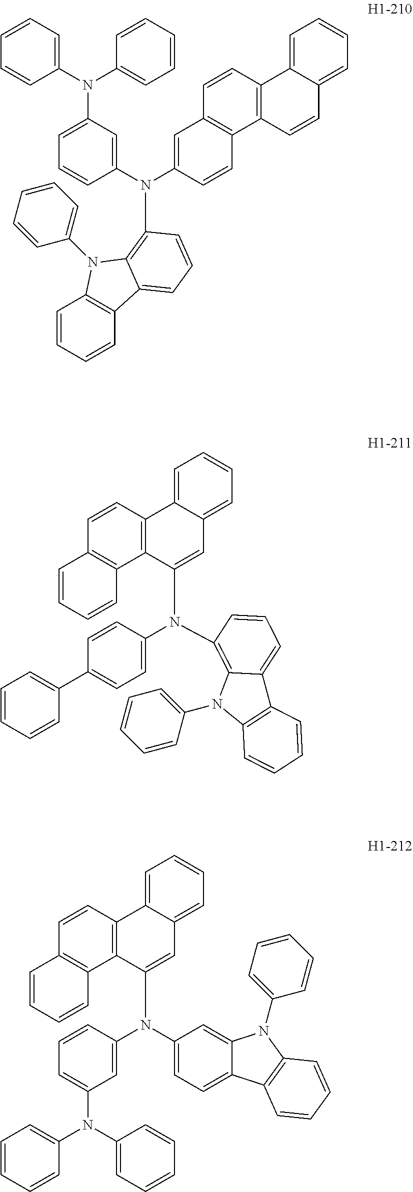 [SMILES: c1ccc(N(c2ccccc2)c2cccc(N(c3ccc4c(ccc5c6ccccc6ccc45)c3)c3cccc4c5ccccc5n(-c5ccccc5)c34)c2)cc1 ; c1ccc(-c2ccc(N(c3cc4ccccc4c4ccc5ccccc5c34)c3cccc4c5ccccc5n(-c5ccccc5)c34)cc2)cc1 ; c1ccc(N(c2ccccc2)c2cccc(N(c3ccc4c5ccccc5n(-c5ccccc5)c4c3)c3cc4ccccc4c4ccc5ccccc5c34)c2)cc1]

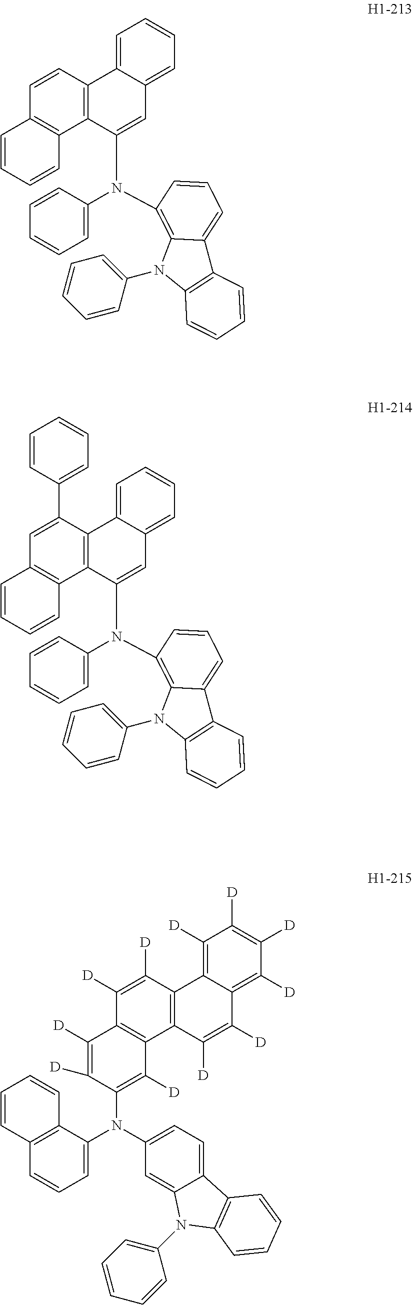 [SMILES: c1ccc(N(c2cc3ccccc3c3ccc4ccccc4c23)c2cccc3c4ccccc4n(-c4ccccc4)c23)cc1 ; c1ccc(-c2cc3ccccc3c3c(N(c4ccccc4)c4cccc5c6ccccc6n(-c6ccccc6)c45)cc4ccccc4c23)cc1 ; [2H]c1c([2H])c([2H])c2c(c1[2H])c([2H])c([2H])c1c3c([2H])c(N(c4ccc5c6ccccc6n(-c6ccccc6)c5c4)c4cccc5ccccc45)c([2H])c([2H])c3c([2H])c([2H])c21]

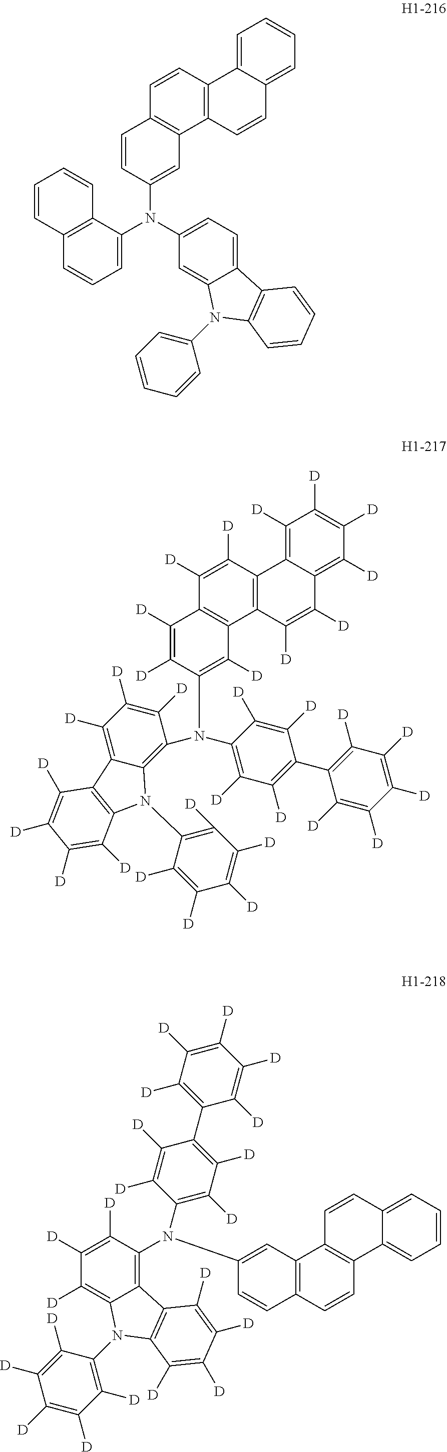

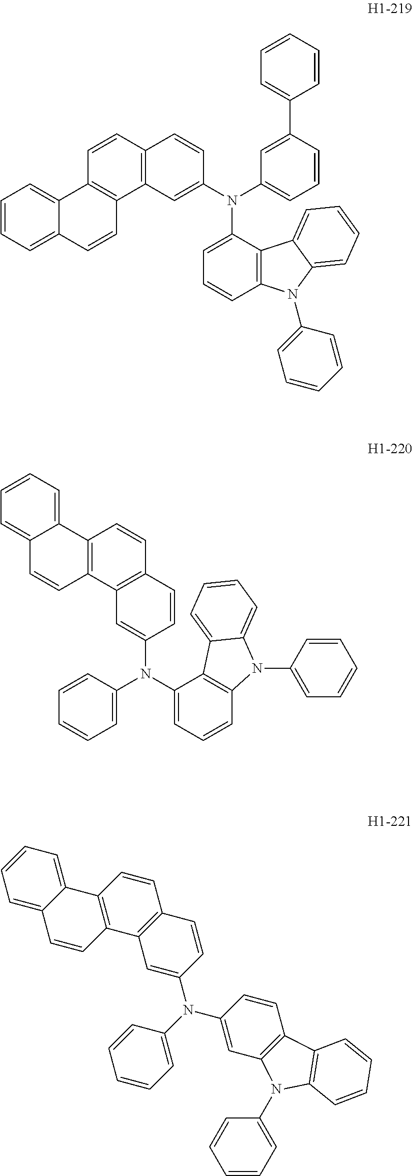 [SMILES: c1ccc(-c2cccc(N(c3ccc4ccc5c6ccccc6ccc5c4c3)c3cccc4c3c3ccccc3n4-c3ccccc3)c2)cc1 ; c1ccc(N(c2ccc3ccc4c5ccccc5ccc4c3c2)c2cccc3c2c2ccccc2n3-c2ccccc2)cc1 ; c1ccc(N(c2ccc3ccc4c5ccccc5ccc4c3c2)c2ccc3c4ccccc4n(-c4ccccc4)c3c2)cc1]

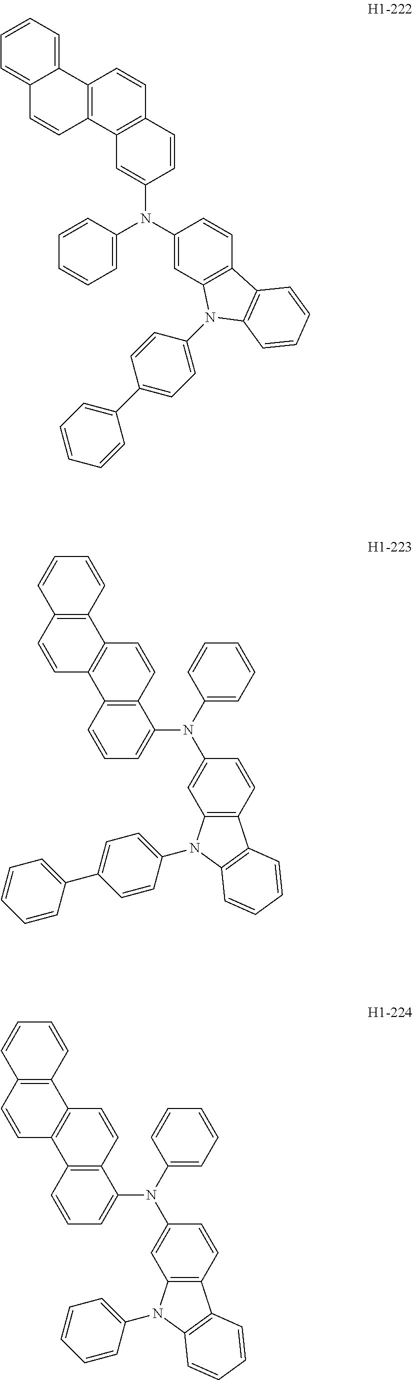 [SMILES: c1ccc(-c2ccc(-n3c4ccccc4c4ccc(N(c5ccccc5)c5ccc6ccc7c8ccccc8ccc7c6c5)cc43)cc2)cc1 ; c1ccc(-c2ccc(-n3c4ccccc4c4ccc(N(c5ccccc5)c5cccc6c5ccc5c7ccccc7ccc65)cc43)cc2)cc1 ; c1ccc(N(c2ccc3c4ccccc4n(-c4ccccc4)c3c2)c2cccc3c2ccc2c4ccccc4ccc32)cc1]

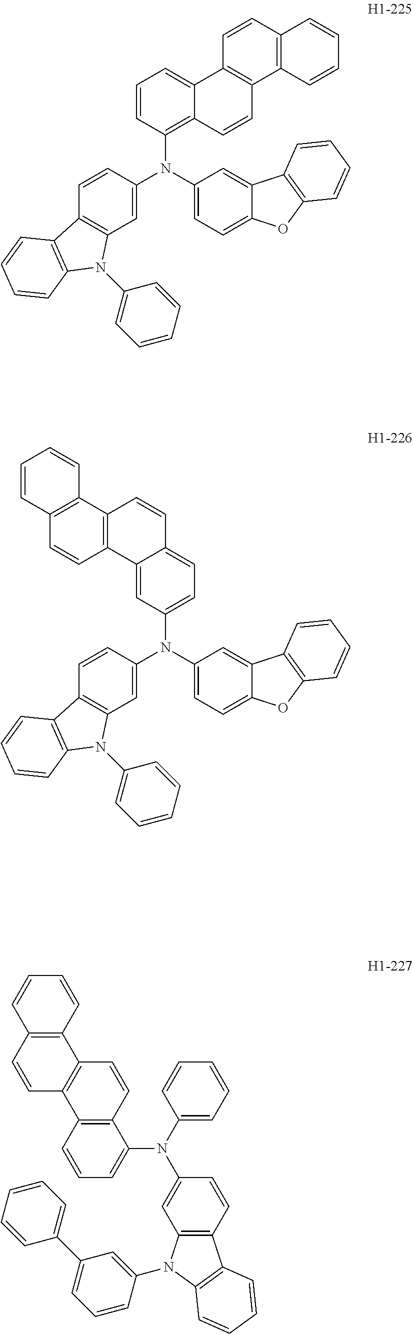 [SMILES: c1ccc(-n2c3ccccc3c3ccc(N(c4ccc5oc6ccccc6c5c4)c4cccc5c4ccc4c6ccccc6ccc54)cc32)cc1 ; c1ccc(-n2c3ccccc3c3ccc(N(c4ccc5ccc6c7ccccc7ccc6c5c4)c4ccc5oc6ccccc6c5c4)cc32)cc1 ; c1ccc(-c2cccc(-n3c4ccccc4c4ccc(N(c5ccccc5)c5cccc6c5ccc5c7ccccc7ccc65)cc43)c2)cc1]

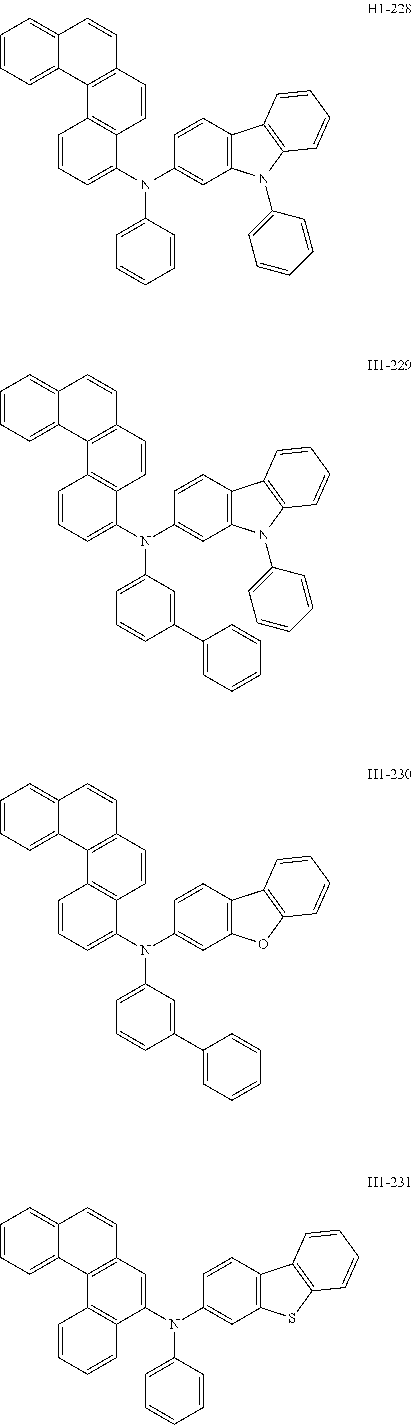 [SMILES: c1ccc(N(c2ccc3c4ccccc4n(-c4ccccc4)c3c2)c2cccc3c2ccc2ccc4ccccc4c23)cc1 ; c1ccc(-c2cccc(N(c3ccc4c5ccccc5n(-c5ccccc5)c4c3)c3cccc4c3ccc3ccc5ccccc5c34)c2)cc1 ; c1ccc(-c2cccc(N(c3ccc4c(c3)oc3ccccc34)c3cccc4c3ccc3ccc5ccccc5c34)c2)cc1 ; c1ccc(N(c2ccc3c(c2)sc2ccccc23)c2cc3ccc4ccccc4c3c3ccccc23)cc1]

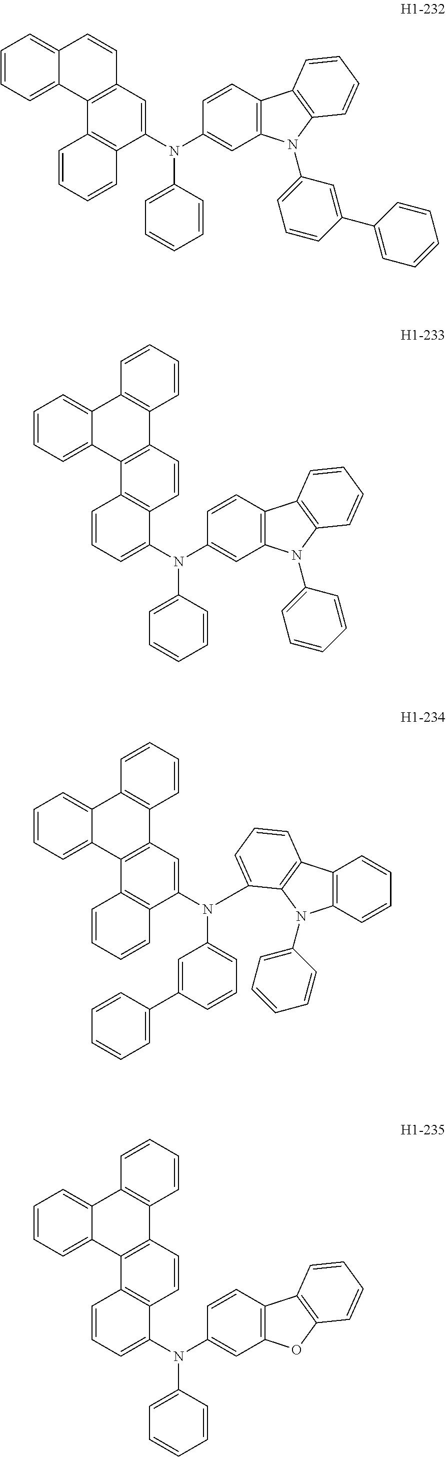 [SMILES: c1ccc(-c2cccc(-n3c4ccccc4c4ccc(N(c5ccccc5)c5cc6ccc7ccccc7c6c6ccccc56)cc43)c2)cc1 ; c1ccc(N(c2ccc3c4ccccc4n(-c4ccccc4)c3c2)c2cccc3c2ccc2c4ccccc4c4ccccc4c32)cc1 ; c1ccc(-c2cccc(N(c3cc4c5ccccc5c5ccccc5c4c4ccccc34)c3cccc4c5ccccc5n(-c5ccccc5)c34)c2)cc1 ; c1ccc(N(c2ccc3c(c2)oc2ccccc23)c2cccc3c2ccc2c4ccccc4c4ccccc4c32)cc1]

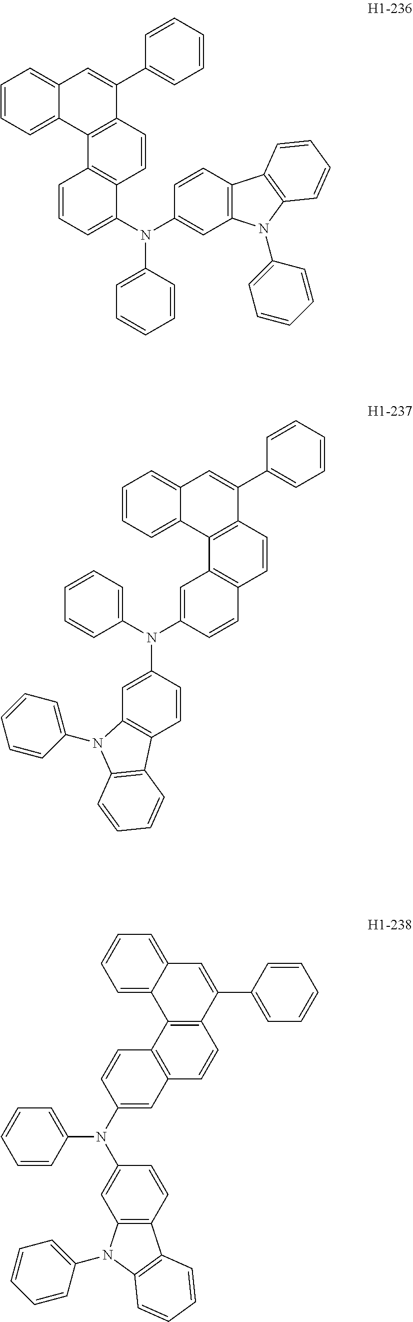 [SMILES: c1ccc(-c2cc3ccccc3c3c2ccc2c(N(c4ccccc4)c4ccc5c6ccccc6n(-c6ccccc6)c5c4)cccc23)cc1 ; c1ccc(-c2cc3ccccc3c3c2ccc2ccc(N(c4ccccc4)c4ccc5c6ccccc6n(-c6ccccc6)c5c4)cc23)cc1 ; c1ccc(-c2cc3ccccc3c3c2ccc2cc(N(c4ccccc4)c4ccc5c6ccccc6n(-c6ccccc6)c5c4)ccc23)cc1]

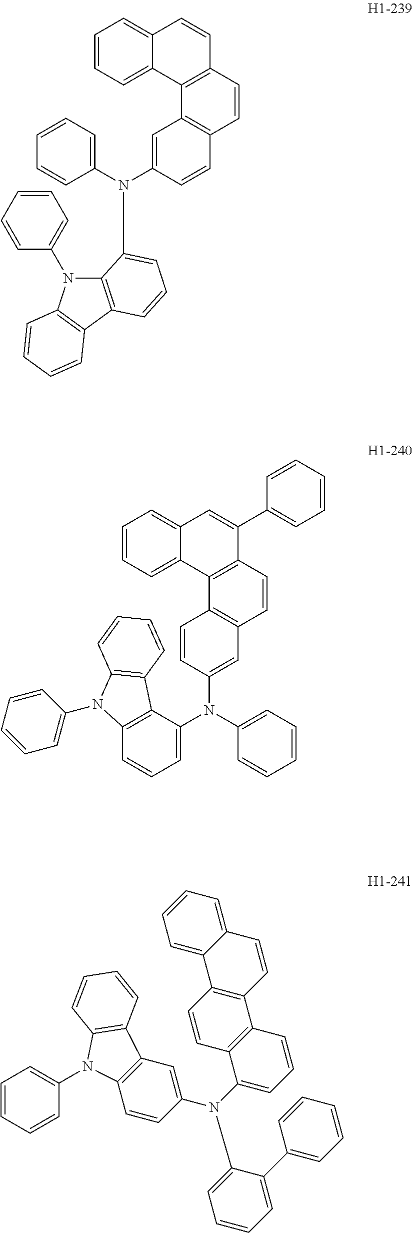 [SMILES: c1ccc(N(c2ccc3ccc4ccc5ccccc5c4c3c2)c2cccc3c4ccccc4n(-c4ccccc4)c23)cc1 ; c1ccc(-c2cc3ccccc3c3c2ccc2cc(N(c4ccccc4)c4cccc5c4c4ccccc4n5-c4ccccc4)ccc23)cc1 ; c1ccc(-c2ccccc2N(c2ccc3c(c2)c2ccccc2n3-c2ccccc2)c2cccc3c2ccc2c4ccccc4ccc32)cc1]

The second host compound as another host material according to one embodiment is represented by the following Formula 3.

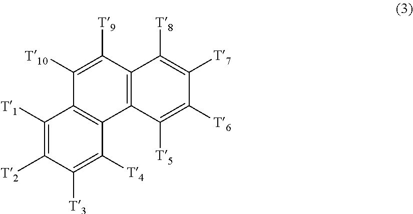

In Formula 3,
T′₅ and T′₆ are connected to each other to form a ring of the following Formula 4; or T′₇ and T′₈ are connected to each other to form a ring of the following Formula 4; or T′₅ and T′₆ are connected to each other to form a ring of the following Formula 4, and T′₇ and T′₈ are connected to each other to form a ring of the following Formula 4;

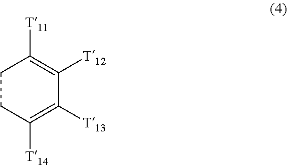

in formulas 3 and 4,
T′₁ to T′₄, T′₉ to T′₁₄, and T′₅ to T′₈, which do not form a ring, each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy a substituted or unsubstituted fused ring of (C3-C30) aliphatic ring and (C6-C30) aromatic ring, —SiR₁₁R₁₂R₁₃, —NR₁₄R₁₅, or -L₂-HAr₁; provided that at least one of T₁ to T′₁₄ is -L₂-HAr₁;
R₁₁ to R₁₅ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
L₂ represents a single bond, a substituted or unsubstituted (C6-C30)arylene, or a substituted or unsubstituted (3- to 30-membered)heteroarylene;
HAr₁ represents a substituted or unsubstituted (3- to 30-membered)heteroaryl comprising at least one nitrogen (N) atom; and
[symbol] represents a site fused with the Formula 3.

In one embodiment, T′₅ and T′₆, in the Formula 3, may be connected to each other to form a ring of the Formula 4.

In one embodiment, T′₇ and T's, in the Formula 3, may be connected to each other to form a ring of the Formula 4.

In one embodiment, each of T′₅ and T′₆, and T′₇ and T's, in the Formula 3, may be connected to each other to form a ring of the Formula 4.

According to one embodiment, the second host compound represented by Formula 3 may be represented by any one of the following formulas 3-1 to 3-3.

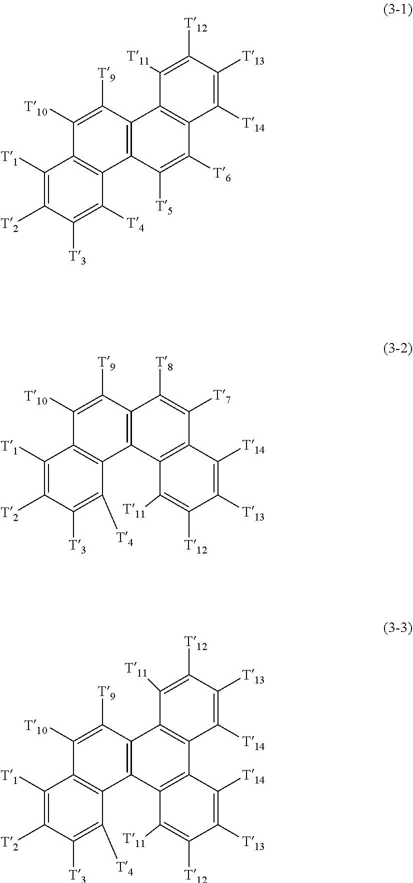

In formulas 3-1 to 3-3,
T′₁ to T′₁₄ are as defined as T′₁ to T′₄, T′₉ to T′₁₄, and T′₅ to T′₈, which do not form a ring, in Formula 3.

In one embodiment, at least one of T′₁ to T′₁₄ may be -L₂-HAr₁, for example, at least one of T′₁₁ to T′₁₄ may be -L₂-HAr₁.

In one embodiment, L₂ may be a single bond or a substituted or unsubstituted (C6-C30)arylene, preferably a single bond or a substituted or unsubstituted (C6-C25)arylene, more preferably a single bond or (C6-C18)arylene unsubstituted or substituted with (C6-C30)aryl. For example, L₂ may be a single bond, phenylene unsubstituted or substituted with phenyl, a substituted or unsubstituted naphthylene, or a substituted or unsubstituted biphenylene.

In one embodiment, HAr₁ may be a substituted or unsubstituted (5- to 30-membered)heteroaryl comprising at least one nitrogen (N) atom, preferably a substituted or unsubstituted (5- to 30-membered)heteroaryl comprising at least two nitrogen (N) atoms, more preferably (5- to 30-membered)heteroaryl comprising at least three nitrogen (N) atoms unsubstituted or substituted with (C6-C30)aryl or (5- to 30-membered)heteroaryl. For example, HAr₁ may be a substituted or unsubstituted pyridyl, a substituted or unsubstituted pyrimidinyl, a substituted or unsubstituted triazinyl, a substituted or unsubstituted pyrazinyl, a substituted or unsubstituted benzimidazolyl, a substituted or unsubstituted quinolinyl, a substituted or unsubstituted quinazolinyl, a substituted or unsubstituted quinoxalinyl, a substituted or unsubstituted naphthyridinyl, a substituted or unsubstituted benzoquinolinyl, a substituted or unsubstituted benzoquinazolinyl, a substituted or unsubstituted benzoquinoxalinyl, a substituted or unsubstituted dibenzoquinolinyl, a substituted or unsubstituted dibenzoquinazolinyl, a substituted or unsubstituted dibenzoquinoxalinyl, a substituted or unsubstituted indenopyridyl, a substituted or unsubstituted indenopyrimidinyl, a substituted or unsubstituted indenopyrazinyl, a substituted or unsubstituted benzofuropyridyl, a substituted or unsubstituted benzofuropyrimidinyl, a substituted or unsubstituted benzofuropyrazinyl, a substituted or unsubstituted benzothiopyridyl, a substituted or unsubstituted benzothiopyrimidinyl, a substituted or unsubstituted benzothiopyrazinyl, or a substituted or unsubstituted benzothienoquinolinyl, for example, a substituted or unsubstituted pyrimidinyl, a substituted or unsubstituted triazinyl, a substituted or unsubstituted benzimidazolyl, a substituted or unsubstituted quinolinyl, a substituted or unsubstituted quinazolinyl, a substituted or unsubstituted quinoxalinyl, a substituted or unsubstituted naphthyridinyl, a substituted or unsubstituted benzoquinolinyl, a substituted or unsubstituted benzoquinoxalinyl, a substituted or unsubstituted dibenzoquinoxalinyl, a substituted or unsubstituted benzofuropyrimidinyl, or a substituted or unsubstituted benzothienoquinolinyl. Wherein, the substituent in the substituted groups may be, for example, phenyl unsubstituted or substituted with least one of cyano; fluorine (F); tert-butyl; cyclohexyl; naphthyl; trimethylsilyl; and triphenylsilyl, naphthyl, p-biphenyl, m-biphenyl, o-biphenyl, p-terphenyl, m-terphenyl, o-terphenyl, phenanthrenyl, chrysenyl, triphenylenyl, anthracenyl, dimethylfluorenyl, diphenylfluorenyl, spirobifluorenyl, pyridyl unsubstituted or substituted with phenyl, dibenzofuranyl unsubstituted or substituted with phenyl or biphenyl, a substituted or unsubstituted dibenzothiophenyl, carbazolyl, phenoxazinyl, benzothiophenyl, or naphthooxazolyl unsubstituted or substituted with phenyl.

According to another embodiment, HAr₁ may be a substituent represents by the following Formula 3-1-1.

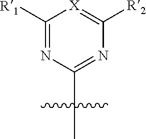

In Formula 3-1-1,
X represents N or CR′;
R′₁ and R′₂ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
R′ represents hydrogen or deuterium.

According to one embodiment, the second host compound represented by Formula 3 may be more specifically illustrated by the following compounds, but is not limited thereto.

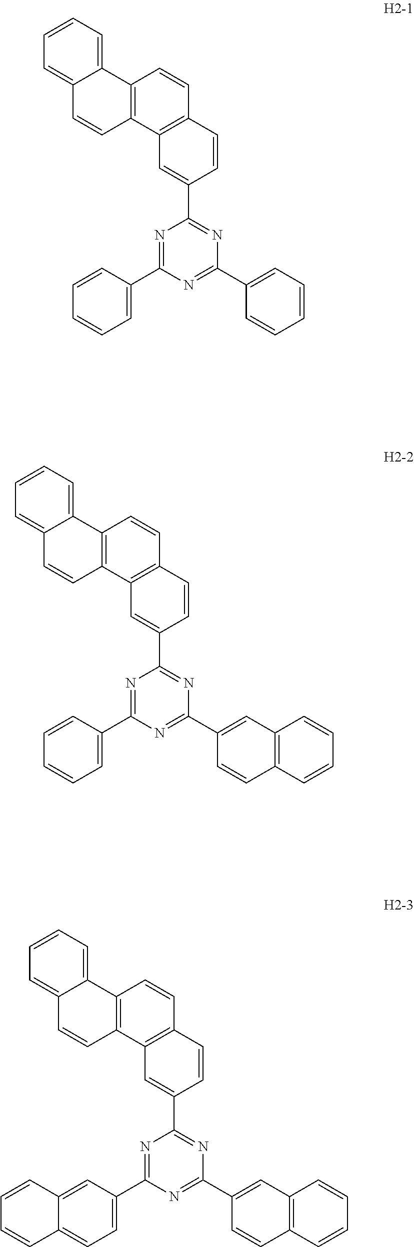 [SMILES: c1ccc(-c2nc(-c3ccccc3)nc(-c3ccc4ccc5c6ccccc6ccc5c4c3)n2)cc1 ; c1ccc(-c2nc(-c3ccc4ccccc4c3)nc(-c3ccc4ccc5c6ccccc6ccc5c4c3)n2)cc1 ; c1ccc2cc(-c3nc(-c4ccc5ccccc5c4)nc(-c4ccc5ccc6c7ccccc7ccc6c5c4)n3)ccc2c1]

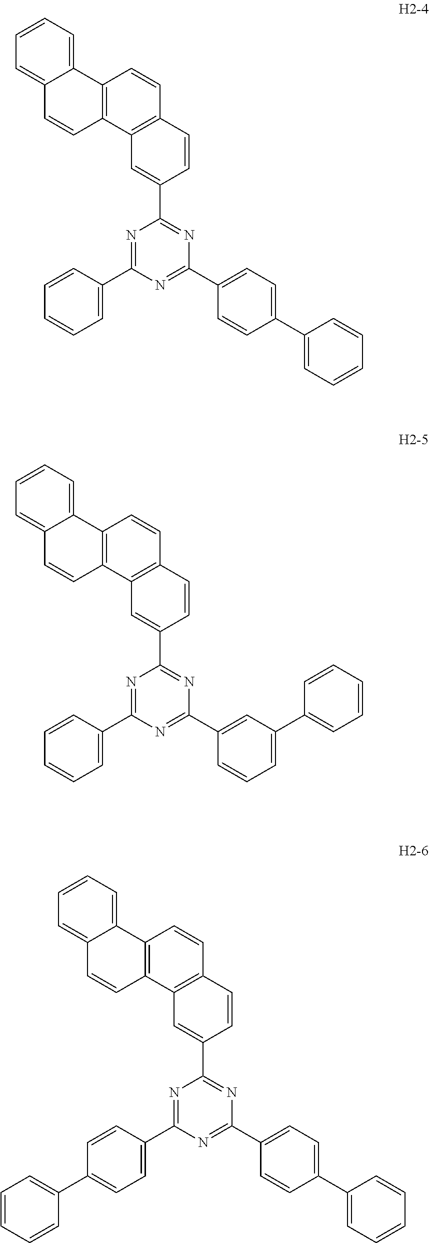 [SMILES: c1ccc(-c2ccc(-c3nc(-c4ccccc4)nc(-c4ccc5ccc6c7ccccc7ccc6c5c4)n3)cc2)cc1 ; c1ccc(-c2cccc(-c3nc(-c4ccccc4)nc(-c4ccc5ccc6c7ccccc7ccc6c5c4)n3)c2)cc1 ; c1ccc(-c2ccc(-c3nc(-c4ccc(-c5ccccc5)cc4)nc(-c4ccc5ccc6c7ccccc7ccc6c5c4)n3)cc2)cc1]

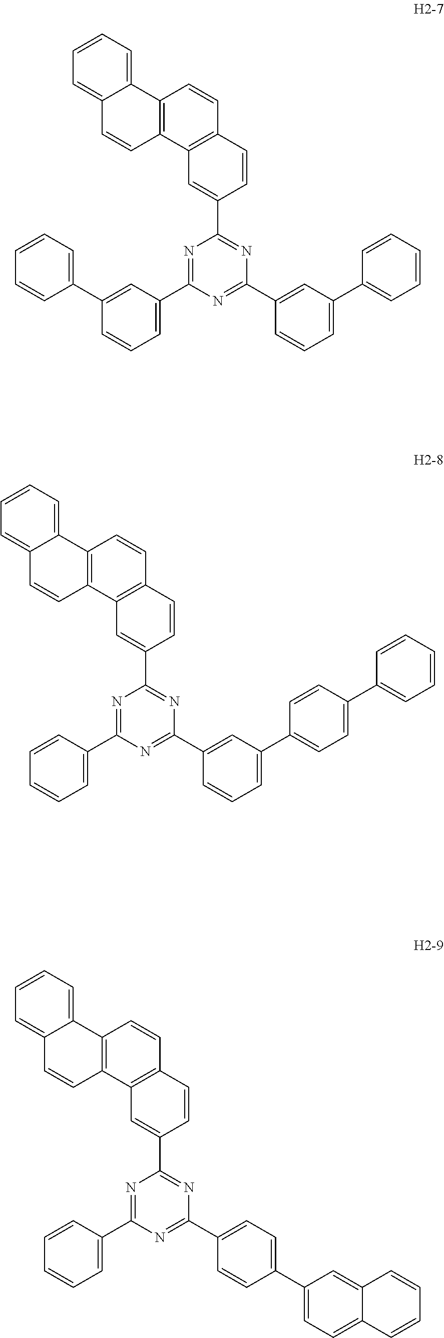 [SMILES: c1ccc(-c2cccc(-c3nc(-c4cccc(-c5ccccc5)c4)nc(-c4ccc5ccc6c7ccccc7ccc6c5c4)n3)c2)cc1 ; c1ccc(-c2ccc(-c3cccc(-c4nc(-c5ccccc5)nc(-c5ccc6ccc7c8ccccc8ccc7c6c5)n4)c3)cc2)cc1 ; c1ccc(-c2nc(-c3ccc(-c4ccc5ccccc5c4)cc3)nc(-c3ccc4ccc5c6ccccc6ccc5c4c3)n2)cc1]

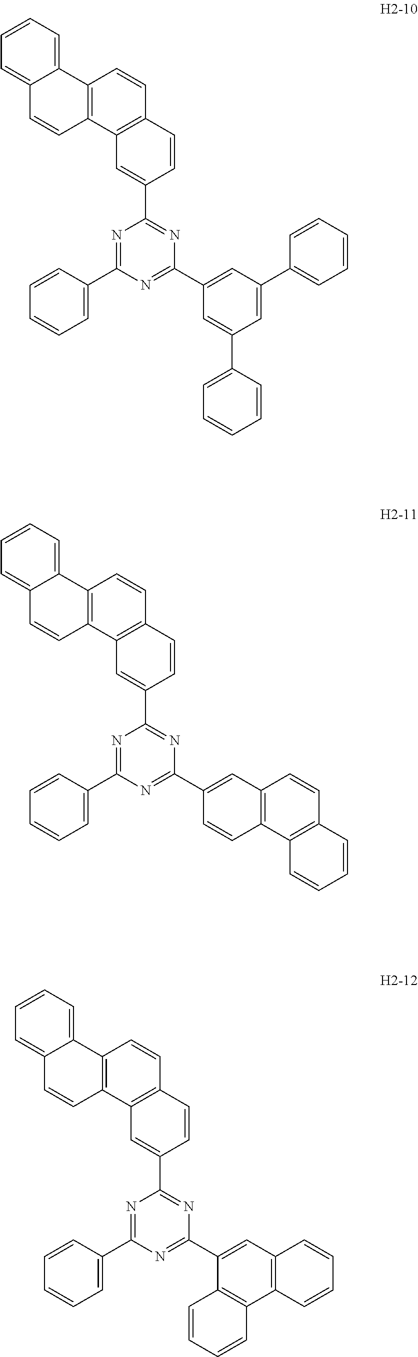 [SMILES: c1ccc(-c2cc(-c3ccccc3)cc(-c3nc(-c4ccccc4)nc(-c4ccc5ccc6c7ccccc7ccc6c5c4)n3)c2)cc1 ; c1ccc(-c2nc(-c3ccc4c(ccc5ccccc54)c3)nc(-c3ccc4ccc5c6ccccc6ccc5c4c3)n2)cc1 ; c1ccc(-c2nc(-c3ccc4ccc5c6ccccc6ccc5c4c3)nc(-c3cc4ccccc4c4ccccc34)n2)cc1]

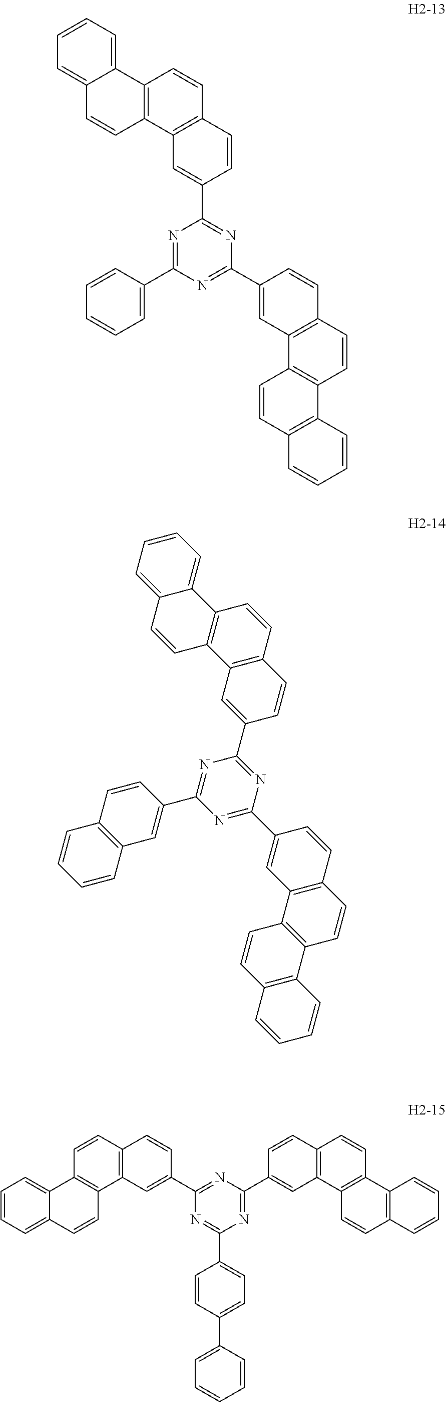 [SMILES: c1ccc(-c2nc(-c3ccc4ccc5c6ccccc6ccc5c4c3)nc(-c3ccc4ccc5c6ccccc6ccc5c4c3)n2)cc1 ; c1ccc2cc(-c3nc(-c4ccc5ccc6c7ccccc7ccc6c5c4)nc(-c4ccc5ccc6c7ccccc7ccc6c5c4)n3)ccc2c1 ; c1ccc(-c2ccc(-c3nc(-c4ccc5ccc6c7ccccc7ccc6c5c4)nc(-c4ccc5ccc6c7ccccc7ccc6c5c4)n3)cc2)cc1]

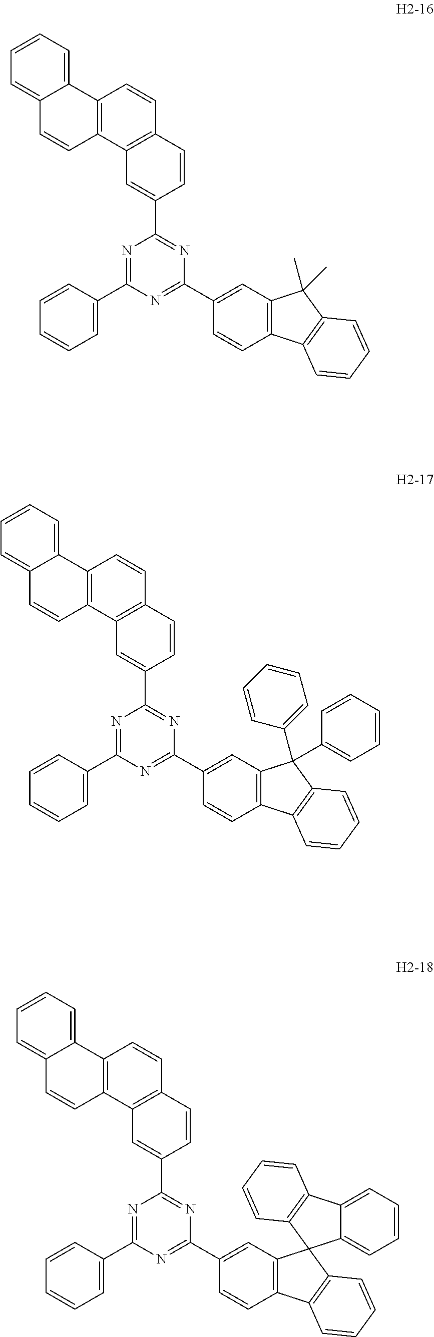 [SMILES: CC1(C)c2ccccc2-c2ccc(-c3nc(-c4ccccc4)nc(-c4ccc5ccc6c7ccccc7ccc6c5c4)n3)cc21 ; c1ccc(-c2nc(-c3ccc4c(c3)C(c3ccccc3)(c3ccccc3)c3ccccc3-4)nc(-c3ccc4ccc5c6ccccc6ccc5c4c3)n2)cc1 ; c1ccc(-c2nc(-c3ccc4c(c3)C3(c5ccccc5-c5ccccc53)c3ccccc3-4)nc(-c3ccc4ccc5c6ccccc6ccc5c4c3)n2)cc1]

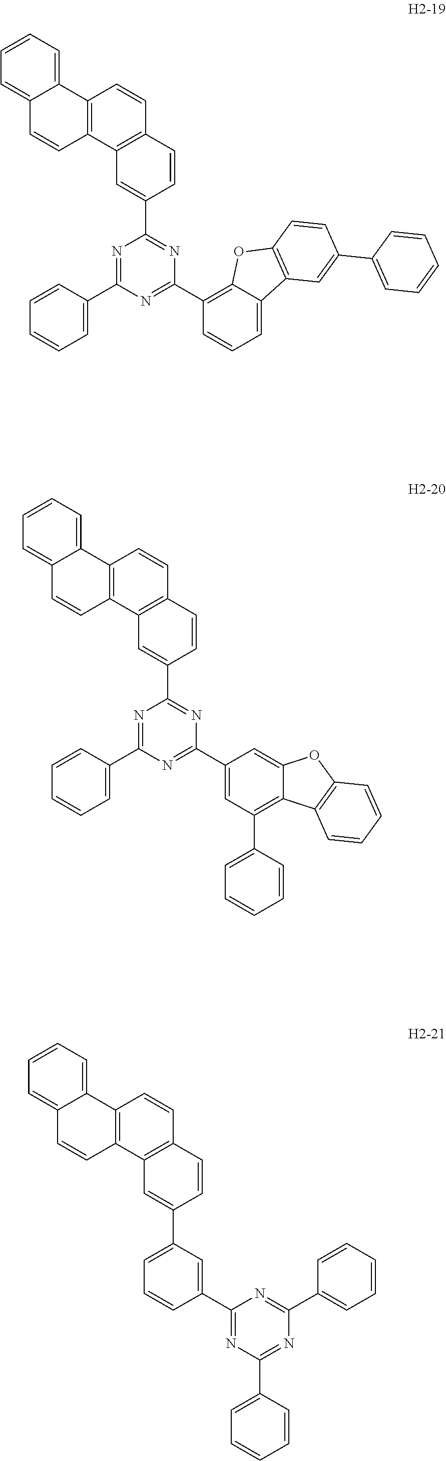 [SMILES: c1ccc(-c2ccc3oc4c(-c5nc(-c6ccccc6)nc(-c6ccc7ccc8c9ccccc9ccc8c7c6)n5)cccc4c3c2)cc1 ; c1ccc(-c2nc(-c3cc(-c4ccccc4)c4c(c3)oc3ccccc34)nc(-c3ccc4ccc5c6ccccc6ccc5c4c3)n2)cc1 ; c1ccc(-c2nc(-c3ccccc3)nc(-c3cccc(-c4ccc5ccc6c7ccccc7ccc6c5c4)c3)n2)cc1]

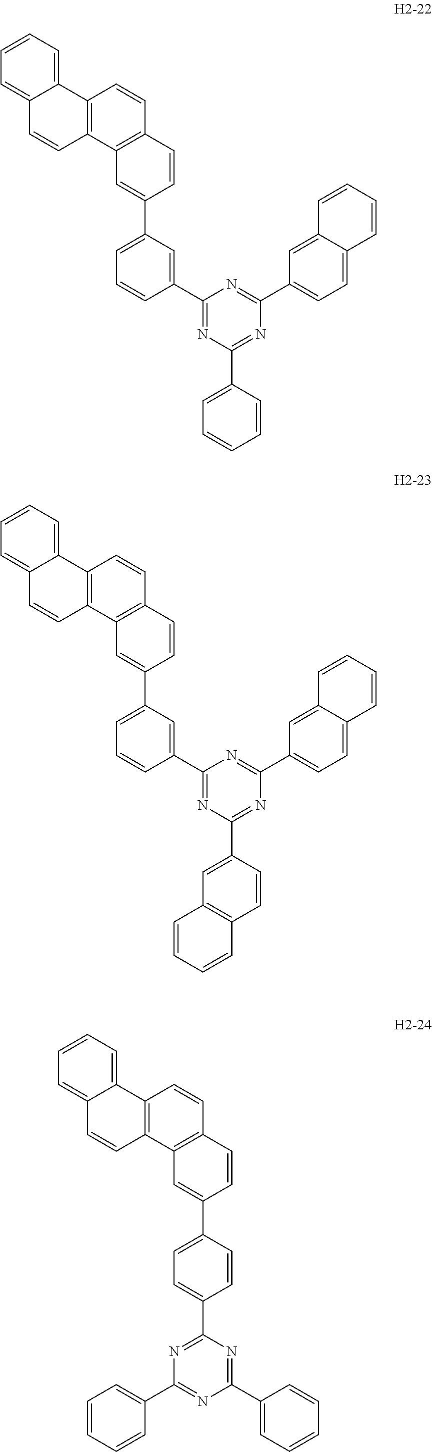 [SMILES: c1ccc(-c2nc(-c3cccc(-c4ccc5ccc6c7ccccc7ccc6c5c4)c3)nc(-c3ccc4ccccc4c3)n2)cc1 ; c1cc(-c2ccc3ccc4c5ccccc5ccc4c3c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5ccc6c7ccccc7ccc6c5c4)cc3)n2)cc1]

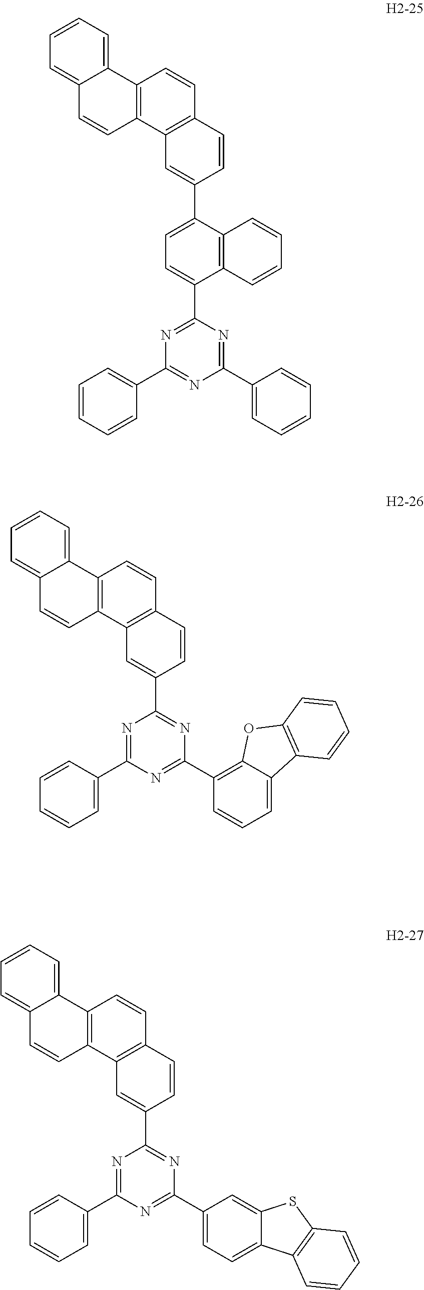 [SMILES: c1ccc(-c2nc(-c3ccccc3)nc(-c3ccc(-c4ccc5ccc6c7ccccc7ccc6c5c4)c4ccccc34)n2)cc1 ; c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1]

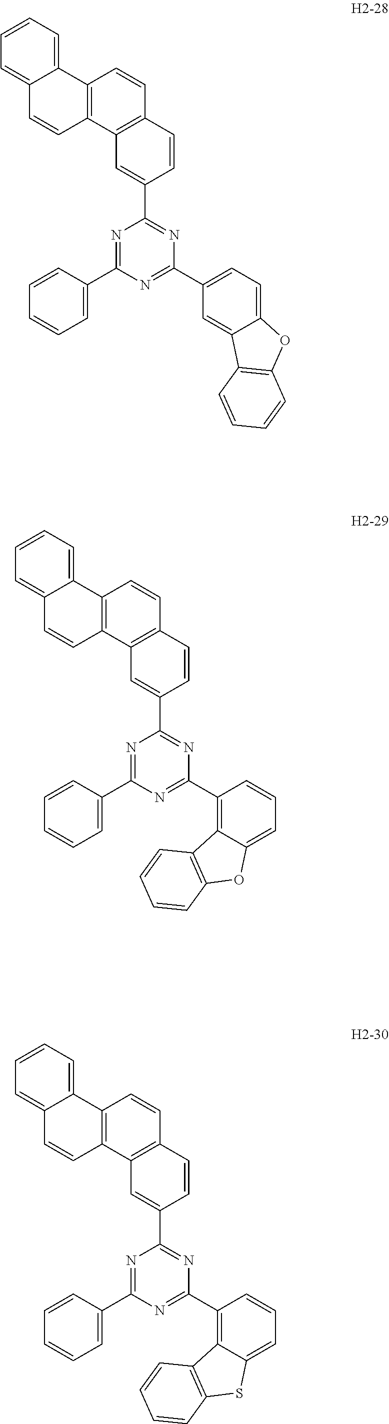 [SMILES: c1ccc(-c2nc(-c3ccc4ccc5c6ccccc6ccc5c4c3)nc(-c3ccc4oc5ccccc5c4c3)n2)cc1 ; c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1]

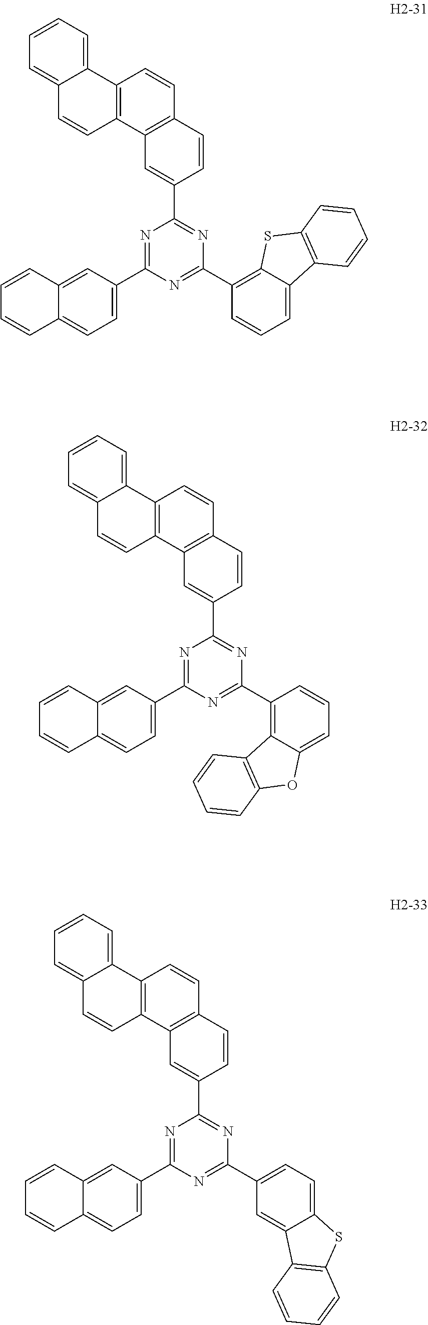 [SMILES: c1ccc2cc(-c3nc(-c4ccc5ccc6c7ccccc7ccc6c5c4)nc(-c4cccc5c4sc4ccccc45)n3)ccc2c1 ; c1ccc2cc(-c3nc(-c4ccc5ccc6c7ccccc7ccc6c5c4)nc(-c4cccc5oc6ccccc6c45)n3)ccc2c1 ; c1ccc2cc(-c3nc(-c4ccc5ccc6c7ccccc7ccc6c5c4)nc(-c4ccc5sc6ccccc6c5c4)n3)ccc2c1]

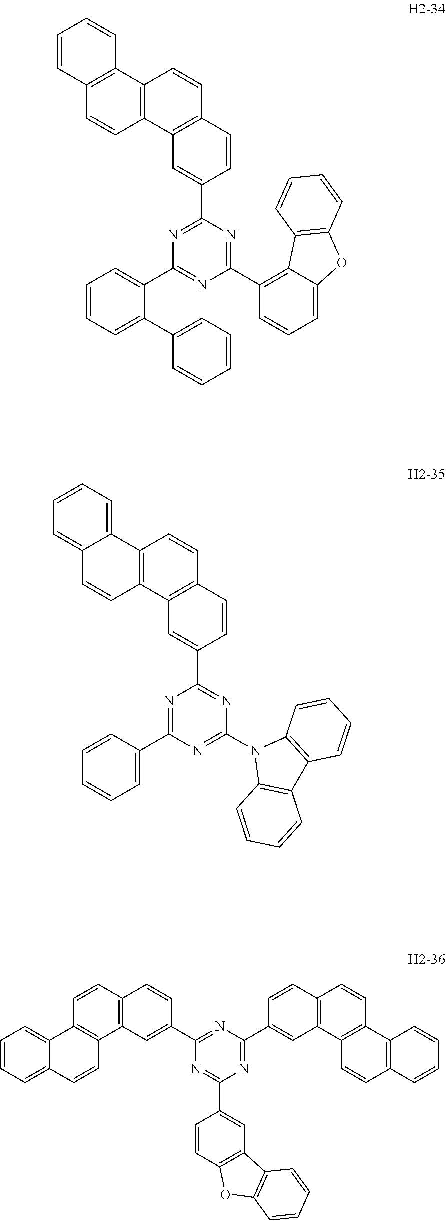 [SMILES: c1ccc(-c2ccccc2-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-n3c4ccccc4c4ccccc43)n2)cc1 ; c1ccc2c(c1)ccc1c3cc(-c4nc(-c5ccc6ccc7c8ccccc8ccc7c6c5)nc(-c5ccc6oc7ccccc7c6c5)n4)ccc3ccc21]

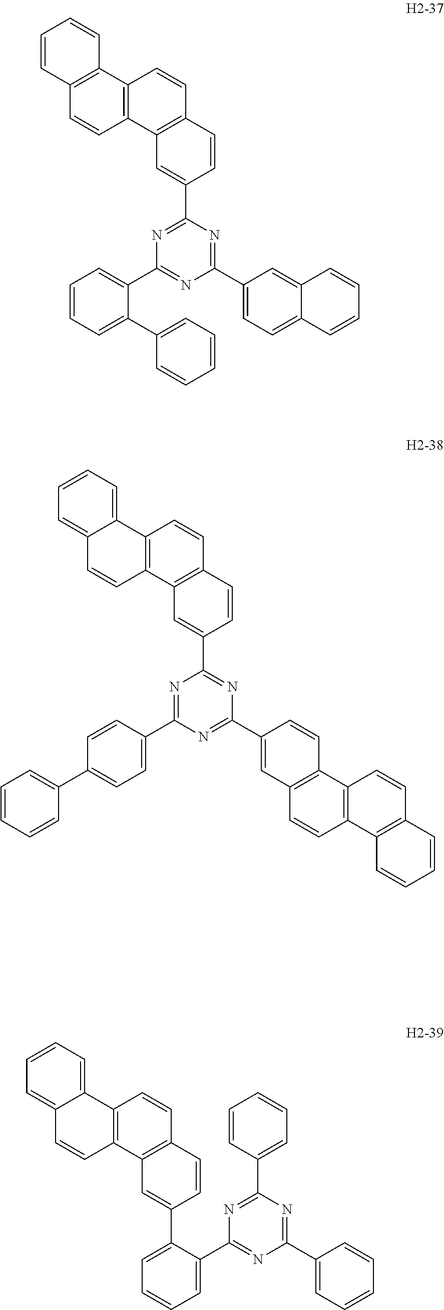 [SMILES: c1ccc(-c2ccccc2-c2nc(-c3ccc4ccccc4c3)nc(-c3ccc4ccc5c6ccccc6ccc5c4c3)n2)cc1 ; c1ccc(-c2ccc(-c3nc(-c4ccc5c(ccc6c7ccccc7ccc56)c4)nc(-c4ccc5ccc6c7ccccc7ccc6c5c4)n3)cc2)cc1 ; c1ccc(-c2nc(-c3ccccc3)nc(-c3ccccc3-c3ccc4ccc5c6ccccc6ccc5c4c3)n2)cc1]

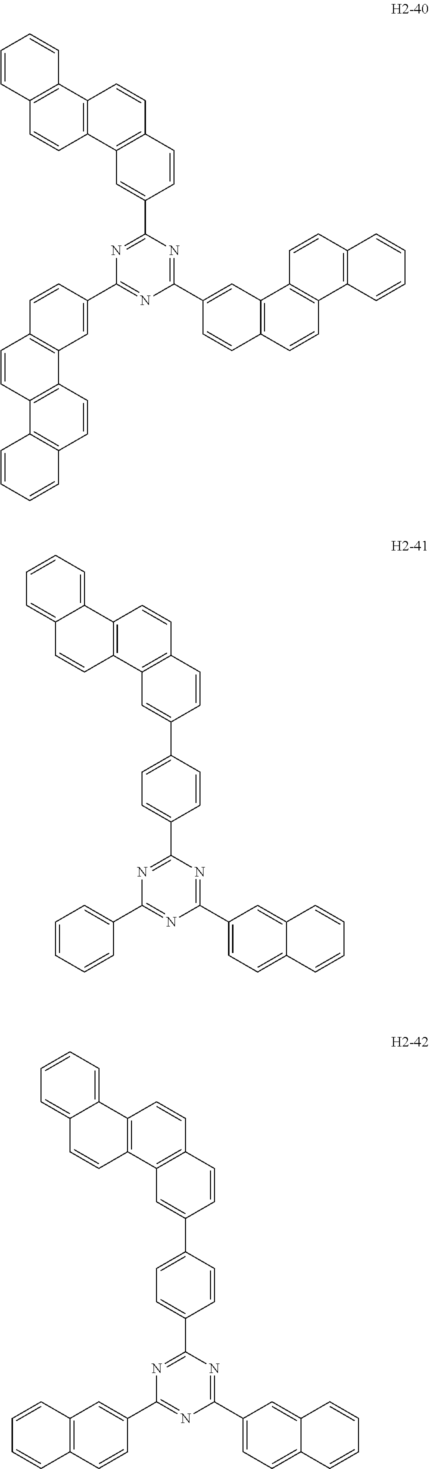 [SMILES: c1ccc2c(c1)ccc1c3cc(-c4nc(-c5ccc6ccc7c8ccccc8ccc7c6c5)nc(-c5ccc6ccc7c8ccccc8ccc7c6c5)n4)ccc3ccc21 ; c1ccc(-c2nc(-c3ccc(-c4ccc5ccc6c7ccccc7ccc6c5c4)cc3)nc(-c3ccc4ccccc4c3)n2)cc1 ; c1ccc2cc(-c3nc(-c4ccc(-c5ccc6ccc7c8ccccc8ccc7c6c5)cc4)nc(-c4ccc5ccccc5c4)n3)ccc2c1]

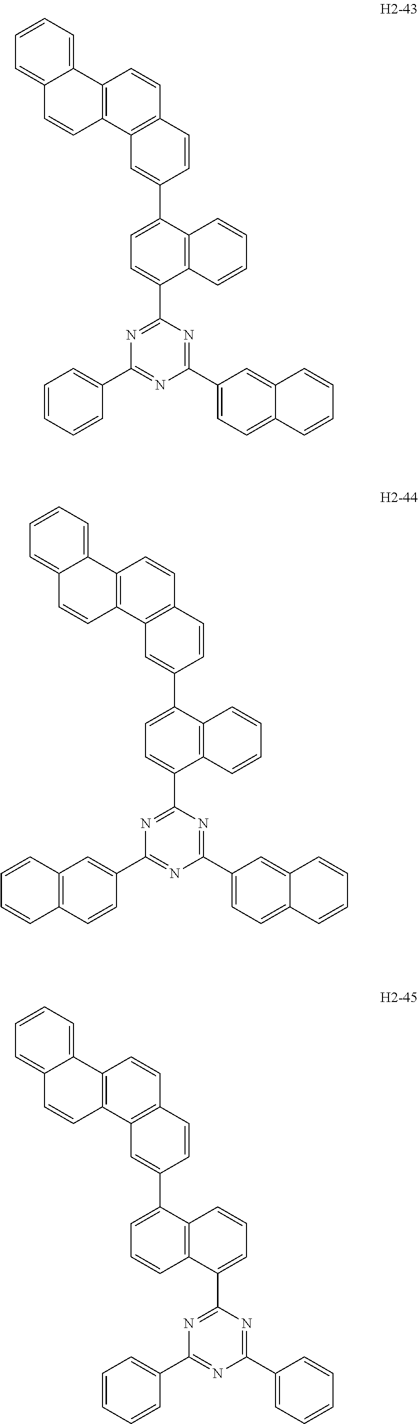 [SMILES: c1ccc(-c2nc(-c3ccc4ccccc4c3)nc(-c3ccc(-c4ccc5ccc6c7ccccc7ccc6c5c4)c4ccccc34)n2)cc1 ; c1ccc2cc(-c3nc(-c4ccc5ccccc5c4)nc(-c4ccc(-c5ccc6ccc7c8ccccc8ccc7c6c5)c5ccccc45)n3)ccc2c1 ; c1ccc(-c2nc(-c3ccccc3)nc(-c3cccc4c(-c5ccc6ccc7c8ccccc8ccc7c6c5)cccc34)n2)cc1]

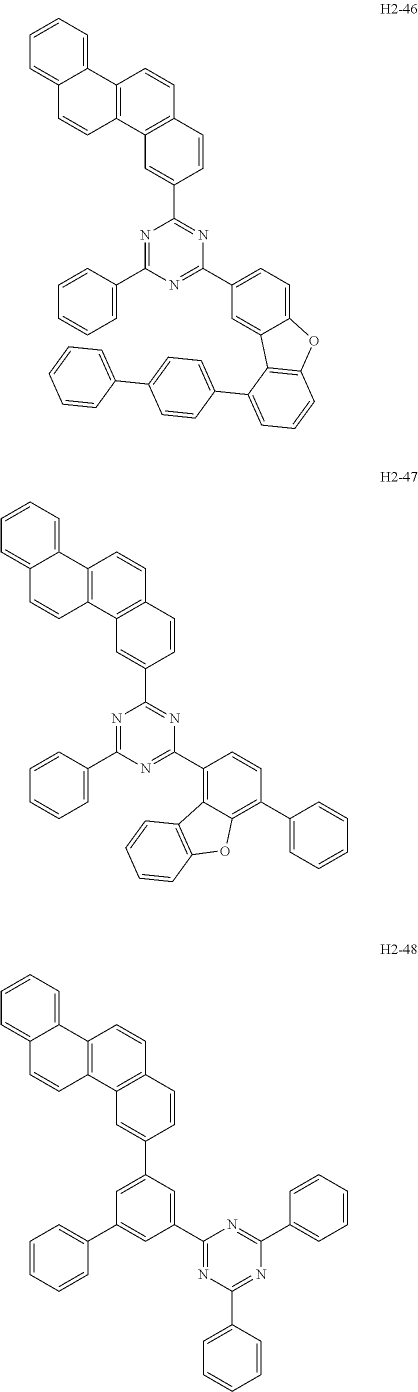 [SMILES: c1ccc(-c2ccc(-c3cccc4oc5ccc(-c6nc(-c7ccccc7)nc(-c7ccc8ccc9c%10ccccc%10ccc9c8c7)n6)cc5c34)cc2)cc1 ; c1ccc(-c2nc(-c3ccc4ccc5c6ccccc6ccc5c4c3)nc(-c3ccc(-c4ccccc4)c4oc5ccccc5c34)n2)cc1 ; c1ccc(-c2cc(-c3ccc4ccc5c6ccccc6ccc5c4c3)cc(-c3nc(-c4ccccc4)nc(-c4ccccc4)n3)c2)cc1]

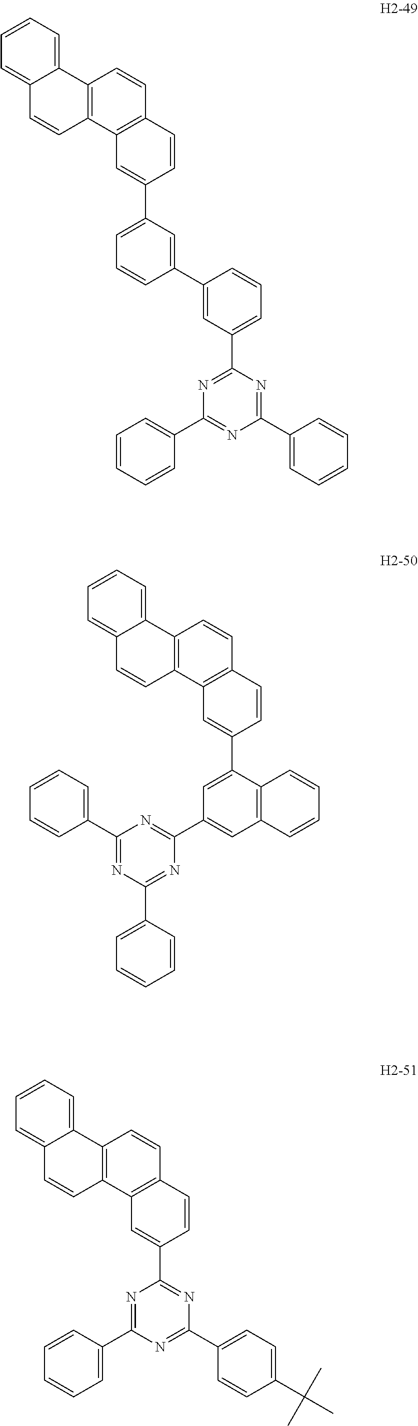 [SMILES: c1ccc(-c2nc(-c3ccccc3)nc(-c3cccc(-c4cccc(-c5ccc6ccc7c8ccccc8ccc7c6c5)c4)c3)n2)cc1 ; c1ccc(-c2nc(-c3ccccc3)nc(-c3cc(-c4ccc5ccc6c7ccccc7ccc6c5c4)c4ccccc4c3)n2)cc1 ; CC(C)(C)c1ccc(-c2nc(-c3ccccc3)nc(-c3ccc4ccc5c6ccccc6ccc5c4c3)n2)cc1]

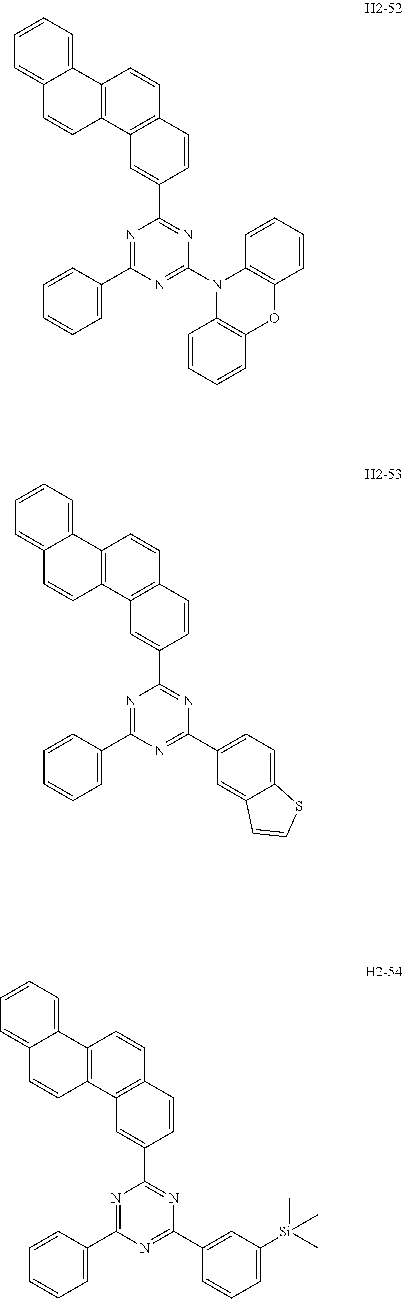 [SMILES: c1ccc(-c2nc(-c3ccc4ccc5c6ccccc6ccc5c4c3)nc(N3c4ccccc4Oc4ccccc43)n2)cc1 ; c1ccc(-c2nc(-c3ccc4sccc4c3)nc(-c3ccc4ccc5c6ccccc6ccc5c4c3)n2)cc1 ; C[Si](C)(C)c1cccc(-c2nc(-c3ccccc3)nc(-c3ccc4ccc5c6ccccc6ccc5c4c3)n2)c1]

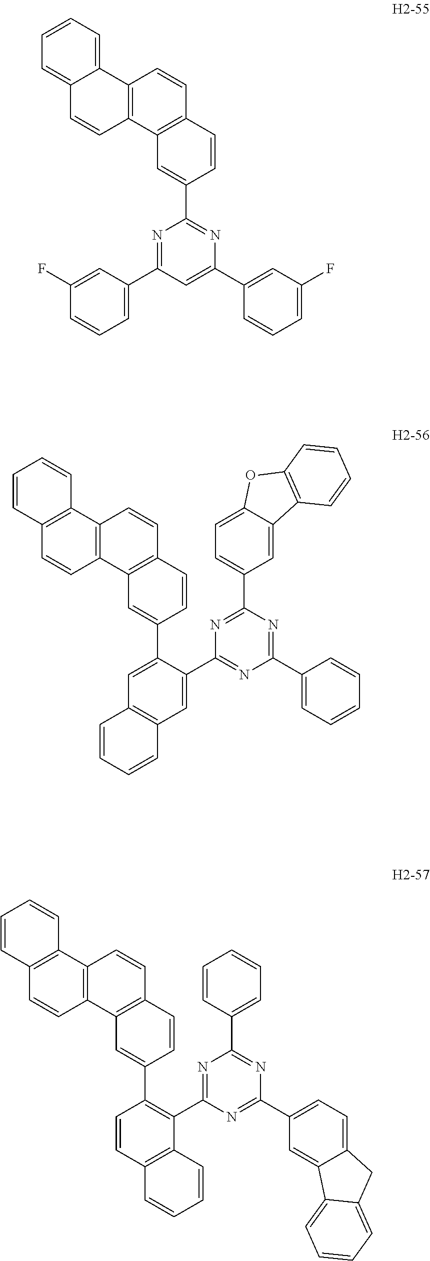 [SMILES: Fc1cccc(-c2cc(-c3cccc(F)c3)nc(-c3ccc4ccc5c6ccccc6ccc5c4c3)n2)c1 ; c1ccc(-c2nc(-c3ccc4oc5ccccc5c4c3)nc(-c3cc4ccccc4cc3-c3ccc4ccc5c6ccccc6ccc5c4c3)n2)cc1 ; c1ccc(-c2nc(-c3ccc4c(c3)-c3ccccc3C4)nc(-c3c(-c4ccc5ccc6c7ccccc7ccc6c5c4)ccc4ccccc34)n2)cc1]

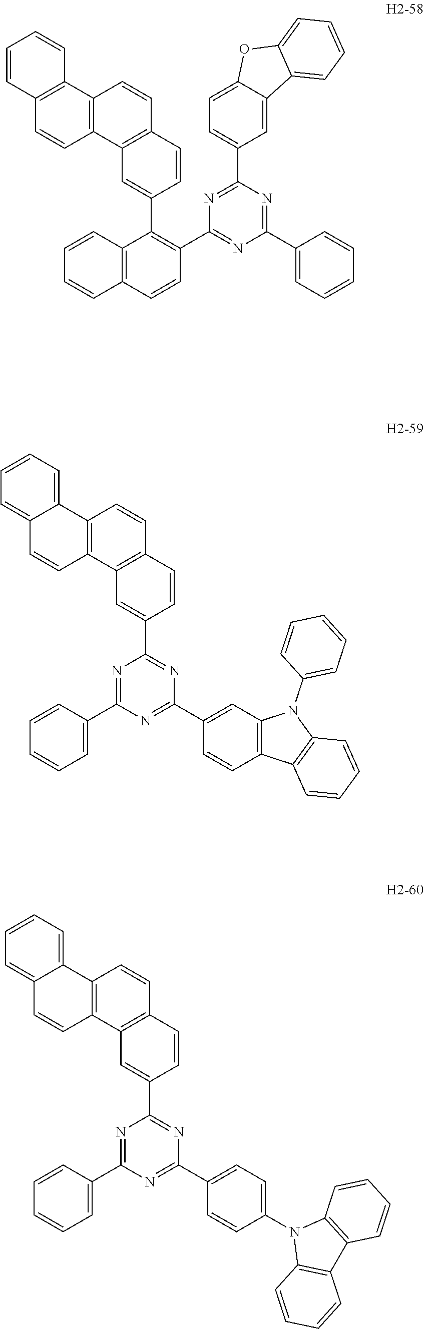 [SMILES: c1ccc(-c2nc(-c3ccc4oc5ccccc5c4c3)nc(-c3ccc4ccccc4c3-c3ccc4ccc5c6ccccc6ccc5c4c3)n2)cc1 ; c1ccc(-c2nc(-c3ccc4ccc5c6ccccc6ccc5c4c3)nc(-c3ccc4c5ccccc5n(-c5ccccc5)c4c3)n2)cc1 ; c1ccc(-c2nc(-c3ccc(-n4c5ccccc5c5ccccc54)cc3)nc(-c3ccc4ccc5c6ccccc6ccc5c4c3)n2)cc1]

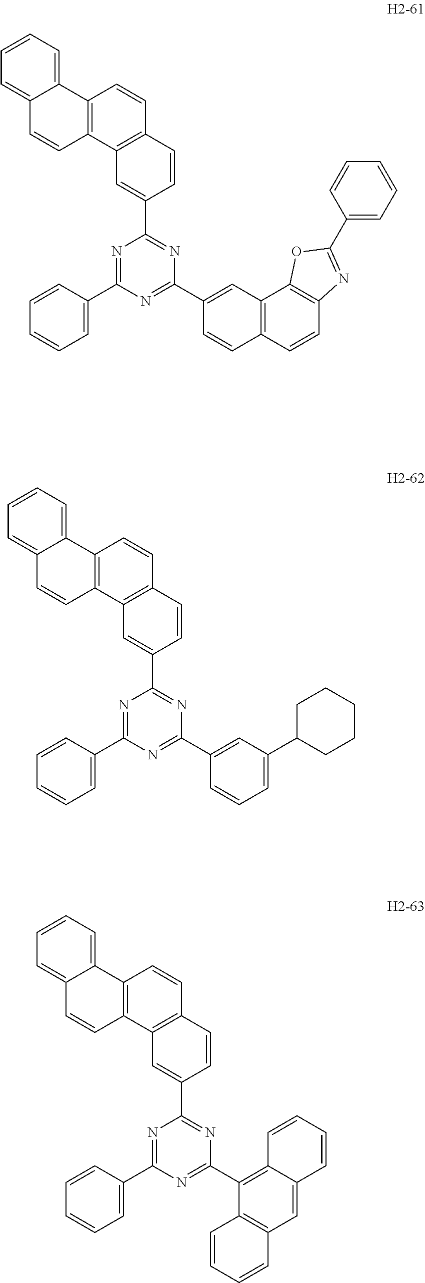 [SMILES: c1ccc(-c2nc(-c3ccc4ccc5c6ccccc6ccc5c4c3)nc(-c3ccc4ccc5nc(-c6ccccc6)oc5c4c3)n2)cc1 ; c1ccc(-c2nc(-c3cccc(C4CCCCC4)c3)nc(-c3ccc4ccc5c6ccccc6ccc5c4c3)n2)cc1 ; c1ccc(-c2nc(-c3ccc4ccc5c6ccccc6ccc5c4c3)nc(-c3c4ccccc4cc4ccccc34)n2)cc1]

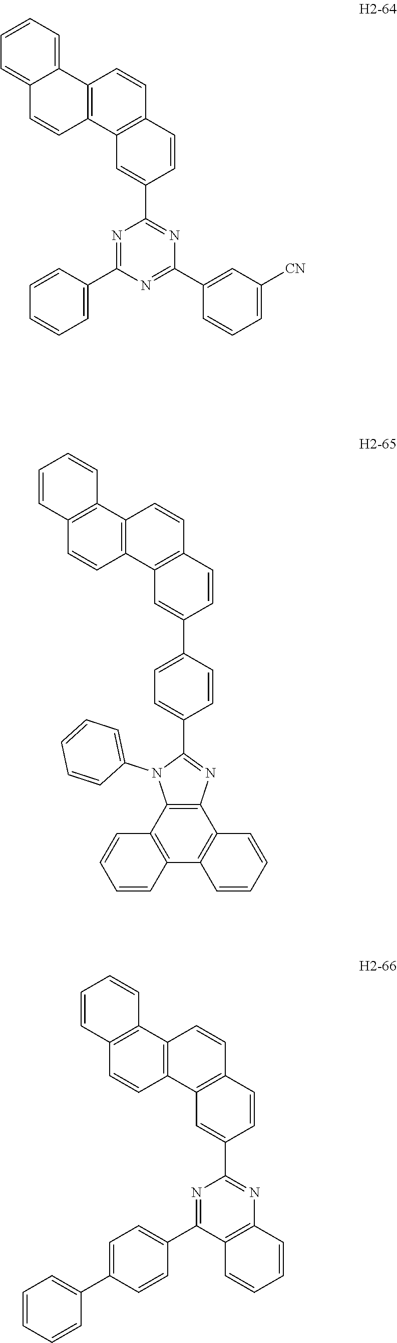 [SMILES: N#Cc1cccc(-c2nc(-c3ccccc3)nc(-c3ccc4ccc5c6ccccc6ccc5c4c3)n2)c1 ; c1ccc(-n2c(-c3ccc(-c4ccc5ccc6c7ccccc7ccc6c5c4)cc3)nc3c4ccccc4c4ccccc4c32)cc1 ; c1ccc(-c2ccc(-c3nc(-c4ccc5ccc6c7ccccc7ccc6c5c4)nc4ccccc34)cc2)cc1]

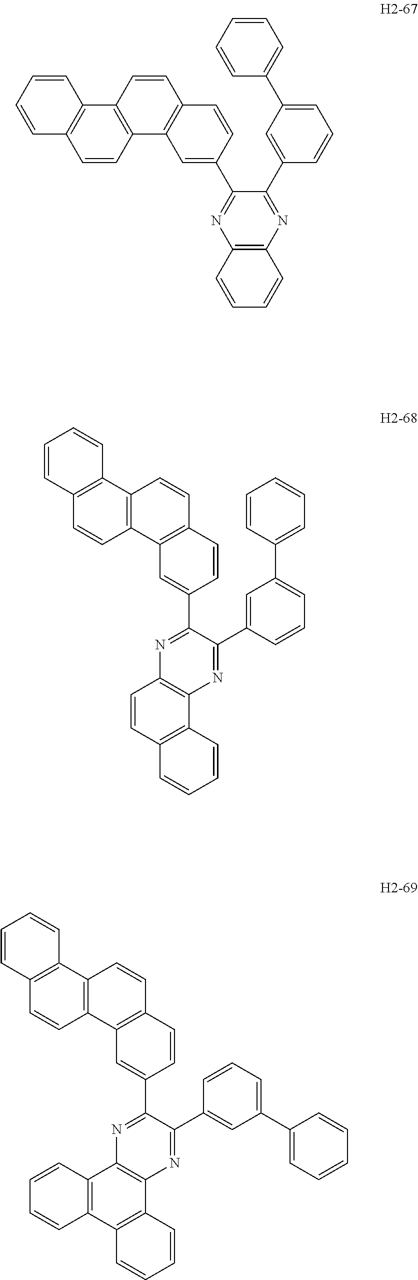 [SMILES: c1ccc(-c2cccc(-c3nc4ccccc4nc3-c3ccc4ccc5c6ccccc6ccc5c4c3)c2)cc1 ; c1ccc(-c2cccc(-c3nc4c(ccc5ccccc54)nc3-c3ccc4ccc5c6ccccc6ccc5c4c3)c2)cc1 ; c1ccc(-c2cccc(-c3nc4c5ccccc5c5ccccc5c4nc3-c3ccc4ccc5c6ccccc6ccc5c4c3)c2)cc1]

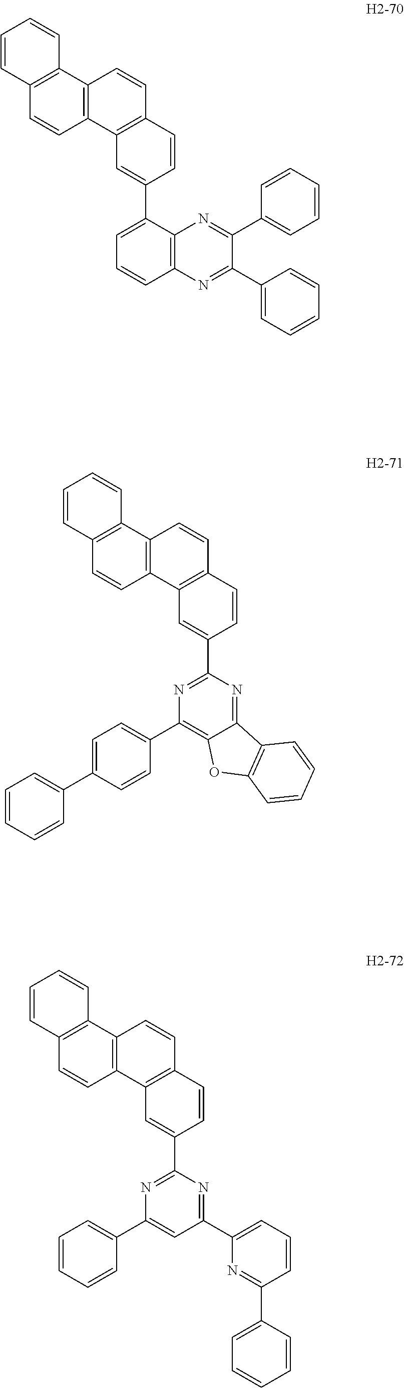 [SMILES: c1ccc(-c2nc3cccc(-c4ccc5ccc6c7ccccc7ccc6c5c4)c3nc2-c2ccccc2)cc1 ; c1ccc(-c2ccc(-c3nc(-c4ccc5ccc6c7ccccc7ccc6c5c4)nc4c3oc3ccccc34)cc2)cc1 ; c1ccc(-c2cccc(-c3cc(-c4ccccc4)nc(-c4ccc5ccc6c7ccccc7ccc6c5c4)n3)n2)cc1]

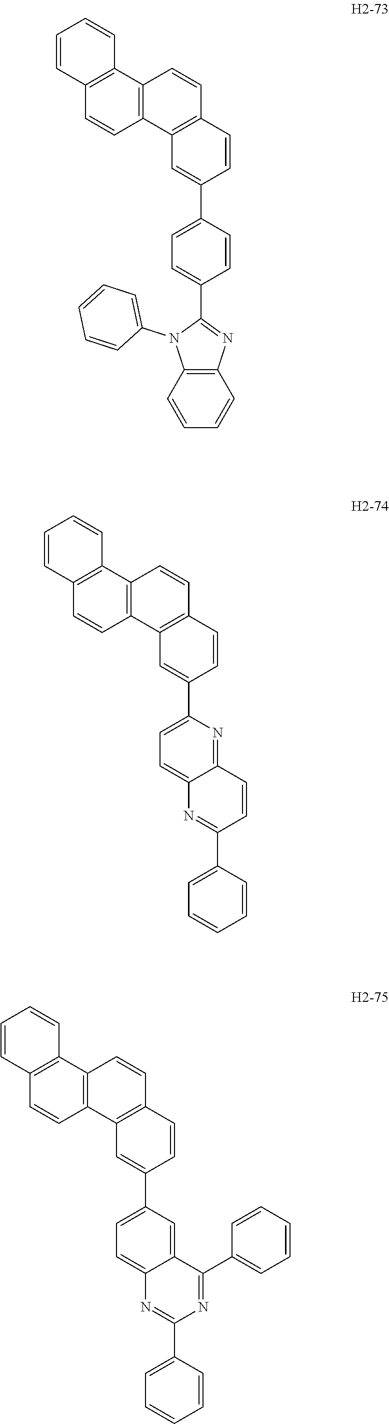 [SMILES: c1ccc(-n2c(-c3ccc(-c4ccc5ccc6c7ccccc7ccc6c5c4)cc3)nc3ccccc32)cc1 ; c1ccc(-c2ccc3nc(-c4ccc5ccc6c7ccccc7ccc6c5c4)ccc3n2)cc1 ; c1ccc(-c2nc(-c3ccccc3)c3cc(-c4ccc5ccc6c7ccccc7ccc6c5c4)ccc3n2)cc1]

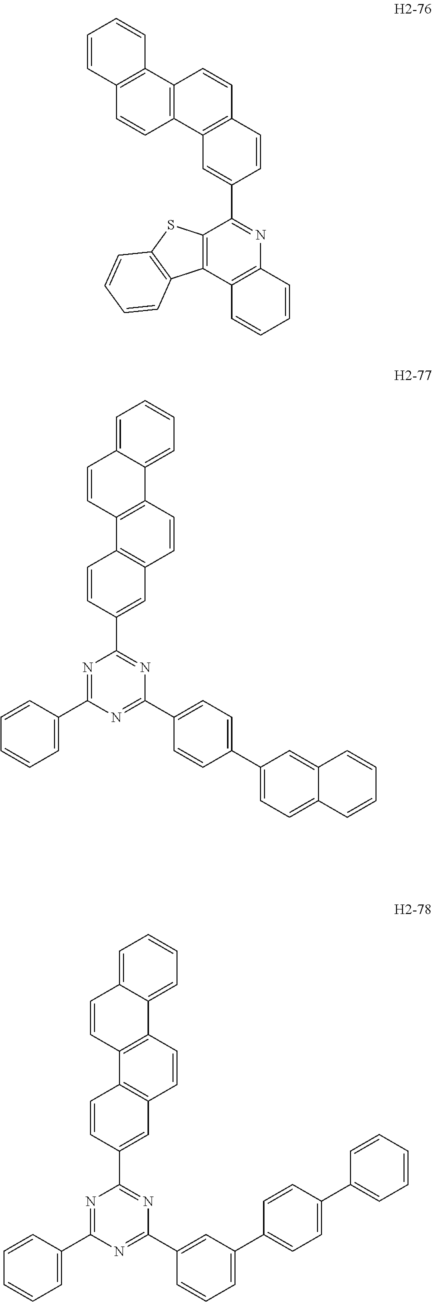 [SMILES: c1ccc2c(c1)ccc1c3cc(-c4nc5ccccc5c5c4sc4ccccc45)ccc3ccc21 ; c1ccc(-c2nc(-c3ccc(-c4ccc5ccccc5c4)cc3)nc(-c3ccc4c(ccc5c6ccccc6ccc45)c3)n2)cc1 ; c1ccc(-c2ccc(-c3cccc(-c4nc(-c5ccccc5)nc(-c5ccc6c(ccc7c8ccccc8ccc67)c5)n4)c3)cc2)cc1]

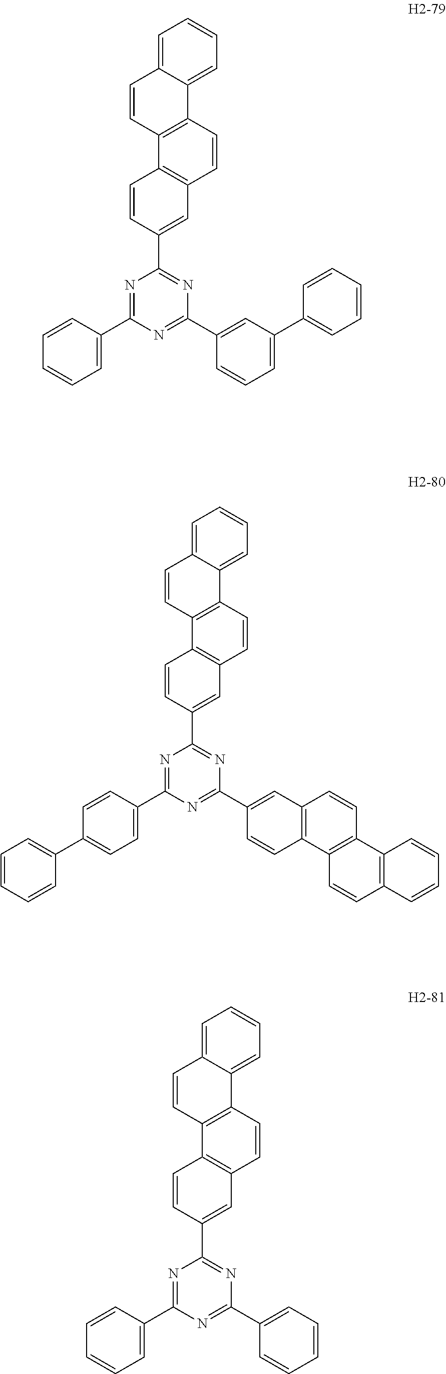 [SMILES: c1ccc(-c2cccc(-c3nc(-c4ccccc4)nc(-c4ccc5c(ccc6c7ccccc7ccc56)c4)n3)c2)cc1 ; c1ccc(-c2ccc(-c3nc(-c4ccc5c(ccc6c7ccccc7ccc56)c4)nc(-c4ccc5c(ccc6c7ccccc7ccc56)c4)n3)cc2)cc1 ; c1ccc(-c2nc(-c3ccccc3)nc(-c3ccc4c(ccc5c6ccccc6ccc45)c3)n2)cc1]

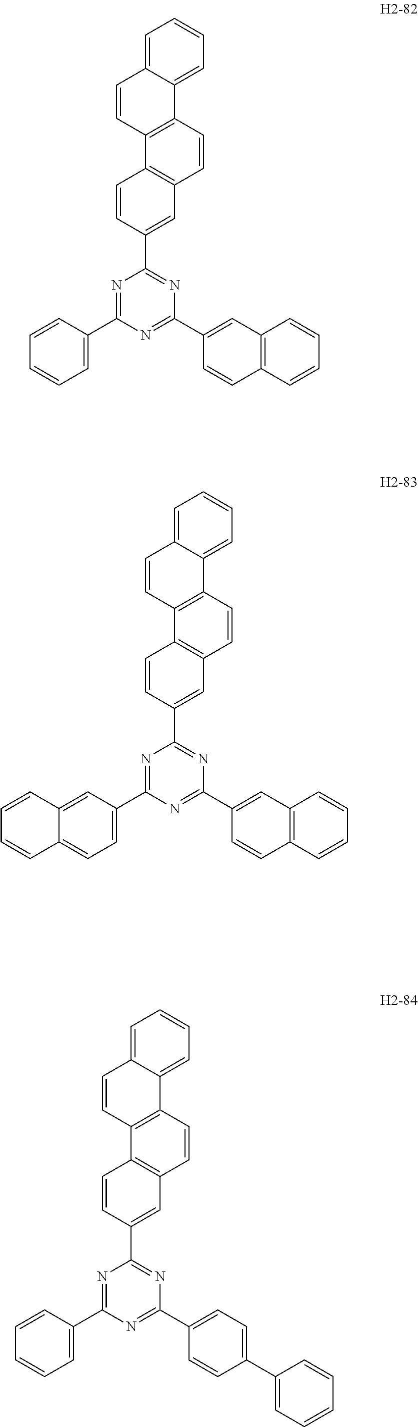 [SMILES: c1ccc(-c2nc(-c3ccc4ccccc4c3)nc(-c3ccc4c(ccc5c6ccccc6ccc45)c3)n2)cc1 ; c1ccc2cc(-c3nc(-c4ccc5ccccc5c4)nc(-c4ccc5c(ccc6c7ccccc7ccc56)c4)n3)ccc2c1 ; c1ccc(-c2ccc(-c3nc(-c4ccccc4)nc(-c4ccc5c(ccc6c7ccccc7ccc56)c4)n3)cc2)cc1]

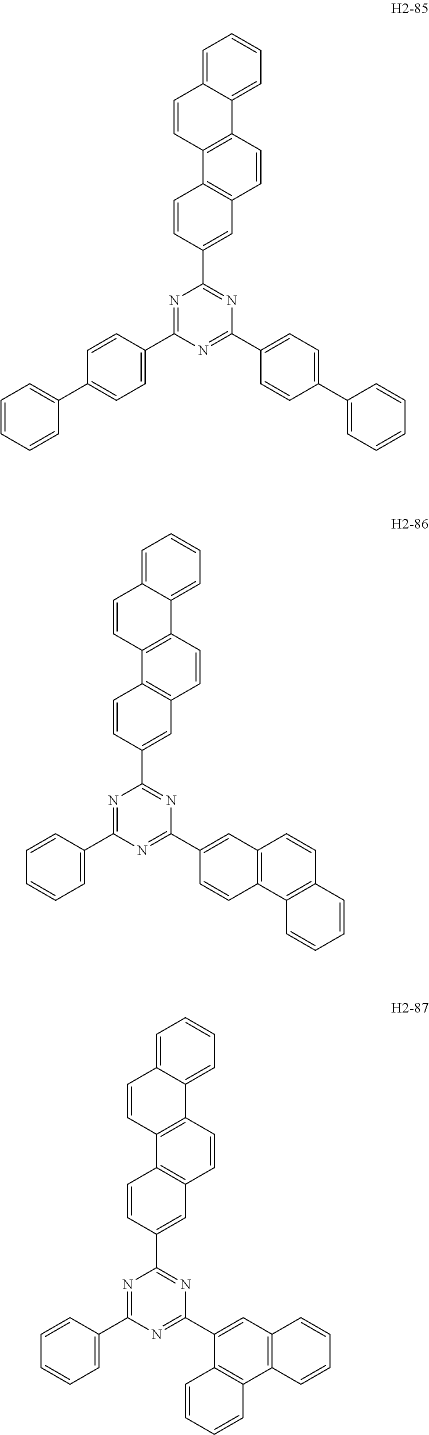 [SMILES: c1ccc(-c2ccc(-c3nc(-c4ccc(-c5ccccc5)cc4)nc(-c4ccc5c(ccc6c7ccccc7ccc56)c4)n3)cc2)cc1 ; c1ccc(-c2nc(-c3ccc4c(ccc5ccccc54)c3)nc(-c3ccc4c(ccc5c6ccccc6ccc45)c3)n2)cc1 ; c1ccc(-c2nc(-c3ccc4c(ccc5c6ccccc6ccc45)c3)nc(-c3cc4ccccc4c4ccccc34)n2)cc1]

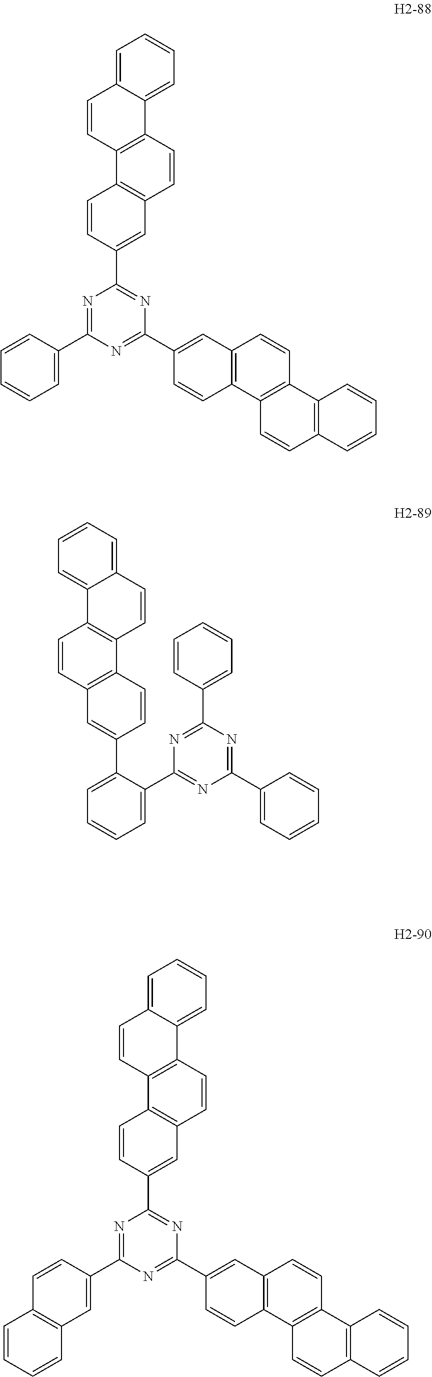 [SMILES: c1ccc(-c2nc(-c3ccc4c(ccc5c6ccccc6ccc45)c3)nc(-c3ccc4c(ccc5c6ccccc6ccc45)c3)n2)cc1 ; c1ccc(-c2nc(-c3ccccc3)nc(-c3ccccc3-c3ccc4c(ccc5c6ccccc6ccc45)c3)n2)cc1 ; c1ccc2cc(-c3nc(-c4ccc5c(ccc6c7ccccc7ccc56)c4)nc(-c4ccc5c(ccc6c7ccccc7ccc56)c4)n3)ccc2c1]

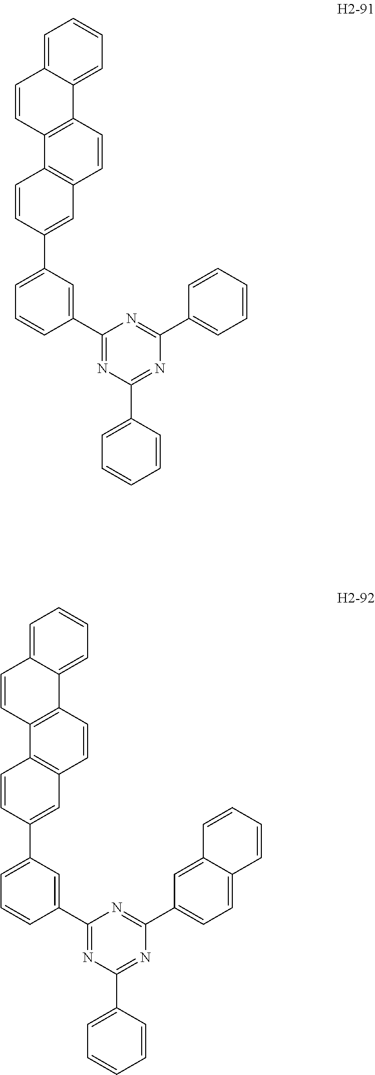 [SMILES: c1ccc(-c2nc(-c3ccccc3)nc(-c3cccc(-c4ccc5c(ccc6c7ccccc7ccc56)c4)c3)n2)cc1 ; c1ccc(-c2nc(-c3cccc(-c4ccc5c(ccc6c7ccccc7ccc56)c4)c3)nc(-c3ccc4ccccc4c3)n2)cc1]

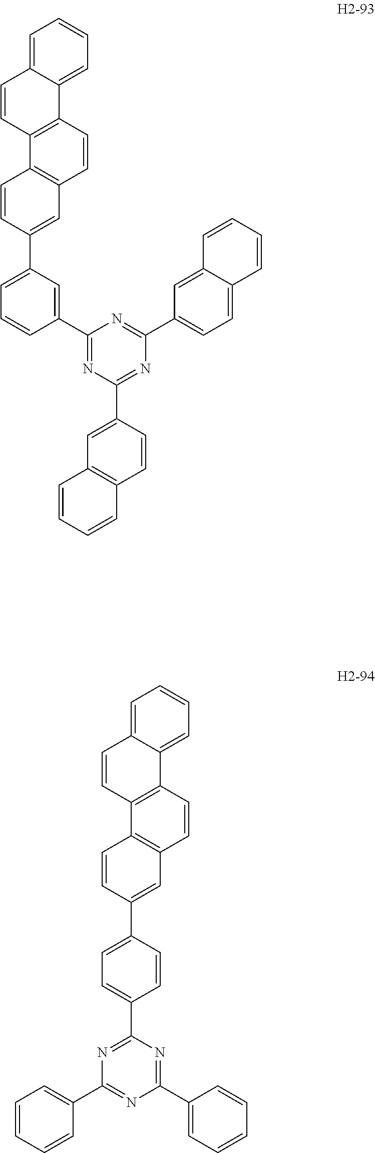 [SMILES: c1cc(-c2ccc3c(ccc4c5ccccc5ccc34)c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5c(ccc6c7ccccc7ccc56)c4)cc3)n2)cc1]

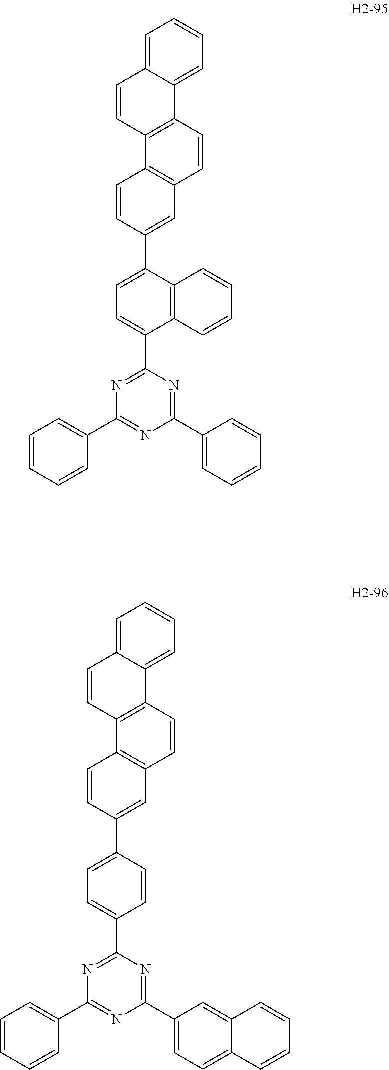 [SMILES: c1ccc(-c2nc(-c3ccccc3)nc(-c3ccc(-c4ccc5c(ccc6c7ccccc7ccc56)c4)c4ccccc34)n2)cc1 ; c1ccc(-c2nc(-c3ccc(-c4ccc5c(ccc6c7ccccc7ccc56)c4)cc3)nc(-c3ccc4ccccc4c3)n2)cc1]

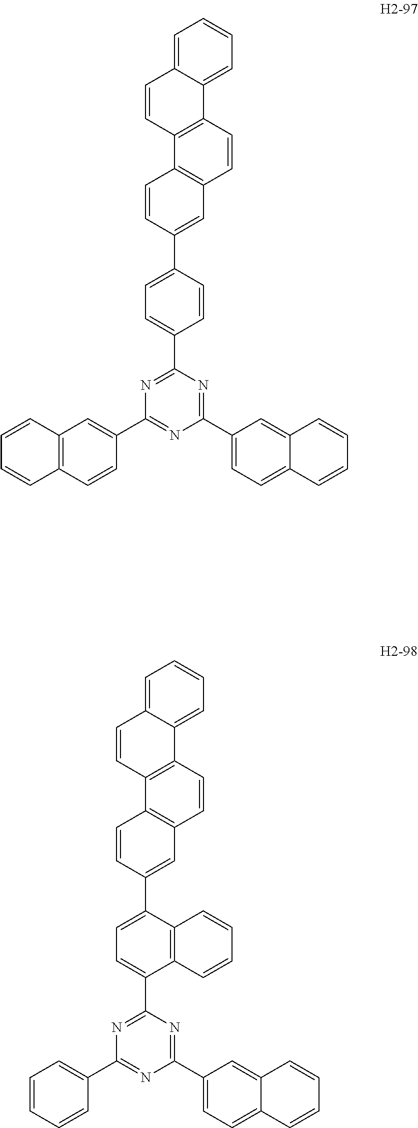 [SMILES: c1ccc2cc(-c3nc(-c4ccc(-c5ccc6c(ccc7c8ccccc8ccc67)c5)cc4)nc(-c4ccc5ccccc5c4)n3)ccc2c1 ; c1ccc(-c2nc(-c3ccc4ccccc4c3)nc(-c3ccc(-c4ccc5c(ccc6c7ccccc7ccc56)c4)c4ccccc34)n2)cc1]

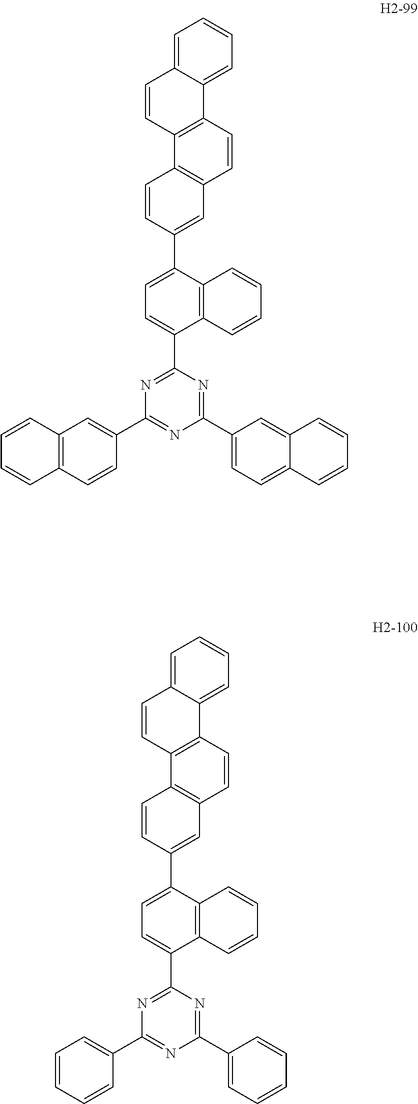 [SMILES: c1ccc2cc(-c3nc(-c4ccc5ccccc5c4)nc(-c4ccc(-c5ccc6c(ccc7c8ccccc8ccc67)c5)c5ccccc45)n3)ccc2c1 ; c1ccc(-c2nc(-c3ccccc3)nc(-c3ccc(-c4ccc5c(ccc6c7ccccc7ccc56)c4)c4ccccc34)n2)cc1]

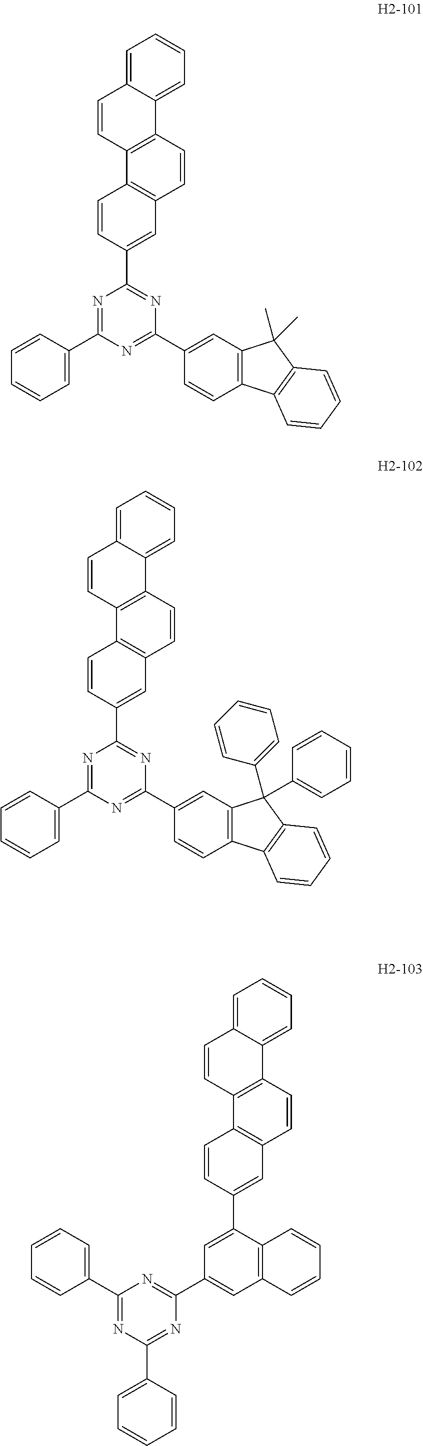 [SMILES: CC1(C)c2ccccc2-c2ccc(-c3nc(-c4ccccc4)nc(-c4ccc5c(ccc6c7ccccc7ccc56)c4)n3)cc21 ; c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1 ; c1ccc(-c2nc(-c3ccccc3)nc(-c3cc(-c4ccc5c(ccc6c7ccccc7ccc56)c4)c4ccccc4c3)n2)cc1]

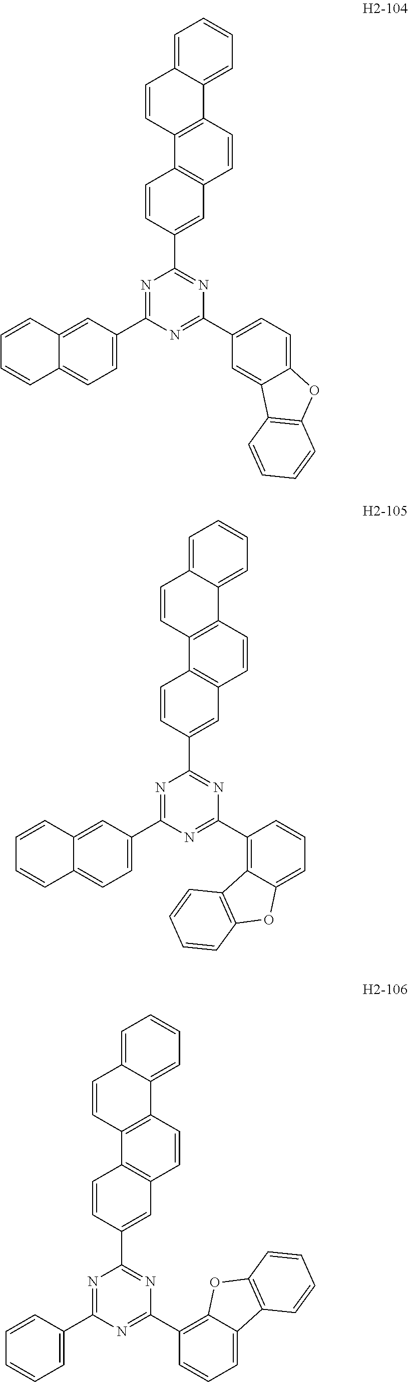 [SMILES: c1ccc2cc(-c3nc(-c4ccc5c(ccc6c7ccccc7ccc56)c4)nc(-c4ccc5oc6ccccc6c5c4)n3)ccc2c1 ; c1ccc2cc(-c3nc(-c4ccc5c(ccc6c7ccccc7ccc56)c4)nc(-c4cccc5oc6ccccc6c45)n3)ccc2c1 ; c1ccc(-c2nc(-c3ccc4c(ccc5c6ccccc6ccc45)c3)nc(-c3cccc4c3oc3ccccc34)n2)cc1]

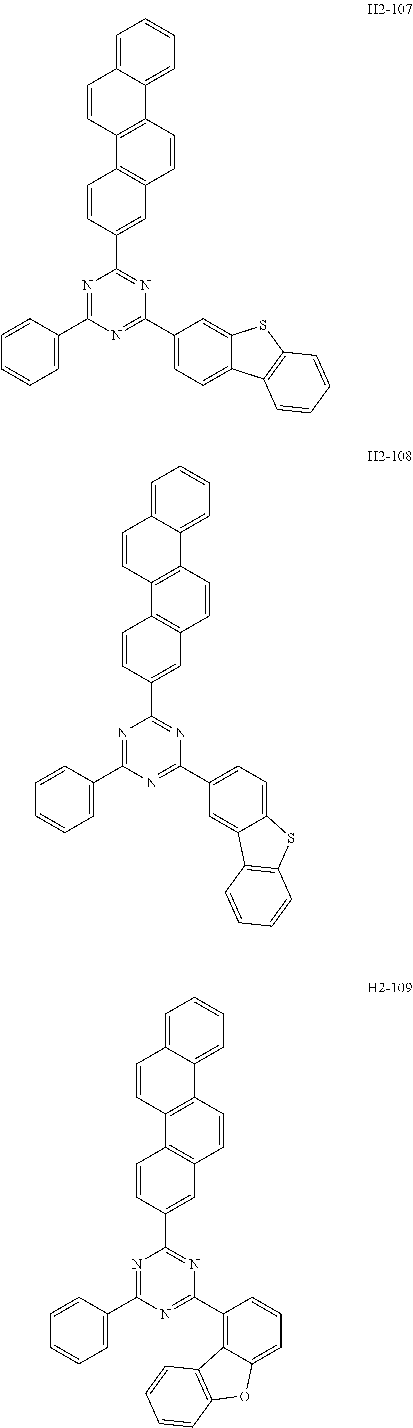 [SMILES: c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1 ; c1ccc(-c2nc(-c3ccc4c(ccc5c6ccccc6ccc45)c3)nc(-c3cccc4oc5ccccc5c34)n2)cc1]

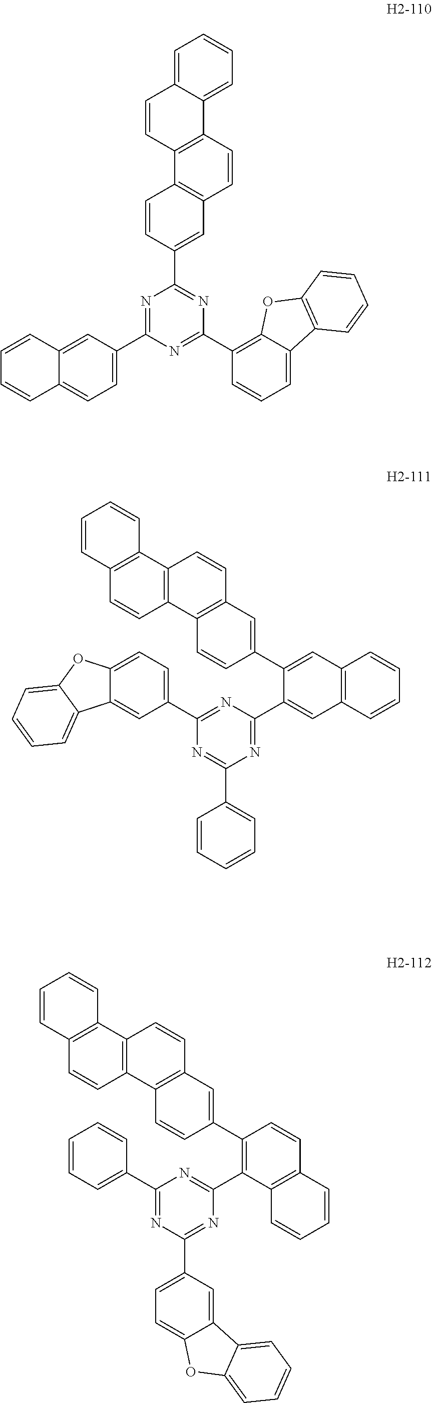 [SMILES: c1ccc2cc(-c3nc(-c4ccc5c(ccc6c7ccccc7ccc56)c4)nc(-c4cccc5c4oc4ccccc45)n3)ccc2c1 ; c1ccc(-c2nc(-c3ccc4oc5ccccc5c4c3)nc(-c3cc4ccccc4cc3-c3ccc4c(ccc5c6ccccc6ccc45)c3)n2)cc1 ; c1ccc(-c2nc(-c3ccc4oc5ccccc5c4c3)nc(-c3c(-c4ccc5c(ccc6c7ccccc7ccc56)c4)ccc4ccccc34)n2)cc1]

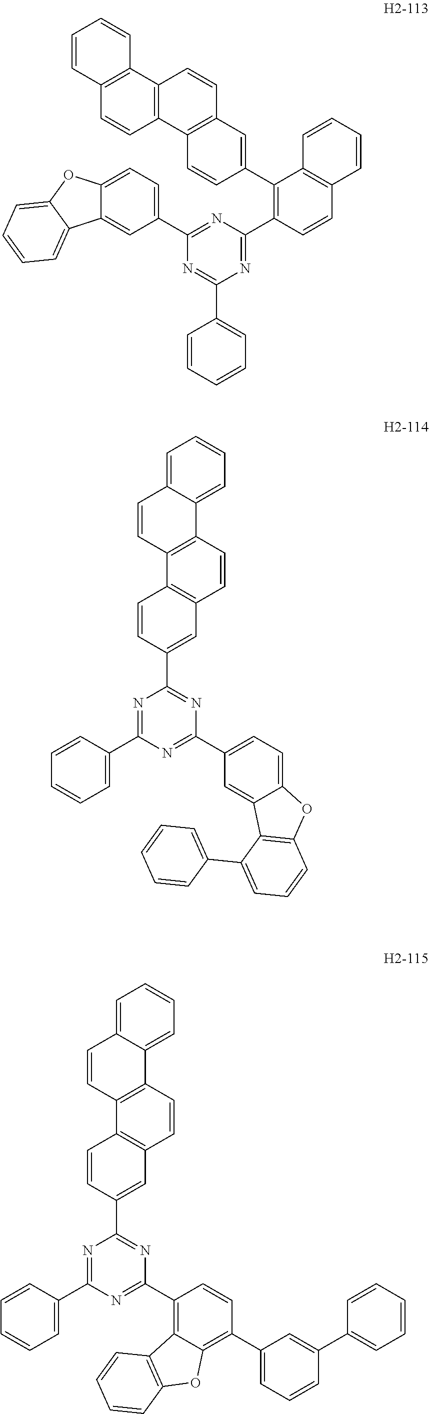 [SMILES: c1ccc(-c2nc(-c3ccc4oc5ccccc5c4c3)nc(-c3ccc4ccccc4c3-c3ccc4c(ccc5c6ccccc6ccc45)c3)n2)cc1 ; c1ccc(-c2nc(-c3ccc4c(ccc5c6ccccc6ccc45)c3)nc(-c3ccc4oc5cccc(-c6ccccc6)c5c4c3)n2)cc1 ; c1ccc(-c2cccc(-c3ccc(-c4nc(-c5ccccc5)nc(-c5ccc6c(ccc7c8ccccc8ccc67)c5)n4)c4c3oc3ccccc34)c2)cc1]

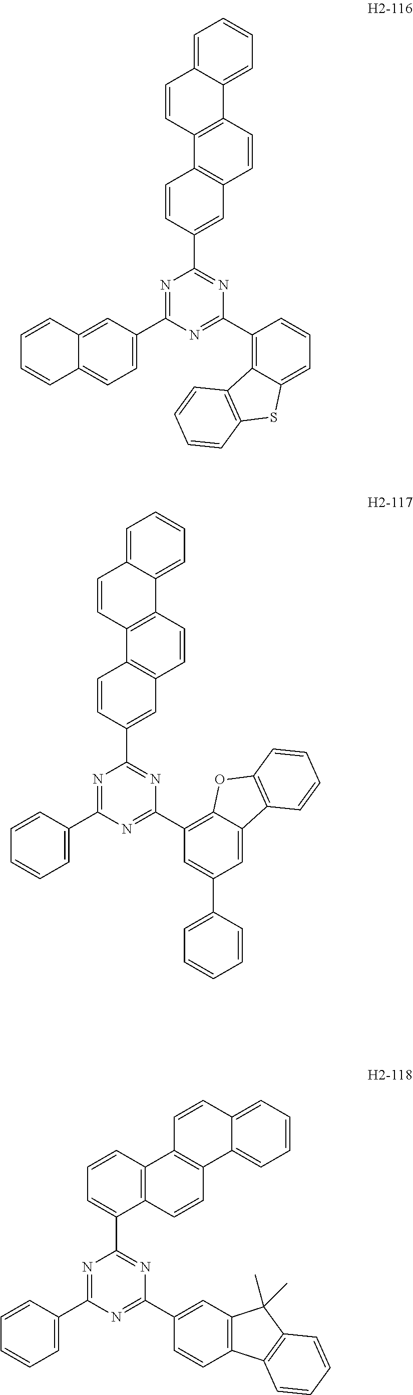 [SMILES: c1ccc2cc(-c3nc(-c4ccc5c(ccc6c7ccccc7ccc56)c4)nc(-c4cccc5sc6ccccc6c45)n3)ccc2c1 ; c1ccc(-c2cc(-c3nc(-c4ccccc4)nc(-c4ccc5c(ccc6c7ccccc7ccc56)c4)n3)c3oc4ccccc4c3c2)cc1 ; CC1(C)c2ccccc2-c2ccc(-c3nc(-c4ccccc4)nc(-c4cccc5c4ccc4c6ccccc6ccc54)n3)cc21]

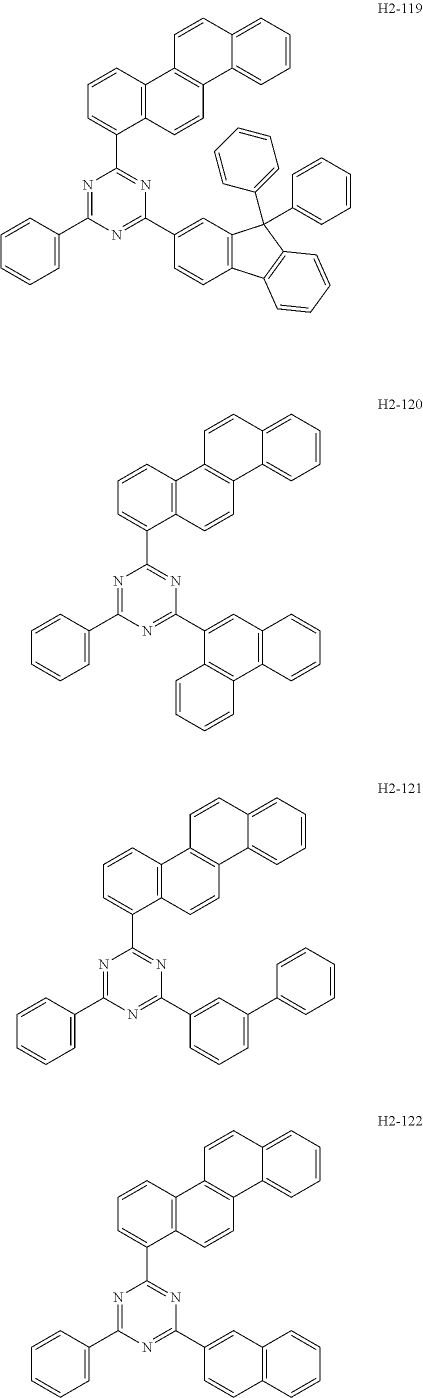 [SMILES: c1ccc(-c2nc(-c3ccc4c(c3)C(c3ccccc3)(c3ccccc3)c3ccccc3-4)nc(-c3cccc4c3ccc3c5ccccc5ccc43)n2)cc1 ; c1ccc(-c2nc(-c3cc4ccccc4c4ccccc34)nc(-c3cccc4c3ccc3c5ccccc5ccc43)n2)cc1 ; c1ccc(-c2cccc(-c3nc(-c4ccccc4)nc(-c4cccc5c4ccc4c6ccccc6ccc54)n3)c2)cc1 ; c1ccc(-c2nc(-c3ccc4ccccc4c3)nc(-c3cccc4c3ccc3c5ccccc5ccc43)n2)cc1]

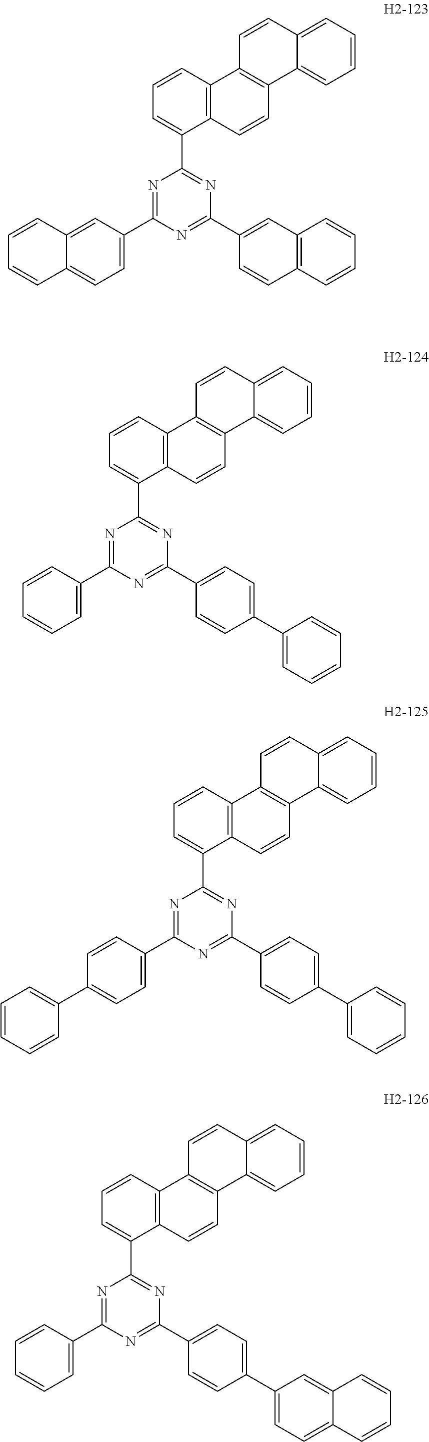 [SMILES: c1ccc2cc(-c3nc(-c4ccc5ccccc5c4)nc(-c4cccc5c4ccc4c6ccccc6ccc54)n3)ccc2c1 ; c1ccc(-c2ccc(-c3nc(-c4ccccc4)nc(-c4cccc5c4ccc4c6ccccc6ccc54)n3)cc2)cc1 ; c1ccc(-c2ccc(-c3nc(-c4ccc(-c5ccccc5)cc4)nc(-c4cccc5c4ccc4c6ccccc6ccc54)n3)cc2)cc1 ; c1ccc(-c2nc(-c3ccc(-c4ccc5ccccc5c4)cc3)nc(-c3cccc4c3ccc3c5ccccc5ccc43)n2)cc1]

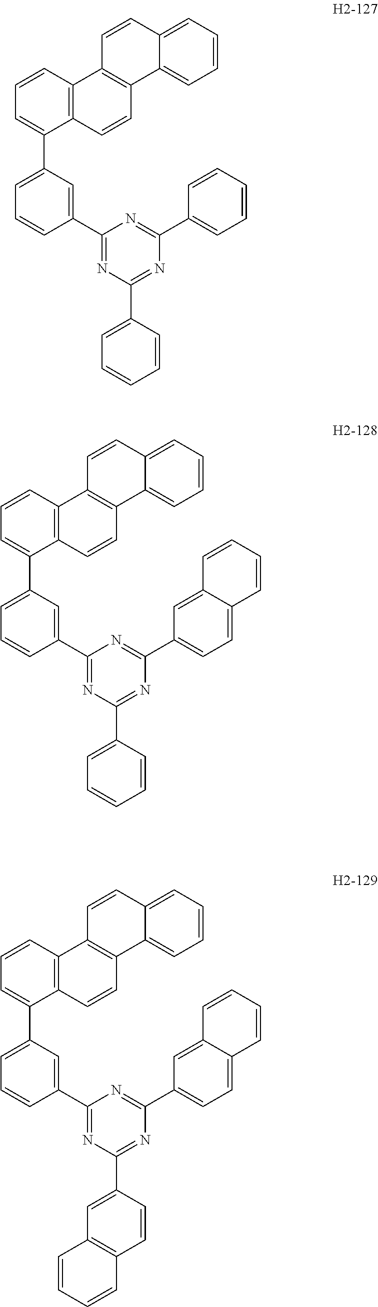 [SMILES: c1ccc(-c2nc(-c3ccccc3)nc(-c3cccc(-c4cccc5c4ccc4c6ccccc6ccc54)c3)n2)cc1 ; c1ccc(-c2nc(-c3cccc(-c4cccc5c4ccc4c6ccccc6ccc54)c3)nc(-c3ccc4ccccc4c3)n2)cc1 ; c1cc(-c2nc(-c3ccc4ccccc4c3)nc(-c3ccc4ccccc4c3)n2)cc(-c2cccc3c2ccc2c4ccccc4ccc32)c1]

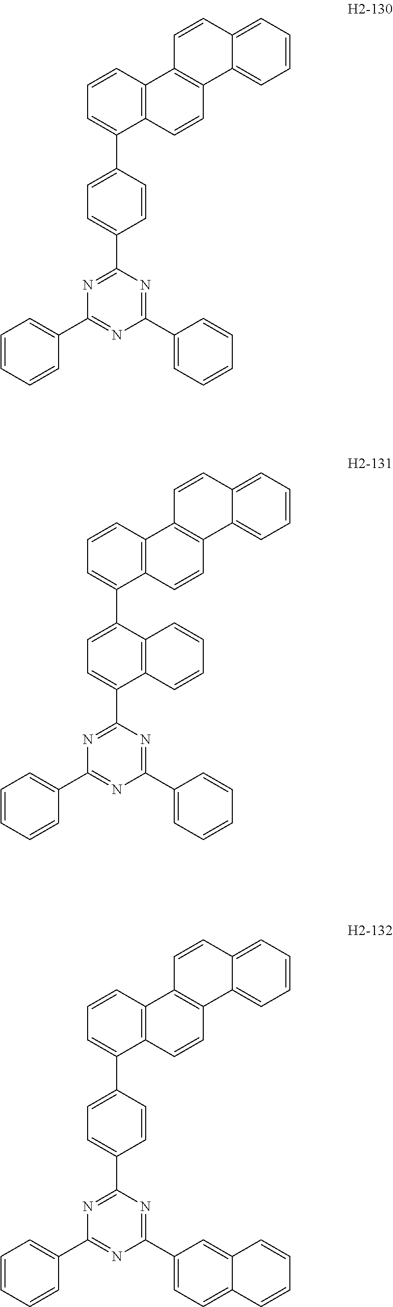 [SMILES: c1ccc(-c2nc(-c3ccccc3)nc(-c3ccc(-c4cccc5c4ccc4c6ccccc6ccc54)cc3)n2)cc1 ; c1ccc(-c2nc(-c3ccccc3)nc(-c3ccc(-c4cccc5c4ccc4c6ccccc6ccc54)c4ccccc34)n2)cc1 ; c1ccc(-c2nc(-c3ccc(-c4cccc5c4ccc4c6ccccc6ccc54)cc3)nc(-c3ccc4ccccc4c3)n2)cc1]

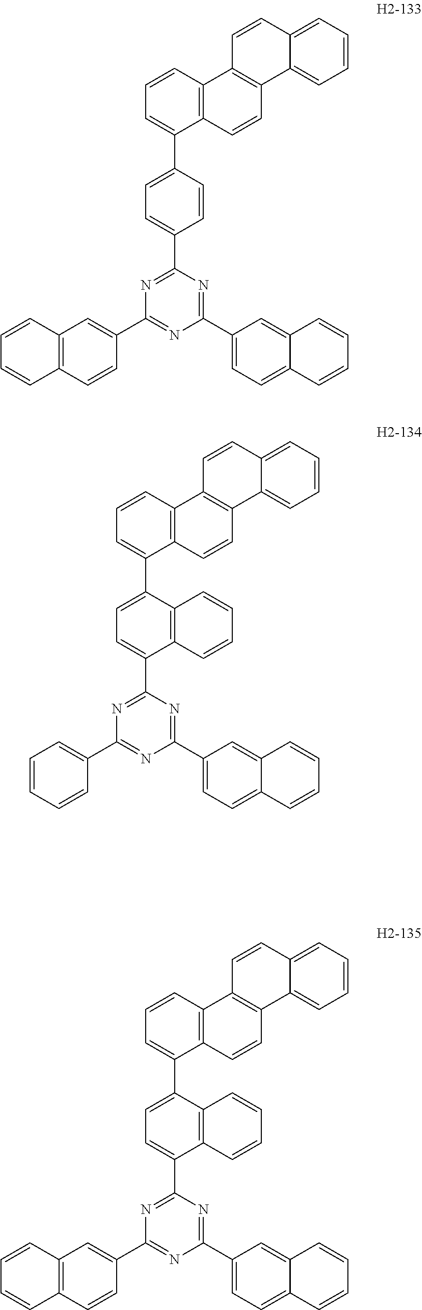 [SMILES: c1ccc2cc(-c3nc(-c4ccc(-c5cccc6c5ccc5c7ccccc7ccc65)cc4)nc(-c4ccc5ccccc5c4)n3)ccc2c1 ; c1ccc(-c2nc(-c3ccc4ccccc4c3)nc(-c3ccc(-c4cccc5c4ccc4c6ccccc6ccc54)c4ccccc34)n2)cc1 ; c1ccc2cc(-c3nc(-c4ccc5ccccc5c4)nc(-c4ccc(-c5cccc6c5ccc5c7ccccc7ccc65)c5ccccc45)n3)ccc2c1]

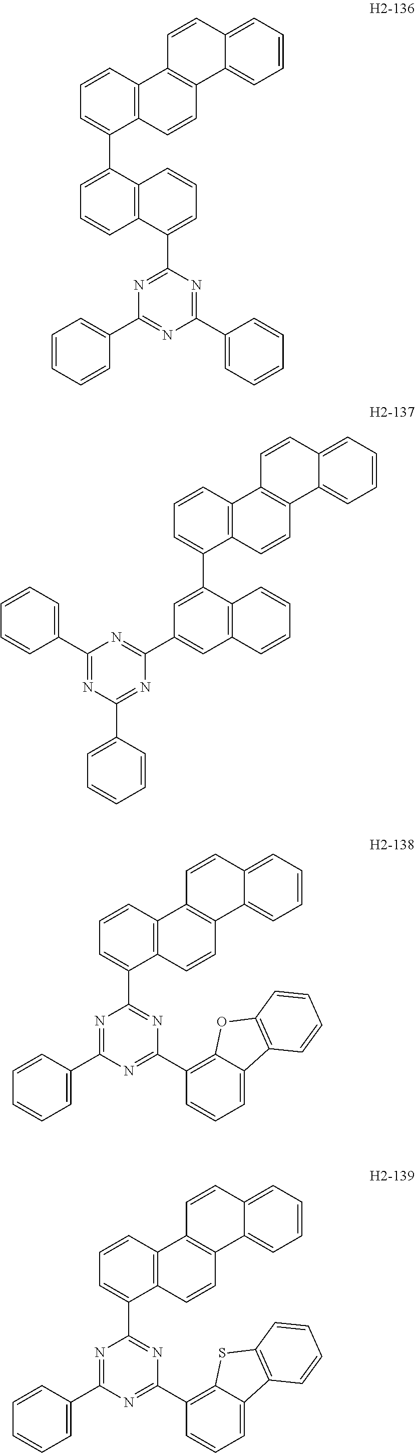 [SMILES: c1ccc(-c2nc(-c3ccccc3)nc(-c3cccc4c(-c5cccc6c5ccc5c7ccccc7ccc65)cccc34)n2)cc1 ; c1ccc(-c2nc(-c3ccccc3)nc(-c3cc(-c4cccc5c4ccc4c6ccccc6ccc54)c4ccccc4c3)n2)cc1 ; c1ccc(-c2nc(-c3cccc4c3ccc3c5ccccc5ccc43)nc(-c3cccc4c3oc3ccccc34)n2)cc1 ; c1ccc(-c2nc(-c3cccc4c3ccc3c5ccccc5ccc43)nc(-c3cccc4c3sc3ccccc34)n2)cc1]

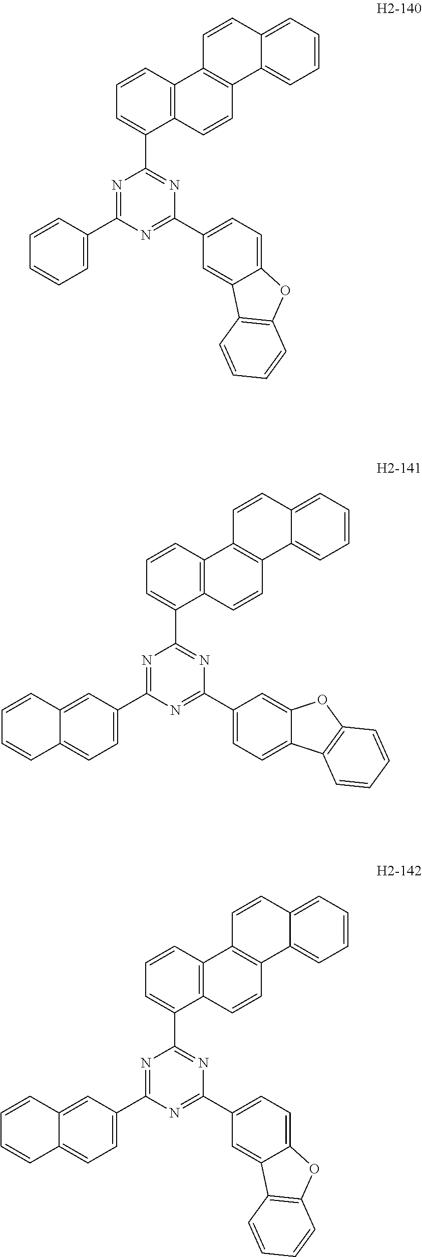 [SMILES: c1ccc(-c2nc(-c3ccc4oc5ccccc5c4c3)nc(-c3cccc4c3ccc3c5ccccc5ccc43)n2)cc1 ; c1ccc2cc(-c3nc(-c4ccc5c(c4)oc4ccccc45)nc(-c4cccc5c4ccc4c6ccccc6ccc54)n3)ccc2c1 ; c1ccc2cc(-c3nc(-c4ccc5oc6ccccc6c5c4)nc(-c4cccc5c4ccc4c6ccccc6ccc54)n3)ccc2c1]

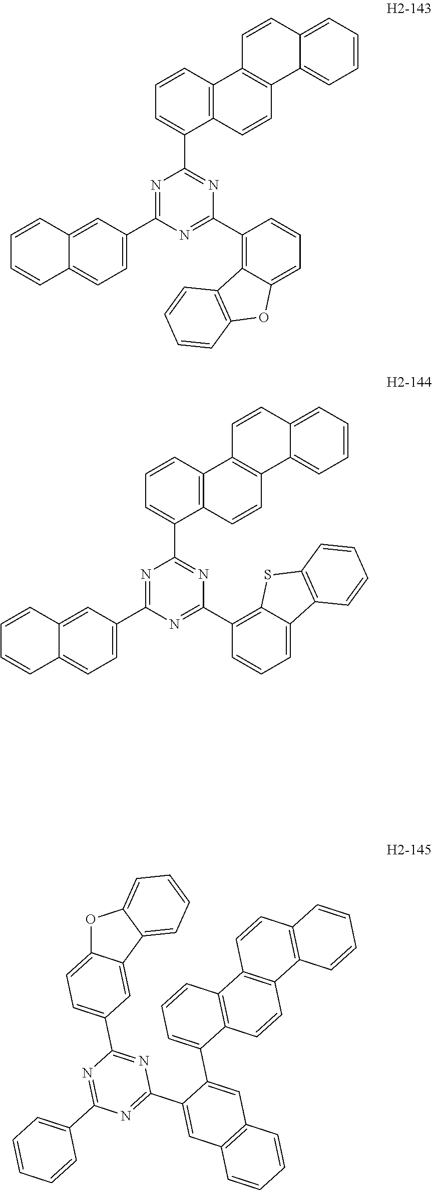 [SMILES: c1ccc2cc(-c3nc(-c4cccc5c4ccc4c6ccccc6ccc54)nc(-c4cccc5oc6ccccc6c45)n3)ccc2c1 ; c1ccc2cc(-c3nc(-c4cccc5c4ccc4c6ccccc6ccc54)nc(-c4cccc5c4sc4ccccc45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1]

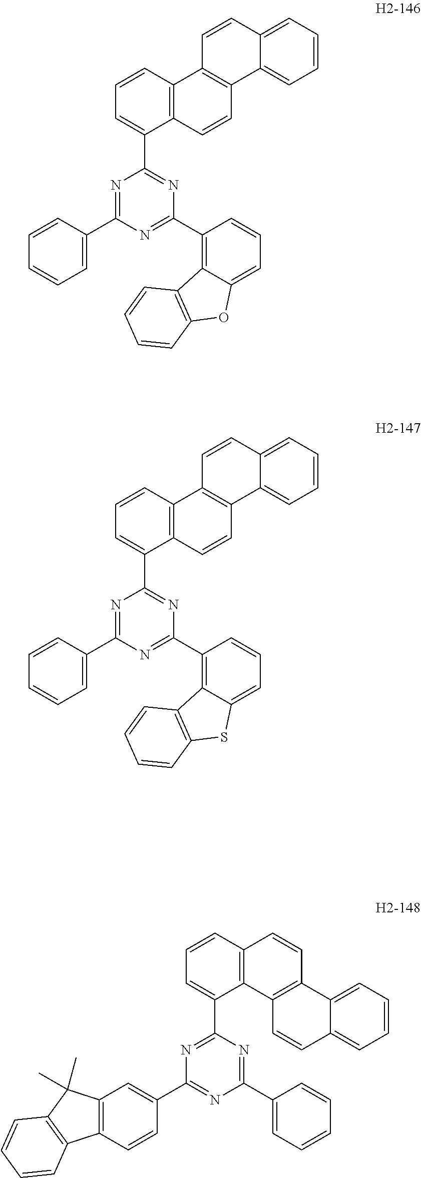 [SMILES: c1ccc(-c2nc(-c3cccc4c3ccc3c5ccccc5ccc43)nc(-c3cccc4oc5ccccc5c34)n2)cc1 ; c1ccc(-c2nc(-c3cccc4c3ccc3c5ccccc5ccc43)nc(-c3cccc4sc5ccccc5c34)n2)cc1 ; CC1(C)c2ccccc2-c2ccc(-c3nc(-c4ccccc4)nc(-c4cccc5ccc6c7ccccc7ccc6c45)n3)cc21]

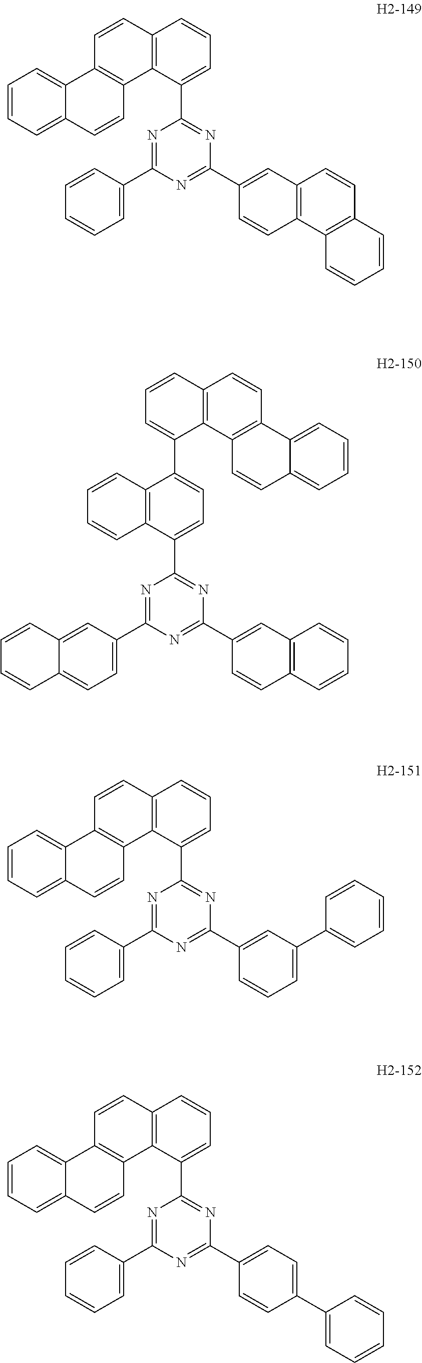 [SMILES: c1ccc(-c2nc(-c3ccc4c(ccc5ccccc54)c3)nc(-c3cccc4ccc5c6ccccc6ccc5c34)n2)cc1 ; c1ccc2cc(-c3nc(-c4ccc5ccccc5c4)nc(-c4ccc(-c5cccc6ccc7c8ccccc8ccc7c56)c5ccccc45)n3)ccc2c1 ; c1ccc(-c2cccc(-c3nc(-c4ccccc4)nc(-c4cccc5ccc6c7ccccc7ccc6c45)n3)c2)cc1 ; c1ccc(-c2ccc(-c3nc(-c4ccccc4)nc(-c4cccc5ccc6c7ccccc7ccc6c45)n3)cc2)cc1]

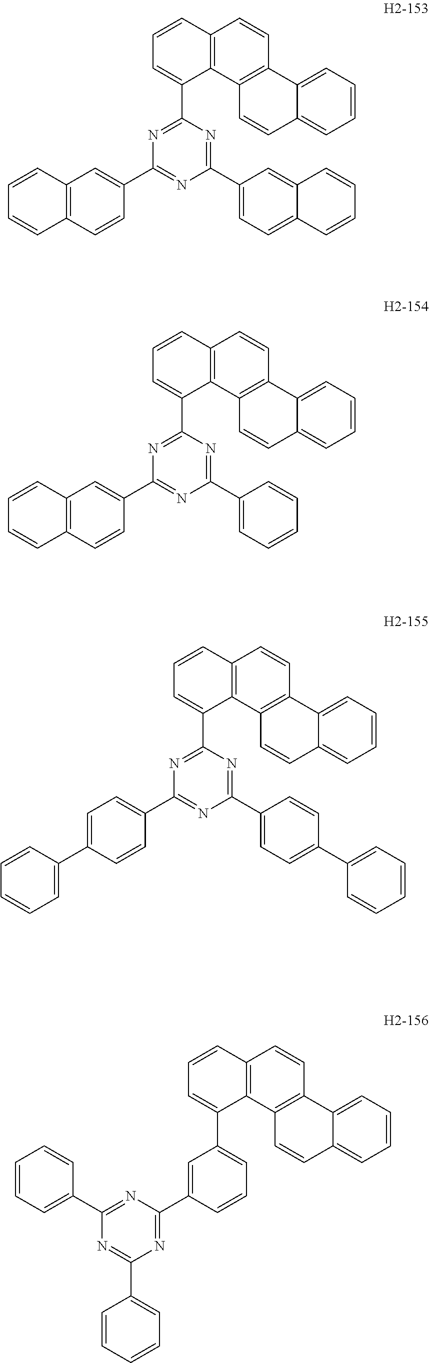 [SMILES: c1ccc2cc(-c3nc(-c4ccc5ccccc5c4)nc(-c4cccc5ccc6c7ccccc7ccc6c45)n3)ccc2c1 ; c1ccc(-c2nc(-c3ccc4ccccc4c3)nc(-c3cccc4ccc5c6ccccc6ccc5c34)n2)cc1 ; c1ccc(-c2ccc(-c3nc(-c4ccc(-c5ccccc5)cc4)nc(-c4cccc5ccc6c7ccccc7ccc6c45)n3)cc2)cc1 ; c1ccc(-c2nc(-c3ccccc3)nc(-c3cccc(-c4cccc5ccc6c7ccccc7ccc6c45)c3)n2)cc1]

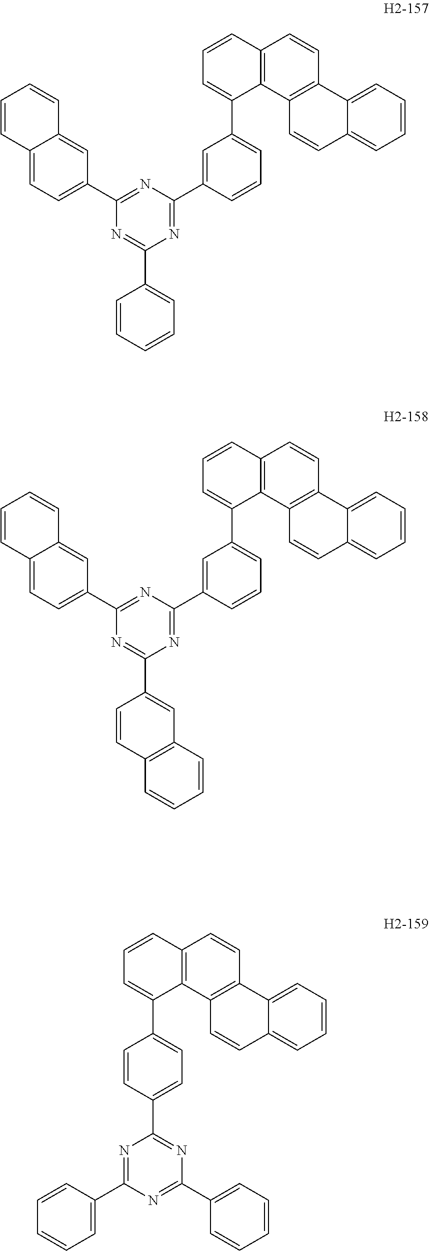 [SMILES: c1ccc(-c2nc(-c3cccc(-c4cccc5ccc6c7ccccc7ccc6c45)c3)nc(-c3ccc4ccccc4c3)n2)cc1 ; c1cc(-c2nc(-c3ccc4ccccc4c3)nc(-c3ccc4ccccc4c3)n2)cc(-c2cccc3ccc4c5ccccc5ccc4c23)c1 ; c1ccc(-c2nc(-c3ccccc3)nc(-c3ccc(-c4cccc5ccc6c7ccccc7ccc6c45)cc3)n2)cc1]

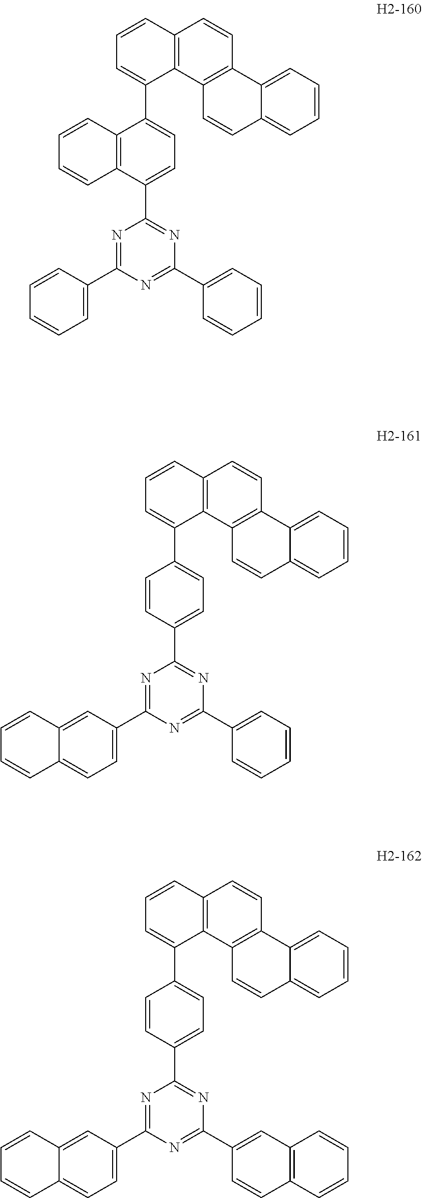 [SMILES: c1ccc(-c2nc(-c3ccccc3)nc(-c3ccc(-c4cccc5ccc6c7ccccc7ccc6c45)c4ccccc34)n2)cc1 ; c1ccc(-c2nc(-c3ccc(-c4cccc5ccc6c7ccccc7ccc6c45)cc3)nc(-c3ccc4ccccc4c3)n2)cc1 ; c1ccc2cc(-c3nc(-c4ccc(-c5cccc6ccc7c8ccccc8ccc7c56)cc4)nc(-c4ccc5ccccc5c4)n3)ccc2c1]

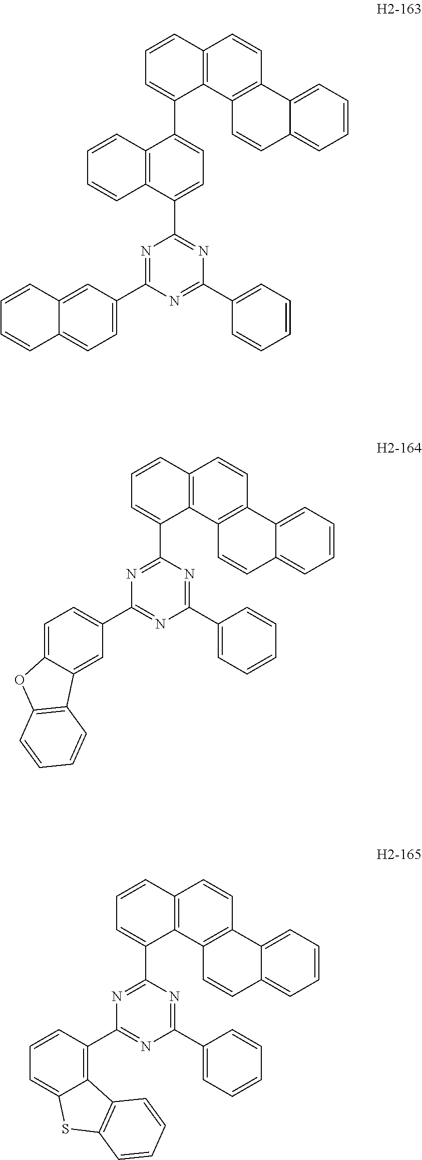 [SMILES: c1ccc(-c2nc(-c3ccc4ccccc4c3)nc(-c3ccc(-c4cccc5ccc6c7ccccc7ccc6c45)c4ccccc34)n2)cc1 ; c1ccc(-c2nc(-c3ccc4oc5ccccc5c4c3)nc(-c3cccc4ccc5c6ccccc6ccc5c34)n2)cc1 ; c1ccc(-c2nc(-c3cccc4ccc5c6ccccc6ccc5c34)nc(-c3cccc4sc5ccccc5c34)n2)cc1]

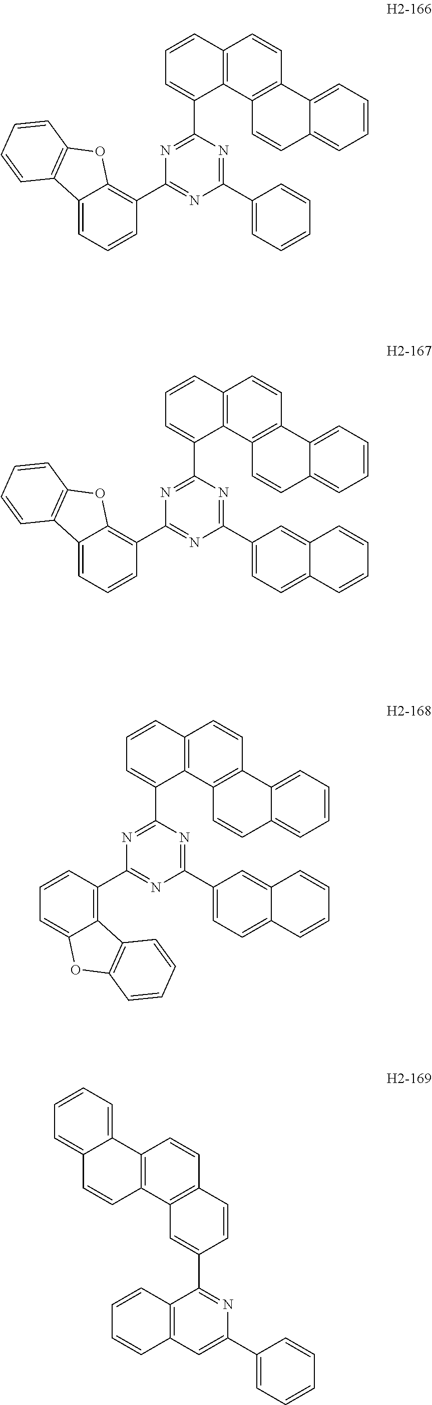 [SMILES: c1ccc(-c2nc(-c3cccc4c3oc3ccccc34)nc(-c3cccc4ccc5c6ccccc6ccc5c34)n2)cc1 ; c1ccc2cc(-c3nc(-c4cccc5c4oc4ccccc45)nc(-c4cccc5ccc6c7ccccc7ccc6c45)n3)ccc2c1 ; c1ccc2cc(-c3nc(-c4cccc5ccc6c7ccccc7ccc6c45)nc(-c4cccc5oc6ccccc6c45)n3)ccc2c1 ; c1ccc(-c2cc3ccccc3c(-c3ccc4ccc5c6ccccc6ccc5c4c3)n2)cc1]

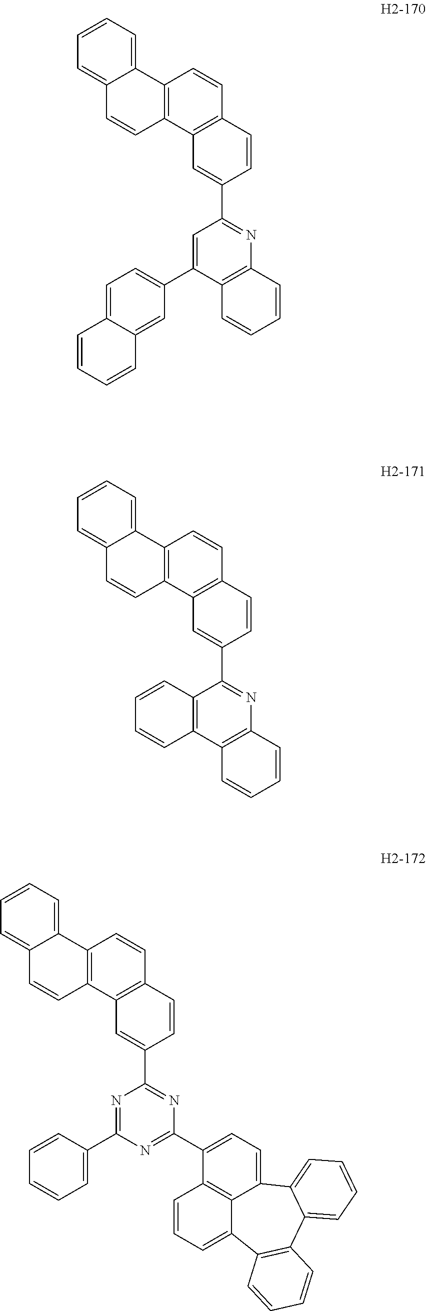 [SMILES: c1ccc2cc(-c3cc(-c4ccc5ccc6c7ccccc7ccc6c5c4)nc4ccccc34)ccc2c1 ; c1ccc2c(c1)ccc1c3cc(-c4nc5ccccc5c5ccccc45)ccc3ccc21 ; c1ccc(-c2nc(-c3ccc4ccc5c6ccccc6ccc5c4c3)nc(-c3ccc4c5c(cccc35)-c3ccccc3-c3ccccc3-4)n2)cc1]

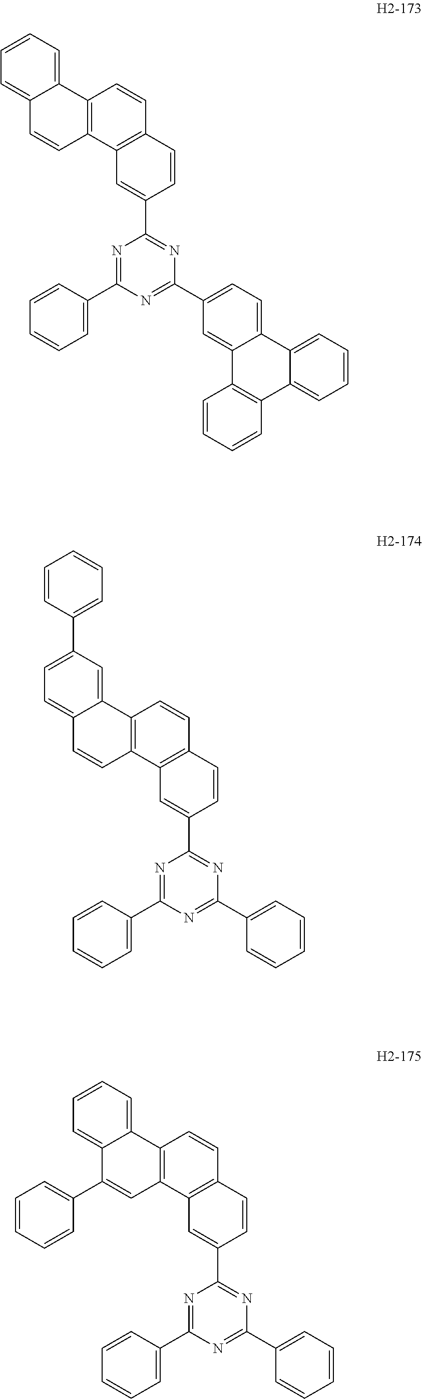 [SMILES: c1ccc(-c2nc(-c3ccc4ccc5c6ccccc6ccc5c4c3)nc(-c3ccc4c5ccccc5c5ccccc5c4c3)n2)cc1 ; c1ccc(-c2ccc3ccc4c5cc(-c6nc(-c7ccccc7)nc(-c7ccccc7)n6)ccc5ccc4c3c2)cc1 ; c1ccc(-c2nc(-c3ccccc3)nc(-c3ccc4ccc5c6ccccc6c(-c6ccccc6)cc5c4c3)n2)cc1]

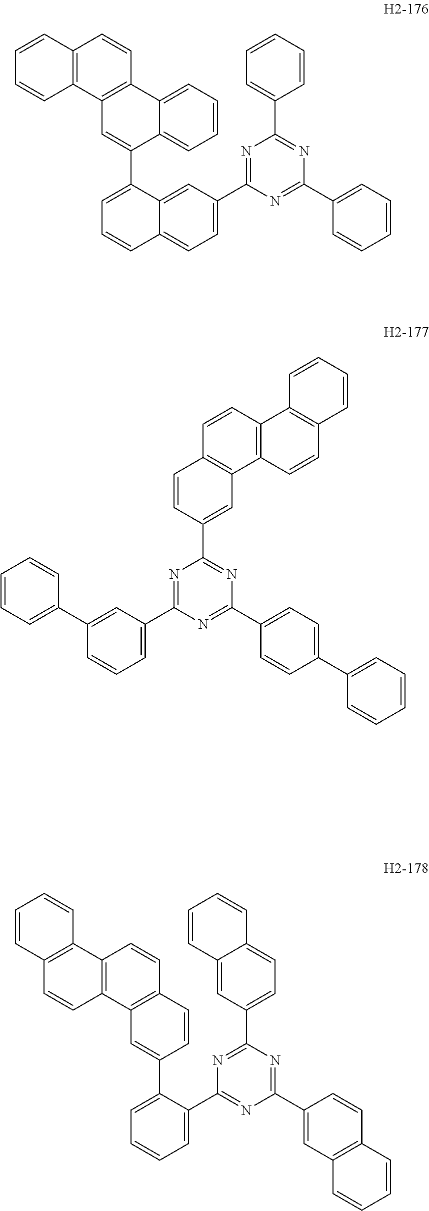 [SMILES: c1ccc(-c2nc(-c3ccccc3)nc(-c3ccc4cccc(-c5cc6c7ccccc7ccc6c6ccccc56)c4c3)n2)cc1 ; c1ccc(-c2ccc(-c3nc(-c4cccc(-c5ccccc5)c4)nc(-c4ccc5ccc6c7ccccc7ccc6c5c4)n3)cc2)cc1 ; c1ccc(-c2nc(-c3ccc4ccccc4c3)nc(-c3ccc4ccccc4c3)n2)c(-c2ccc3ccc4c5ccccc5ccc4c3c2)c1]

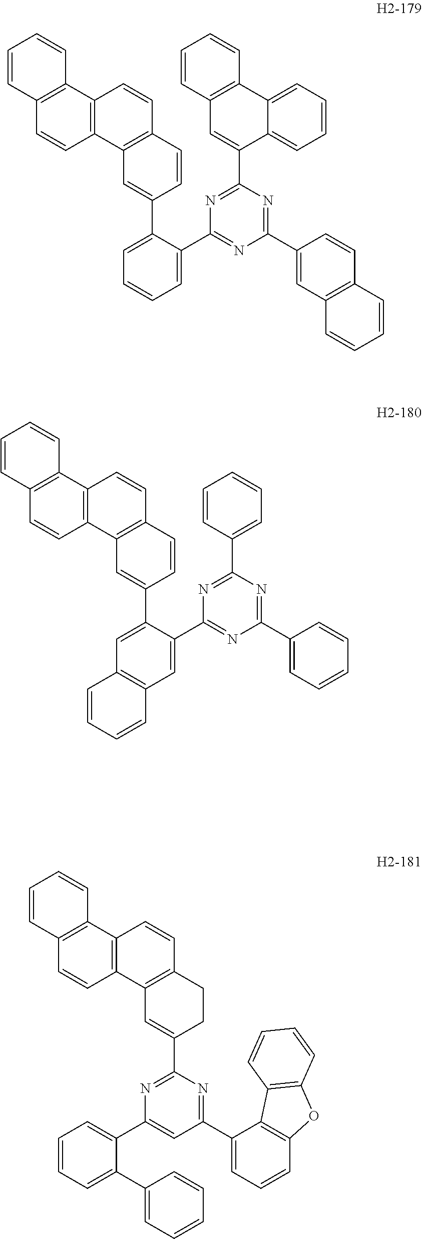 [SMILES: c1ccc(-c2nc(-c3ccc4ccccc4c3)nc(-c3cc4ccccc4c4ccccc34)n2)c(-c2ccc3ccc4c5ccccc5ccc4c3c2)c1 ; c1ccc(-c2nc(-c3ccccc3)nc(-c3cc4ccccc4cc3-c3ccc4ccc5c6ccccc6ccc5c4c3)n2)cc1 ; C1=C(c2nc(-c3ccccc3-c3ccccc3)cc(-c3cccc4oc5ccccc5c34)n2)CCc2ccc3c(ccc4ccccc43)c21]

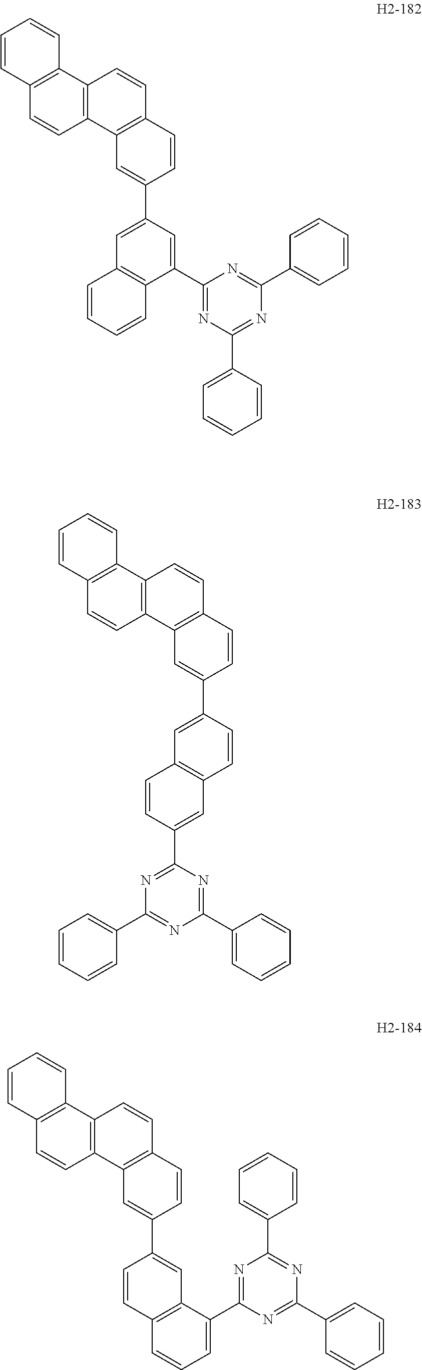 [SMILES: c1ccc(-c2nc(-c3ccccc3)nc(-c3cc(-c4ccc5ccc6c7ccccc7ccc6c5c4)cc4ccccc34)n2)cc1 ; c1ccc(-c2nc(-c3ccccc3)nc(-c3ccc4cc(-c5ccc6ccc7c8ccccc8ccc7c6c5)ccc4c3)n2)cc1 ; c1ccc(-c2nc(-c3ccccc3)nc(-c3cccc4ccc(-c5ccc6ccc7c8ccccc8ccc7c6c5)cc34)n2)cc1]

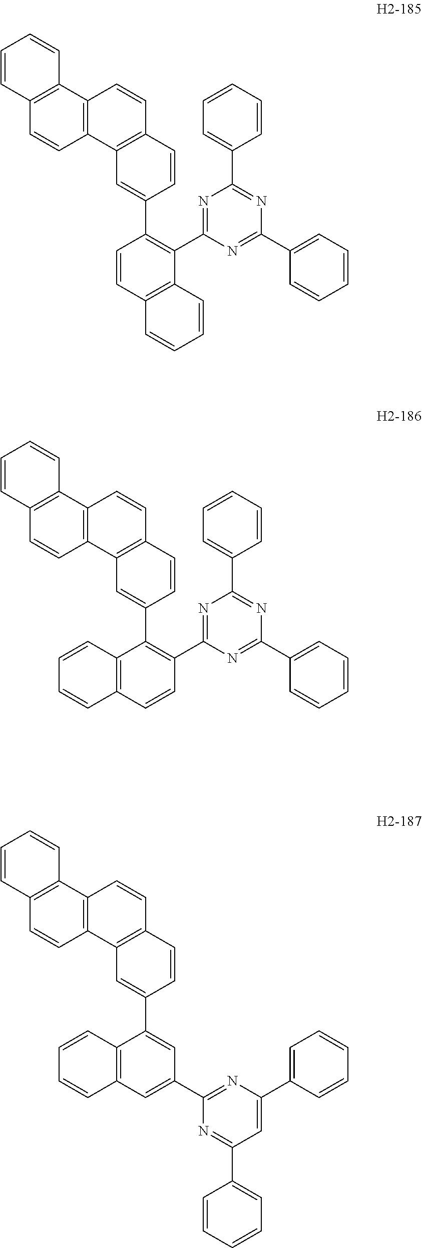 [SMILES: c1ccc(-c2nc(-c3ccccc3)nc(-c3c(-c4ccc5ccc6c7ccccc7ccc6c5c4)ccc4ccccc34)n2)cc1 ; c1ccc(-c2nc(-c3ccccc3)nc(-c3ccc4ccccc4c3-c3ccc4ccc5c6ccccc6ccc5c4c3)n2)cc1 ; c1ccc(-c2cc(-c3ccccc3)nc(-c3cc(-c4ccc5ccc6c7ccccc7ccc6c5c4)c4ccccc4c3)n2)cc1]

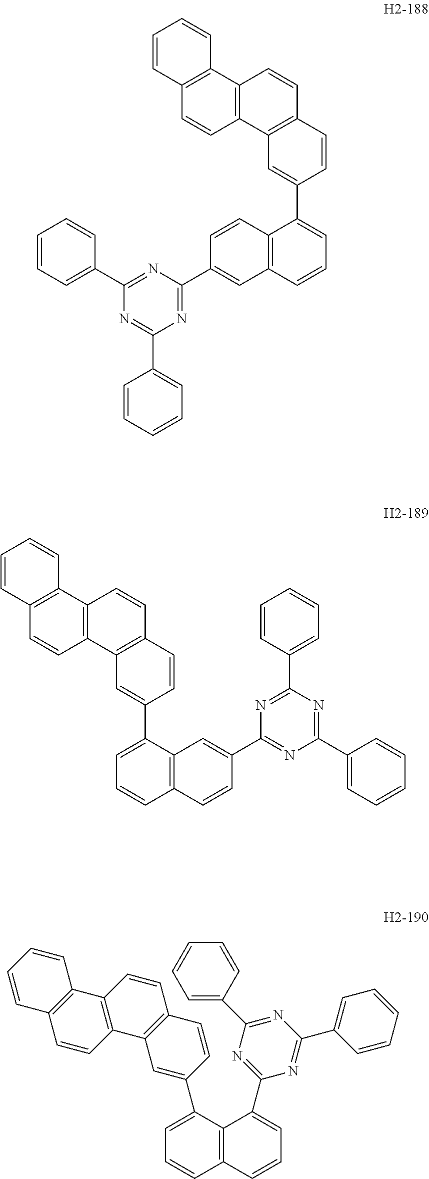 [SMILES: c1ccc(-c2nc(-c3ccccc3)nc(-c3ccc4c(-c5ccc6ccc7c8ccccc8ccc7c6c5)cccc4c3)n2)cc1 ; c1ccc(-c2nc(-c3ccccc3)nc(-c3ccc4cccc(-c5ccc6ccc7c8ccccc8ccc7c6c5)c4c3)n2)cc1 ; c1ccc(-c2nc(-c3ccccc3)nc(-c3cccc4cccc(-c5ccc6ccc7c8ccccc8ccc7c6c5)c34)n2)cc1]

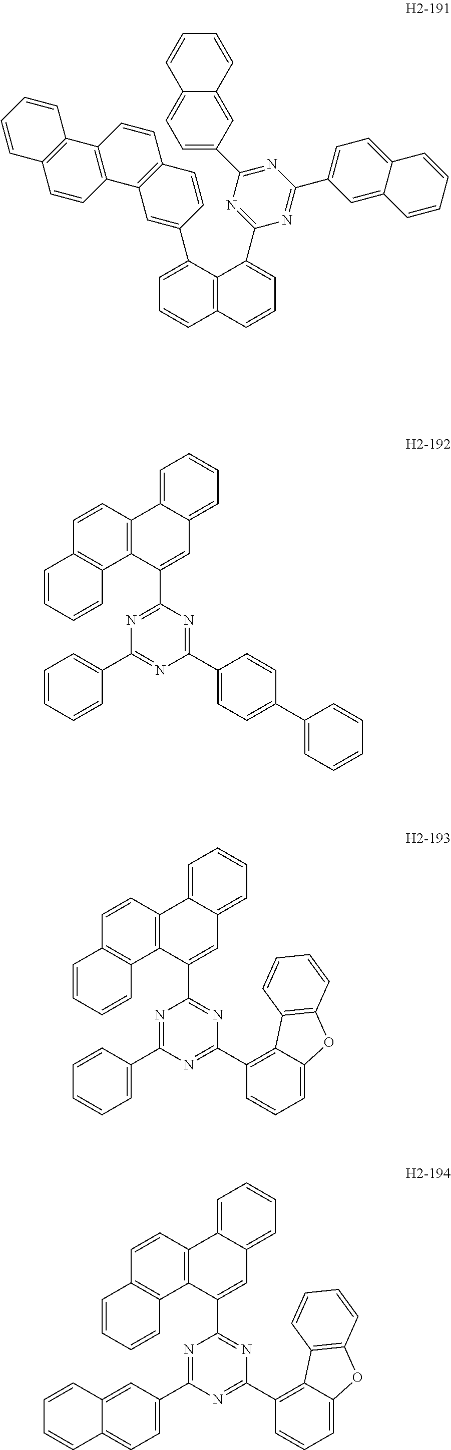 [SMILES: c1ccc2cc(-c3nc(-c4ccc5ccccc5c4)nc(-c4cccc5cccc(-c6ccc7ccc8c9ccccc9ccc8c7c6)c45)n3)ccc2c1 ; c1ccc(-c2ccc(-c3nc(-c4ccccc4)nc(-c4cc5ccccc5c5ccc6ccccc6c45)n3)cc2)cc1 ; c1ccc(-c2nc(-c3cccc4oc5ccccc5c34)nc(-c3cc4ccccc4c4ccc5ccccc5c34)n2)cc1 ; c1ccc2cc(-c3nc(-c4cccc5oc6ccccc6c45)nc(-c4cc5ccccc5c5ccc6ccccc6c45)n3)ccc2c1]

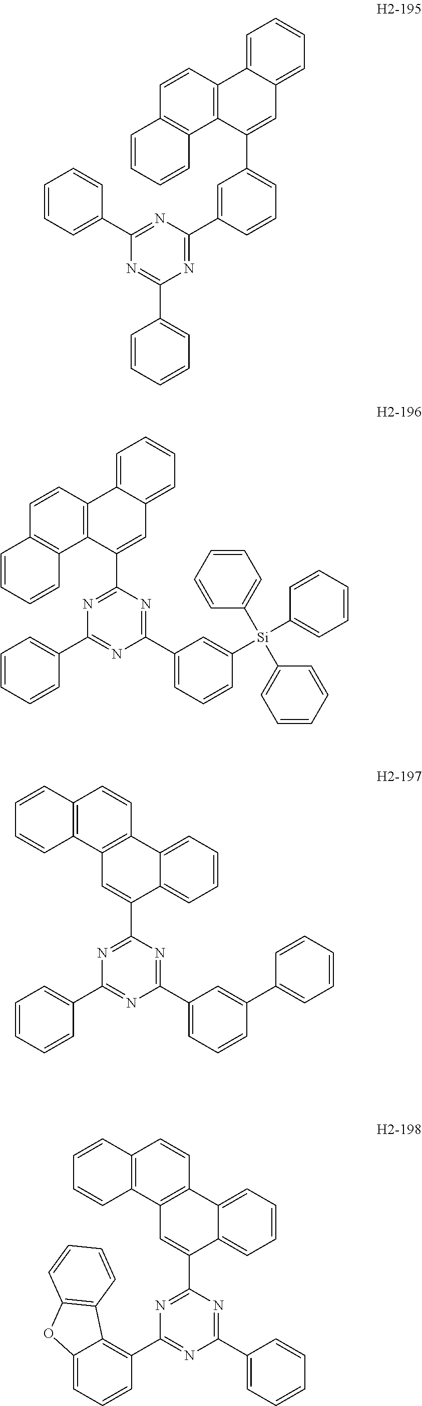 [SMILES: c1ccc(-c2nc(-c3ccccc3)nc(-c3cccc(-c4cc5ccccc5c5ccc6ccccc6c45)c3)n2)cc1 ; c1ccc(-c2nc(-c3cccc([Si](c4ccccc4)(c4ccccc4)c4ccccc4)c3)nc(-c3cc4ccccc4c4ccc5ccccc5c34)n2)cc1 ; c1ccc(-c2cccc(-c3nc(-c4ccccc4)nc(-c4cc5c6ccccc6ccc5c5ccccc45)n3)c2)cc1 ; c1ccc(-c2nc(-c3cc4c5ccccc5ccc4c4ccccc34)nc(-c3cccc4oc5ccccc5c34)n2)cc1]

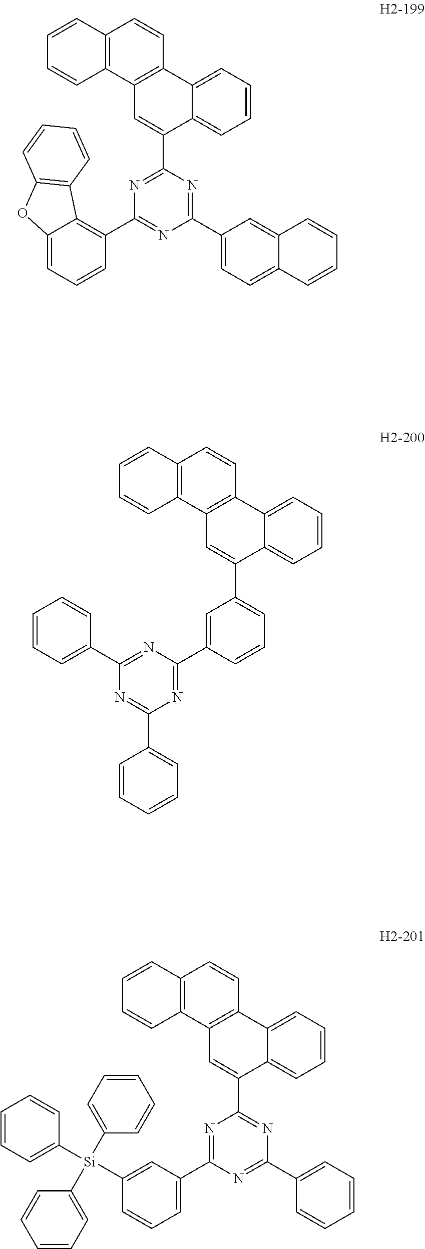 [SMILES: c1ccc2cc(-c3nc(-c4cc5c6ccccc6ccc5c5ccccc45)nc(-c4cccc5oc6ccccc6c45)n3)ccc2c1 ; c1ccc(-c2nc(-c3ccccc3)nc(-c3cccc(-c4cc5c6ccccc6ccc5c5ccccc45)c3)n2)cc1 ; c1ccc(-c2nc(-c3cccc([Si](c4ccccc4)(c4ccccc4)c4ccccc4)c3)nc(-c3cc4c5ccccc5ccc4c4ccccc34)n2)cc1]

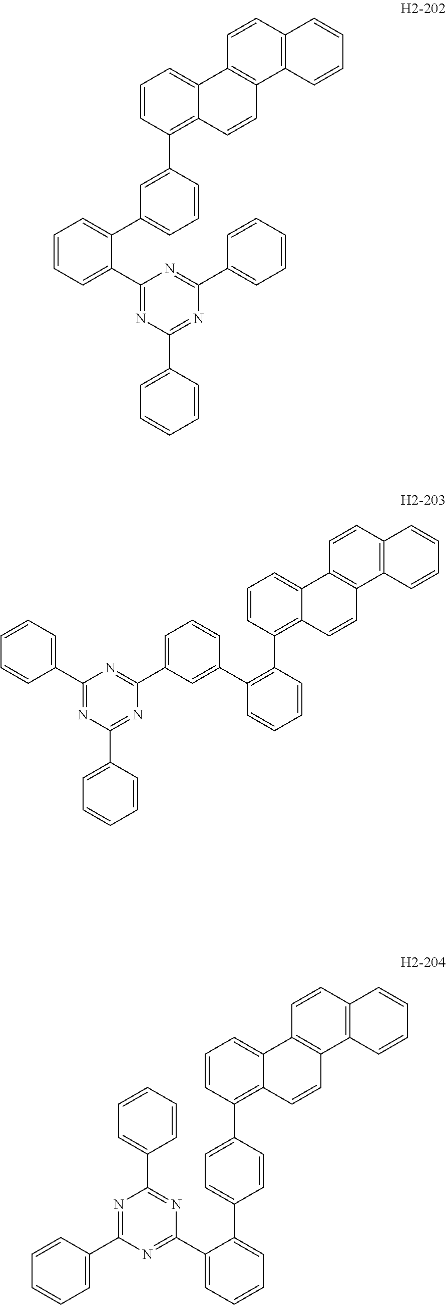 [SMILES: c1ccc(-c2nc(-c3ccccc3)nc(-c3ccccc3-c3cccc(-c4cccc5c4ccc4c6ccccc6ccc54)c3)n2)cc1 ; c1ccc(-c2nc(-c3ccccc3)nc(-c3cccc(-c4ccccc4-c4cccc5c4ccc4c6ccccc6ccc54)c3)n2)cc1 ; c1ccc(-c2nc(-c3ccccc3)nc(-c3ccccc3-c3ccc(-c4cccc5c4ccc4c6ccccc6ccc54)cc3)n2)cc1]

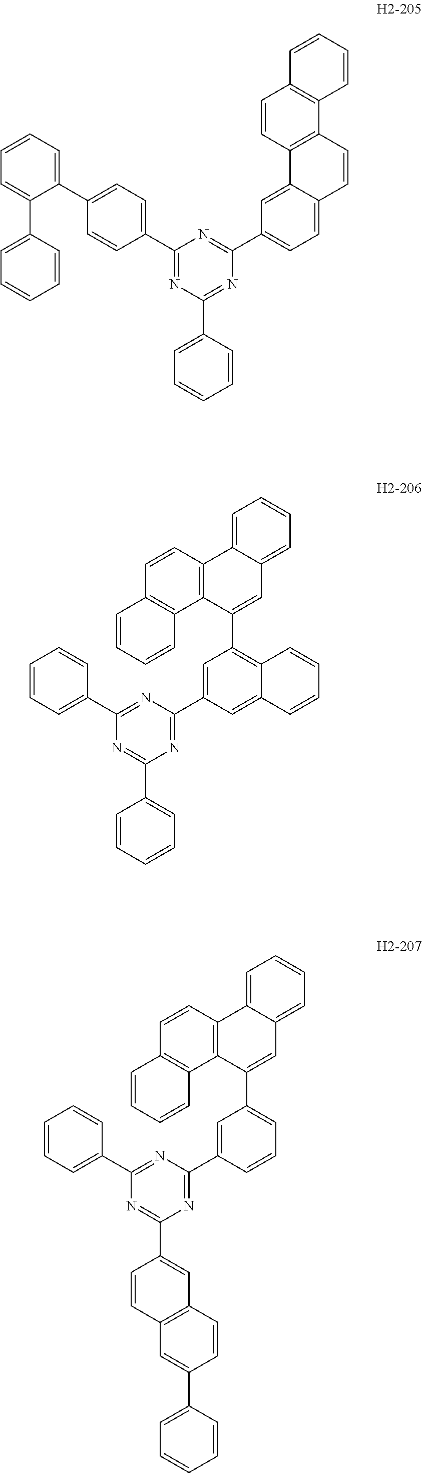 [SMILES: c1ccc(-c2nc(-c3ccc(-c4ccccc4-c4ccccc4)cc3)nc(-c3ccc4ccc5c6ccccc6ccc5c4c3)n2)cc1 ; c1ccc(-c2nc(-c3ccccc3)nc(-c3cc(-c4cc5ccccc5c5ccc6ccccc6c45)c4ccccc4c3)n2)cc1 ; c1ccc(-c2ccc3cc(-c4nc(-c5ccccc5)nc(-c5cccc(-c6cc7ccccc7c7ccc8ccccc8c67)c5)n4)ccc3c2)cc1]

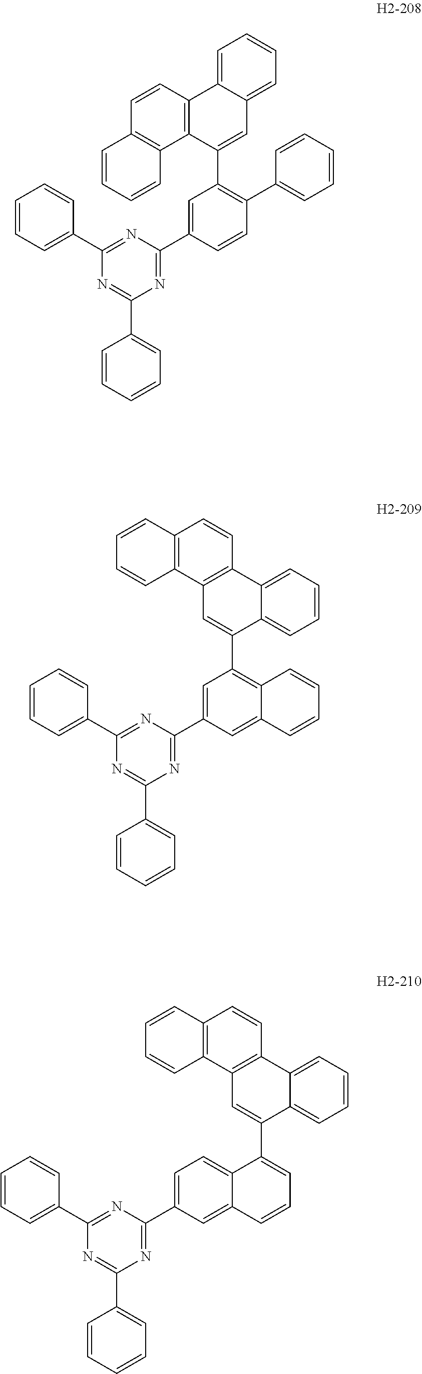 [SMILES: c1ccc(-c2nc(-c3ccccc3)nc(-c3ccc(-c4ccccc4)c(-c4cc5ccccc5c5ccc6ccccc6c45)c3)n2)cc1 ; c1ccc(-c2nc(-c3ccccc3)nc(-c3cc(-c4cc5c6ccccc6ccc5c5ccccc45)c4ccccc4c3)n2)cc1 ; c1ccc(-c2nc(-c3ccccc3)nc(-c3ccc4c(-c5cc6c7ccccc7ccc6c6ccccc56)cccc4c3)n2)cc1]

The host compounds represented by Formulas 1 and 3 according to the present disclosure can be prepared by a synthetic method known to one skilled in the art. For example, they may be prepared by referring to the synthesis method disclosed in Korean Patent Application Laid-open Nos. 2021-0098316 and 2022-0055411, etc.

Hereinafter, an organic electroluminescent device to which the aforementioned plurality of host materials is applied, will be described.

The organic electroluminescent device according to one embodiment includes a first electrode; a second electrode; and at least one organic layer(s) interposed between the first electrode and the second electrode. The organic layer may include a light-emitting layer, and the light-emitting layer may comprise a plurality of host materials comprising at least one first host material represented by Formula 1 and at least one second host material represented by Formula 3.

According to one embodiment, the organic electroluminescent material of the present disclosure comprises at least one compound(s) of compounds H1-1 to H1-241, which is a first host material, and at least one compound(s) of compounds H2-1 to H2-210, which is a second host material. The plurality of host materials may be included in the same organic layer, for example the same light-emitting layer, or may be included in different light-emitting layers, respectively.

The organic layer may further comprise at least one layer selected from a hole injection layer, a hole transport layer, a hole auxiliary layer, a light-emitting auxiliary layer, an electron transport layer, an electron injection layer, an interlayer, a hole blocking layer, an electron blocking layer, and an electron buffer layer, in addition to the light-emitting layer. The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, or an electron blocking material. Also, the electron transport layer, the electron injection layer, the electron buffer layer, or the hole blocking layer may contain the azine-based compound as an electron transport material, an electron injection material, an electron buffer material, or a hole blocking material. Also, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

The plurality of host materials according to one embodiment may be used as light-emitting materials for a white organic light-emitting device. The white organic light-emitting device has suggested various structures such as a parallel side-by-side arrangement method, a stacking arrangement method, or CCM (color conversion material) method, etc., according to the arrangement of R (Red), G (Green), YG (yellowish green), or B (blue) light-emitting units. In addition, the plurality of host materials according to one embodiment may also be applied to the organic electroluminescent device comprising a QD (quantum dot).

One of the first electrode and the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a both-sides emission type according to the kinds of the material forming the first electrode and the second electrode.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. Also, the hole injection layer may be doped as a p-dopant. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layers, and wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer may be placed between the electron transport layer (or electron injection layer) and the light-emitting layer, and blocks the arrival of holes to the cathode, thereby improving the probability of recombination of electrons and holes in the light-emitting layer. The hole blocking layer or the electron transport layer may also be multi-layers, wherein each layer may use a plurality of compounds. Also, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiO_{X}(1≤X≤2), AlO_{X} (1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

The organic electroluminescent device according to one embodiment of the present disclosure may be an organic electroluminescent device having a tandem structure. In the case of a tandem organic electroluminescent device according to one embodiment, a single light-emitting unit (light-emitting unit) may be formed in a structure in which two or more units are connected by a charge generation layer. The organic electroluminescent device may include a plurality of two or more light emitting units, for example, a plurality of three or more light-emitting units, having first and second electrodes opposed to each other on a substrate and a light emitting layer that is stacked between the first and second electrodes and emits light in a specific wavelength range. It may include a plurality of light-emitting units, and each of the light-emitting units may include a hole transport band, a light-emitting layer, and an electron transport band, and the hole transport band may include a hole injection layer and a hole transport layer, and the electron transfer zone may include an electron transport layer and an electron injection layer. According to one embodiment, three or more light emitting layers may be included in the light emitting unit. A plurality of light emitting units may emit the same color or different colors. Additionally, one light emitting unit may include one or more light emitting layers, and the plurality of light emitting layers may be light emitting layers of the same or different colors. It may include one or more charge generation layers located between each light-emitting unit. The charge generation layer refers to the layer in which holes and electrons are generated when voltage is applied. When there are three or more light-emitting units, a charge generation layer may be located between each light-emitting unit. At this time, the plurality of charge generation layers may be the same or different from each other. By disposing the charge generation layer between light-emitting units, current efficiency is increased in each light-emitting unit and charges can be smoothly distributed. Specifically, the charge generation layer is provided between two adjacent stacks and can serve to drive a tandem organic electroluminescent device using only a pair of anodes and cathodes without a separate internal electrode located between the stacks.

The charge generation layer may be composed of an N-type charge generation layer and a P-type charge generation layer, and the N-type charge generation layer may be doped with an alkali metal, an alkaline earth metal, or a compound of an alkali metal and an alkaline earth metal, The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof. The P-type charge generation layer may be made of a metal or an organic material doped with a P-type dopant. For example, the metal may be made of one or two or more alloys selected from the group consisting of Al, Cu, Fe, Pb, Zn, Au, Pt, W, In, Mo, Ni, and Ti. Additionally, commonly used materials may be used as the P-type dopant and host materials used in the P-type doped organic material.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. Also, a reductive dopant layer may be employed as a charge generating layer to prepare an organic electroluminescent device having two or more light-emitting layers and emitting white light.

An organic electroluminescent device according to one embodiment may further comprise at least one dopant in the light-emitting layer.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may be preferably a metallated complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant comprised in the organic electroluminescent device of the present disclosure may use the compound represented by the following Formula 101, but is not limited thereto.

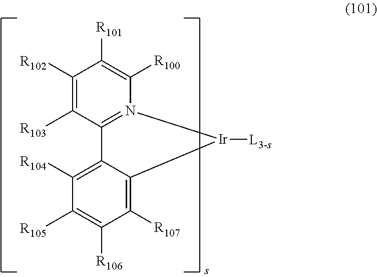

in Formula 101,
L is selected from any one of the following structures 1 to 3,

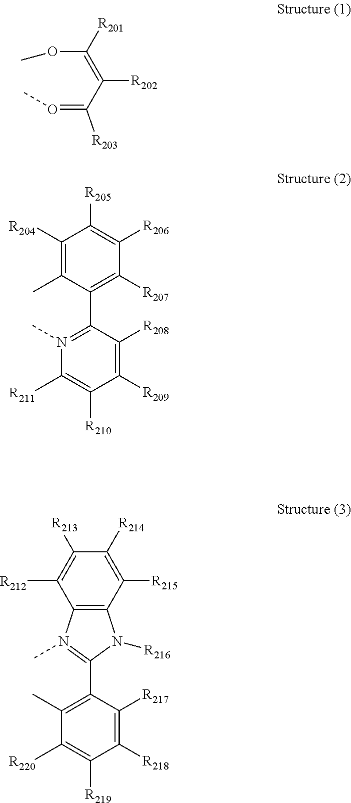

R₁₀₀ to R₁₀₃ each independently represent, hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, cyano, a substituted or unsubstituted (3- to 30-membered)heteroaryl, or a substituted or unsubstituted (C1-C30)alkoxy; or may be linked to the adjacent substituents to form a ring(s), for example, to form a ring(s) with a pyridine, e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;
R₁₀₄ to R₁₀₇ each independently represent, hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, a substituted or unsubstituted (3- to 30-membered)heteroaryl, cyano, or a substituted or unsubstituted (C1-C30)alkoxy; or may be linked to the adjacent substituents to form a substituted or unsubstituted ring(s), for example, to form a substituted or unsubstituted ring(s) with a benzene, e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;
R₂₀₁ to R₂₂₀ each independently represent, hydrogen, deuterium, halogen, (C1-C30)alkyl unsubstituted or substituted with deuterium and/or halogen, a substituted or unsubstituted (C3-C30)cycloalkyl, or a substituted or unsubstituted (C6-C30)aryl; or may be linked to the adjacent substituents to form a substituted or unsubstituted ring(s); and
s represents an integer of 1 to 3.

Specifically, the specific examples of the dopant compound include the following, but are not limited thereto.

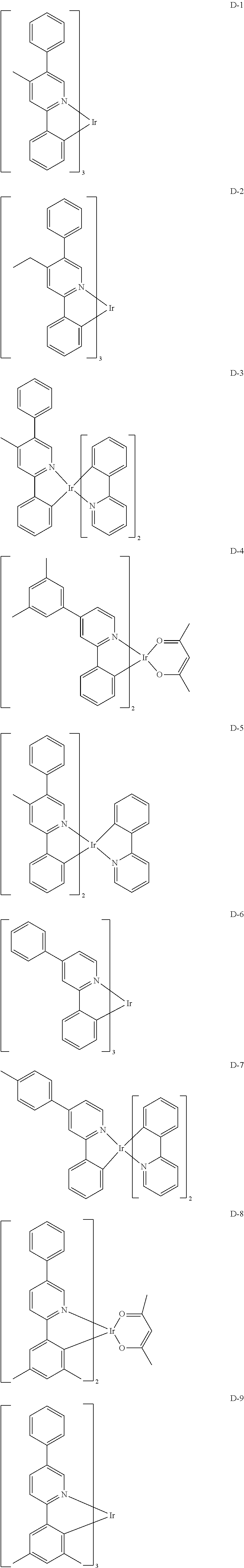

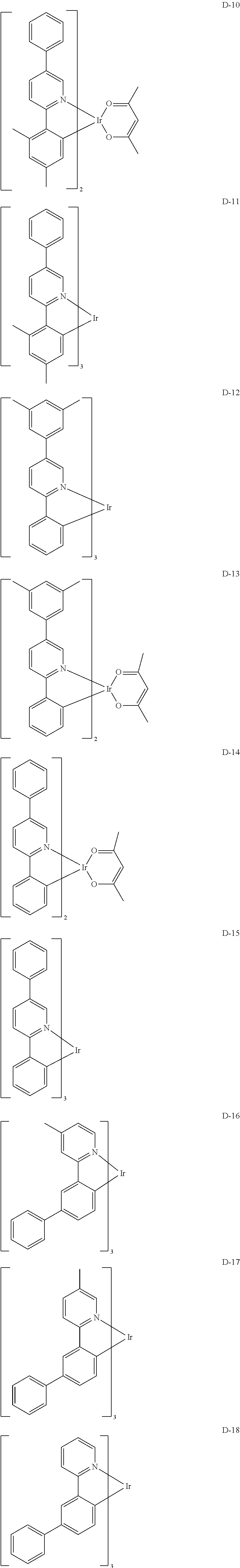

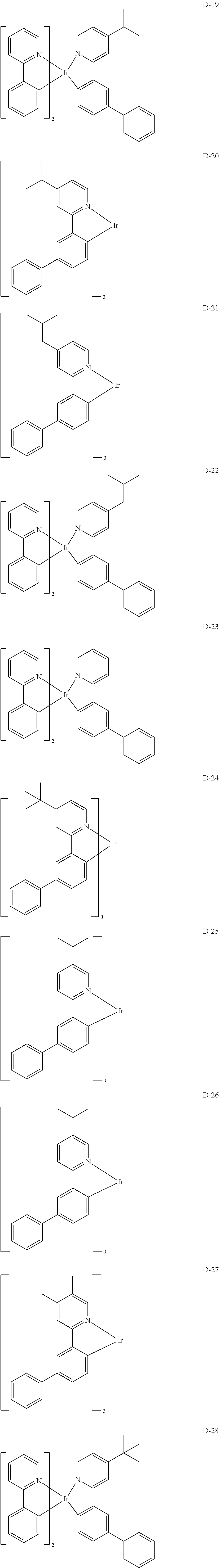

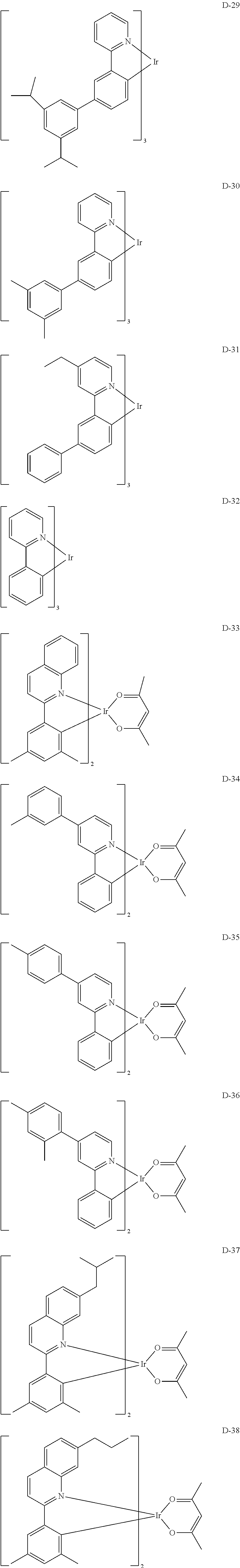

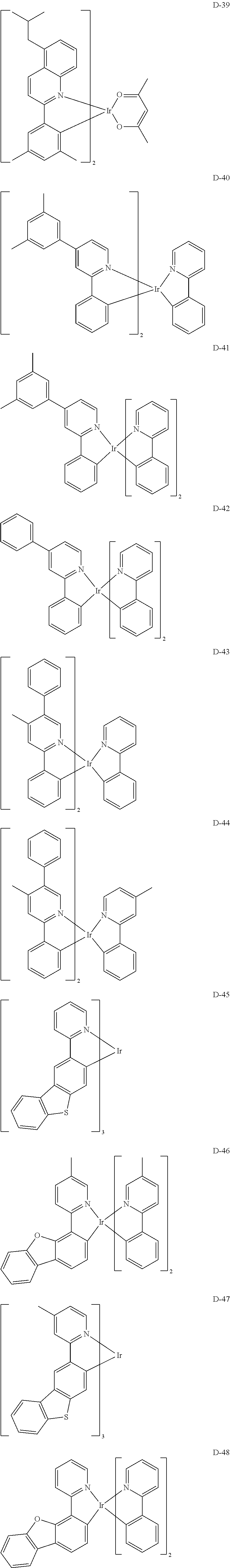

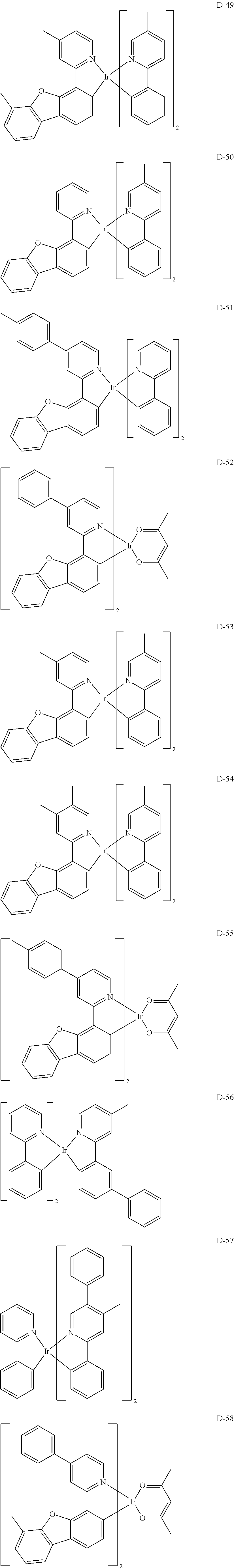

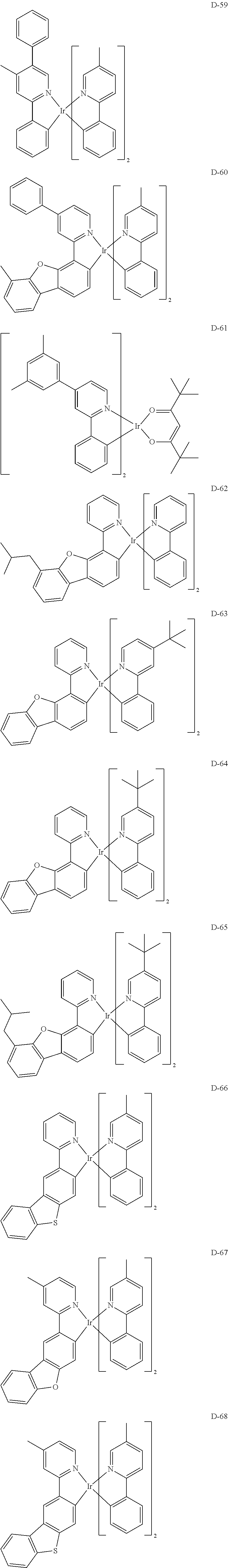

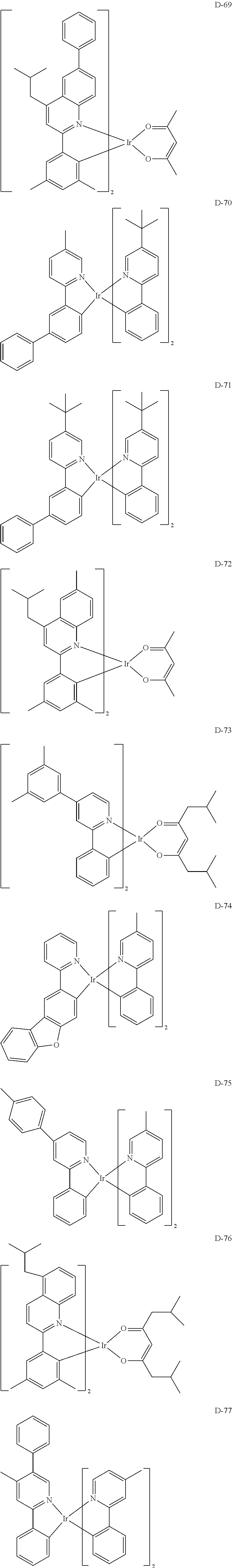

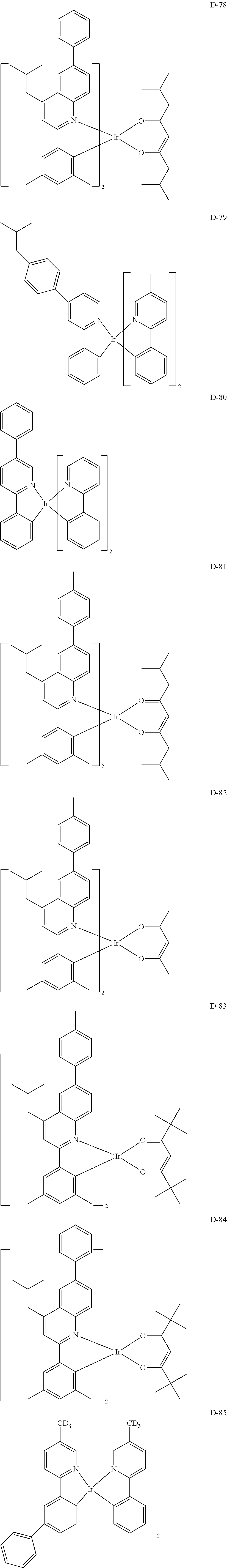

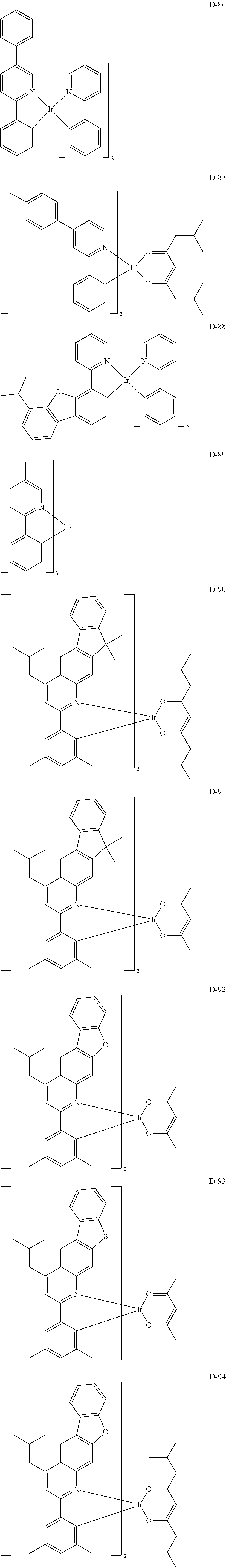

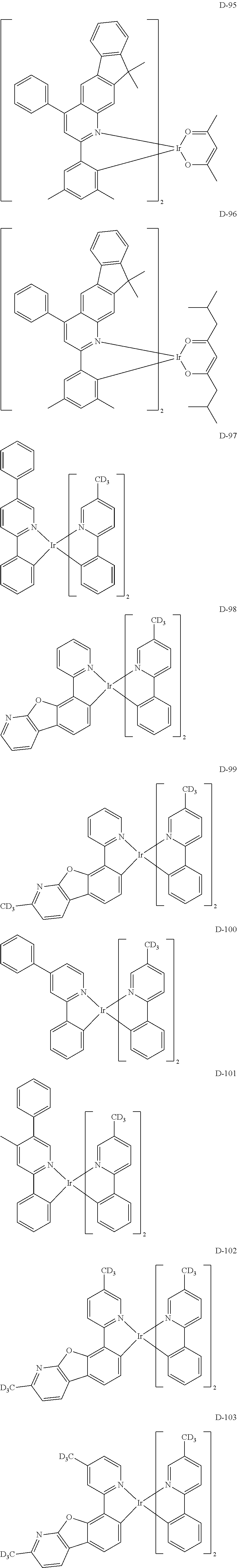

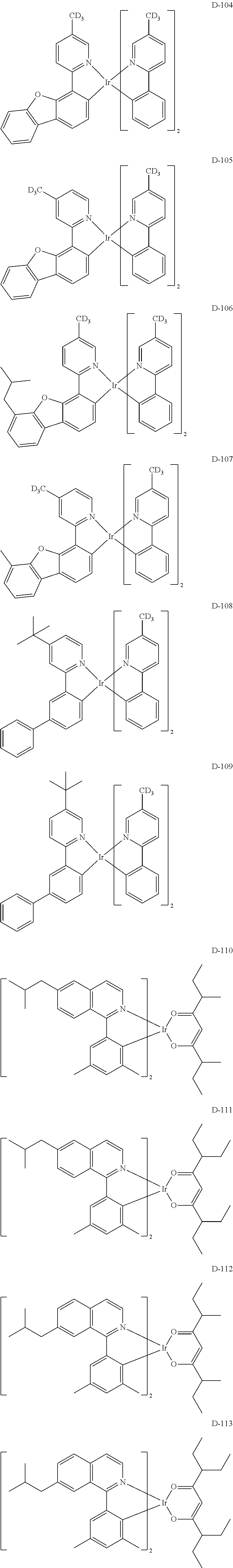

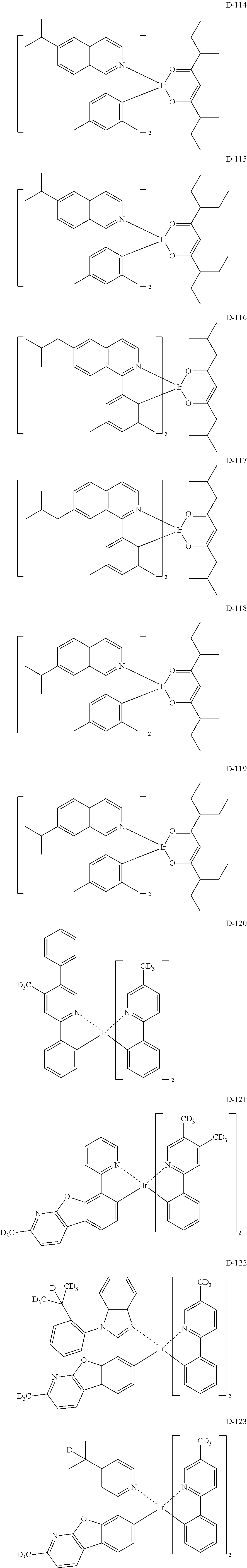

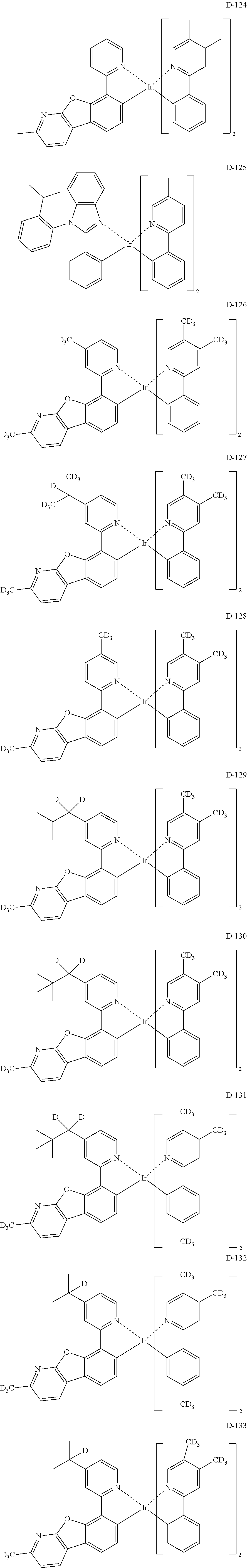

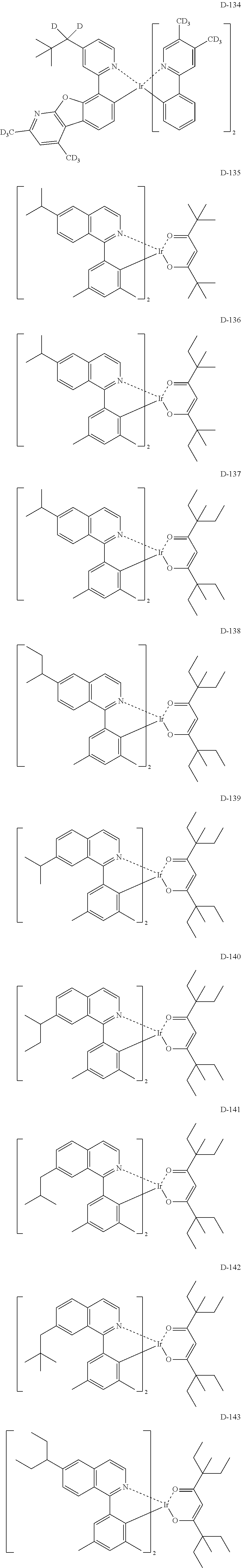

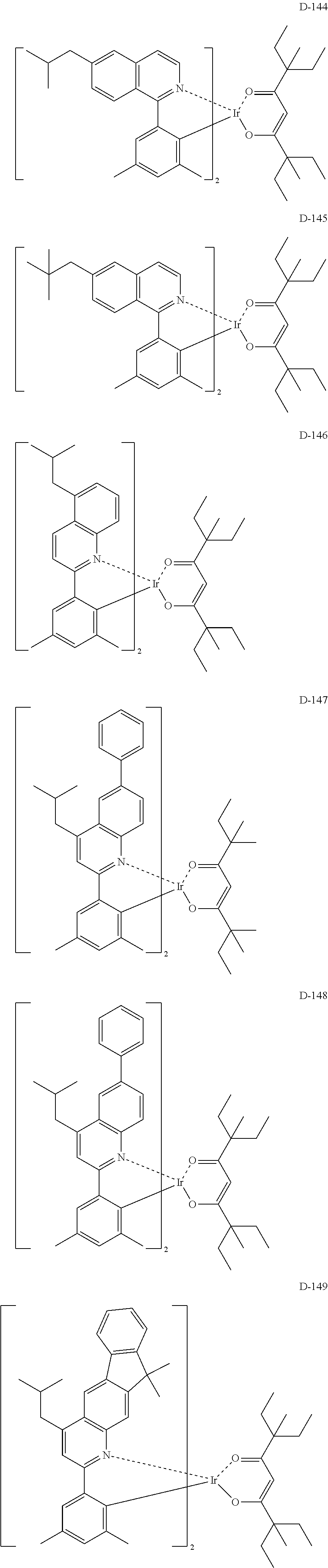

In order to form each layer of the organic electroluminescent device of the present disclosure, dry film-forming methods such as vacuum evaporation, sputtering, plasma, ion plating methods, etc., or wet film-forming methods such as spin coating, dip coating, flow coating methods, etc., can be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the first host compound and the second host compound according to one embodiment, the layer can be formed by the above-listed methods, and can often be formed by co-deposition or mixture-deposition. The co-deposition is a mixed deposition method in which two or more materials are put into respective individual crucible sources and a current is applied to both cells simultaneously to evaporate the materials; and the mixed deposition is a method in which two or more materials are mixed in one crucible source before the deposition, and then a current is applied to one cell to evaporate the materials.

According to one embodiment, when the first host compound and the second host compound are present in the same layer or different layers in the organic electroluminescent device, the two host compounds may be individually formed. For example, after depositing the first host compound, a second host compound may be deposited.

According to one embodiment, the present disclosure can provide display devices comprising a plurality of host materials including a first host compound represented by Formula 1 and a second host compound represented by Formula 3. In addition, by using the organic electroluminescent device of the present disclosure, display devices such as smartphones, tablets, notebooks, PCs, TVs, or display devices for vehicles, or lighting devices such as outdoor or indoor lighting can be prepared.

Hereinafter, the preparation method of the host compound according to the present disclosure will be explained with reference to the synthesis method of a representative compound or intermediate compound in order to understand the present disclosure in detail.

### [Example 1] Preparation of Compound H1-159

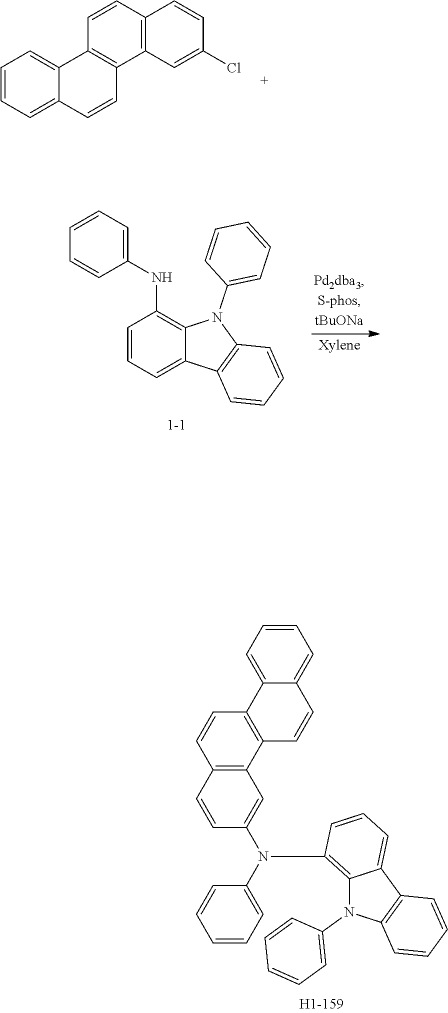 [SMILES: Clc1ccc2ccc3c4ccccc4ccc3c2c1 ; c1ccc(Nc2cccc3c4ccccc4n(-c4ccccc4)c23)cc1 ; c1ccc(N(c2ccc3ccc4c5ccccc5ccc4c3c2)c2cccc3c4ccccc4n(-c4ccccc4)c23)cc1]

3-Chlorochrysene (5 g, 19 mmol), compound 1-1 (7.6 g, 22.8 mol), tris(dibenzylideneacetone)dipalladium(0)(Pd₂(dba)₃) (870 m g, 0.95 mmol), 2-dicyclohexylphosphino-2′,6′-dimethoxybiphenyl(S-Phos) (780 mg, 1.9 mmol), and tBuONa(sodium tert-butoxide) (2.73 g, 28.5 mmol) were dissolved in 450 mL of xylene, and then stirred at 150° C. for 0.5 hour. After the reaction was completed, the mixture was cooled to room temperature, filtered through Celite filter to make a solid, and then separated by column chromatography to obtain Compound H1-159 (4.7 g, yield: 44%).

| | MW | M.P |
|---|---|---|
| H1-159 | 560.69 | 223.8° C. |

### [Example 2] Preparation of Compound H1-189

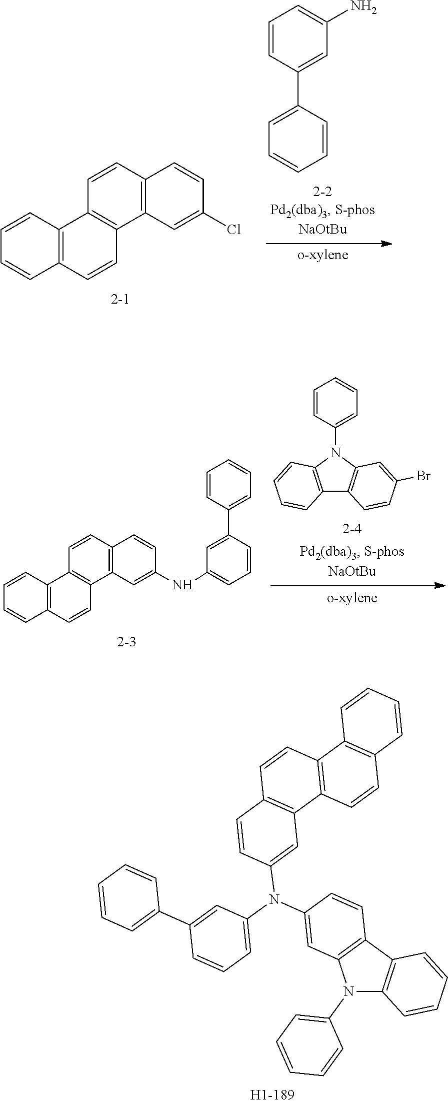

### 1) Synthesis of Compound 2-3

Compound 2-1 (22.0 g, 83.74 mmol), compound 2-2 (17.0 g, 100.48 mmol), Pd₂(dba)₃ (3.8 g, 4.18 mmol), S-Phos (3.4 g, 8.37 mmol), and NaOtBu (12.0 g, 125.61 mmol) were dissolved in 418 mL of o-xylene, and then stirred at 150° C. for 2 hours. After the reaction was completed, the mixture was cooled to room temperature, and the solid was made by filtering through celite and then silica filter to obtain Compound 2-3 (21.5 g, yield: 64.9%).

### 2) Synthesis of Compound H1-189

Compound 2-3 (6.5 g, 16.44 mmol), Compound 2-4 (6.4 g, 19.72 mmol), Pd₂(dba)₃ (0.8 g, 0.82 mmol), S-Phos (0.7 g, 1.64 mmol), and NaOtBu (2.4 g, 24.66 mmol) were dissolved in 82 mL of o-xylene, and then stirred under reflux at 150° C. for 1 hour. After the reaction was completed, the mixture was cooled to room temperature, and the solid was made by filtering through celite and then silica filter, and then recrystallized to obtain Compound H1-189 (5.5 g, yield: 52.5%).

| | MW | M.P |
|---|---|---|
| H1-189 | 636.78 | 199° C. |

### [Example 3] Preparation of Compound H1-144

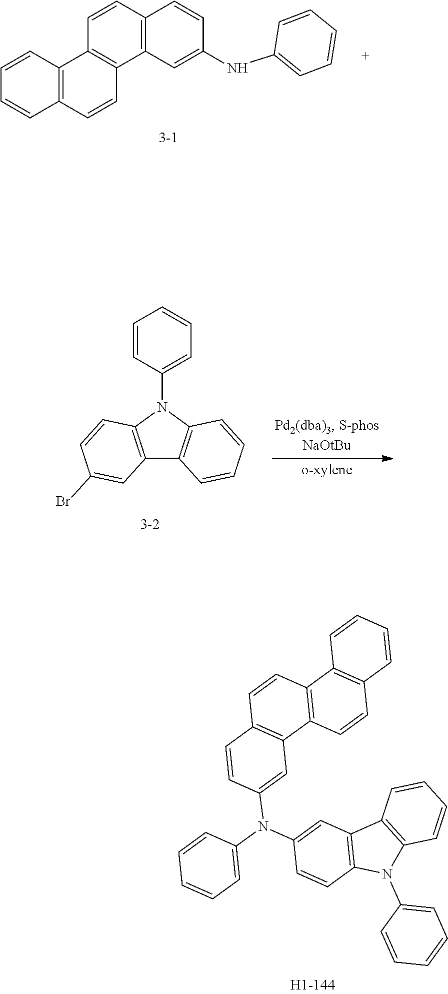 [SMILES: c1ccc(Nc2ccc3ccc4c5ccccc5ccc4c3c2)cc1 ; Brc1ccc2c(c1)c1ccccc1n2-c1ccccc1 ; c1ccc(N(c2ccc3ccc4c5ccccc5ccc4c3c2)c2ccc3c(c2)c2ccccc2n3-c2ccccc2)cc1]

Compound 3-1 (5.7 g, 17.85 mmol), Compound 3-2 (6.9 g, 21.41 mmol), Pd₂(dba)₃ (0.8 g, 0.89 mmol), S-Phos (0.7 g, 1.78 mmol), and NaOtBu (2.6 g, 26.77 mmol) were dissolved in 90 mL of o-xylene, and then stirred under reflux at 150° C. for 1 hour. After the reaction was completed, the mixture was cooled to room temperature, the layers were separated, and the solid was made by filtering through celite and then silica filter, and then recrystallized to obtain Compound H1-144 (4.1 g, yield: 41.0%).

| | MW | M.P |
|---|---|---|
| H1-144 | 560.70 | 213° C. |

### [Example 4] Preparation of Compound H1-186

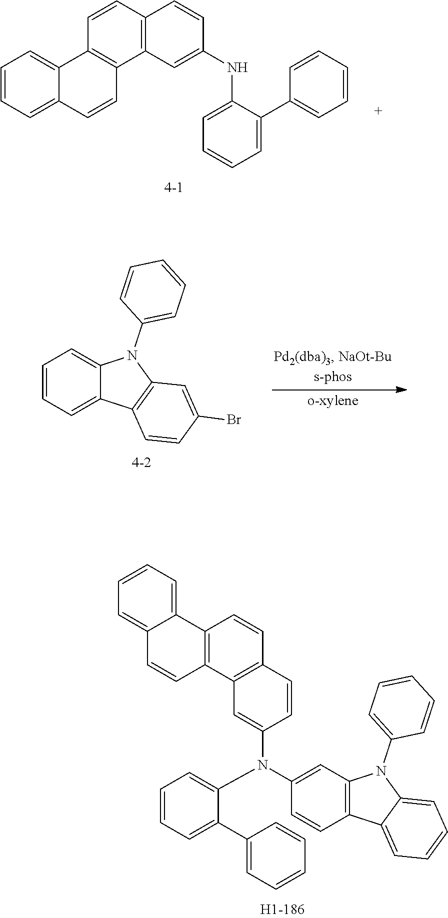 [SMILES: c1ccc(-c2ccccc2Nc2ccc3ccc4c5ccccc5ccc4c3c2)cc1 ; Brc1ccc2c3ccccc3n(-c3ccccc3)c2c1 ; c1ccc(-c2ccccc2N(c2ccc3ccc4c5ccccc5ccc4c3c2)c2ccc3c4ccccc4n(-c4ccccc4)c3c2)cc1]

Compound 4-1 (5 g, 12.64 mmol), Compound 4-2 (4.9 g, 15.17 mmol), Pd₂(dba)₃ (0.58 g, 0.632 mmol), NaOt-Bu (1.8 g, 18.96 mmol), and S-Phos (0.52 g, 1.264 mmol) were added to 65 mL of xylene, and then stirred under reflux at 150° C. for 1 hour. After the reaction was completed, the mixture was cooled to room temperature and filtered through Celite. Next, it was distillated under reduced pressure, and then separated by column chromatography to obtain Compound H1-186 (4 g, yield: 49%).

| | MW | M.P |
|---|---|---|
| H1-186 | 636.7 | 159.5° C. |

### [Example 5] Preparation of Compound H2-29

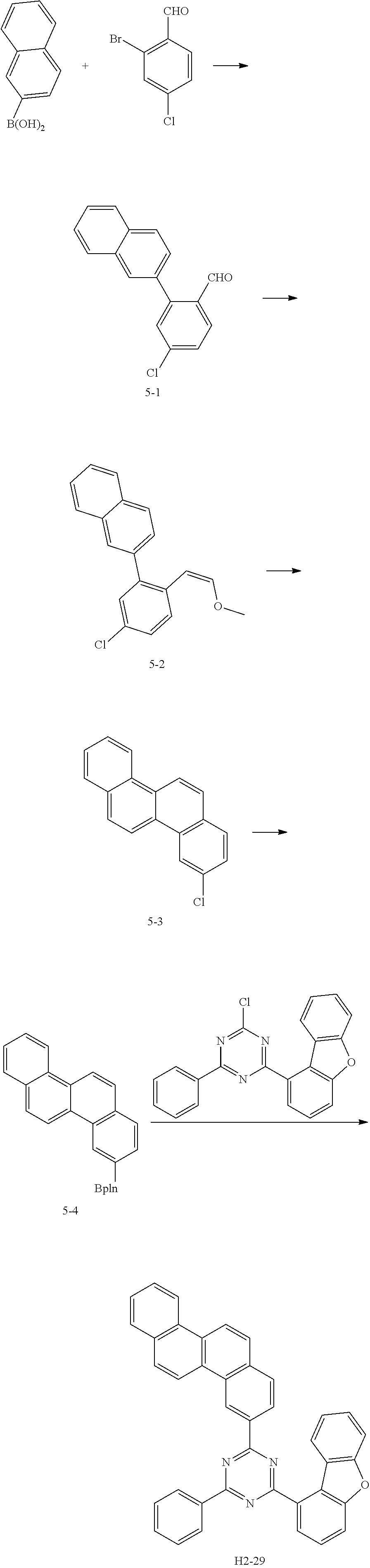

### 1) Synthesis of Compound 5-1

Naphthalene-2-yl boronic acid (50 g, 291 mmol), 2-bromo-4-chlorobenzaldehyde (63 g, 291 mmol), tetrakis(triphenylphosphine)palladium (Pd(PPh₃)₄) (16.8 g, 14.5 mmol), sodium carbonate (77 g, 727 mmol), toluene (1080 mL), ethanol (240 mL) and distilled water (360 mL) were added to a reaction vessel, and then stirred at 140° C. for 5 hours. After the reaction was completed, the precipitated solid was washed with distilled water and methanol. Next, it was purified by column chromatography to obtain Compound 5-1 (71 g, yield: 92%).

### 2) Synthesis of Compound 5-2

Compound 5-1 (71 g, 268 mmol), (methoxymethyl)triphenylphosphonium chloride (110 g, 321 mmol) and tetrahydrofuran (THF) (1,300 mL) were added to a reaction vessel, the reaction mixture was stirred for 10 minutes, and then potassium tert-butoxide (KOtBu) (1M in THF, 300 mL) was slowly added dropwise under a condition of 0° C. The temperature was slowly raised and stirred at room temperature for 3 hours. The reaction was terminated by adding distilled water to the reaction solution, and the organic layer was extracted with ethyl acetate. The extracted organic layer was dried with magnesium sulfate and the solvent was removed using a rotary evaporator. Next, it was purified by column chromatography to obtain Compound 5-2 (71 g, yield: 90%).

### 3) Synthesis of Compound 5-3

Compound 5-2 (70 g, 238 mmol), Eaton's reagent (7 mL) and chlorobenzene (1,180 mL) were added to a reaction vessel, and then refluxed for 1 hour. After the reaction was completed, the mixture was cooled to room temperature and extracted with methylene chloride (MC). The extracted organic layer was dried with magnesium sulfate and the solvent was removed using a rotary evaporator. Next, it was purified by column chromatography to obtain Compound 5-3 (60 g, yield: 96%).

### 4) Synthesis of Compound 5-4

Compound 5-3 (35 g, 133.2 mmol), bis(pinacolato)diborane (44 g, 173 mmol), Pd₂(dba)₃ (6.1 g, 6.66 mmol), S-Phos (5.5 g, 13.3 mmol), potassium acetate (KOAc) (39.2 g, 400 mmol) and 1,4-dioxane (666 mL) were added to a reaction vessel, and then stirred at 150° C. for 3 hours. After the reaction was completed, the mixture was cooled to room temperature and the organic layer was extracted with ethyl acetate. The extracted organic layer was dried with magnesium sulfate and the solvent was removed using a rotary evaporator. Next, it was purified by column chromatography to obtain Compound 5-4 (38 g, yield: 81%).

### 5) Synthesis of Compound H2-29

Compound 5-4 (5 g, 14.1 mmol), 2-chloro-4-(dibenzo[b,d]furan-1-yl)-6-phenyl-1,3,5-triazine (6.6 g, 18.3 mmol), Pd(PPh₃)₄ (0.8 g, 0.7 mmol), potassium carbonate (K₂CO₃) (3.9 g, 28.2 mmol), toluene (42 mL), ethanol (10 mL), and distilled water (14 mL) were added to a reaction vessel, and then stirred at 140° C. for 8 hours. After the reaction was completed, the mixture was added dropwise to methanol, and the resulting solid was filtered. The produced solid was purified by column chromatography to obtain Compound H2-29 (6.8 g, yield: 88%).

| | MW | M.P. |
|---|---|---|
| H2-29 | 549.6 | 267° C. |

### [Example 6] Preparation of Compound H2-32

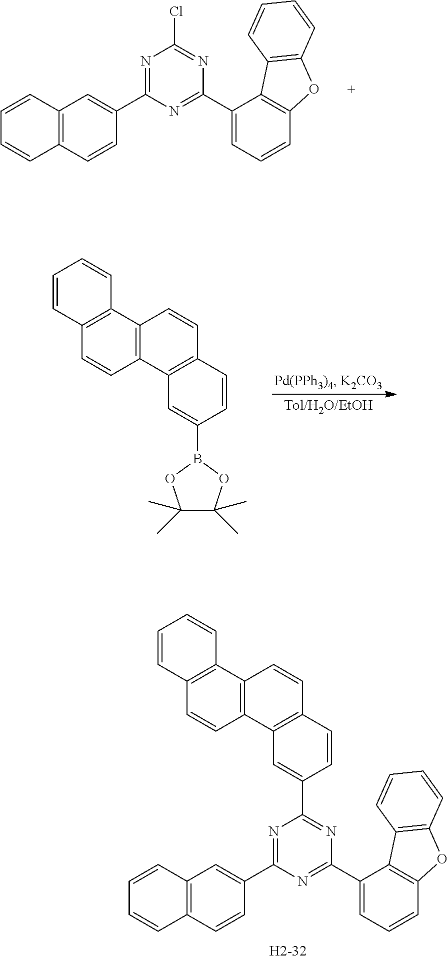 [SMILES: Clc1nc(-c2ccc3ccccc3c2)nc(-c2cccc3oc4ccccc4c23)n1 ; CC1(C)OB(c2ccc3ccc4c5ccccc5ccc4c3c2)OC1(C)C ; c1ccc2cc(-c3nc(-c4ccc5ccc6c7ccccc7ccc6c5c4)nc(-c4cccc5oc6ccccc6c45)n3)ccc2c1]

2-Chloro-4-(dibenzo[b,d]furan-1-yl)-6-(naphthalene-2-yl)-1,3,5-triazine (3.8 g, 9.4 mmol), 2-chryshen-3-yl-4,4,5,5-teteramethyl-(1,3,2-dioxaborolane) (4 g, 11.3 mmol), Pd(PPh₃)₄ (0.54 g, 0.47 mmol), and K₂CO₃ (3.3 g, 23.5 mmol) in a flask were dissolved in 50 mL of toluene, 25 mL of ethanol, and 25 mL of distilled water, and then refluxed at 130° C. for 4 hours. After the reaction was completed, the organic layer was extracted with ethyl acetate and residual moisture was removed using magnesium sulfate. Next, it was dried and separated using column chromatography to obtain Compound H2-32 (5 g, yield: 94%).

| | MW | M.P. |
|---|---|---|
| H2-32 | 559.30 | 292.8° C. |

### [Example 7] Preparation of H2-146

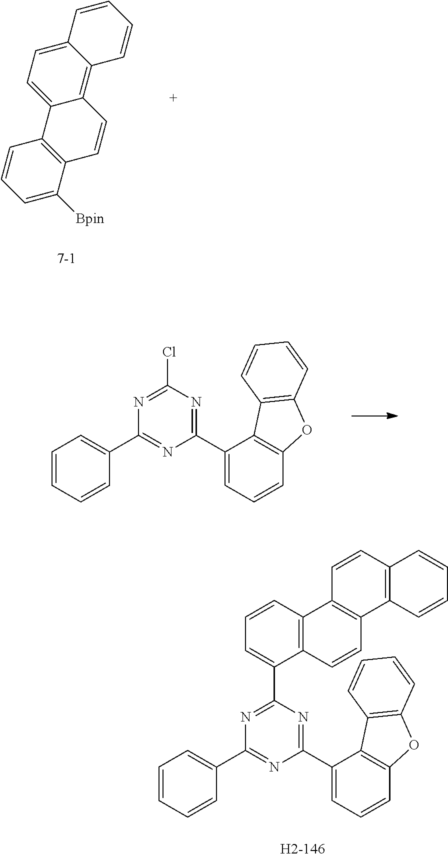

Compound 7-1 (8.8 g, 24.8 mmol), 2-chloro-4-(dibenzo[b,d]furan-1-yl)-6-phenyl-1,3,5-triazine (11.6 g, 32.3 mmol), Pd(PPh₃)₄ (1.4 g, 1.24 mmol), K₂CO₃ (6.9 g, 49.68 mmol), toluene (125 mL), ethanol (31 mL) and distilled water (41 mL) were added to a reaction vessel, and then stirred at 130° C. for 2 hours. After the reaction was completed, the precipitated solid was washed with distilled water and methanol. Next, it was purified by column chromatography to obtain Compound H2-146 (10.4 g, yield: 76%).

| | MW | M.P. |
|---|---|---|
| H2-146 | 549.6 | 281° C. |

### [Example 8] Preparation of Compound H1-219

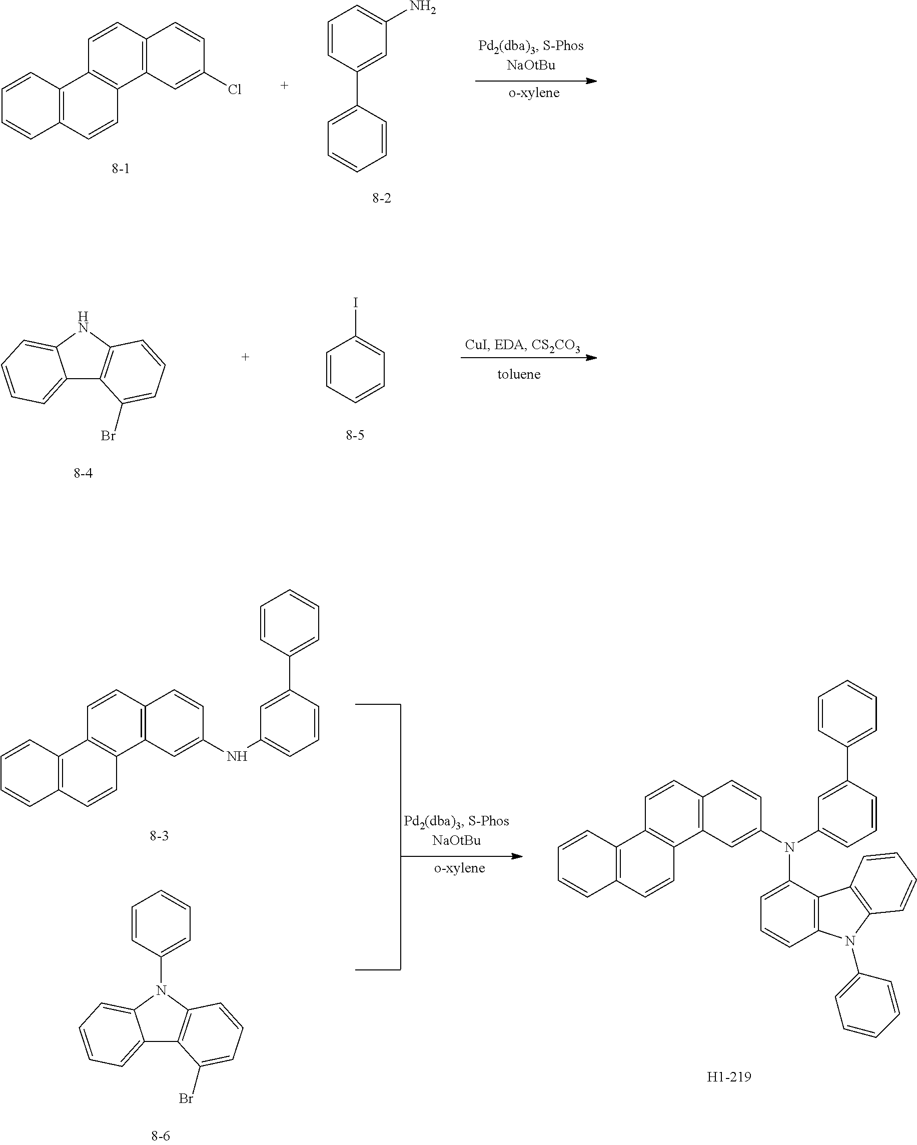

### 1) Synthesis of Compound 8-3

Compound 8-1 (22.0 g, 83.74 mmol), Compound 8-2 (17.0 g, 100.48 mmol), Pd₂(dba)₃ (3.8 g, 4.18 mmol), S-Phos (3.4 g, 8.37 mmol), NaOtBu (12.0 g, 125.61 mmol) were dissolved in 418 mL of o-xylene, and then stirred under reflux at 150° C. for 2 hours. After the reaction was completed, the mixture was cooled to room temperature, and the solid was made by filtering through Celite and silica, to obtain Compound 8-3 (21.5 g, yield: 64.9%).

### 2) Synthesis of Compound 8-6

Compound 8-4 (15.0 g, 50.65 mmol), Compound 8-5 (20.7 g, 101.30 mmol, density 1.83), CuI (4.8 g, 25.32 mmol), EDA (1.5 g, 25.32 mmol, density 0.897), and CS₂CO₃ (49.5 g, 151.95 mmol) were dissolved in 260 mL of toluene, and then stirred under reflux at 130° C. for 24 hours. After the reaction was completed, the mixture was cooled to room temperature, separated into layers, filtered through celite, and then filtered through silica to obtain Compound 8-6 (12.9 g, yield: 76.1%) in a liquid state.

### 3) Synthesis of Compound H1-219

Compound 8-3 (13.2 g, 33.36 mmol), Compound 8-6 (12.9 g, 40.03 mmol), Pd₂(dba)₃ (1.5 g, 1.67 mmol), S-Phos (1.4 g, 8.37 mmol), and NaOtBu (4.8 g, 50.04 mmol) were dissolved in 170 mL of o-xylene, and then stirred under reflux at 150° C. for 1 hour. After the reaction was completed, the mixture was cooled to room temperature, filtered through celite, and then filtered through silica. Next, it was separated by column chromatography, and recrystallized to obtain Compound H1-219 (4.4 g, yield: 20.7%).

| | MW | M.P |
|---|---|---|
| H1-219 | 636.78 | 231° C. |

### [Example 9] Preparation of Compound H1-148

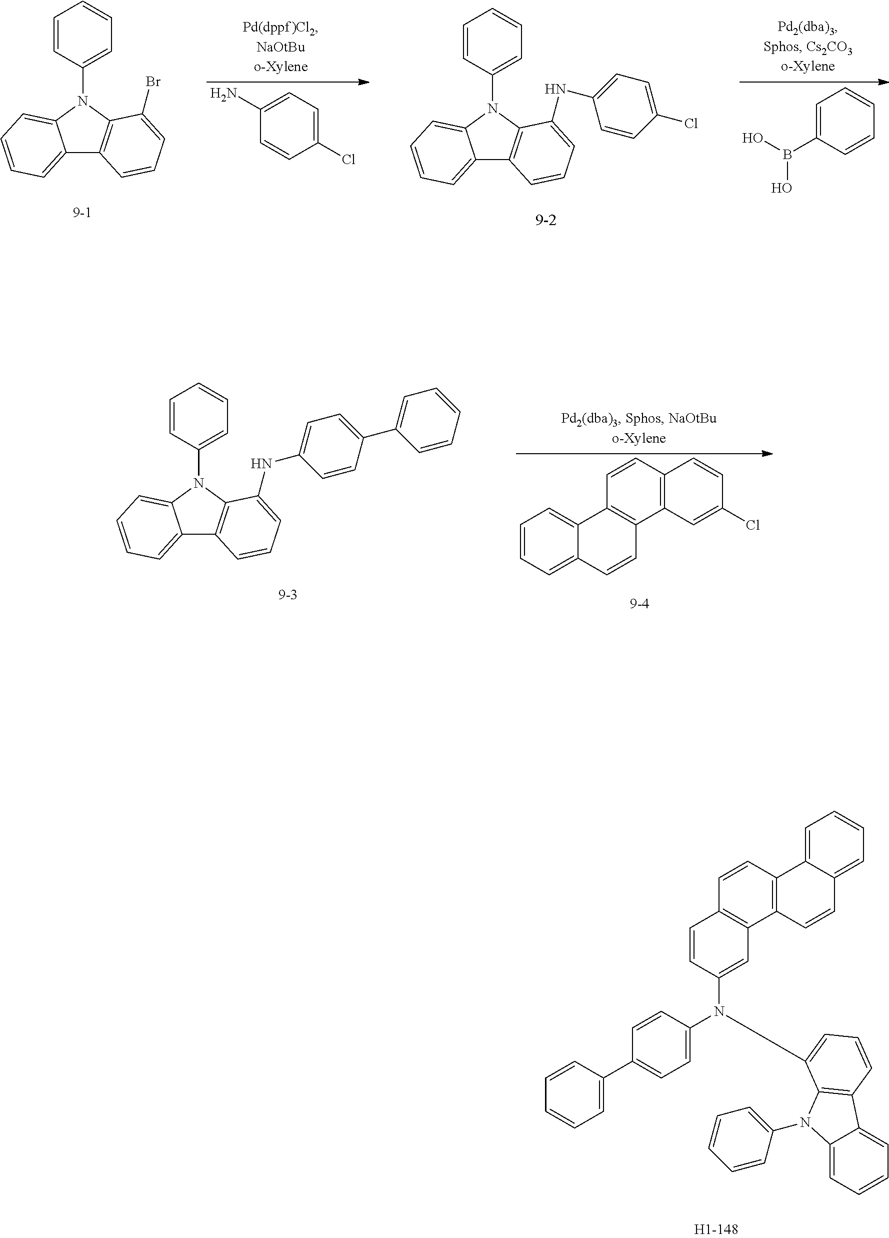

### 1) Synthesis of Compound 9-2

Compound 9-1 (16 g, 49.66 mmol), 4-chloroaniline (7.6 g, 59.59 mmol), Pd(dppf)Cl₂ (3.66 g, 4.97 mmol), and NaOtBu (7.16 g, 74.49 mmol) were added to 248 mL of o-xylene, and then, stirred under reflux at 130° C. for 3 hours. After the reaction was completed, the mixture was cooled to room temperature and filtered through Celite. Next, it was distillated under reduced pressure and then separated by column chromatography to obtain Compound 9-2 (13.5 g, yield: 73.7%).

### 2) Synthesis of Compound 9-3

Compound 9-2 (12.4 g, 33.62 mmol), phenylboronic acid (4.92 g, 40.35 mmol) Pd₂(dba)₃ (3.08 g, 3.36 mmol), S-phos (2.76 g, 6.72 mmol), and CS₂CO₃ (32.86 g, 100.87 mmol) were added to 168 mL of o-xylene, and then stirred under reflux at 130° C. for 3.5 hours. After the reaction was completed, the mixture was cooled to room temperature and filtered through Celite. Next, it was distillated under reduced pressure and then separated by column chromatography to obtain Compound 9-3 (10.5 g, yield: 76.08%).

### 3) Synthesis of Compound H1-148

Compound 9-4 (6.4 g, 24.36 mmol), Compound 9-3 (5.0 g, 12.18 mmol), Pd₂(dba)₃ (0.56 g, 0.61 mmol), S-phos (0.5 g, 1.22 mmol), and NaOtBu (1.76 g, 18.27 mmol) were added to 61 mL of o-xylene, and then stirred under reflux at 130° C. for 4 hours. After the reaction was completed, the mixture was cooled to room temperature and filtered through Celite. Next, the solid was dissolved in dichlorobenzene and separated by column chromatography to obtain Compound H1-148 (2.0 g, yield: 25.79%).

| | MW | M.P |
|---|---|---|
| H1-148 | 636.8 | 235.8° C. |

### [Example 10] Preparation of Compound H1-220

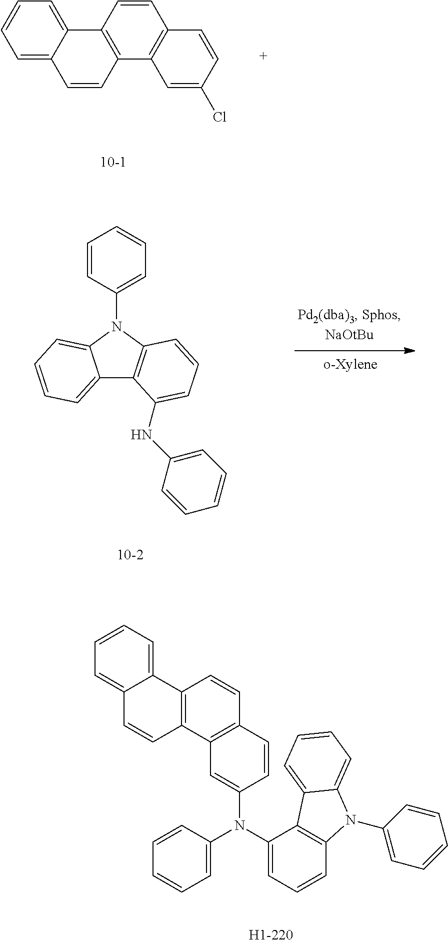 [SMILES: Clc1ccc2ccc3c4ccccc4ccc3c2c1 ; c1ccc(Nc2cccc3c2c2ccccc2n3-c2ccccc2)cc1 ; c1ccc(N(c2ccc3ccc4c5ccccc5ccc4c3c2)c2cccc3c2c2ccccc2n3-c2ccccc2)cc1]

Compound 10-1 (3.8 g, 14.46 mmol), Compound 10-2 (4.84 g, 14.46 mmol), Pd₂(dba)₃ (0.66 g, 0.72 mmol), S-phos (0.59 g, 1.45 mmol), and NaOtBu (2.08 g, 21.69 mmol) were added to 72 mL of o-xylene, and then stirred under reflux at 130° C. for 2.5 hours. After the reaction was completed, the mixture was cooled to room temperature and filtered through Celite. Next, it was distilled under reduced pressure, dissolved in methylene chloride, and separated by column chromatography to obtain Compound H1-220 (4.6 g, yield: 56.7%).

| | MW | M.P |
|---|---|---|
| H1-220 | 560.7 | 242.2° C. |

### [Example 11] Preparation of Compound H1-219

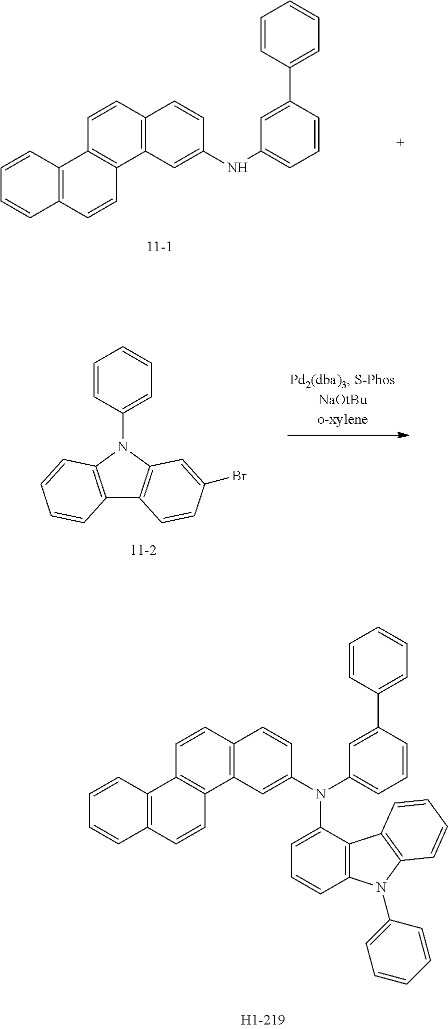 [SMILES: c1ccc(-c2cccc(Nc3ccc4ccc5c6ccccc6ccc5c4c3)c2)cc1 ; Brc1ccc2c3ccccc3n(-c3ccccc3)c2c1 ; c1ccc(-c2cccc(N(c3ccc4ccc5c6ccccc6ccc5c4c3)c3cccc4c3c3ccccc3n4-c3ccccc3)c2)cc1]

Compound 11-1 (13.2 g, 33.36 mmol), Compound 11-2 (12.9 g, 40.03 mmol), Pd₂(dba)₃ (1.5 g, 1.67 mmol), S-Phos (1.4 g, 8.37 mmol), and NaOtBu (4.8 g, 50.04 mmol) were dissolved in 170 mL of o-xylene, and then stirred under reflux at 160° C. for 1 hour. After the reaction was completed, the mixture was cooled to room temperature, filtered through Celite, filtered through silica, and recrystallized by column chromatography to obtain Compound H1-219 (4.4 g, yield: 20.7%).

| | MW | M.P |
|---|---|---|
| H1-219 | 636.78 | 231° C. |

### [Example 11] Preparation of Compound H1-188

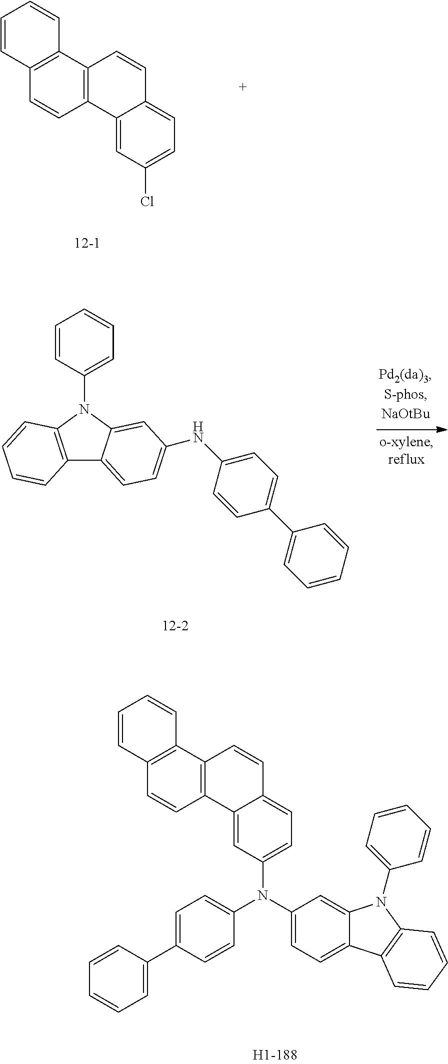 [SMILES: Clc1ccc2ccc3c4ccccc4ccc3c2c1 ; c1ccc(-c2ccc(Nc3ccc4c5ccccc5n(-c5ccccc5)c4c3)cc2)cc1 ; c1ccc(-c2ccc(N(c3ccc4ccc5c6ccccc6ccc5c4c3)c3ccc4c5ccccc5n(-c5ccccc5)c4c3)cc2)cc1]

Compound 12-1 (8.0 g, 30.45 mmol), Compound 12-2 (11.4 g, 27.68 mmol), Pd₂(dba)₃ (1.3 g, 1.38 mmol), S-phos (1.2 g, 2.77 mmol), and NaOtBu (4.0 g, 41.52 mmol) were dissolved in 140 mL of o-xylene, and then stirred under reflux at 120° C. for 1 hour. After the reaction was completed, the mixture was cooled to room temperature, and then separated into layers (EA/H2O). Next, the solid was maded by filtering through Celite, and then through silica to obtain Compound H1-188 (8.2 g, yield: 46%).

| | MW | M.P |
|---|---|---|
| H1-188 | 636.8 | 219° C. |

### [Example 12] Preparation of Compound H1-221

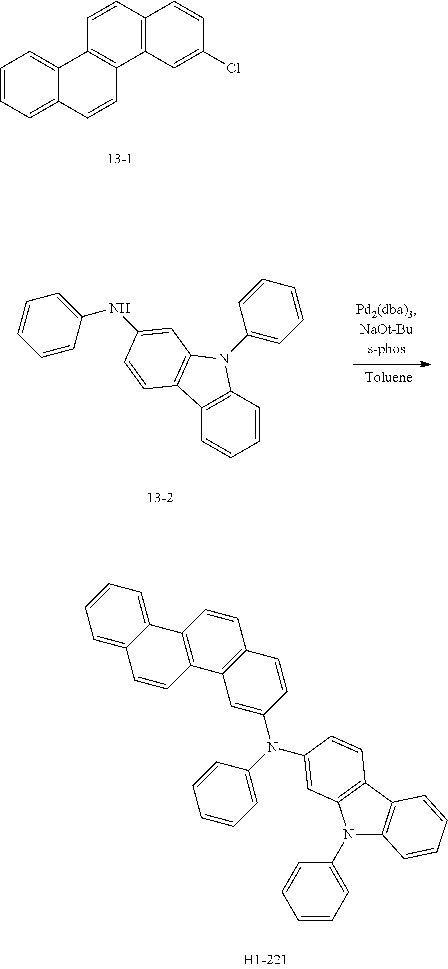 [SMILES: Clc1ccc2ccc3c4ccccc4ccc3c2c1 ; c1ccc(Nc2ccc3c4ccccc4n(-c4ccccc4)c3c2)cc1 ; c1ccc(N(c2ccc3ccc4c5ccccc5ccc4c3c2)c2ccc3c4ccccc4n(-c4ccccc4)c3c2)cc1]

Compound 13-1 (15 g, 44.85 mmol), Compound 13-2 (12.4 g, 47.09 mmol), Pd₂(dba)₃ (2.1 g, 2.24 mmol), NaOt-Bu (6.5 g, 67.28 mmol), and S-Phos (1.5 g, 3.59 mmol) were added to 225 mL of toluene, and then stirred at 120° C. for 2 hours. After the reaction was completed, the mixture was cooled to room temperature and filtered through Celite. Next, it was distillated under reduced pressure, and then separated by column chromatography to obtain Compound H1-221 (9.1 g, yield: 36%).

| | MW | M.P |
|---|---|---|
| H1-221 | 560.6 | 204.9° C. |

### [Example 13] Preparation of Compound H1-222

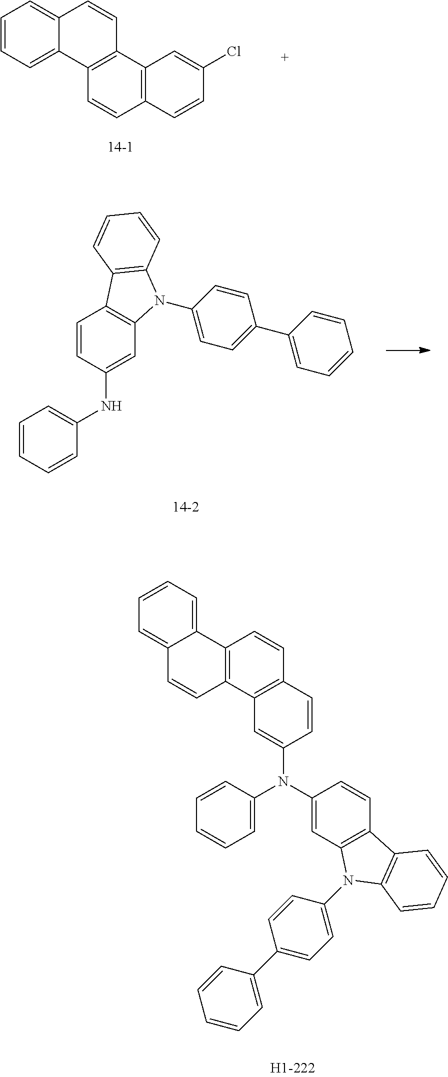 [SMILES: Clc1ccc2ccc3c4ccccc4ccc3c2c1 ; c1ccc(Nc2ccc3c4ccccc4n(-c4ccc(-c5ccccc5)cc4)c3c2)cc1 ; c1ccc(-c2ccc(-n3c4ccccc4c4ccc(N(c5ccccc5)c5ccc6ccc7c8ccccc8ccc7c6c5)cc43)cc2)cc1]

Compound 14-1 (5.6 g, 13.64 mmol), Compound 14-2 (4.3 g, 16.37 mmol), Pd₂(dba)₃ (620 mg, 0.68 mmol), S-Phos (560 mg, 1.36 mmol), NaOt-Bu (1.97 g, 20.46 mmol), and 91 mL of o-oxylene were added to a flask and dissolved, and then stirred under reflux at 180° C. for 1 hour. After the reaction was completed, the organic layer was extracted, the residual moisture was removed with magnesium sulfate, and then separated by column chromatography to obtain Compound H1-222 (3.2 g, yield: 36.84%).

| | MW | M.P |
|---|---|---|
| H1-222 | 636.78 | 247.4° C. |

### [Example 14] Preparation of Compound H1-223

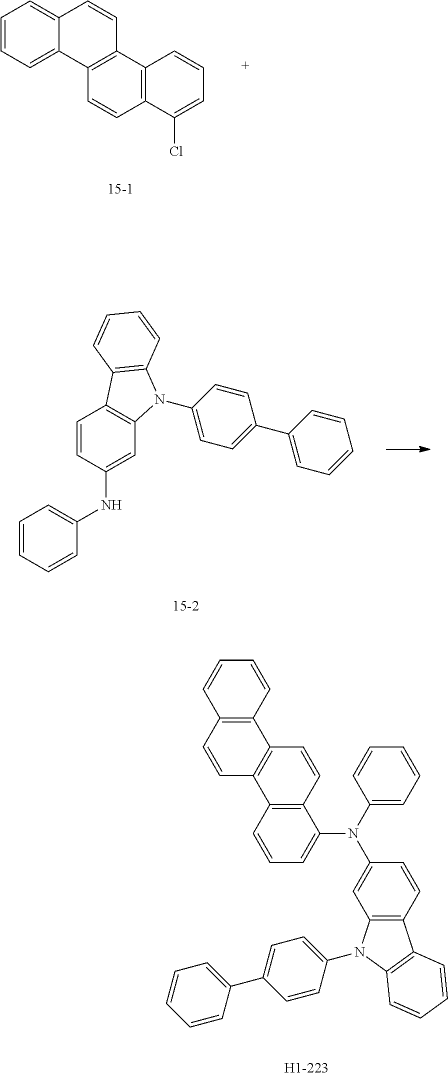 [SMILES: Clc1cccc2c1ccc1c3ccccc3ccc21 ; c1ccc(Nc2ccc3c4ccccc4n(-c4ccc(-c5ccccc5)cc4)c3c2)cc1 ; c1ccc(-c2ccc(-n3c4ccccc4c4ccc(N(c5ccccc5)c5cccc6c5ccc5c7ccccc7ccc65)cc43)cc2)cc1]

Compound 15-1 (4.3 g, 10.47 mmol), Compound 15-2 (3.3 g, 12.57 mmol), Pd₂(dba)₃ (480 mg, 0.52 mmol), S-Phos (430 mg, 1.05 mmol), NaOt-Bu (1.51 g, 15.71 mmol), and 70 mL of o-xylene were added to a flask and dissolved, and then stirred under reflux at 180° C. for 1 hour. After the reaction was completed, the organic layer was extracted, the residual moisture was removed with magnesium sulfate, and then separated by column chromatography to obtain Compound H1-223 (1.9 g, yield: 28.4%).

| | MW | M.P |
|---|---|---|
| H1-223 | 636.78 | 268.5° C. |

### [Example 15] Preparation of Compound H1-115

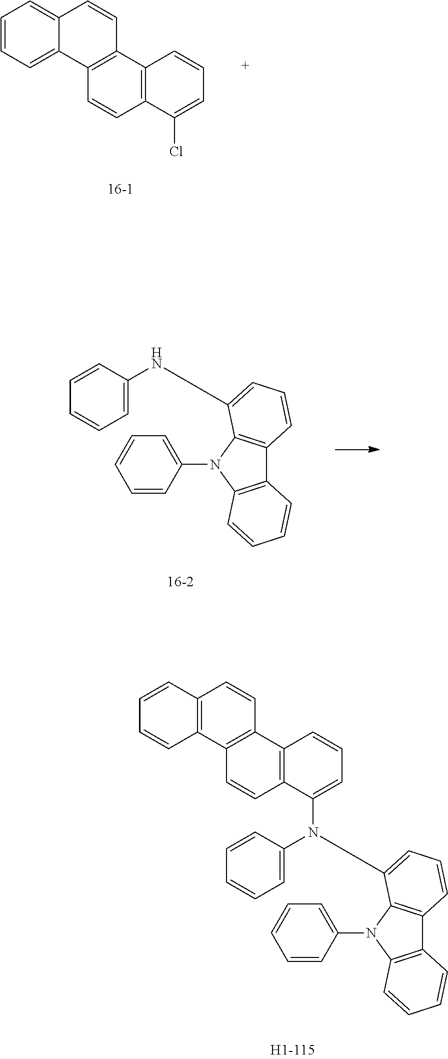 [SMILES: Clc1cccc2c1ccc1c3ccccc3ccc21 ; c1ccc(Nc2cccc3c4ccccc4n(-c4ccccc4)c23)cc1 ; c1ccc(N(c2cccc3c2ccc2c4ccccc4ccc32)c2cccc3c4ccccc4n(-c4ccccc4)c23)cc1]

Compound 16-1 (7.54 g, 28.71 mmol), Compound 16-2 (8 g, 23.92 mmol), Pd₂(dba)₃ (1.1 mg, 1.2 mmol), S-Phos (980 mg, 2.39 mmol), NaOt-Bu (3.45 g, 35.88 mmol), and 159 mL of o-xylene were added to a flask and dissolved, and then stirred under reflux at 180° C. for 1 hour. After the reaction was completed, the organic layer was extracted, the residual moisture was removed with magnesium sulfate, and then separated by column chromatography to obtain Compound H1-115 (3.1 g, yield: 23.1%).

| | MW | M.P |
|---|---|---|
| H1-115 | 560.70 | 326.8° C. |

### [Example 16] Preparation of Compound H1-224

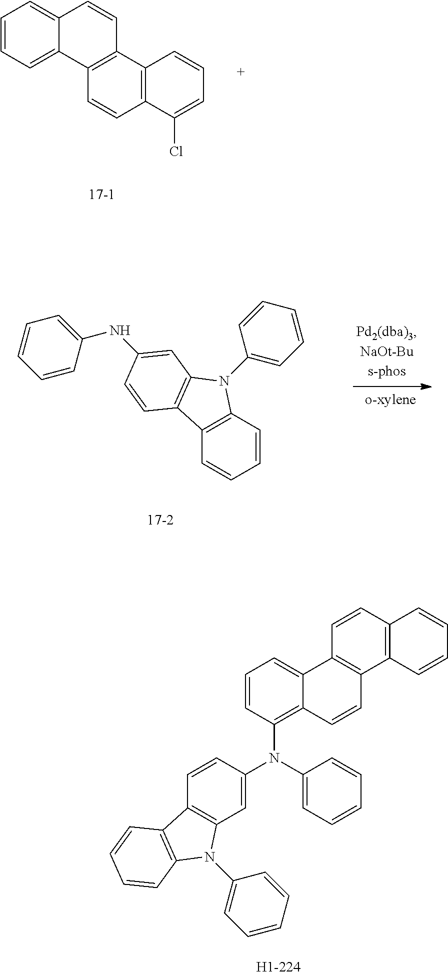 [SMILES: Clc1cccc2c1ccc1c3ccccc3ccc21 ; c1ccc(Nc2ccc3c4ccccc4n(-c4ccccc4)c3c2)cc1 ; c1ccc(N(c2ccc3c4ccccc4n(-c4ccccc4)c3c2)c2cccc3c2ccc2c4ccccc4ccc32)cc1]

95 mL of O-xylene was added to Compound 17-1 (5 g, 19 mmol), Compound 17-2 (7 g, 20.9 mmol), Pd₂(dba)₃ (0.9 g, 0.95 mmol), NaOt-Bu (2.8 g, 28.5 mmol), S-Phos (0.6 g, 1.52 mmol), and then stirred at 120° C. for 2 hours. After the reaction was completed, the mixture was cooled to room temperature and filtered through Celite. After distillation under reduced pressure, it was separated by column chromatography to obtain Compound H1-224 (7.8 g, yield: 73%).

| | MW | M.P |
|---|---|---|
| H1-224 | 560.6 | 278.9° C. |

### [Example 17] Preparation of Compound H1-225

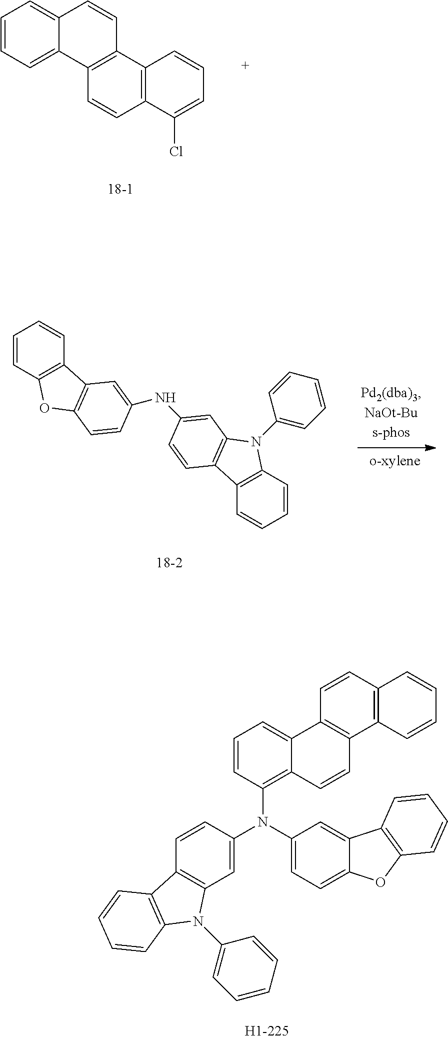 [SMILES: Clc1cccc2c1ccc1c3ccccc3ccc21 ; c1ccc(-n2c3ccccc3c3ccc(Nc4ccc5oc6ccccc6c5c4)cc32)cc1 ; c1ccc(-n2c3ccccc3c3ccc(N(c4ccc5oc6ccccc6c5c4)c4cccc5c4ccc4c6ccccc6ccc54)cc32)cc1]

60 mL of O-xylene was added to Compound 18-1 (3.7 g, 14.13 mmol), Compound 18-2 (5 g, 11.78 mmol), Pd₂(dba)₃ (0.54 g, 0.589 mmol), NaOt-Bu (1.7 g, 17.67 mmol), S-Phos (0.48 g, 1.178 mmol), and then stirred at 120° C. for 2 hours. After the reaction was completed, the mixture was cooled to room temperature and filtered through Celite. After distillation under reduced pressure, it was separated by column chromatography to obtain Compound H1-225 (6 g, yield: 78%).

| | MW | M.P |
|---|---|---|
| H1-225 | 650.7 | 183.2° C. |

### [Example 18] Preparation of Compound H1-226

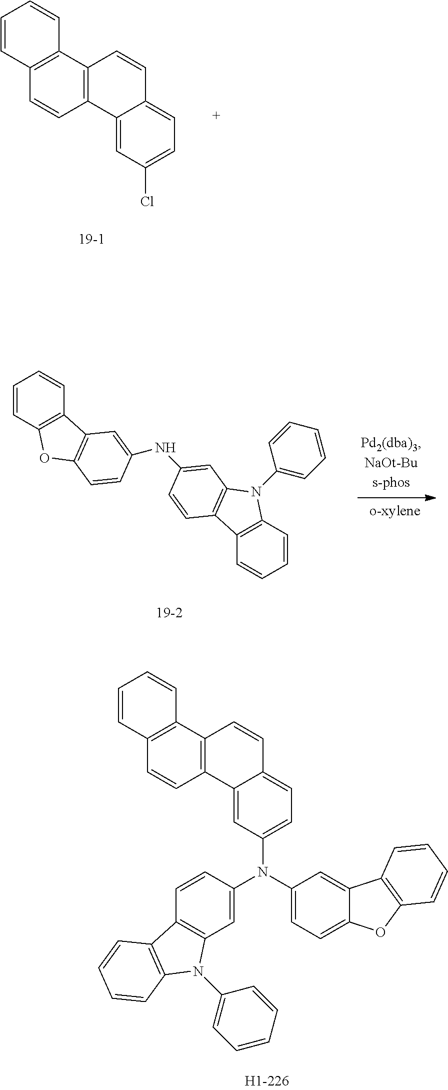 [SMILES: Clc1ccc2ccc3c4ccccc4ccc3c2c1 ; c1ccc(-n2c3ccccc3c3ccc(Nc4ccc5oc6ccccc6c5c4)cc32)cc1 ; c1ccc(-n2c3ccccc3c3ccc(N(c4ccc5ccc6c7ccccc7ccc6c5c4)c4ccc5oc6ccccc6c5c4)cc32)cc1]

60 mL of o-xylene was added to Compound 19-1 (3.7 g, 14.13 mmol), Compound 19-2 (5 g, 11.78 mmol), Pd₂(dba)₃ (0.54 g, 0.589 mmol), NaOt-Bu (1.7 g, 17.67 mmol), and S-Phos (0.48 g, 1.178 mmol), and then stirred at 120° C. for 2 hours. After the reaction was completed, the mixture was cooled to room temperature and filtered through Celite. After distillation under reduced pressure, it was separated by column chromatography to obtain Compound H1-226 (4 g, yield: 52%).

| | MW | M.P |
|---|---|---|
| H1-226 | 650.7 | 173.2° C. |

### [Example 19] Preparation of Compound H1-227

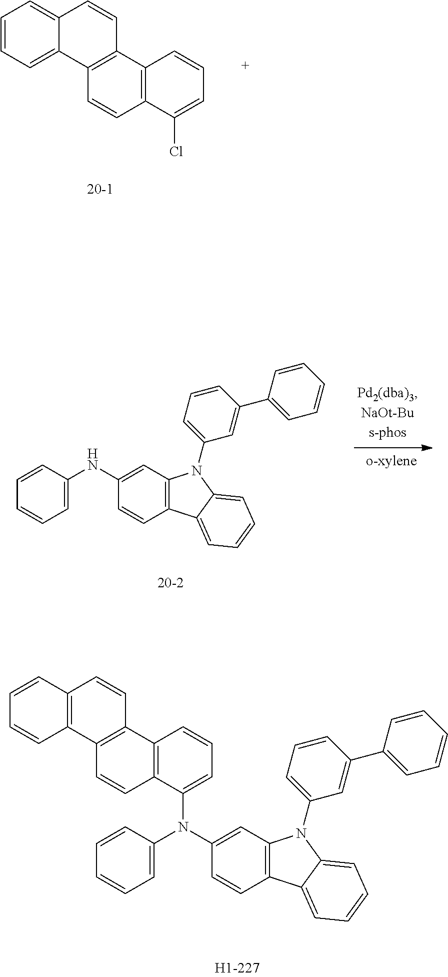 [SMILES: Clc1cccc2c1ccc1c3ccccc3ccc21 ; c1ccc(Nc2ccc3c4ccccc4n(-c4cccc(-c5ccccc5)c4)c3c2)cc1 ; c1ccc(-c2cccc(-n3c4ccccc4c4ccc(N(c5ccccc5)c5cccc6c5ccc5c7ccccc7ccc65)cc43)c2)cc1]

Compound 20-1 (4 g, 15.5 mmol), Compound 20-2 (7 g, 17.05 mmol), Pd₂(dba)₃ (709 mg, 0.775 mmol), S-Phos (509 mg, 1.24 mmol), NaOtBu (3.4 g, 35.65 mmol), and 77.5 mL of o-xylene were added to a flask, and then stirred under reflux at 160° C. and 1 hour. After the reaction was completed, the mixture was cooled to room temperature, the organic layer was extracted with ethyl acetate, the residual moisture was removed using magnesium sulfate, dried, and separated by column chromatography to obtain Compound H1-227 (3.1 g, yield: 30%).

| | MW | M.P |
|---|---|---|
| H1-227 | 636.8 | 240.8° C. |

### [Example 20] Preparation of Compound H1-241

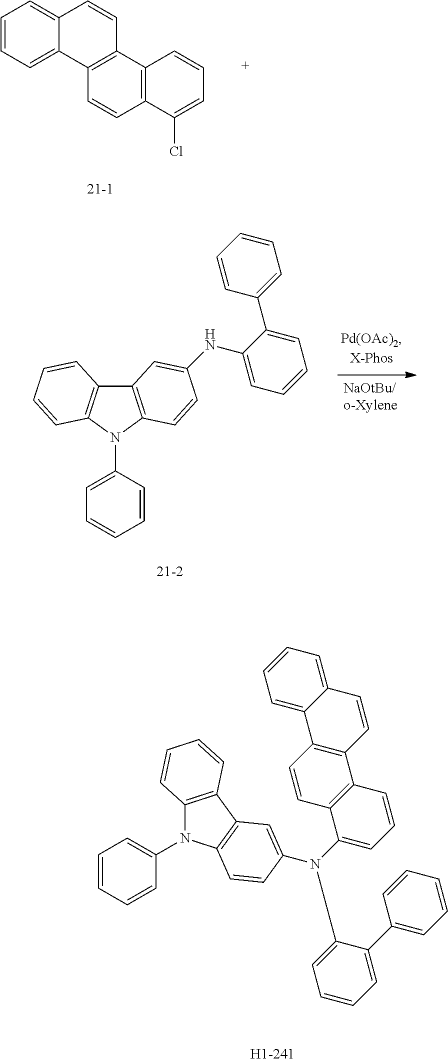 [SMILES: Clc1cccc2c1ccc1c3ccccc3ccc21 ; c1ccc(-c2ccccc2Nc2ccc3c(c2)c2ccccc2n3-c2ccccc2)cc1 ; c1ccc(-c2ccccc2N(c2ccc3c(c2)c2ccccc2n3-c2ccccc2)c2cccc3c2ccc2c4ccccc4ccc32)cc1]

Compound 21-1 (30 g, 114 mmol), Compound 21-2 (19.2 g, 46.7 mol), Pd(OAc)₂ (80 mg, 0.035 mmol), X-Phos (350 mg, 0.7 mmol), and NaOtBu (8.7 g, 90 mmol) were added to 450 mL of o-xylene and dissolved, and then stirred for 0.5 hours at 150° C. After the reaction was completed, the mixture was cooled to room temperature and filtered through Celite. After distillation under reduced pressure, it was separated by column chromatography to obtain Compound H1-241 (4.7 g, yield: 24%).

| | MW | M.P |
|---|---|---|
| H1-241 | 636.26 | 220.8° C. |

Hereinafter, the preparation method of an organic electroluminescent device comprising the plurality of host materials according to the present disclosure, and the device property thereof will be explained in order to understand the present disclosure in detail.

### [Device Examples 1 to 10] Preparation of OLEDs Co-Deposited with the First Host Compound and the Second Host Compound According to the Present Disclosure

OLEDs according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, Compound HI-1 was introduced into a cell of the vacuum vapor deposition apparatus, and Compound HT-1 was introduced into another cell. The two materials were evaporated at different rates and Compound HI-1 was deposited in a doping amount of 3 wt % based on the total amount of compounds HI-1 and HT-1 to form a hole injection layer having a thickness of 10 nm. Next, Compound HT-1 was deposited as a first hole transport layer having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: each of the first host compound and the second host compound described in the following Table 1 were introduced into two cells of the vacuum vapor deposition apparatus as hosts, respectively, and Compound D-39 was introduced into another cell as a dopant. The two host materials were evaporated at a rate of 1:1 and the dopant material was evaporated at a different rate, simultaneously, and was deposited in a doping amount of 3 wt % based on the total amount of the hosts and dopant to form a light-emitting layer having a thickness of 40 nm on the second hole transport layer. Next, compounds ET-1 and EI-1 as electron transport materials were deposited at a weight ratio of 50:50 to form an electron transport layer having a thickness of 35 nm on the light-emitting layer. After depositing Compound EI-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials were purified by vacuum sublimation under 10⁻⁶ torr.

### [Device Comparative Example 1] Preparation of an OLED Comprising a Single Host Compound

An OLED was manufactured in the same manner as in Device Example 1, except that the second host compound described in the following Table 1 as the host of the light-emitting layer was used alone.

The driving voltage, current efficiency, and the luminous color at a luminance of 5,000 nits and the time taken for luminance to decrease from 100% to 95% at a luminance of 10,000 nits (lifespan: T95) of the OLED devices of Device Examples 1 to 10 and Device Comparative Example 1 produced as described above, are measured, and the results thereof are shown in the following Table 1.

TABLE 1

| | | | | Current | | |
|---|---|---|---|---|---|---|
| | | | Driving | Effi- | Lumi- | |
| | First | Second | Voltage | ciency | nous | Lifespan |
| | Host | Host | (V) | (cd/A) | Color | T95(hr) |
| Device | H1-159 | H2-146 | 3.7 | 33.4 | Red | 354 |
| Example 1 | | | | | | |
| Device | H1-186 | H2-146 | 3.6 | 31.8 | Red | 341 |
| Example 2 | | | | | | |
| Device | H1-189 | H2-146 | 3.8 | 33.6 | Red | 141 |
| Example 3 | | | | | | |
| Device | H1-219 | H2-146 | 4.5 | 32.7 | Red | 194 |
| Example 4 | | | | | | |
| Device | H1-188 | H2-146 | 3.7 | 33.7 | Red | 378 |
| Example 5 | | | | | | |
| Device | H1-221 | H2-146 | 3.7 | 33.4 | Red | 269 |
| Example 6 | | | | | | |
| Device | H1-223 | H2-146 | 3.9 | 32.6 | Red | 412 |
| Example 7 | | | | | | |
| Device | H1-222 | H2-146 | 3.8 | 32.6 | Red | 364 |
| Example 8 | | | | | | |
| Device | H1-224 | H2-146 | 3.7 | 33.2 | Red | 483 |
| Example 9 | | | | | | |
| Device | H1-225 | H2-146 | 3.7 | 30.9 | Red | 338 |
| Example 10 | | | | | | |
| Device | — | H2-146 | 4.5 | 25.8 | Red | 17.7 |
| Comparative | | | | | | |
| Example 1 | | | | | | |

From Table 1 above, it can be seen that organic electroluminescent devices comprising the specific combination of compounds according to the present disclosure as host materials have low driving voltage, high luminous efficiency, and in particular, significantly improved lifespan characteristics.

The compounds used in the Device Examples and the Device Comparative Example above are shown in the following Table 2:

TABLE 2

| | |
|---|---|
| Hole Injection Layer/Hole Transport Layer | 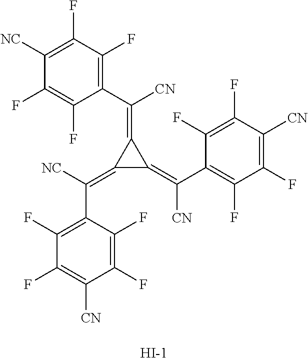 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 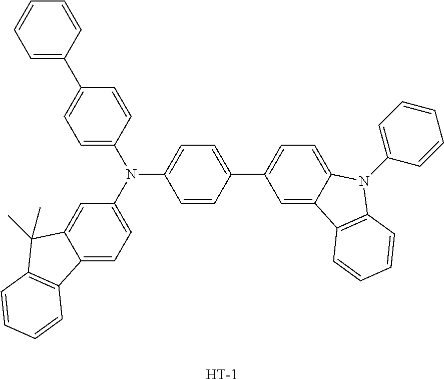 |
| | 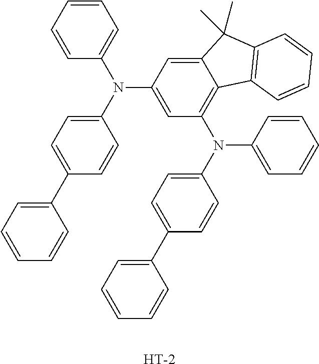 |
| Light- Emitting Layer | 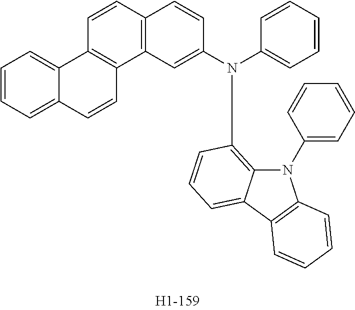 [SMILES: c1ccc(N(c2ccc3ccc4c5ccccc5ccc4c3c2)c2cccc3c4ccccc4n(-c4ccccc4)c23)cc1] |
| | 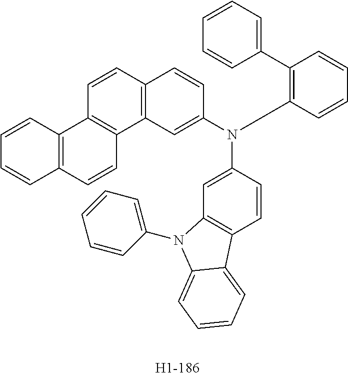 [SMILES: c1ccc(-c2ccccc2N(c2ccc3ccc4c5ccccc5ccc4c3c2)c2ccc3c4ccccc4n(-c4ccccc4)c3c2)cc1] |
| | 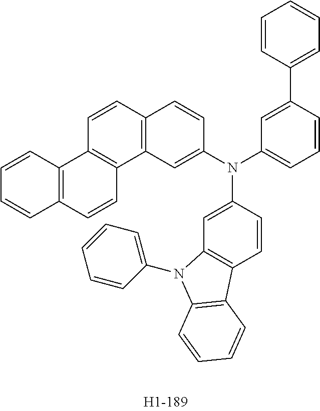 [SMILES: c1ccc(-c2cccc(N(c3ccc4ccc5c6ccccc6ccc5c4c3)c3ccc4c5ccccc5n(-c5ccccc5)c4c3)c2)cc1] |
| | 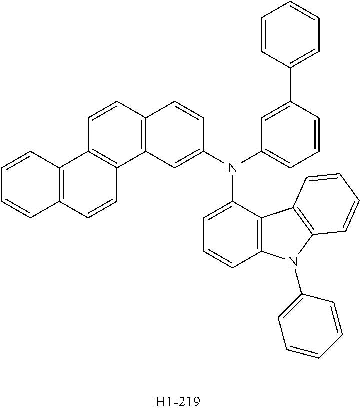 [SMILES: c1ccc(-c2cccc(N(c3ccc4ccc5c6ccccc6ccc5c4c3)c3cccc4c3c3ccccc3n4-c3ccccc3)c2)cc1] |
| | 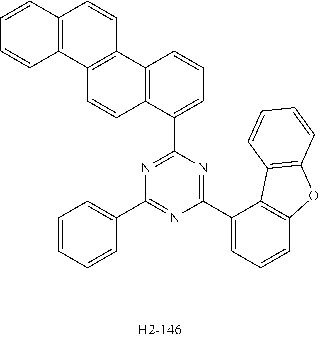 [SMILES: c1ccc(-c2nc(-c3cccc4c3ccc3c5ccccc5ccc43)nc(-c3cccc4oc5ccccc5c34)n2)cc1] |
| | 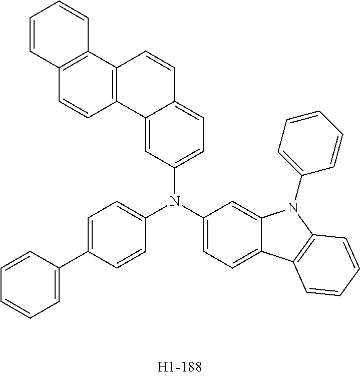 [SMILES: c1ccc(-c2ccc(N(c3ccc4ccc5c6ccccc6ccc5c4c3)c3ccc4c5ccccc5n(-c5ccccc5)c4c3)cc2)cc1] |
| | 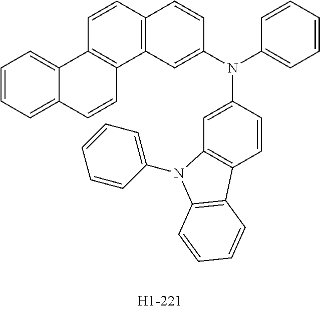 [SMILES: c1ccc(N(c2ccc3ccc4c5ccccc5ccc4c3c2)c2ccc3c4ccccc4n(-c4ccccc4)c3c2)cc1] |
| | 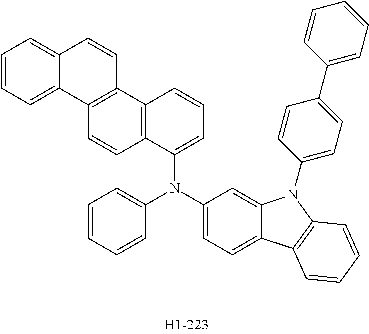 [SMILES: c1ccc(-c2ccc(-n3c4ccccc4c4ccc(N(c5ccccc5)c5cccc6c5ccc5c7ccccc7ccc65)cc43)cc2)cc1] |
| | 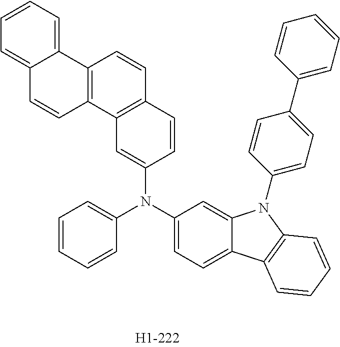 [SMILES: c1ccc(-c2ccc(-n3c4ccccc4c4ccc(N(c5ccccc5)c5ccc6ccc7c8ccccc8ccc7c6c5)cc43)cc2)cc1] |
| | 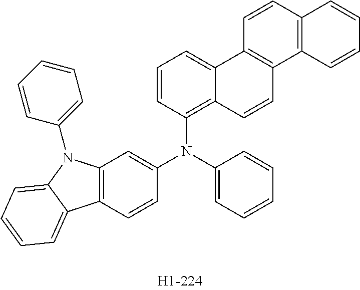 [SMILES: c1ccc(N(c2ccc3c4ccccc4n(-c4ccccc4)c3c2)c2cccc3c2ccc2c4ccccc4ccc32)cc1] |
| | 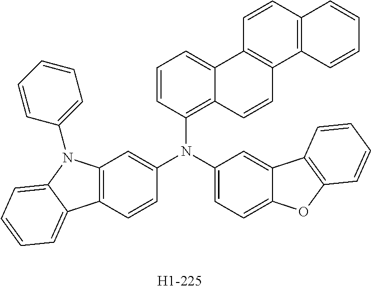 [SMILES: c1ccc(-n2c3ccccc3c3ccc(N(c4ccc5oc6ccccc6c5c4)c4cccc5c4ccc4c6ccccc6ccc54)cc32)cc1] |
| | 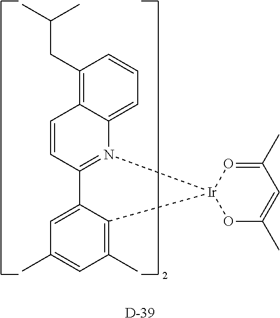 |
| Electron Transport Layer/ Electron Injection Layer | 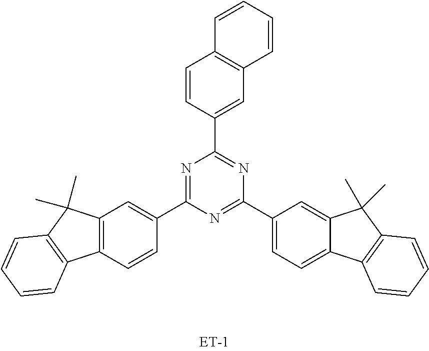 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 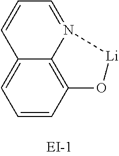 |

1. A plurality of host materials comprising at least one first host compound and at least one second host compound, which is different from the first host compound, wherein the first host compound is represented by the following Formula 1 and the second host compound is represented by the following Formula 3: 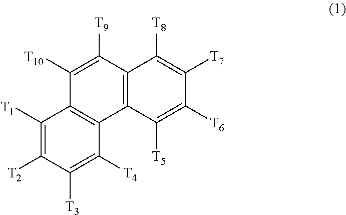
wherein,
T₅ and T₆ are connected to each other to form a ring of the following Formula 2; or T₇ and T₈ are connected to each other to form a ring of the following Formula 2; or T₅ and T₆ are connected to each other to form a ring of the following Formula 2, and T₇ and T₈ are connected to each other to form a ring of the following Formula 2; 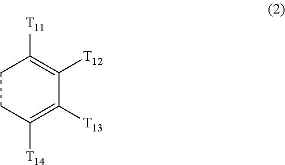
wherein,
T₁ to T₄, T₉ to T₁₄, and T₅ to T₈, which do not form a ring, each independently represent, hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of (C3-C30) aliphatic ring and (C6-C30) aromatic ring, a substituted or unsubstituted (C6-C30)aryl, a substituted or unsubstituted (3- to 30-membered)heteroaryl, —SiR₁₁R₁₂R₁₃, or -L₁-NAr₁Ar₂; provided that at least one of T₁ to T₁₄ is -L₁-NAr₁Ar₂;
L₁ represents a single bond, a substituted or unsubstituted (C6-C30)arylene, or a substituted or unsubstituted (3- to 30-membered)heteroarylene;
R₁₁ to R₁₃, Ar₁, and Ar₂ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and [symbol]
represents a site fused with the Formula 1; 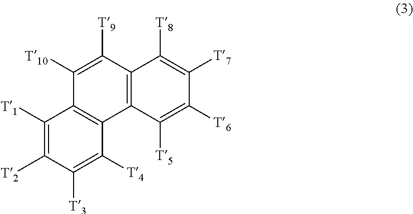
wherein,
T′₅ and T′₆ are connected to each other to form a ring of the following Formula 4; or T′₇ and T′₈ are connected to each other to form a ring of the following Formula 4; or T′₅ and T′₆ are connected to each other to form a ring of the following Formula 4, and T′₇ and T′₈ are connected to each other to form a ring of the following Formula 4; 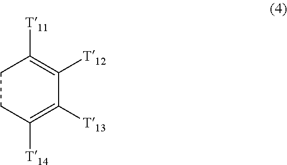
wherein,
T′₁ to T′₄, T′₉ to T′₁₄, and T′₅ to T′₈, which do not form a ring, each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted fused ring of (C3-C30) aliphatic ring and (C6-C30) aromatic ring, —SiR₁₁R₁₂R₁₃, —NR₁₄R₁₅, or -L₂-HAr₁; provided that at least one of T′₁ to T′₁₄ is -L₂-HAr₁;
R₁₁ to R₁₅ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
L₂ represents a single bond, a substituted or unsubstituted (C6-C30)arylene, or a substituted or unsubstituted (3- to 30-membered)heteroarylene;
HAr₁ represents a substituted or unsubstituted (3- to 30-membered)heteroaryl comprising at least one nitrogen (N) atom; and [symbol]
represents a site fused with the Formula 3.

2. The plurality of host materials according to claim 1, wherein the substituted alkyl, the substituted alkenyl, the substituted aryl(ene), the substituted heteroaryl(ene), the substituted cycloalkyl, the substituted heterocycloalkyl, the substituted alkoxy or the substituted fused ring of aliphatic ring and aromatic ring each independently is substituted with at least one selected from the group consisting of deuterium; halogen; cyano; carboxyl; nitro; hydroxyl; phosphine oxide; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (3- to 30-membered)heteroaryl unsubstituted or substituted with at least one of (C1-C30)alkyl and (C6-C30)aryl; (C6-C30)aryl unsubstituted or substituted with at least one of deuterium, cyano, (C1-C30)alkyl, (C3-C30)cycloalkyl, tri(C1-C30)alkylsilyl and (3- to 30-membered)heteroaryl; tri(C1-C30)alkylsilyl; tri(C6-C30)arylsilyl; di(C1-C30)alkyl(C6-C30)arylsilyl; (C1-C30)alkyldi(C6-C30)arylsilyl; a fused ring of (C3-C30) aliphatic ring and (C6-C30) aromatic ring; amino; mono- or di-(C1-C30)alkylamino; mono- or di-(C2-C30)alkenylamino; (C1-C30)alkyl(C2-C30)alkenylamino; mono- or di-(C6-C30)arylamino; (C1-C30)alkyl(C6-C30)arylamino; mono- or di-(3- to 30-membered)heteroarylamino; (C1-C30)alkyl(3- to 30-membered)heteroarylamino; (C2-C30)alkenyl(C6-C30)arylamino; (C2-C30)alkenyl(3- to 30-membered)heteroarylamino; (C6-C30)aryl(3- to 30-membered)heteroarylamino; (C1-C30)alkylcarbonyl; (C1-C30)alkoxycarbonyl; (C6-C30)arylcarbonyl; (C6-C30)arylphosphine; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl.

3. The plurality of host materials according to claim 1, wherein T₇ and T₈ in Formula 1 are connected to each other to form a ring of the Formula 2.

4. The plurality of host materials according to claim 1, wherein at least one of Ar₁ and Ar₂ in the first host compound is a substituted or unsubstituted 1-carbazolyl, a substituted or unsubstituted 2-carbazolyl, a substituted or unsubstituted 3-carbazolyl, a substituted or unsubstituted 4-carbazolyl, or a substituted or unsubstituted 9-carbazolyl.

5. The plurality of host materials according to claim 1, wherein at least one of T₁₁ to T₁₄ in the first host compound is -L₁-NAr₁Ar₂ (wherein L₁, Ar₁ and Ar₂ are as defined in claim 1).

6. The plurality of host materials according to claim 1, wherein T′₇ and T′₈ in Formula 3 are connected to each other to form a ring of the Formula 4.

7. The plurality of host materials according to claim 1, wherein HAr₁ in Formula 3 is represented by the following Formula 3-1-1. 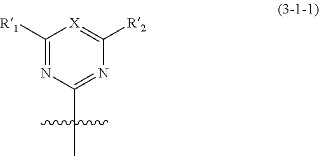
wherein,
X represents N or CR′;
R′₁ and R′₂ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and
R′ represents hydrogen or deuterium.

8. The plurality of host materials according to claim 1, wherein at least one of T′₁₁ to T′₁₄ in the second host compound is -L₂-HAr₁ (wherein L₂ and HAr₁ are as defined in claim 1).

9. The plurality of host materials according to claim 1, wherein the first host compound represented by the Formula 1 is selected from the following compounds:

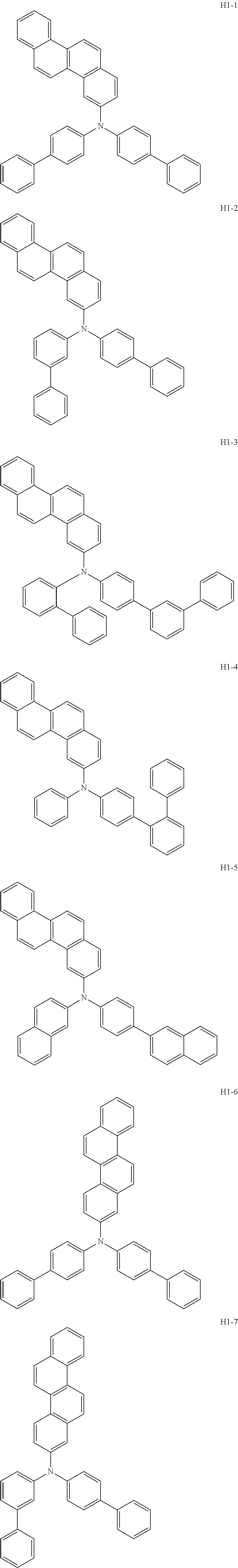

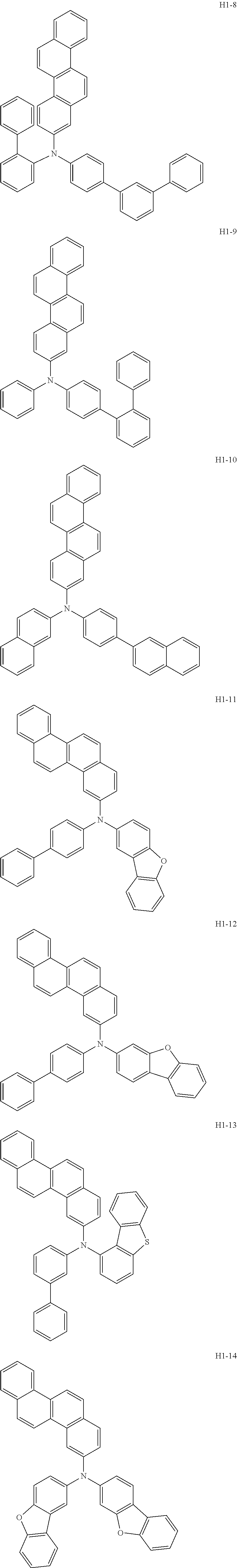

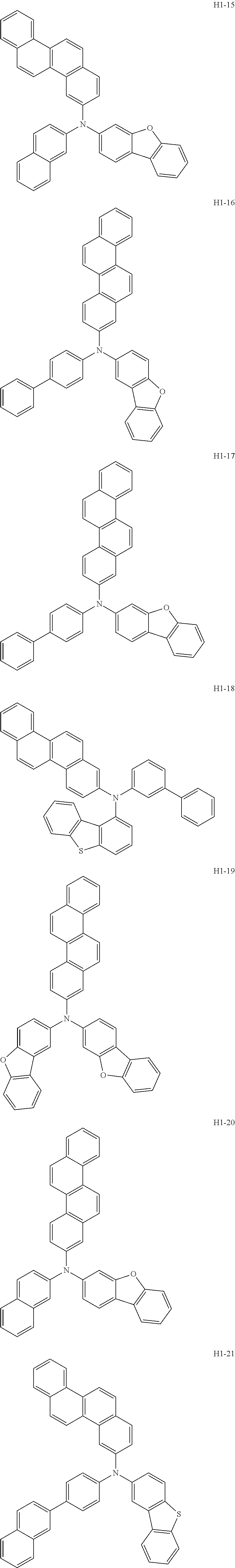

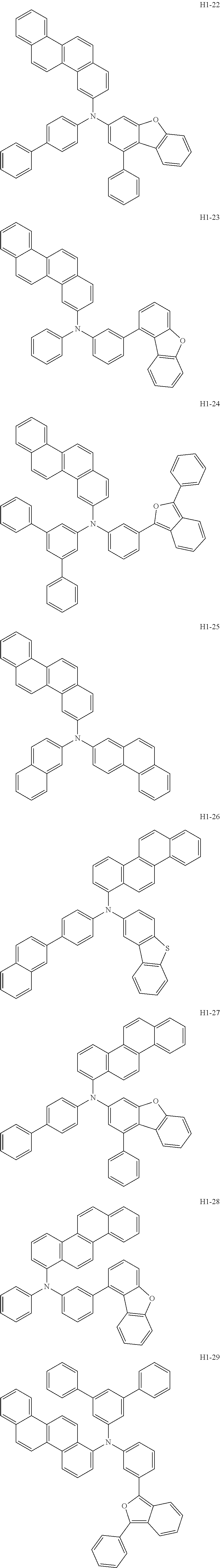

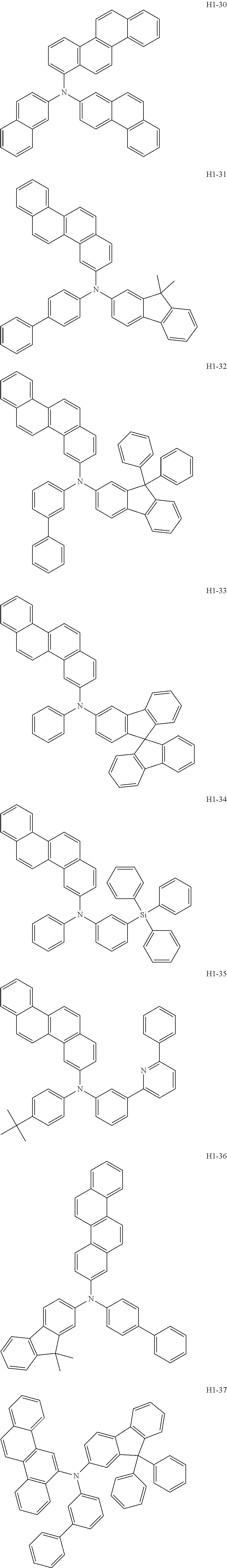

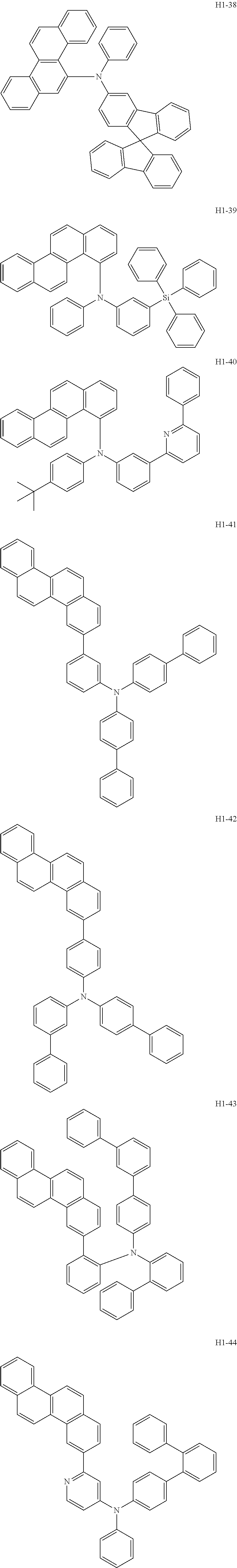

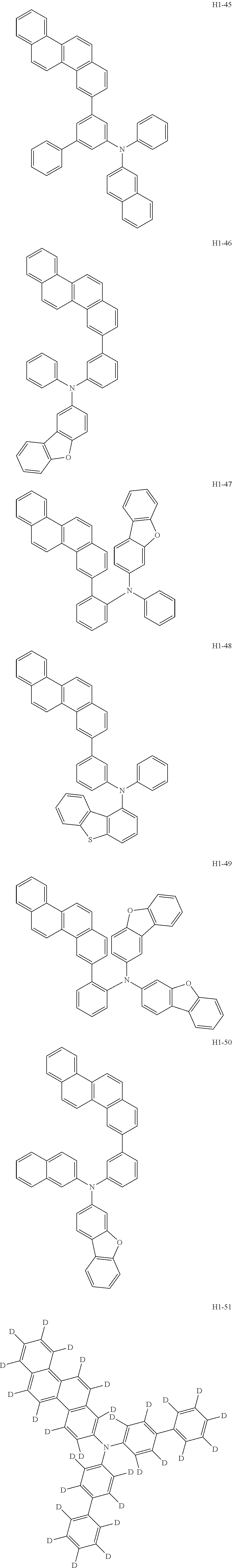

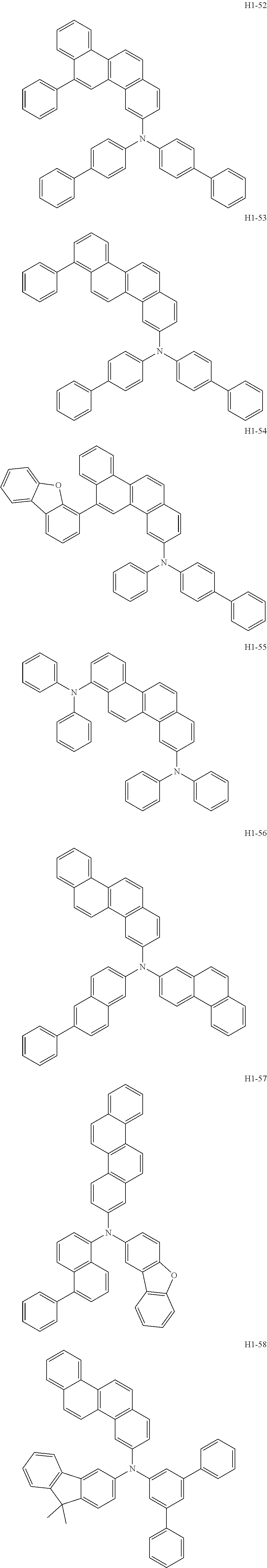

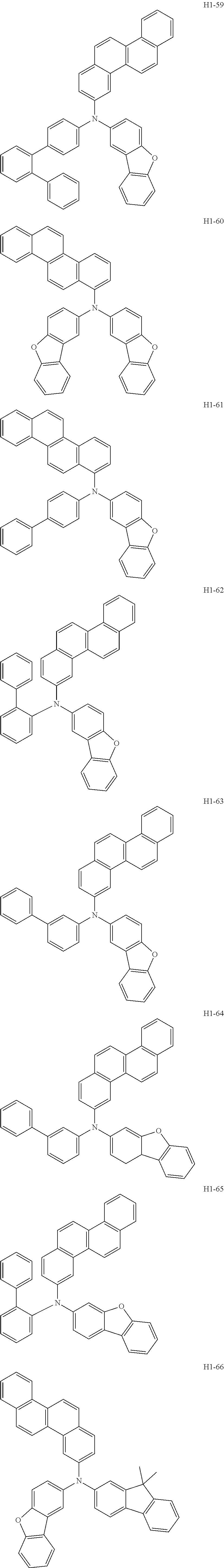

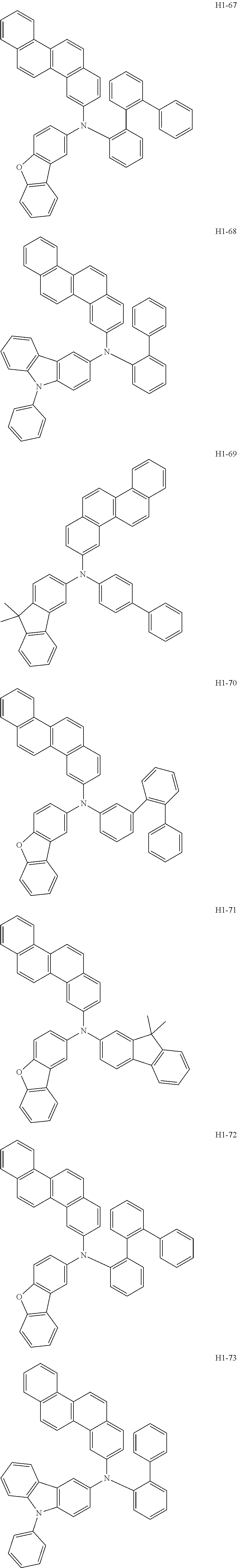

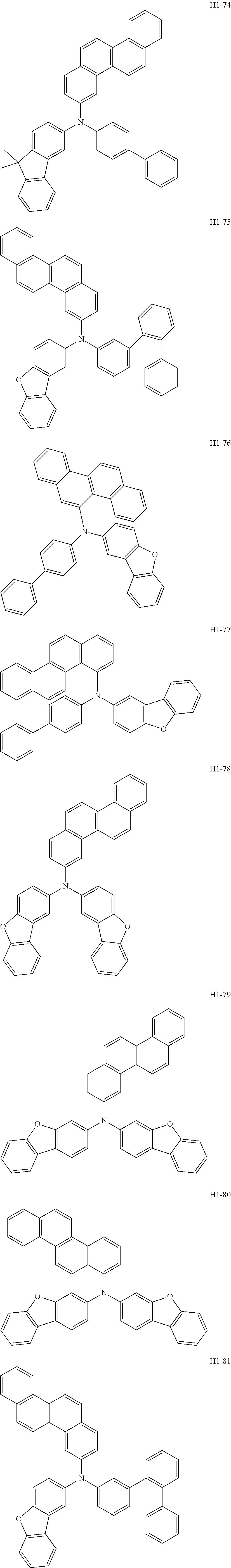

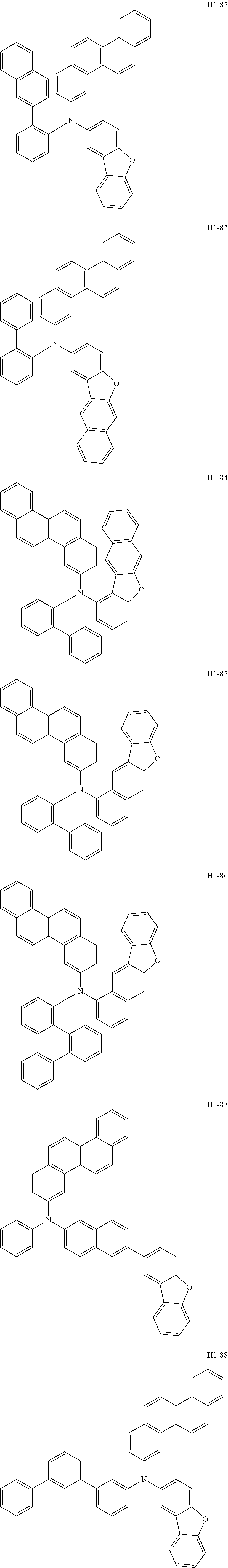

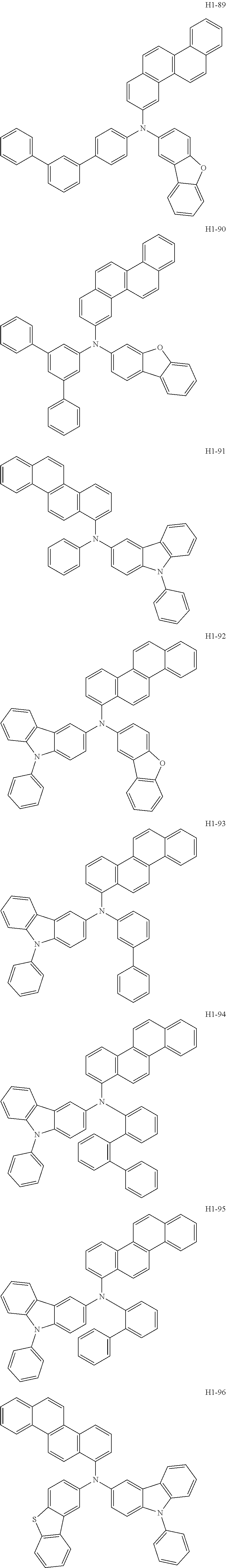

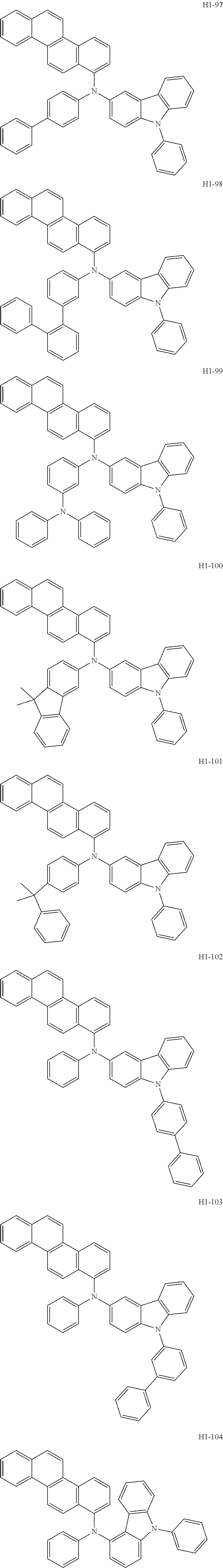

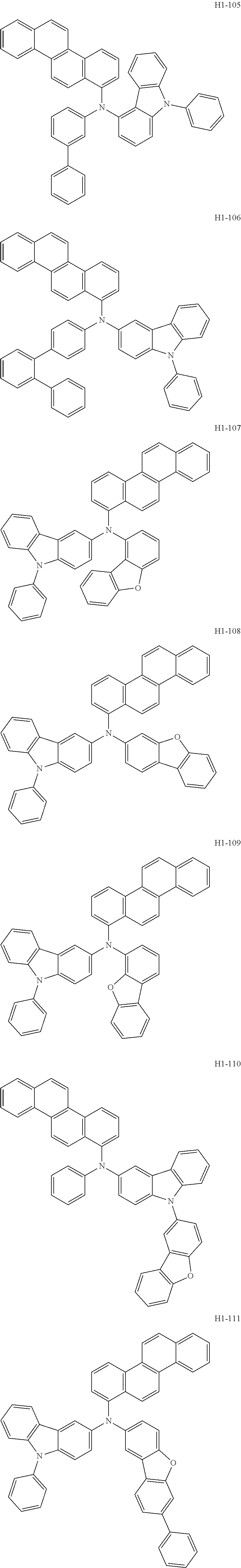

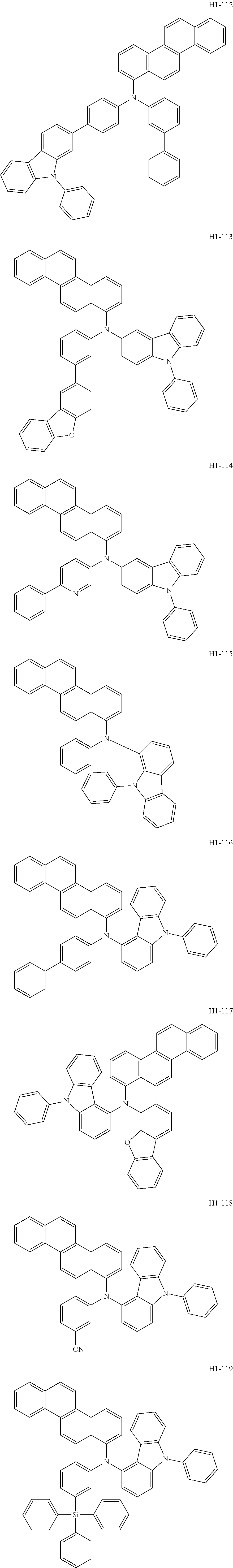

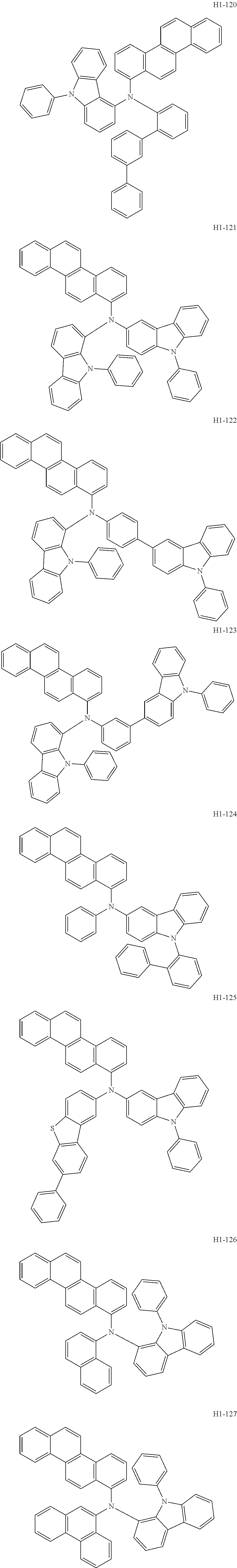

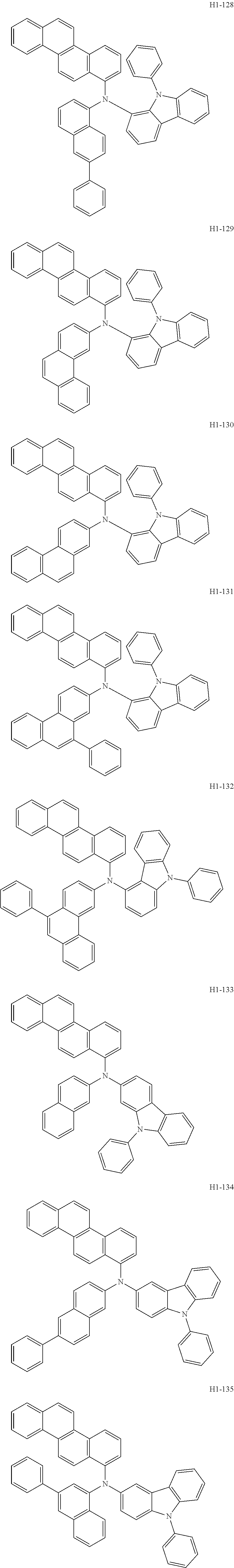

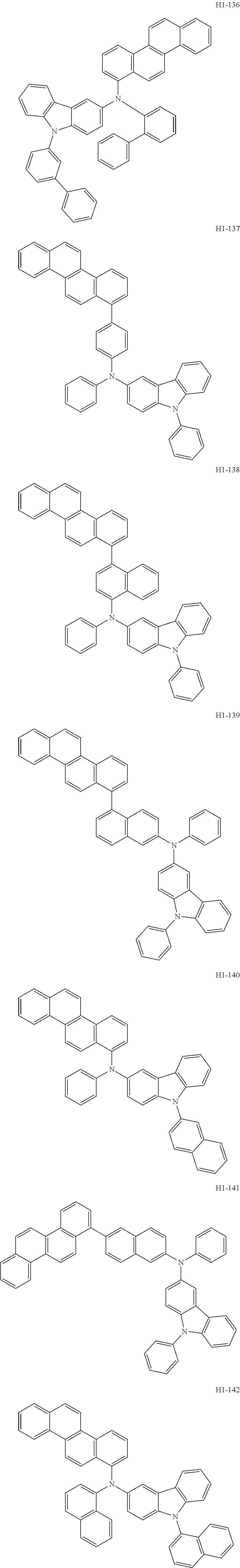

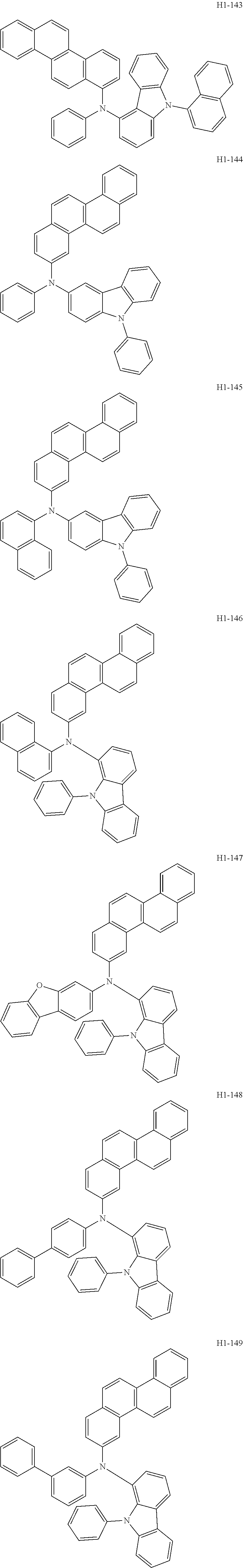

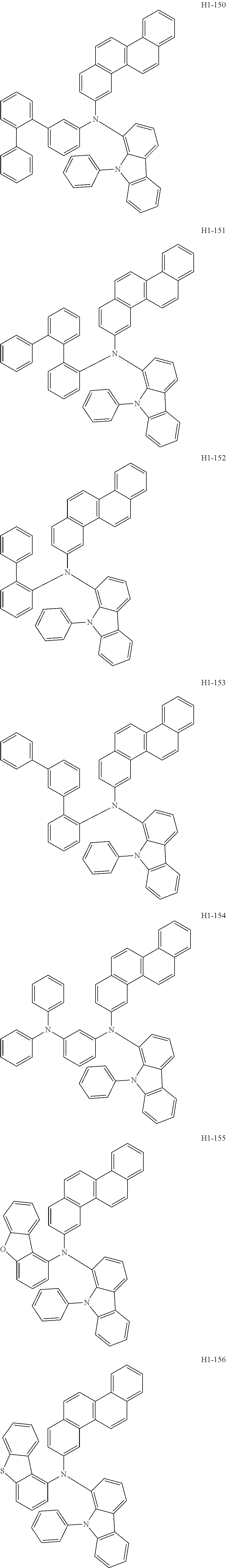

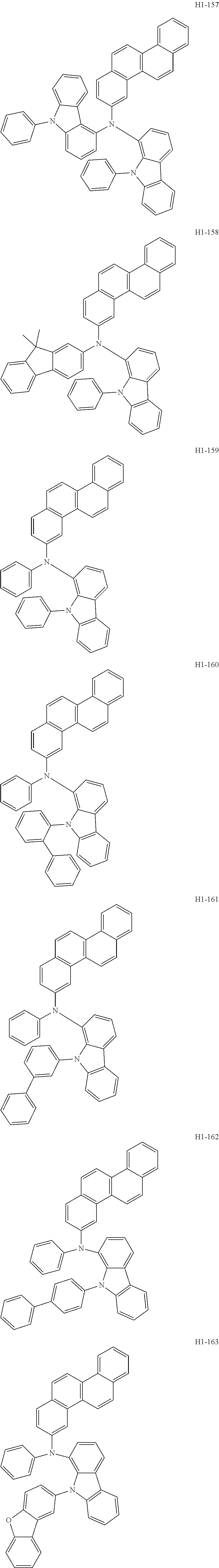

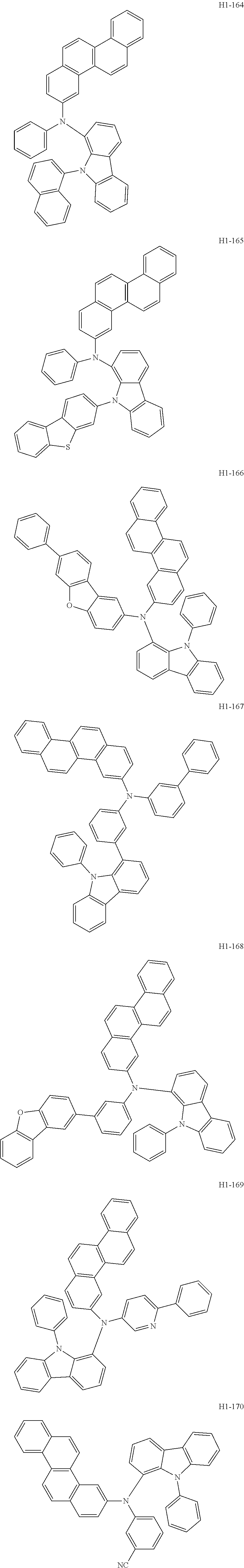

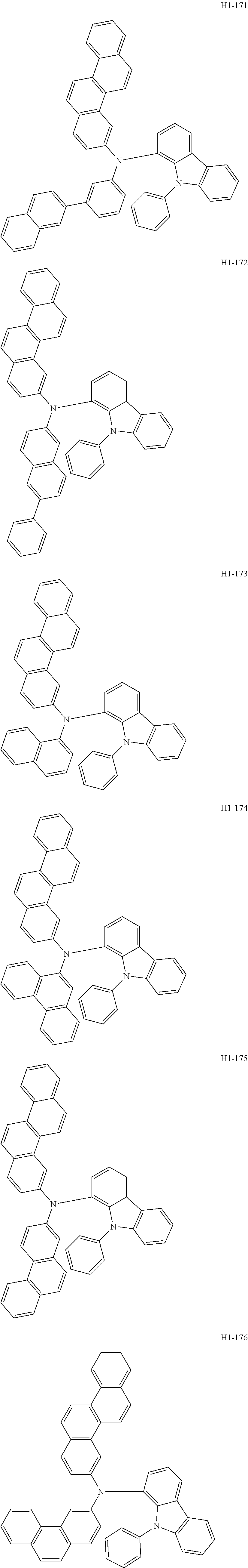

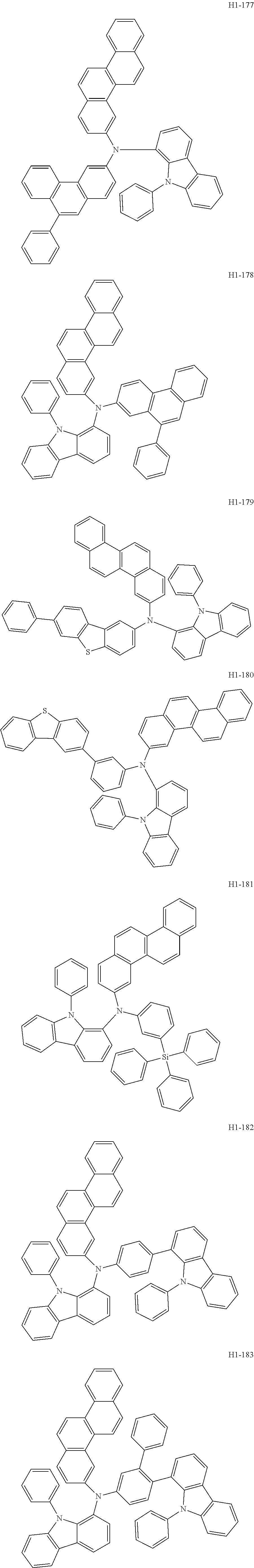

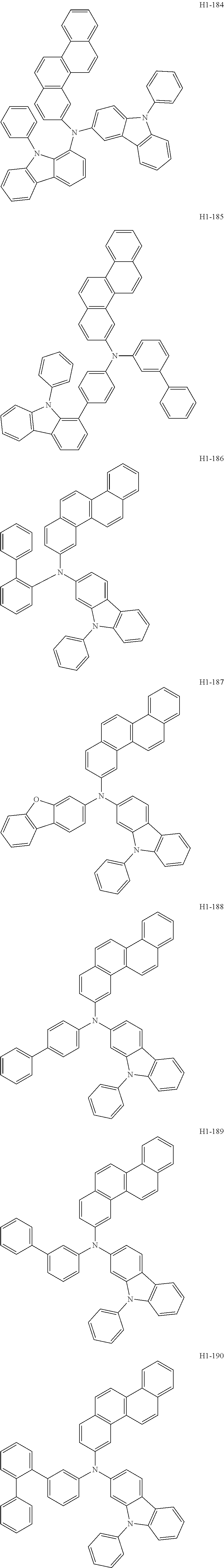

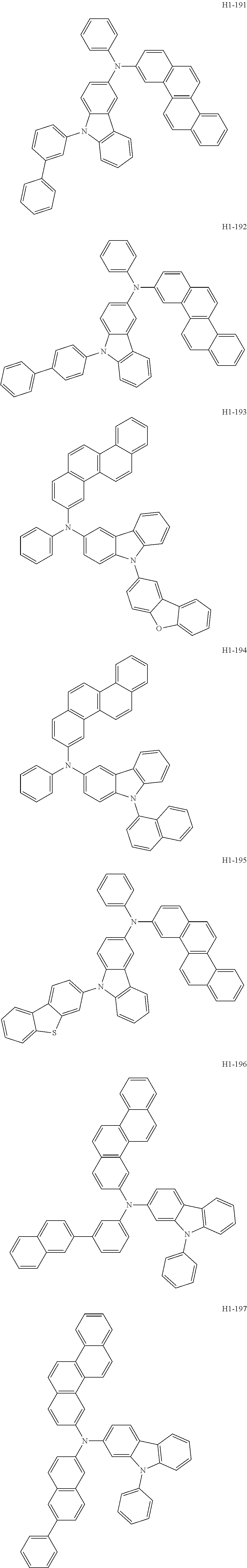

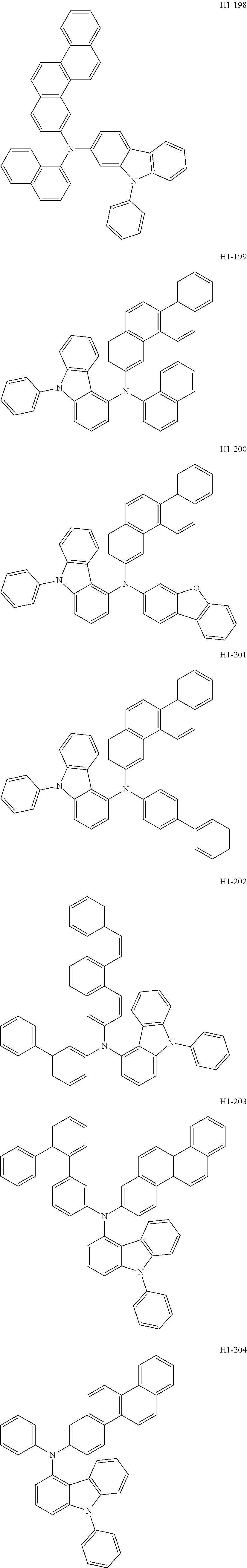

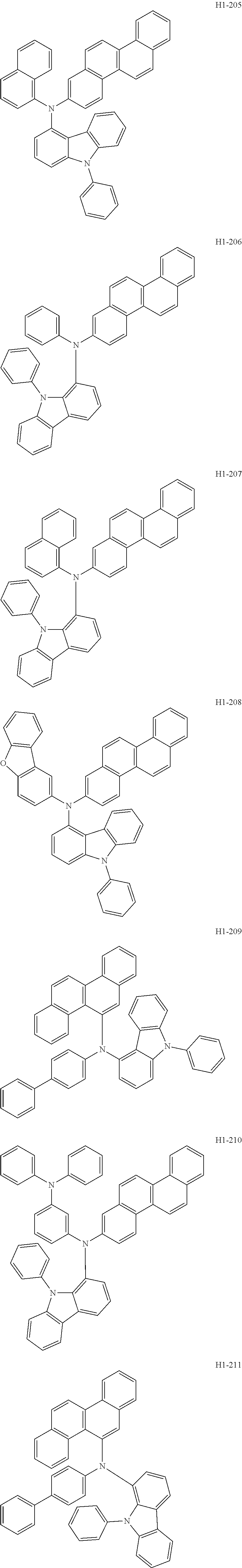

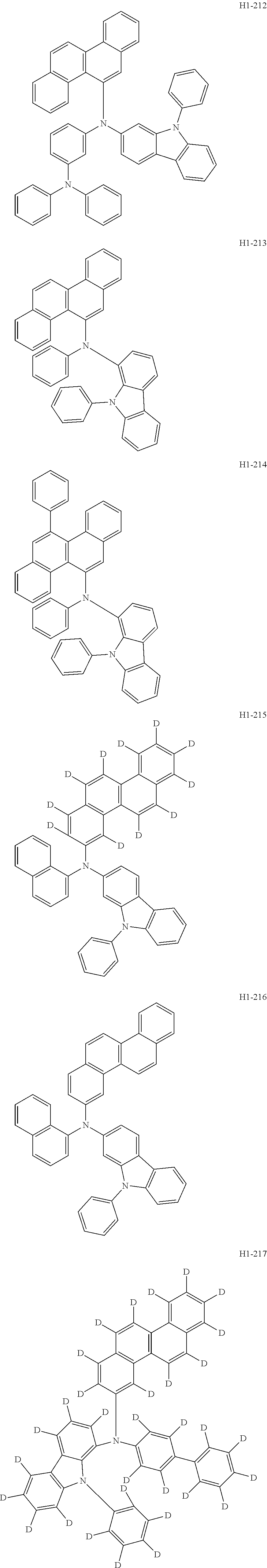

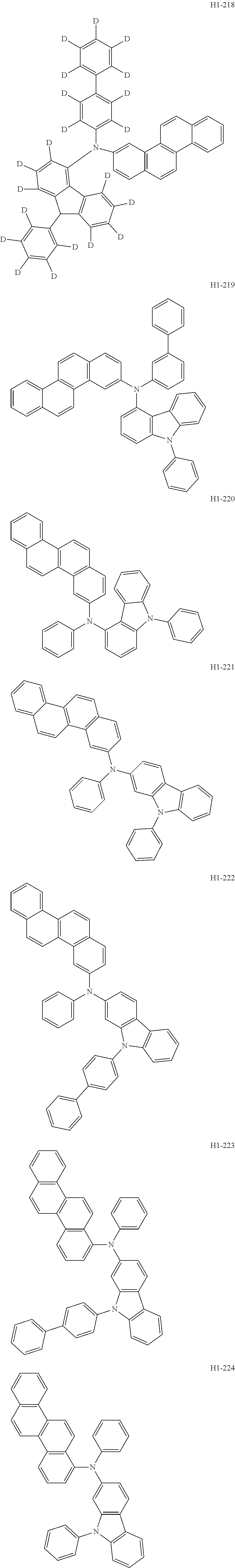

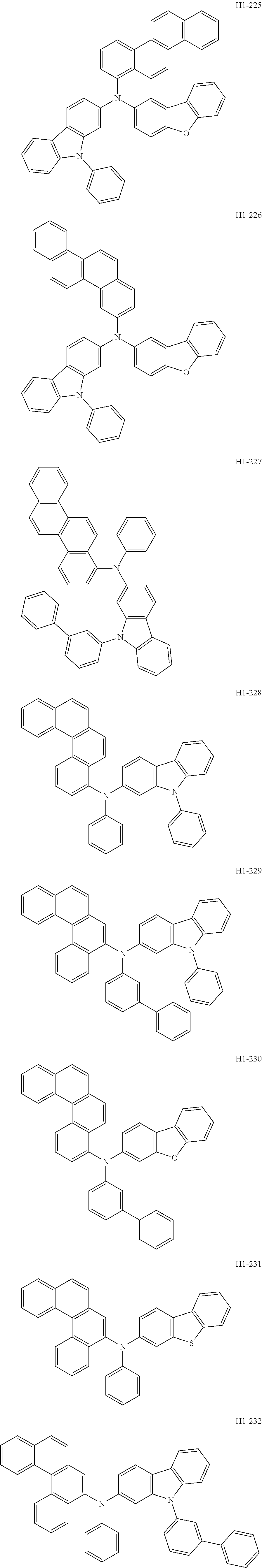

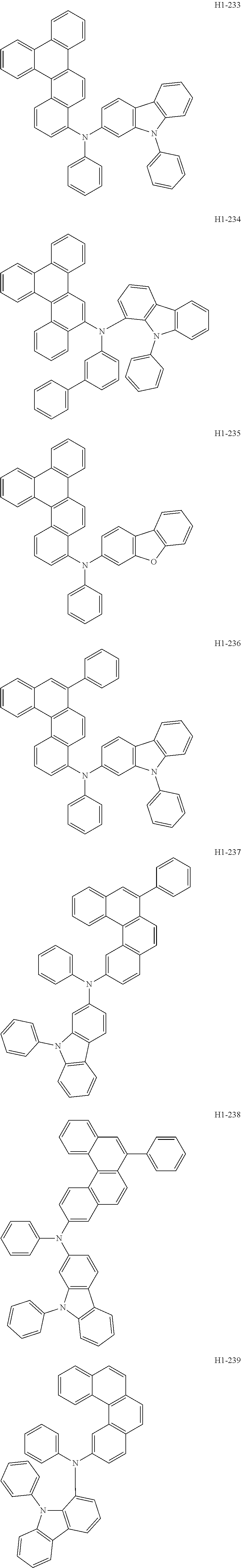

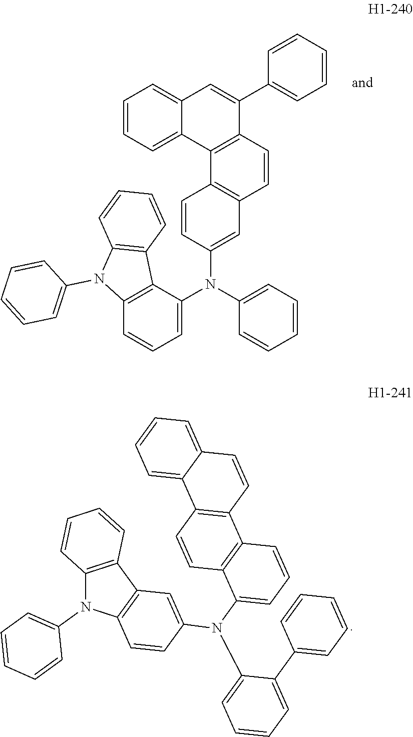 [SMILES: c1ccc(-c2cc3ccccc3c3c2ccc2cc(N(c4ccccc4)c4cccc5c4c4ccccc4n5-c4ccccc4)ccc23)cc1 ; c1ccc(-c2ccccc2N(c2ccc3c(c2)c2ccccc2n3-c2ccccc2)c2cccc3c2ccc2c4ccccc4ccc32)cc1]

10. The plurality of host materials according to claim 1, wherein the second host compound represented by the Formula 3 is selected from the following compounds:

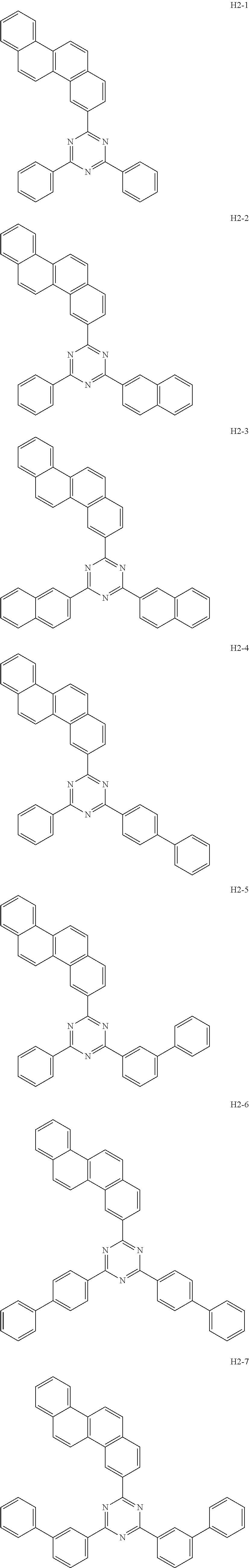

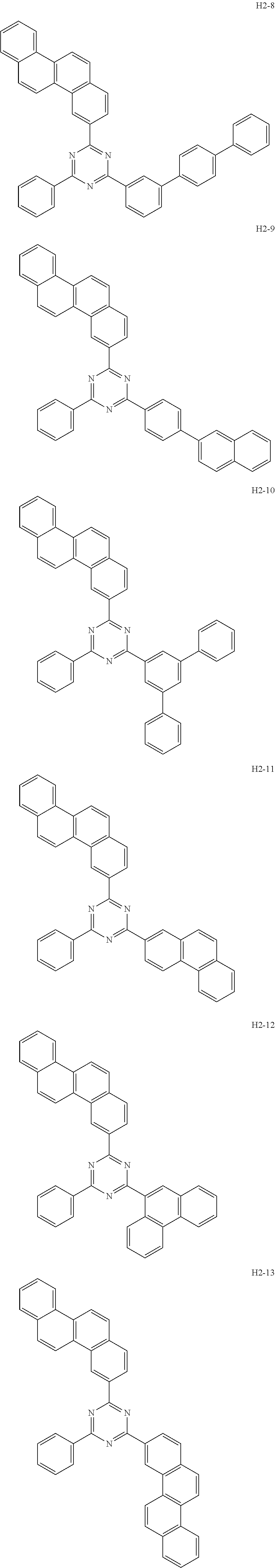

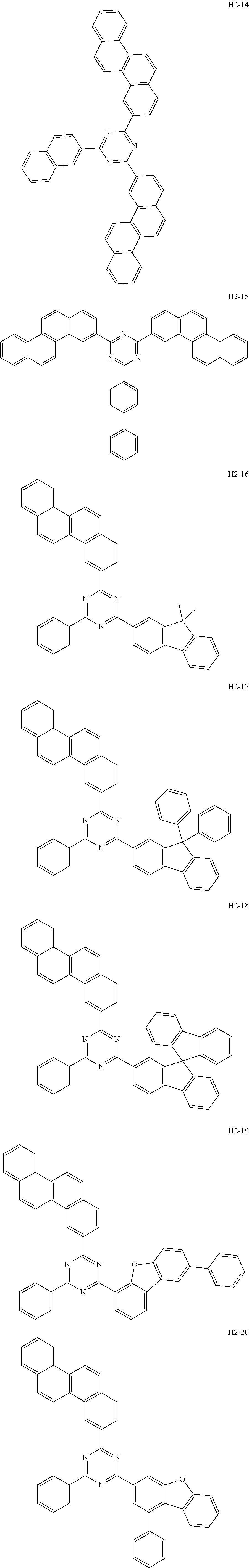

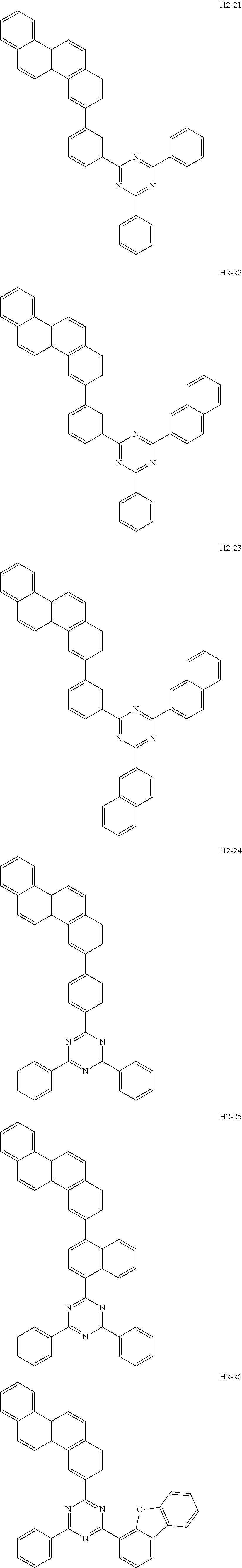

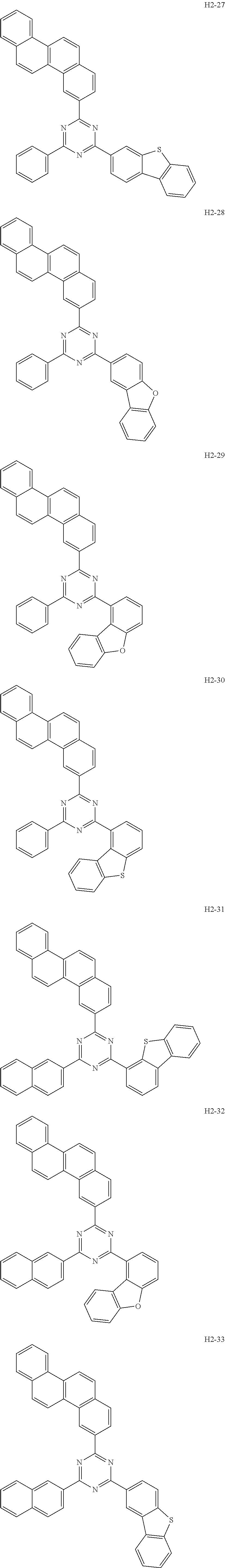

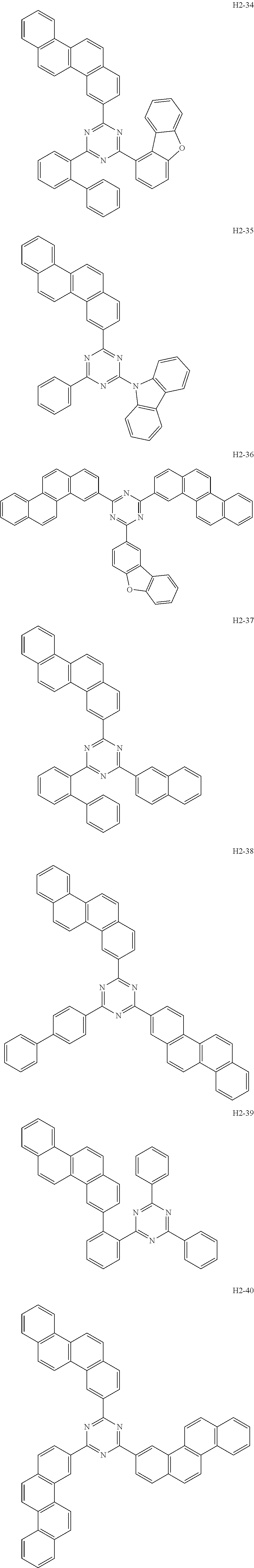

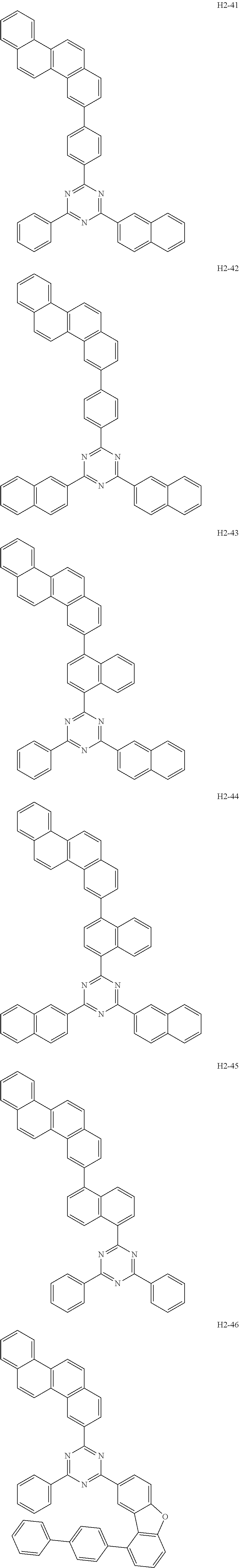

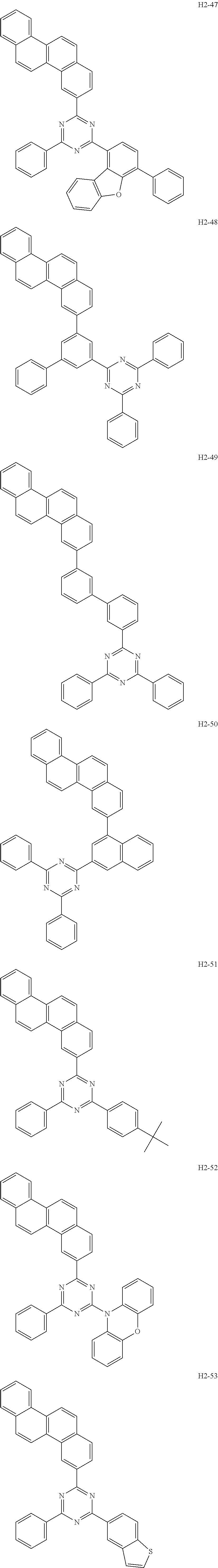

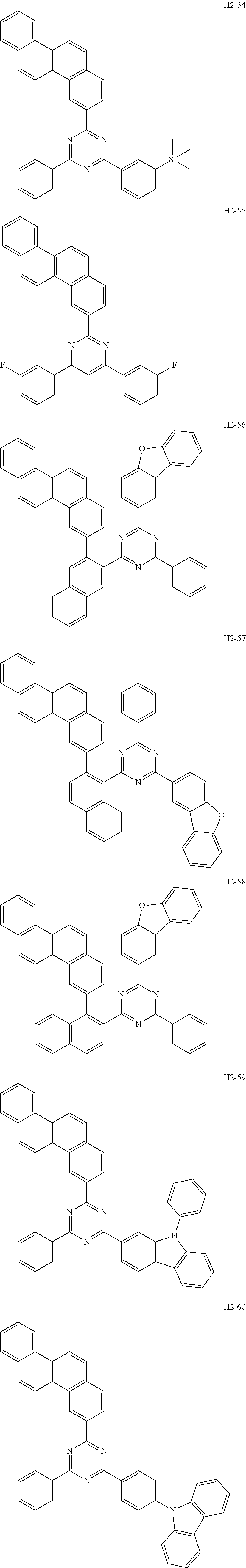

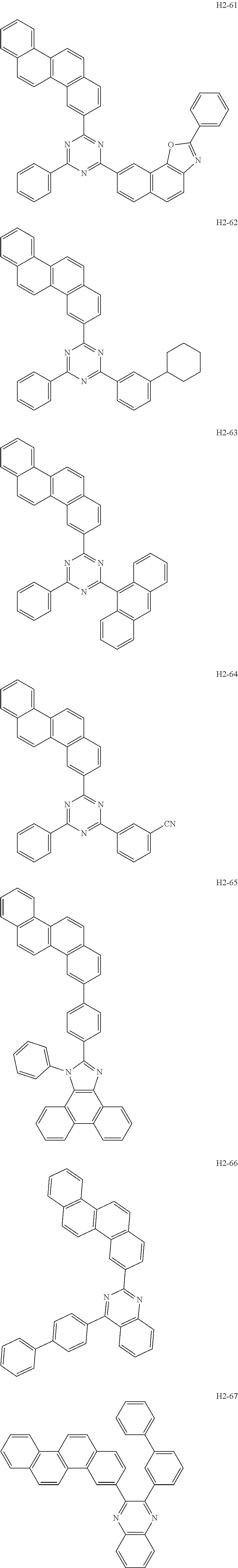

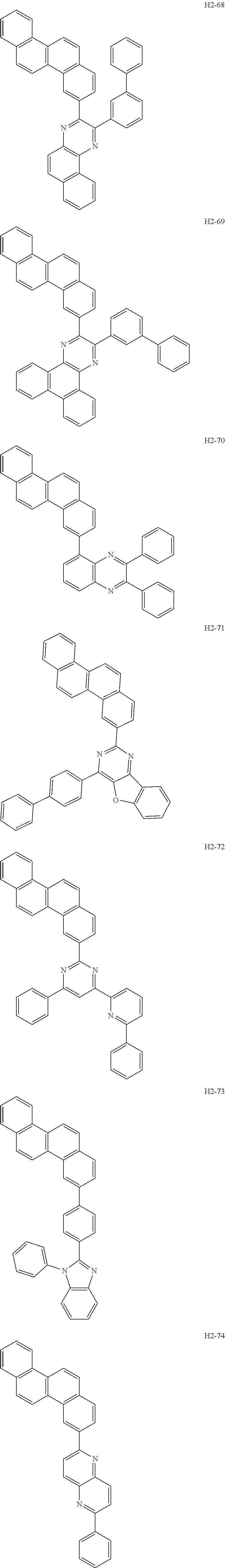

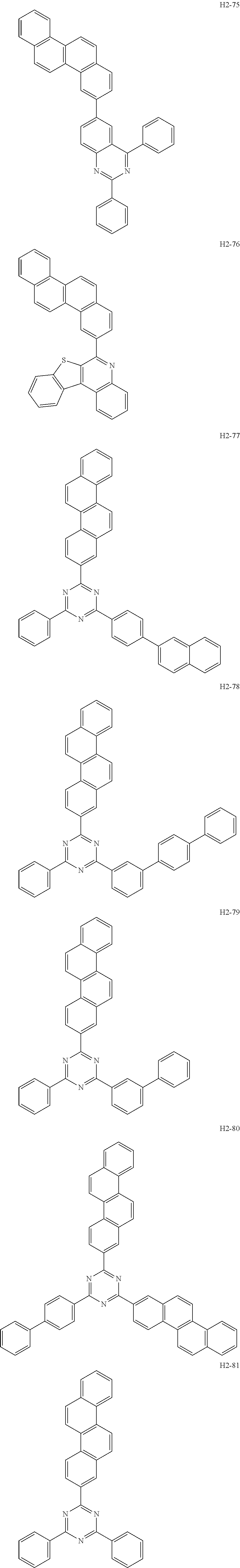

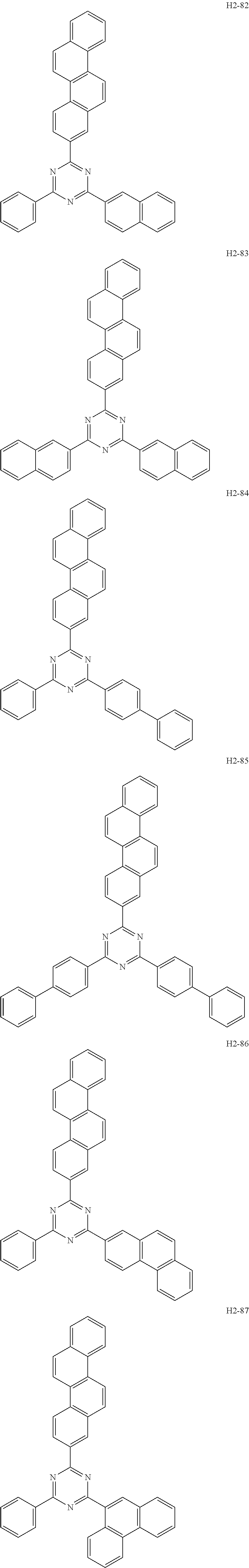

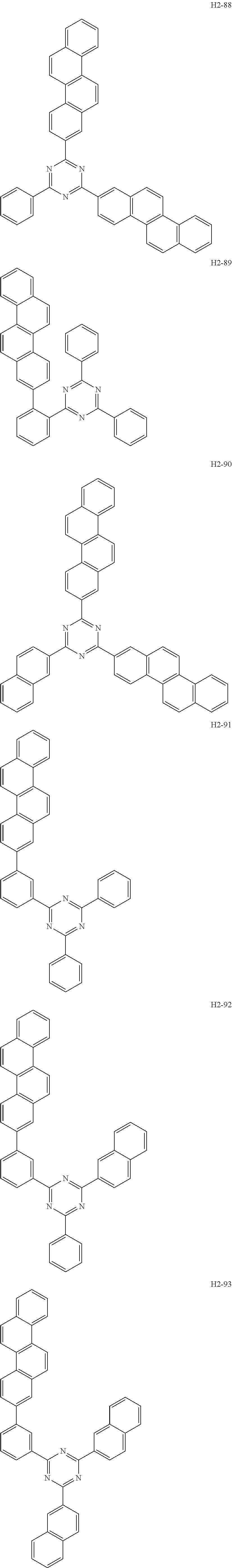

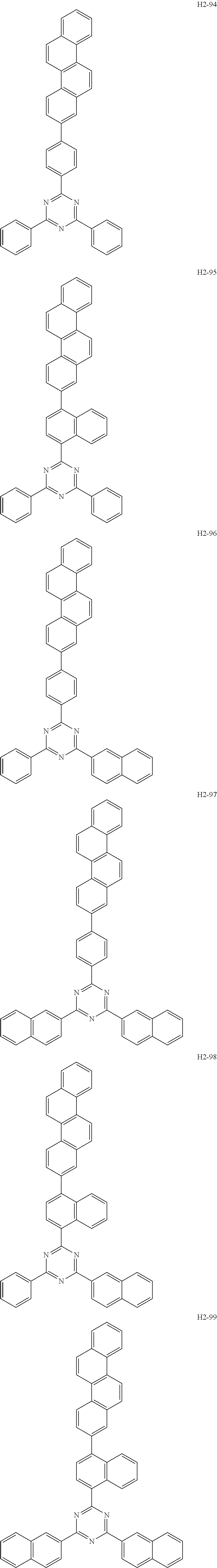

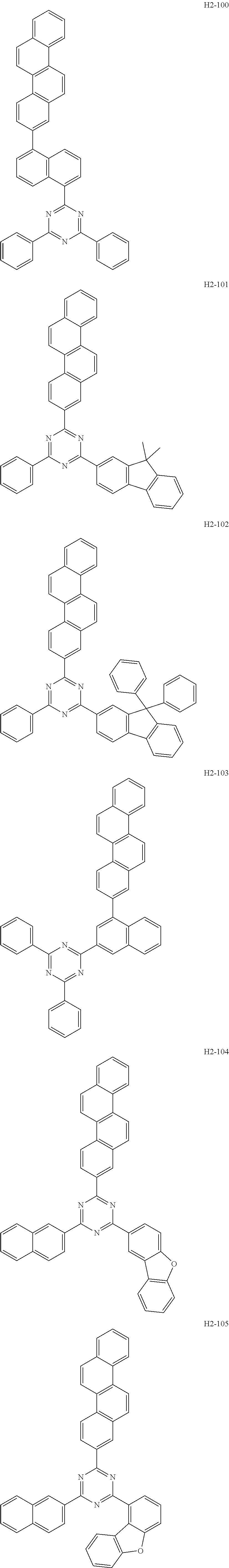 [SMILES: c1ccc(-c2nc(-c3ccccc3)nc(-c3cccc4c(-c5ccc6c(ccc7c8ccccc8ccc67)c5)cccc34)n2)cc1 ; CC1(C)c2ccccc2-c2ccc(-c3nc(-c4ccccc4)nc(-c4ccc5c(ccc6c7ccccc7ccc56)c4)n3)cc21 ; c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1 ; c1ccc(-c2nc(-c3ccccc3)nc(-c3cc(-c4ccc5c(ccc6c7ccccc7ccc56)c4)c4ccccc4c3)n2)cc1 ; c1ccc2cc(-c3nc(-c4ccc5c(ccc6c7ccccc7ccc56)c4)nc(-c4ccc5oc6ccccc6c5c4)n3)ccc2c1 ; c1ccc2cc(-c3nc(-c4ccc5c(ccc6c7ccccc7ccc56)c4)nc(-c4cccc5oc6ccccc6c45)n3)ccc2c1]

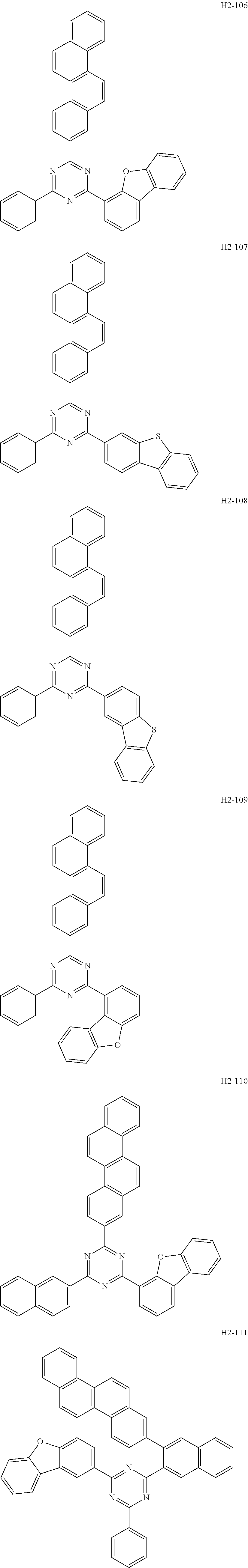 [SMILES: c1ccc(-c2nc(-c3ccc4c(ccc5c6ccccc6ccc45)c3)nc(-c3cccc4c3oc3ccccc34)n2)cc1 ; c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1 ; c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5c4oc4ccccc45)n3)ccc2c1 ; c1ccc(-c2nc(-c3ccc4oc5ccccc5c4c3)nc(-c3cc4ccccc4cc3-c3ccc4c(ccc5c6ccccc6ccc45)c3)n2)cc1]

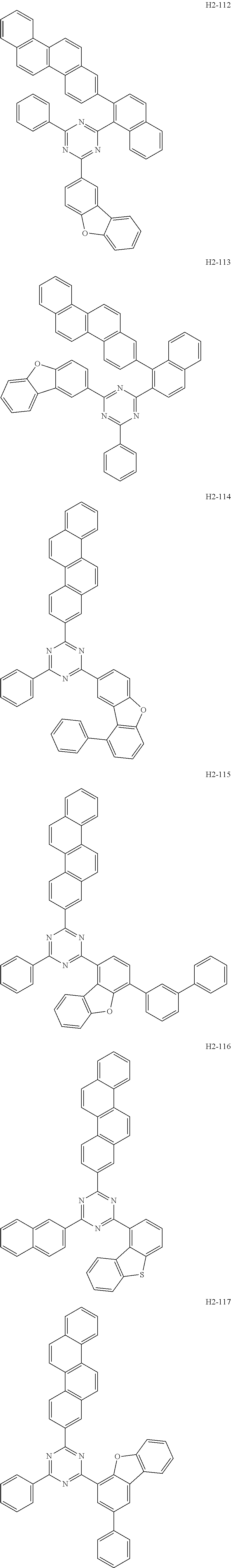

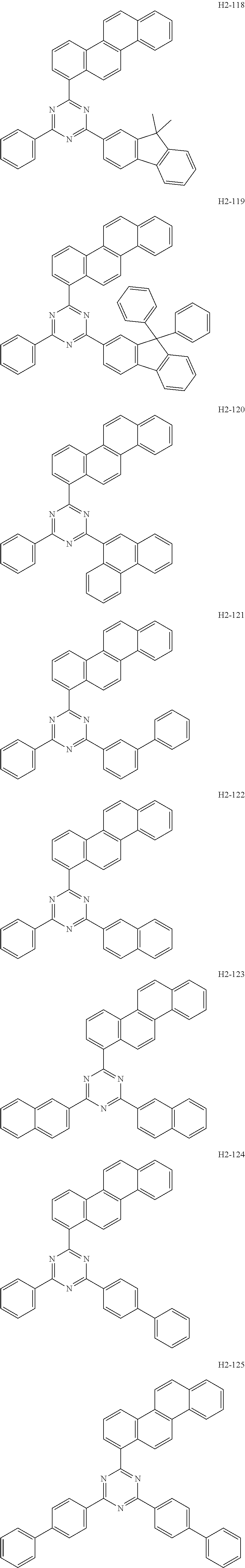

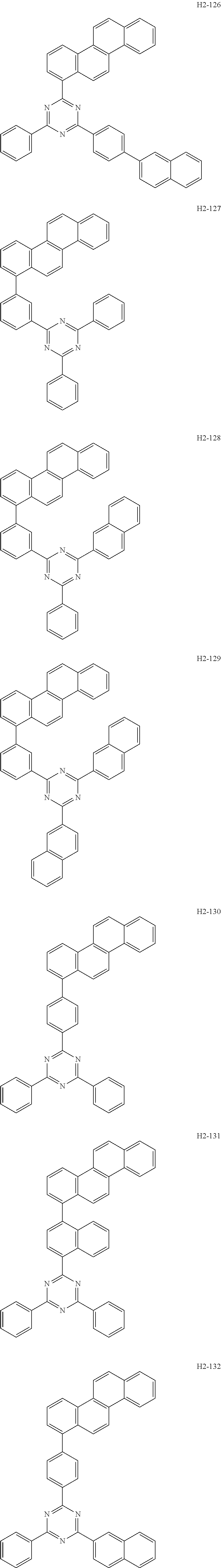

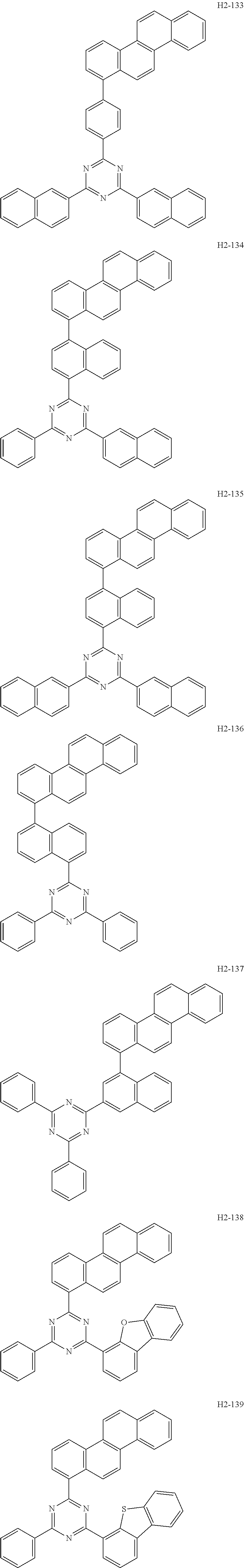

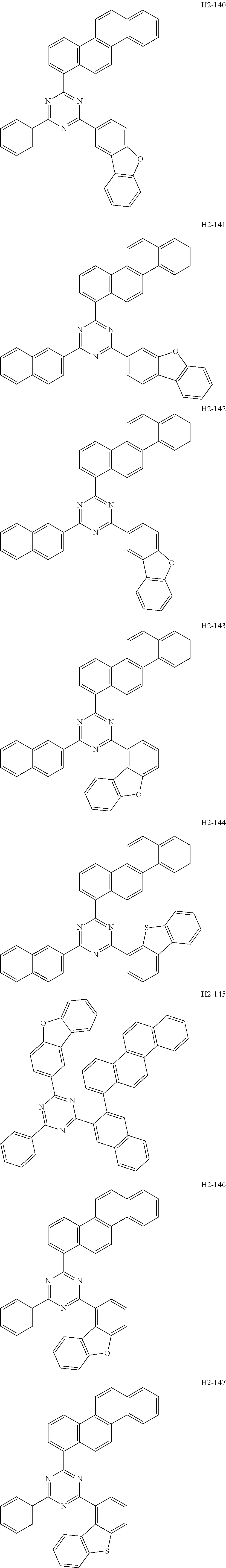

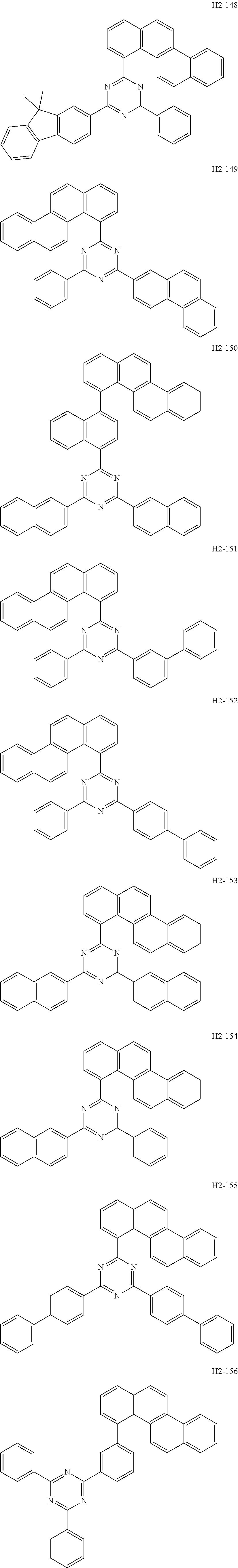

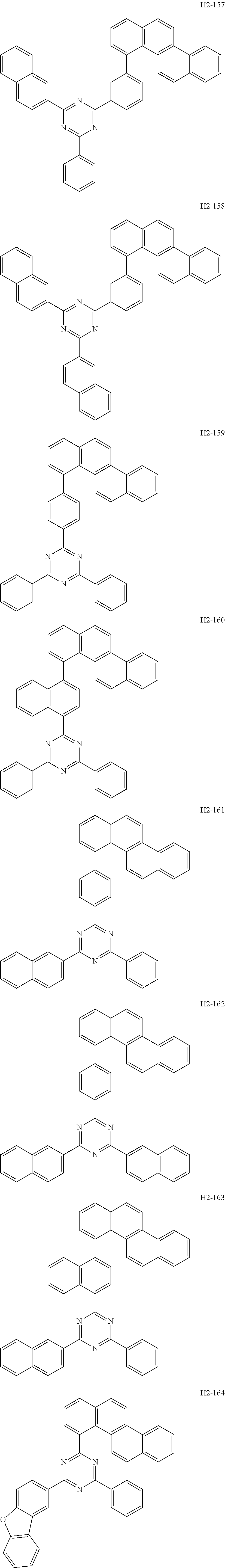

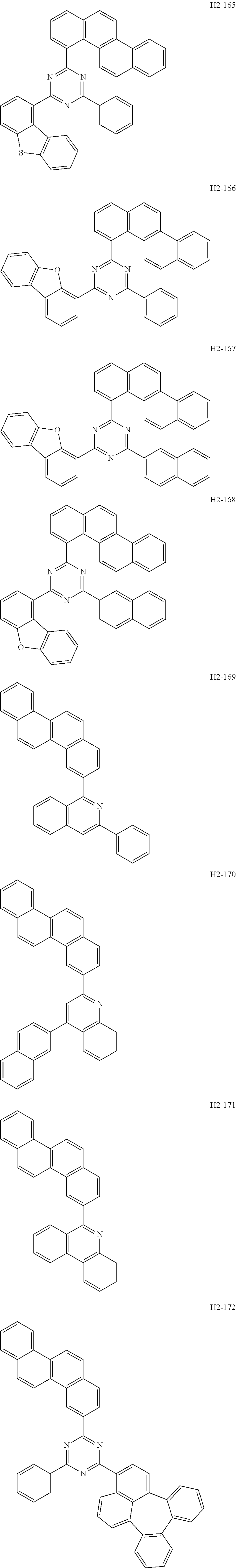

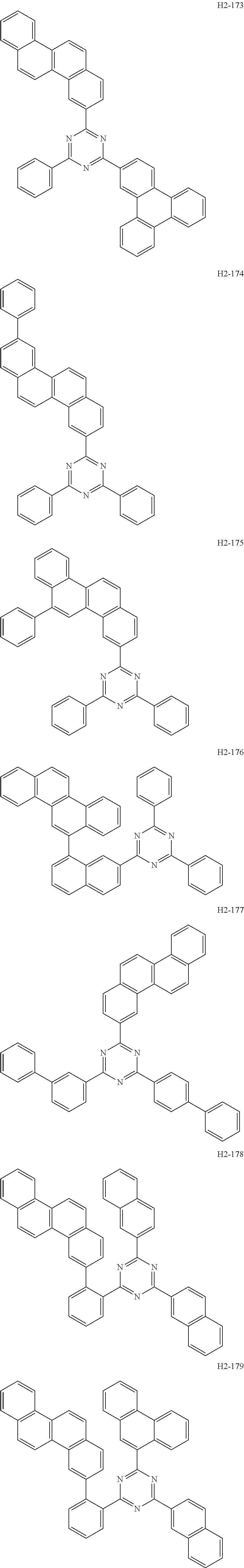

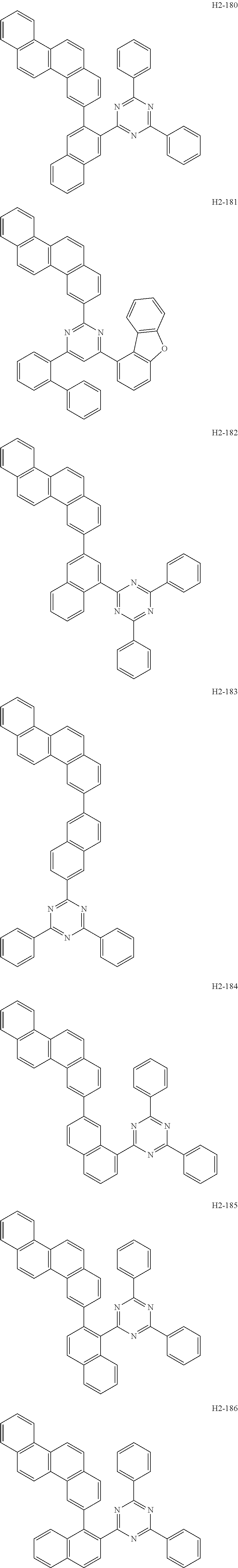

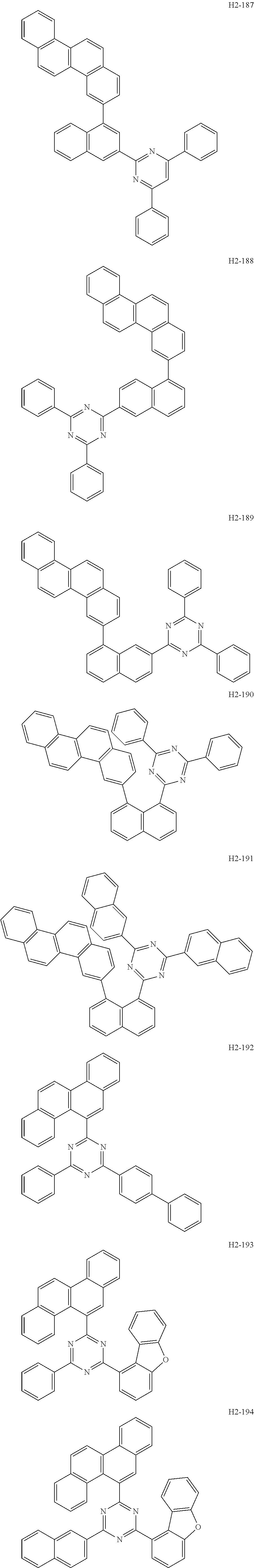

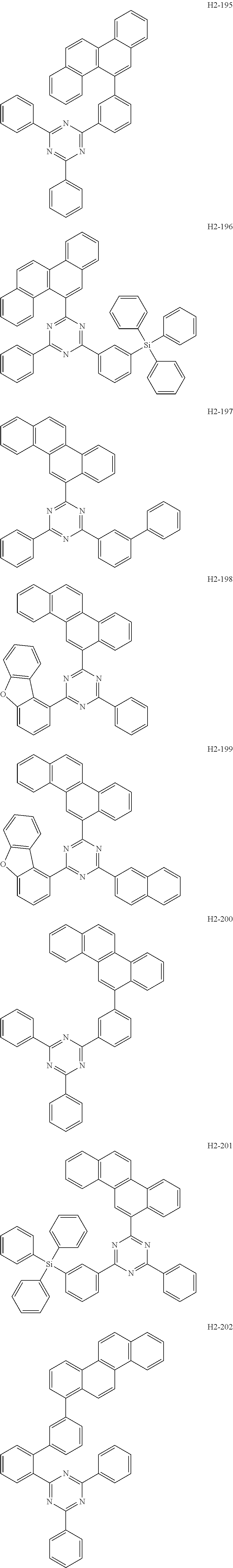

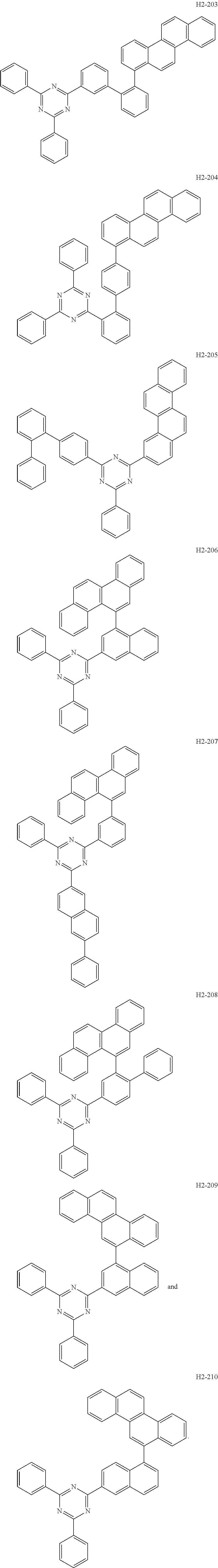

11. An organic electroluminescent device comprising: a first electrode; a second electrode; and at least one light-emitting layer(s) between the first electrode and the second electrode, wherein the at least one light-emitting layer(s) comprises the plurality of host materials according to claim 1.
